(19) United States
(12) Patent Application Publication
(10) Pub. No.: US 2004/0082585 A1
(43) Pub. Date: Apr. 29, 2004

(54) Prodrugs of substituted polycyclic compounds useful for selective inhibition of the coagulation cascade

(75) Inventors: Michael S. South, St. Louis, MO (US); Ronald K. Webber, St. Charles, MO (US); Horng-Chih Huang, Chesterfield, MO (US); Mihaly V. Toth, St. Louis, MO (US); Alan E. Moormann, Weldon Spring, MO (US); Jeffery S. Snyder, Manchester, MO (US); Jeffrey A. Scholten, Chesterfield, MO (US); Danny J. Garland, Ballwin, MO (US); Melvin L. Rueppel, St. Louis, MO (US); William L. Neumann, St. Louis, MO (US); Scott Long, Ballwin, MO (US); Wei Huang, Wildwood, MO (US); John Trujillo, St. Peters, MO (US); John J. Parlow, Arnold, MO (US); Darin E. Jones, Ballwin, MO (US); Brenda Case, St. Louis, MO (US); Michael J. Hayes, St. Louis, MO (US); Qingping Zeng, Thousand Oaks, CA (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/263,418

(22) Filed: Oct. 3, 2002

Related U.S. Application Data
(60) Provisional application No. 60/326,721, filed on Oct. 3, 2001.
(60) Provisional application No. 60/338,623, filed on Oct. 24, 2001.
(60) Provisional application No. 60/332,857, filed on Nov. 6, 2001.
(60) Provisional application No. 60/350,052, filed on Nov. 7, 2001.
(60) Provisional application No. 60/344,957, filed on Nov. 7, 2001.
(60) Provisional application No. 60/333,292, filed on Nov. 14, 2001.
(60) Provisional application No. 60/332,107, filed on Nov. 21, 2001.
(60) Provisional application No. 60/332,104, filed on Nov. 21, 2001.
(60) Provisional application No. 60/332,014, filed on Nov. 21, 2001.
(60) Provisional application No. 60/331,891, filed on Nov. 21, 2001.

(51) Int. Cl. A61K 31/497; A61K 31/513; C07D 213/70; C07C 50/12

(52) U.S. Cl. 514/252.1; 514/269; 514/460; 514/690; 544/298; 514/408; 546/290; 514/345; 549/273; 552/296

(57) ABSTRACT

The present invention relates to prodrug compounds, compositions and methods useful for preventing and treating thrombotic conditions in mammals. The prodrug compounds of the present invention selectively inhibit certain proteases of the coagulation cascade.

### CROSS REFERENCE TO RELATED APPLICATIONS

This application claims priority from Provisional Application Serial Nos. 60/326,721 filed Oct. 3, 2001, 60/338,623 filed Oct. 24, 2001, 60/332,857 filed Nov. 6, 2001, 60/350,052 and 60/344,957 both filed on Nov. 7, 2001, 60/333,292 filed on Nov. 14, 2001, 60/332,104, 60/332,014 and 60/331,891 all filed on Nov. 21, 2001, which are all hereby incorporated by reference in their entirety.

### FIELD OF THE INVENTION

The present invention relates to compounds, compositions and methods for preventing and treating thrombotic conditions such as coronary artery and cerebrovascular disease. More particularly, the invention relates to prodrugs of compounds that inhibit serine proteases of the coagulation cascade.

### BACKGROUND OF THE INVENTION

Hemorrhage, intravascular thrombosis, and embolism are common clinical manifestations of many diseases (see R. I. Handin in Harrison's Principles of Internal Medicine (J. D. Wilson, et al. eds., 12th ed. 1991) New York, McGraw-Hill Book Co., pp. 348-351). The normal hemostatic system limits blood loss by precisely regulated interactions between components of the vessel wall, circulating blood platelets, and plasma proteins. Unregulated activation of the of the hemostatic system, however, may cause thrombosis, which can reduce blood flow to critical organs like the brain and myocardium.

Physiological systems control the fluidity of blood in mammals (see P. W. Majerus, et al. in Goodman & Gilman's The Pharmacological Basis of Therapeutics (J. G. Hardman & L. E. Limbird, eds., 9th ed. 1996) New York, McGraw-Hill Book Co., pp. 1341-1343). Blood must remain fluid within the vascular systems and yet quickly be able to undergo hemostasis. Hemostasis, or clotting, begins when platelets first adhere to macromolecules in subendothelian regions of injured and/or damaged blood vessels. These platelets aggregate to form the primary hemostatic plug and stimulate local activation of plasma coagulation factors leading to generation of a fibrin clot that reinforces the aggregated platelets.

Plasma coagulation factors, also referred to as protease zymogens, include factors II, V, VII, VIII, IX, X, XI, and XII. These coagulation factors or protease zymogens are activated by serine proteases leading to coagulation in a so called “coagulation cascade” or chain reaction.

Coagulation or clotting occurs in two ways through different pathways. An intrinsic or contact pathway leads from XII to XIIa to XIa to IXa and to the conversion of X to Xa. Xa with factor Va converts prothrombin (II) to thrombin (IIa) leading to conversion of fibrinogen to fibrin. Polymerization of fibrin leads to a fibrin clot. An extrinsic pathway is initiated by the conversion of coagulation factor VII to VIIa by Xa. Factor VIIa, a plasma protease, is exposed to, and combines with its essential cofactor tissue factor (TF) which resides constitutively beneath the endothelium. The resulting factor VIIa/TF complex proteolytically activates its substrates, factors IX and X, triggering a cascade of reactions that leads to the generation of thrombin and a fibrin clot as described above.

While clotting as a result of an injury to a blood vessel is a critical physiological process for mammals, clotting can also lead to disease states. A pathological process called thrombosis results when platelet aggregation and/or a fibrin clot blocks (i.e., occludes) a blood vessel. Arterial thrombosis may result in ischemic necrosis of the tissue supplied by the artery. When the thrombosis occurs in a coronary artery, a myocardial infarction or heart attack can result. A thrombosis occurring in a vein may cause tissues drained by the vein to become edematous and inflamed. Thrombosis of a deep vein may be complicated by a pulmonary embolism. Preventing or treating clots in a blood vessel may be therapeutically useful by inhibiting formation of blood platelet aggregates, inhibiting formation of fibrin, inhibiting thrombus formation, inhibiting embolus formation, and for treating or preventing unstable angina, refractory angina, myocardial infarction, transient ischemic attacks, atrial fibrillation, thrombotic stroke, embolic stroke, deep vein thrombosis, disseminated intravascular coagulation, ocular build up of fibrin, and reocclusion or restenosis of recanalized vessels.

In order to treat such conditions, researchers have sought to discover chemical compounds that efficaciously and selectively control the clotting process. In addition, such compounds may provide a better understanding of the pathways involved in the coagulation process.

Thus far, the compounds that have been discovered often possess a polar or basic functional group which is integrally responsible for the desired biological activity. Frequently, this polar functional group is a nitrogen atom of, for example, a guanidine, alkyl-amidine or aryl-amidine group. Because these functionalities are highly basic, they remain protonated at physiologically relevant pH's. The ionic nature of such protonated species hinders their permeability across lipophilic membranes, which can reduce bioavailability when the pharmaceutical agent is administered orally.

In order to circumvent such a problem, it is often advantageous to perform a derivatization or chemical modification of the polar functionality such that the pharmaceutical agent becomes neutrally charged and more lipophilic, thereby facilitating absorption of the drug. However, for the derivatization to be useful, the derivatization must be bioconvertable at the target site or sites of desired pharmacological activity and cleaved under normal physiological conditions to yield the biologically active drug. The term “prodrug” has been used to denote such a chemically modified intermediate.

### SUMMARY OF THE INVENTION

Among the several aspects of the present invention, therefore, is the provision of prodrug compounds useful for selective inhibition of certain enzymes that act upon the coagulation cascade thereby preventing and treating thrombotic conditions in mammals. Generally speaking, these prodrug compounds undergo hydrolysis, oxidation, reduction or elimination at a derivatized amidine group to yield the active compound.

Briefly, therefore, the present invention is directed to the prodrug compound, per se, to pharmaceutical compositions comprising the prodrug compound and a pharmaceutically acceptable carrier, and to methods of use.

One aspect of the invention provides compounds that correspond to formula (I):

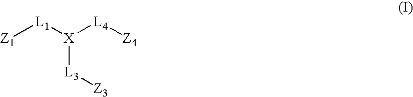

wherein

X comprises a 5- or 6-membered heterocyclic or aromatic ring, the ring atoms being X₁, X₂, X₃, X₄, and X₅ for 5-membered heterocyclic rings and X₁, X₂, X₃, X₄, X₅ and X₆ for 6-membered heterocyclic or aromatic rings, wherein X₂ is alpha to each of X₁ and X₃, X₃ is alpha to each of X₂ and X₄, X₄ is alpha to each of X₃ and X₅, X₅ is alpha to X₄ and alpha to X₁ if X is a 5-membered ring or to X₆ if X is a 6-membered ring, and X6, when present, is alpha to each of X₁ and X₅, wherein X₁, X₂, X₃, X₄, X₅ and X₆ are carbon, nitrogen, oxygen or sulfur;

L₁, L₃ and L₄ are linkages through which Z₁, Z₃, and Z₄, respectively, are covalently bonded to different ring atoms of the 5- or 6-membered heterocyclic or aromatic ring of X, wherein Z₁ is covalently bonded to X₁, Z₃ is covalently bonded to X₃, and Z₄ is covalently bonded to X₄, each of L₁, L₃ and L₄ independently being a covalent bond or comprising one or more atoms through which Z₁, Z₃, and Z₄ are covalently bonded to X₁, X₃ and X₄, respectively;

Z₁ is hydrocarbyl or substituted hydrocarbyl;

Z₃ comprises a 5- or 6-membered heterocyclic or aromatic ring substituted with a derivatized amidine which, upon hydrolysis, oxidation, reduction or elimination yields an amidine group, and optionally further substituted with a halogen or hydroxy, the ring atoms of the 5- or 6-membered heterocyclic or aromatic ring of Z₃ being carbon, sulfur, nitrogen, or oxygen;

Z₄ comprises a 5- or 6-membered heterocyclic or carbocyclic ring having two substituents, R₄₂ and R₄₄, and two ring atoms each of which is in the beta position relative to the ring atom of Z₄ through which Z₄ is covalently bonded to X, wherein one of R₄₂ and R₄₄ is covalently bonded to one of said beta positions and the other of R₄₂ and R₄₄ is covalently bonded to the other of said beta positions, the ring atoms of the 5- or 6-membered heterocyclic or carbocyclic ring of Z₄ being carbon, nitrogen, oxygen, or sulfur;

R₄₂ is amino; and

R₄₄ is selected from the group consisting of hydrogen, hydrocarbyl, substituted hydrocarbyl, heterocyclo, halogen, or an optionally substituted heteroatom selected from nitrogen, oxygen, sulfur and phosphorus; provided, however, the derivatized amidine is other than amidine derivatized with t-butoxycarbonyl.

Another aspect of the invention provides compounds corresponding to formula II:

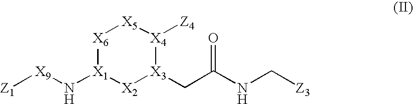

wherein:

each of X₁, X₂, X₃, X₄, X₅ and X₆ is carbon or nitrogen;

X₂ is a hydrogen bond acceptor;

X₉ is a direct bond or —(CH₂)ₘ— where m is 1 to 5;

R₄₂ and R₄₄ are as defined for formula (I); and

Z₁, Z₃, and Z₄ are as defined for formula (I).

Other aspects and features of this invention will be in part apparent and in part pointed out hereafter.

Abbreviations and Definitions

The term “elimination” is generally meant to encompass any one or more of the following reactions: (1) a reaction that results in a compound fragmenting into two or more compounds; and (2) a reaction that results in one or more groups being removed from a compound without being replaced by other groups.

The term “oxidation” is generally meant to encompass any one or more of the following reactions: (1) a reaction that results in an increase in the oxidation number of an atom in a compound, whether the atom is uncharged or charged and whether free or covalently bound; (2) a reaction that results in the loss of hydrogen from a compound; (3) a reaction that results in the loss or removal of one or more electrons from a compound, with or without concomitant loss or removal of a proton or protons; (4) the action or process of reacting a compound with oxygen; and (5) a reaction that results in the addition of one or more oxygen atoms to a compound.

The term “reduction” is generally meant to encompass any one or more of the following reactions: (1) any reaction which results in a decrease in the oxidation number of an atom in a compound; and (2) any reaction that results in oxygen being withdrawn from, hydrogen being added to, or an electron being added to (with or without the addition of a proton) a compound.

The term “hydrolysis” is generally meant to encompass any one or more of the following reactions: (1) any reaction which results in the addition of a nucleophile to a compound to form a new bond with concurrent loss of a group from the compound; (2) any reaction which results in the addition of water to a compound; and (3) any reaction that results in the rupture of one or more chemical bonds by reaction with, and involving the addition of, the elements of water.

The term “physiological conditions” are those conditions characteristic to an organism's (to a human beings) healthy or normal functioning.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The “substituted hydrocarbyl” moieties described herein are hydrocarbyl moieties which are substituted with at least one atom other than carbon, including moieties in which a carbon chain atom is substituted with a heteroatom such as nitrogen, oxygen, silicon, phosphorus, boron, sulfur, or a halogen atom. Exemplary substituted hydrocarbyl moieties include, heterocyclo, alkoxyalkyl, alkenyloxyalkyl, alkynyloxyalkyl, aryloxyalkyl, hydroxyalkyl, protected hydroxyalkyl, keto, acyl, nitroalkyl, aminoalkyl, cyano, alkylthioalkyl, arylthioalkyl, ketals, acetals, amides, acids, esters and the like.

The term “heteroatom” shall mean atoms other than carbon and hydrogen.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The terms “halogen” or “halo” as used herein alone or as part of another group refer to chlorine, bromine, fluorine, and iodine.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The term “heteroaromatic” as used herein alone or as part of another group denote optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The term “acyl,” as used herein alone or as part of another group, denotes the moiety formed by removal of the hydroxyl group from the group —COOH of an organic carboxylic acid, e.g., RC(O)—, wherein R is hydrogen, R¹, R¹O—, R¹R²N—, or R¹S—, R² is hydrocarbyl, heterosubstituted hydrocarbyl, or heterocyclo, and R² is hydrogen, hydrocarbyl or substituted hydrocarbyl.

The term “acyloxy,” as used herein alone or as part of another group, denotes an acyl group as described above bonded through an oxygen linkage (—O—), e.g., RC(O)O— wherein R is as defined in connection with the term “acyl.”

The term “acetamidyl” as used herein describes a chemical moiety represented by the formula NR₁C(O)R₂.

The term “carboxamido” as used herein, describes a chemical moiety represented by the formula C(O)NR₁R₂.

The term “alkoxycarbonyl” as used herein describes a chemical moiety represented by the formula C(O)OR.

The term “sulfonamido” as used herein describes a chemical moiety represented by the formula SO₂NR₁R₂.

The term “alkylsulfonyl” as used herein describes a chemical moiety represented by the formula SO₂R.

The term “sulfonamidyl” as used herein describes a chemical moiety represented by the formula NRSO₂R.

As described herein for the terms “acetamidyl”, “carboxamido”, “alkocycarbonyl”, “sulfonamido”, “alkylsulfonyl”, and “sulfonamidyl”, R, R₁ and R₂ are independently hydrogen, alkyl, aryl, and arylakyl, optionally substituted with halogen, hydroxy or alkoxy.

### DESCRIPTION OF THE PREFERRED EMBODIMENT

One aspect of the invention embraces compounds that correspond to formula (I)

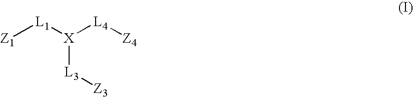

wherein:

X comprises a 5- or 6-membered heterocyclic or aromatic ring, the ring atoms being X₁, X₂, X₃, X₄, and X₅ for 5-membered heterocyclic rings and X₁, X₂, X₃, X₄, X₅ and X₆ for 6-membered heterocyclic or aromatic rings, wherein X₂ is alpha to each of X₁ and X₃, X₃ is alpha to each of X₂ and X₄, X₄ is alpha to each of X₃ and X₅, X₅ is alpha to X₄ and alpha to X₁ if X is a 5-membered ring or to X₆ if X is a 6-membered ring, and X₆, when present, is alpha to each of X₁ and X₅. wherein X₁, X₂, X₃, X₄, X₅ and X₆ are carbon, nitrogen, oxygen or sulfur;

L₁, L₃ and L₄ are linkages through which Z₁, Z₃, and Z₄, respectively, are covalently bonded to different ring atoms of the 5- or 6-membered heterocyclic or aromatic ring of X, wherein Z, is covalently bonded to X₁, Z₃ is covalently bonded to X₃, and Z₄ is covalently bonded to X₄, each of L₁, L₃ and L₄ independently being a covalent bond or comprising one or more atoms through which Z₁, Z₃, and Z₄ are covalently bonded to X₁, X₃ and X₄, respectively;

Z₁ is hydrocarbyl or substituted hydrocarbyl;

Z₃ comprises a 5- or 6-membered heterocyclic or aromatic ring substituted with a derivatized amidine which, upon hydrolysis, oxidation, reduction or elimination yields an amidine group, and optionally further substituted with a halogen or hydroxy, the ring atoms of the 5- or 6-membered heterocyclic or aromatic ring of Z₃ being carbon, sulfur, nitrogen, or oxygen;

Z₄ comprises a 5- or 6-membered heterocyclic or carbocyclic ring having two substituents, R₄₂ and R₄₄, and two ring atoms each of which is in the beta position relative to the ring atom of Z₄ through which Z₄ is covalently bonded to X, wherein one of R₄₂ and R₄₄ is covalently bonded to one of said beta positions and the other of R₄₂ and R₄₄ is covalently bonded to the other of said beta positions, the ring atoms of the 5- or 6-membered heterocyclic or carbocyclic ring of Z₄ being carbon, nitrogen, oxygen, or sulfur;

R₄₂ is amino; and

R₄₄ is selected from the group consisting of hydrogen, hydrocarbyl, substituted hydrocarbyl, heterocyclo, halogen, or a substituted or unsubstituted heteroatom selected from nitrogen, oxygen, sulfur and phosphorus; provided, however, the derivatized amidine is other than amidine derivatized with t-butoxycarbonyl.

In another embodiment for compounds having formula (I), each of X₁, X₂, X₃, X₄, X₅ and X₆ is carbon or nitrogen;

X₂ is a hydrogen bond acceptor;

L₁ is —X₉NH— wherein X₉ is covalently bonded directly to Z₁ and X₉ is a direct bond or —(CH₂)ₘ— wherein m is 1 to 5;

L₃ is a glycine derivative;

L₄ is a direct bond;

Z₁ is selected from the group consisting of C₁-C₈ alkyl, C₂-C₈ alkenyl, and C₂-C₈ alkynyl, the alkyl, alkenyl, or alkynyl being optionally substituted at any substitutable position with a halogen;

Z₃ comprises a phenyl, furanyl or thienyl ring, the phenyl, furanyl or thienyl ring being substituted with a derivatized amidine which, upon hydrolysis, oxidation, reduction or elimination yields an amidine group, and optionally further substituted with fluorine or hydroxy;

Z₄ comprises a phenyl or thienyl ring having two substituents, R₄₂ and R₄₄, and two ring atoms each of which is in the beta position relative to the ring atom of Z₄ through which Z₄ is covalently bonded to X, wherein one of R₄₂ and R₄₄ is covalently bonded to one of said beta positions and the other of R₄₂ and R₄₄ is covalently bonded to the other of said beta positions;

R₄₂ is amino; and

R₄₄ is selected from the group consisting of hydrocarbyl, substituted hydrocarbyl, heterocyclo, halogen, and an optionally substituted heteroatom selected from nitrogen, oxygen, sulfur and phosphorus.

In one embodiment for compounds having formula (I), the L₁ linkage is —X₉NH— where X₉ is covalently bonded directly to Z₁ and is a direct bond or an alkylene chain having the formula (CH₂)ₘ wherein m is 0 to 5. In an alternative embodiment, m is 0 to 2. In another embodiment, the L₁ linkage is a bond.

In another embodiment for compounds having formula (I), the L₃ linkage is a glycine derivative, an alanine derivative, an amino derivative, or a sulfonyl derivative. In an alternative embodiment, the L₃ linkage is a glycine derivative. In still another embodiment, the L₃ linkage is —CH₂CONHCH₂— where Z₃ is covalently bonded to the methylene bonded to the amine nitrogen of L₃.

In a further embodiment for compounds having formula (I), the L₄ linkage is a direct bond, methylene, ethylene or an optionally substituted heteroatom selected from the group nitrogen, oxygen, sulfur or phosphorus. In another embodiment, the L₄ linkage is a direct bond.

In yet another embodiment for compounds having formula (I), Z, is a C₁-C₅ alkyl optionally substituted at any substitutable position with fluorine, hydroxy, carboxy or alkoxycarbonyl. Generally speaking, the C₁-C₅ alkyl is a cyclopropyl, isopropyl, methyl, ethyl, cyclobutyl, isobutyl, tert-butyl and sec-butyl optionally substituted at any substitutable position with fluorine, hydroxy, carboxy, or alkoxycarbonyl. In another embodiment, the C₁-C₅ alkyl is isopropyl or cyclobutyl optionally substituted at any substitutable position with fluorine, hydroxy, carboxy, or alkoxycarbonyl.

A further embodiment embraces compounds having formula (I) where Z₃ is —R₃₀₀C(═NR₃₀₁)NR₃₀₂R₃₀₃, wherein R₃₀₀ is a 6-membered carbocyclic aromatic ring, R₃₀₁, R₃₀₂, R₃₀₃ are independently selected from the group consisting of hydrogen, halogen, optionally substituted hydrocarbyl, and an optionally substituted heteroatom selected from the group consisting of oxygen, nitrogen, phosphorus and sulfur, provided at least one of R₃₀₁, R₃₀₂, R₃₀₃ is other than hydrogen. In yet another embodiment, Z₃ is —R₃₀₀C(═NR₃₀₁)NR₃₀₂R₃₀₃, wherein R₃₀₀, is a 6-membered carbocyclic aromatic ring, and at least two of R₃₀₁, R₃₀₂, R₃₀₃ are ring atoms of a heterocyclic ring. In an alternative embodiment, Z₃ is —R₃₀₀C(═NR₃₀₁)NR₃₀₂R₃₀₃, R₃₀₀ is a 6-membered carbocyclic aromatic ring, and at least one of R₃₀₁, R₃₀₂, R₃₀₃ are ring atoms of a heterocyclic ring fused to R₃₀₀.

Yet another embodiment encompasses compounds having formula (I) where Z₃ is a benzamidine derivatized with one or more groups selected from carbonyl, thiocarbonyl, imino, enamino, phosphorus, and sulfur, where the benzamidine derivative hydrolyzes under physiological conditions to form benzamidine. In a further embodiment, Z₃ is a benzamidine derivatized with one or more groups selected from optionally substituted hydrocarbyl, provided that the carbon atom directly bonded to the amidine is sp³ hybridized and aryl, where the benzamidine derivative is oxidized under physiological conditions to form benzamidine. In yet another embodiment, Z₃ is a benzamidine derivatized with one or more heteroatoms selected from oxygen, nitrogen in its most reduced state, and sulfur in its most reduced state, where the benzamidine derivative is reduced under physiological conditions to form benzamidine. In still another embodiment, Z₃ is a benzamidine derivatized with one or more substituents selected from a hydrocarbyl substituted at the beta carbon with carbonyl, sulfonyl, sulfinyl, cyano, nitro and an alkyl, aryl, or heterocyclic group substituted with oxygen, nitrogen, or sulfur at the carbon directly bonded to the amidine group, where the benzamidine derivative undergoes elimination at physiological conditions to form benzamidine.

In a further embodiment for compounds having formula (I), Z₃ corresponds to formula (a)

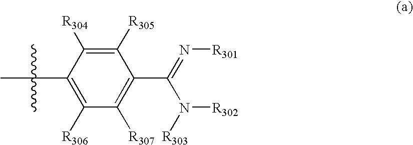

wherein:

R₃₀₁, R₃₀₂, and R₃₀₃ are independently selected from the group consisting of:

(i) hydrogen, —C(═O)Rₐ, —C(═O)ORₐ, —S(═O)ORₐ, —S(═O)SRₐ, —S(═O)₂ORₐ, —S(═O)₂SRₐ and alkenyl, wherein Rₐ is selected from the group consisting of hydrocarbyl, substituted hydrocarbyl, and heterocyclo, provided, however, that the carbon atom of R₃₀₁, R₃₀₂, and R₃₀₃ directly bonded to the amidine is sp² hybridized when R³⁰¹, R³⁰², and R³⁰³ is alkenyl,

(ii) hydrogen, optionally substituted hydrocarbyl and aryl, provided, however, the carbon atom of R³⁰¹, R³⁰², and R³⁰³ directly bonded to the amidine is sp³ hybridized when R³⁰¹, R³⁰², and R³⁰³ is optionally substituted hydrocarbyl,

(iii) hydrogen, —OR_{b}, —SR_{b}, —NR_{b}, or —N(R_{b})₂, wherein each R_{b} is independently optionally substituted hydrocarbyl, and heterocyclo, and

(iv) hydrogen, substituted hydrocarbyl wherein the carbon bonded to the amidine group is substituted with —OR_{c}, —SR_{c}, —NR_{c}, or —N(R_{c})₂, wherein each R_{c} is independently —C(O)R_{d}, —C(O)NR_{d}, —C(O)OR_{d}, —C(O)N(R_{d})₂ and each R_{d} is independently hydrocarbyl, substituted hydrocarbyl or heterocyclo, and substituted alkyl with the carbon atom beta to the point of attachment to the amidine group being an unsaturated electron withdrawing group, provided, however, at least one of R₃₀₁, R₃₀₂, and R₃₀₃ is other than hydrogen;

R₃₀₄ is selected from the group consisting of halogen, hydrogen, hydroxyl, alkyl, sulfhydryl, alkoxy, and alkylthio;

R₃₀₅ is selected from the group consisting of oxygen, sulfur, halogen, hydrogen, hydroxyl, alkyl, sulfhydryl, alkoxy, and alkylthio;

R₃₀₆ is selected from the group consisting of halogen, hydrogen, hydroxyl, alkyl, sulfhydryl, alkoxy, and alkylthio; and

R₃₀₇ is selected from the group consisting of oxygen, sulfur, halogen, hydrogen, hydroxyl, alkyl, sulfhydryl, alkoxy, and alkylthio.

In one embodiment, the benzamidine derivative is hydrolyzed under physiological conditions to form benzamidine when Z₃ is a benzamidine derivative having formula (a) and R₃₀₁, R₃₀₂, and R₃₀₃ are independently selected from hydrogen, —C(═O)Rₐ, —C(═O)ORₐ, —S(═O)ORₐ, —S(═O)SRₐ, —S(═O)₂ORₐ, —S(═O)₂SRₐ and alkenyl, wherein Rₐ is selected from the group consisting of hydrocarbyl, substituted hydrocarbyl, and heterocyclo, provided, however, that the carbon atom of R₃₀₁, R₃₀₂, and R₃₀₃ directly bonded to the amidine is sp² hybridized when R₃₀₁, R₃₀₂, and R₃₀₃ is alkenyl.

In a further embodiment, the benzamidine derivative is oxidized under physiological conditions to form benzamidine when Z₃ is a benzamidine derivative having formula (a) and R₃₀₁, R₃₀₂, and R₃₀₃ are independently selected from hydrogen, optionally substituted hydrocarbyl and aryl, provided, however, the carbon atom of R₃₀₁, R₃₀₂, and R₃₀₃ directly bonded to the amidine is sp³ hybridized when R₃₀₁, R₃₀₂, and R₃₀₃ is optionally substituted hydrocarbyl.

In still another embodiment, the benzamidine derivative is reduced under physiological conditions to form benzamidine when Z₃ is a benzamidine derivative having formula (a) and R₃₀₁, R₃₀₂, and R₃₀₃ are independently selected from hydrogen, —OR_{b}, —SR_{b}, —NR_{b}, or —N(R_{b})₂, wherein each R_{b} is independently optionally substituted hydrocarbyl, and heterocyclo.

In an alternative embodiment, the benzamidine derivative undergoes elimination at physiological conditions to form benzamidine when Z₃ is a benzamidine derivative having formula (a) and R₃₀₁, R₃₀₂, and R₃₀₃ are independently selected from hydrogen, substituted hydrocarbyl wherein the carbon bonded to the amidine group is substituted with —OR_{c}, —SR_{c}, —NR_{c}, or —N(R_{c})₂, wherein each R_{c} is independently —C(O)R_{d}, —C(O)NR_{d}, —C(O)OR_{d}, —C(O)N(R_{d})₂ and each R_{d} is independently hydrocarbyl, substituted hydrocarbyl or heterocyclo, and substituted alkyl with the carbon atom beta to the point of attachment to the amidine group being an unsaturated electron withdrawing group.

In a further embodiment for compounds having formula (I), Z₃ may be any of the benzamidine derivatives illustrated in Table 1 or 3 below.

A further embodiment embraces compounds having formula (I) where Z₄ is a substituted, 6-membered carbocyclic aromatic ring. In an alternative of this embodiment, Z₄ corresponds to formula (b)

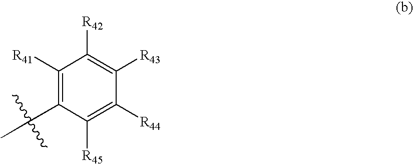

wherein:

R₄₂ is amino;

R₄₄ is selected from the group consisting of hydrocarbyl, substituted hydrocarbyl, halogen and an optionally substituted heteroatom selected from the group consisting of oxygen, nitrogen, and sulfur; and

R₄₁, R₄₃ and R₄₅ are independently selected from the group consisting of hydrogen, halogen, hydrocarbyl, substituted hydrocarbyl, and an optionally substituted heteroatom selected from the group consisting of oxygen, nitrogen, and sulfur.

In another embodiment of compounds wherein Z₄ corresponds to formula (b) and R₄₂ is amino, R₄₄ is selected from hydrocarbyl, substituted hydrocarbyl, acetamido, alkoxy, hydroxy, amino, alkylsulfonyl, haloalkyl, haloalkoxy, haloalkylthio, carboalkoxy, carboxy, carboxamidoalkyl, and carboxamidoalkylaryl. In an alternative of this embodiment, R₄₄ is selected from hydrogen, hydrocarbyl, substituted hydrocarbyl, heteroaryl, heterocyclo, halogen, acetamido, guanidino, hydroxy, nitro, amino, amidosulfonyl, acylamido, hydrocarbyloxy, substituted hydrocarbyloxy, hydrocarbylthio, substituted hydrocarbylthio, hydrocarbylsulfonyl, and substituted hydrocarbylsulfonyl. In yet another alternative of this embodiment, R₄₄ is selected from the group consisting of hydroxy, isobutylsulfonyl, trifluoromethyl, carboxamidobenzyl, carboxamidobutyl-2-yl, isobutyramido, isobutoxy, carboethoxy, carboxyl, amino, 3-aminomethylthiophene, benzylamine, phenethylamine, isobutylamine, methoxyethylamide, 1-carboxylbenzylamide, p-fluorobenzylamide, cyclobutylamide, m-fluorobenzylamide, 1-methylbenzylamide, sec-butylamide, benzylacylamine, isobutylamide, sec-pentylamine, cyclopentylacylamine, 1-carboxyl-2-methylbutylamide, isobutylacylamine, isobutylsulfoxyl, 2-cyclohexylamide, methoxy, sulfonamide, isobutylsulfonamide, aminoacyltrifluoromethyl, and carbmethoxy. In still another alternative of this embodiment R₄₁, R₄₃ and R₄₅ are independently selected from the group consisting of hydrogen, halogen, alkoxy, or hydroxy and R₄₄ is as defined in any of the alternative embodiments above. In yet another alternative of this embodiment, R₄₁, R₄₃ and R₄₅ are independently selected from the group consisting of hydrogen and halogen and R₄₄ is as defined in any of the alternative embodiments above.

In another embodiment for compounds wherein Z₄ corresponds to formula (b) and R₄₂ is amino, R₄₅ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine. In another alternative of this embodiment (i.e., when Z₄ corresponds to formula (b) and R₄₂ is amino), R₄₅ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, sulfonamido, carboxamido and sulfonamidyl, optionally substituted with fluorine. In still another alternative of this embodiment, R₄₅ is selected from the group consisting of hydroxy, carboxy, carboxamido, alkoxy, alkylsulfonyl, sulfonamido, and alkoxycarbonyl. In yet another alternative of this embodiment, R₄₅ is selected from the group consisting of sec-butylamide, carboxy, ethoxycarbonyl, isopropoxycarbonyl, butoxycarbonyl, isopropylamide and hydroxy. In still another alternative of this embodiment, R₄₁, R₄₃ and R₄₄ are independently selected from the group consisting of hydrogen, halogen, alkoxy, or hydroxy and R₄₅ is as defined in any of the alternative embodiments above. In yet another alternative of this embodiment, R₄₁, R₄₃ and R₄₄ are independently selected from the group consisting of hydrogen and halogen and R₄₅ is as defined in any of the alternative embodiments above.

In yet another embodiment for compounds wherein Z₄ corresponds to formula (b) and R₄₂ is amino, R₄₃ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine. In another alternative of this embodiment (i.e., when Z₄ corresponds to formula (b) and R₄₂ is amino), R₄₃ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, sulfonamido, carboxamido and sulfonamidyl, optionally substituted with fluorine. In still another alternative of this embodiment, R₄₃ is selected from the group consisting of hydroxy, carboxy, carboxamido, alkoxy, alkylsulfonyl, sulfonamido, and alkoxycarbonyl. In yet another alternative of this embodiment, R₄₃ is selected from the group consisting of sec-butylamide, carboxy, ethoxycarbonyl, isopropoxycarbonyl, butoxycarbonyl, isopropylamide and hydroxy. In still another alternative of this embodiment, R₄₁, R₄₄ and R₄₅ are independently selected from the group consisting of hydrogen, halogen, alkoxy, or hydroxy and R₄₃ is as defined in any of the alternative embodiments above. In yet another alternative of this embodiment, R₄₁, R₄₄ and R₄₅ are independently selected from the group consisting of hydrogen and halogen and R₄₃ is as defined in any of the alternative embodiments above.

In still another embodiment for compounds wherein Z₄ corresponds to formula (b) and R₄₂ is amino, R₄₁ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, carboxy, sulfonamido, carboxamido and sulfonamidyl, optionally substituted with fluorine. In another alternative of this embodiment (i.e., when Z₄ corresponds to formula (b) and R₄₂ is amino), R₄₁ is selected from the group consisting of hydrocarbyl, substituted hydrocarbyl, acetamidyl, alkoxy, hydroxy, amino, alkylsulfonyl, haloalkoxy, haloalkythio, alkoxycarbonyl, sulfonamido, carboxamido and sulfonamidyl, optionally substituted with fluorine. In still another alternative of this embodiment, R₄₁ is selected from the group consisting of hydroxy, carboxy, carboxamido, alkoxy, alkylsulfonyl, sulfonamido, and alkoxycarbonyl. In yet another alternative of this embodiment, R₄₁ is selected from the group consisting of sec-butylamide, carboxy, ethoxycarbonyl, isopropoxycarbonyl, butoxycarbonyl, isopropylamide and hydroxy. In still another alternative of this embodiment, R₄₃, R₄₄ and R₄₅ are independently selected from the group consisting of hydrogen, halogen, alkoxy, or hydroxy and R₄₁ is as defined in any of the alternative embodiments above. In yet another alternative of this embodiment, R₄₃, R₄₄ and R₄₅ are independently selected from the group consisting of hydrogen and halogen and R₄₁ is as defined in any of the alternative embodiments above.

In yet another embodiment, Z₄ is a five-membered ring having formula (c)

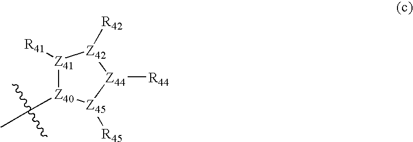

wherein:

Z₄₀, Z₄₁, Z₄₂, Z₄₄, and Z₄₅ are independently selected from the group consisting of carbon, nitrogen, oxygen and sulfur;

R₄₂ is amino;

R₄₄ is selected from the group consisting of is selected from the group consisting of hydrocarbyl, substituted hydrocarbyl, halogen and an optionally substituted heteroatom selected from the group consisting of oxygen, nitrogen, and sulfur; and

R₄₁ and R₄₅ are independently selected from the group consisting of hydrogen, halogen, hydrocarbyl, substituted hydrocarbyl, and an optionally substituted heteroatom selected from the group consisting of oxygen, nitrogen, and sulfur.

In another embodiment of compounds wherein Z₄ corresponds to formula (c) and R₄₂ is amino, R₄₄ is selected from hydrocarbyl, substituted hydrocarbyl, acetamido, alkoxy, hydroxy, amino, alkylsulfonyl, haloalkyl, haloalkoxy, haloalkylthio, carboalkoxy, carboxy, carboxamidoalkyl, and carboxamidoalkylaryl. In an alternative of this embodiment, R₄₄ is selected from hydrogen, hydrocarbyl, substituted hydrocarbyl, heteroaryl, heterocyclo, halogen, acetamido, guanidino, hydroxy, nitro, amino, amidosulfonyl, acylamido, hydrocarbyloxy, substituted hydrocarbyloxy, hydrocarbylthio, substituted hydrocarbylthio, hydrocarbylsulfonyl, and substituted hydrocarbylsulfonyl. In yet another alternative of this embodiment, R₄₄ is selected from the group consisting of hydroxy, isobutylsulfonyl, trifluoromethyl, carboxamidobenzyl, carboxamidobutyl-2-yl, isobutyramido, isobutoxy, carboethoxy, carboxyl, amino, 3-aminomethylthiophene, benzylamine, phenethylamine, isobutylamine, methoxyethylamide, 1-carboxylbenzylamide, p-fluorobenzylamide, cyclobutylamide, m-fluorobenzylamide, 1-methylbenzylamide, sec-butylamide, benzylacylamine, isobutylamide, sec-pentylamine, cyclopentylacylamine, 1-carboxyl-2-methylbutylamide, isobutylacylamine, isobutylsulfoxyl, 2-cyclohexylamide, methoxy, sulfonamide, isobutylsulfonamide, aminoacyltrifluoromethyl, and carbmethoxy. In still another alternative of this embodiment, R₄₁ and R₄₅ are independently selected from the group consisting of hydrogen, halogen, alkoxy, or hydroxy and R₄₄ is as defined in any of the alternative embodiments above. In yet another alternative of this embodiment, R₄₁ and R₄₅ are independently selected from the group consisting of hydrogen and halogen and R₄₄ is as defined in any of the alternative embodiments above.

Still another embodiment provides compounds having formula (I) where X₂ or X₅ are hydrogen bond acceptors. In another embodiment, both X₂ and X₅ are hydrogen bond acceptors. Generally speaking, the phrase “hydrogen bond acceptor” encompasses heteroatoms having a lone pair of electrons available for hydrogen bonding. Suitable hydrogen bond acceptors, when taken with the carbon to which Z₂ is attached, are typically selected from the group consisting of C(O), C(S), C(Cl), C(Br), C(F), C(OH), COCH₃, COR, C(SH), CSR, and CNR₁R₂ wherein R, R₁ and R₂ are independently hydrogen, alkyl, aryl, and arylakyl, optionally substituted with halogen, hydroxy or alkoxy. Suitable X₂ groups include carbon substituted with hydrogen, fluorine, oxygen, or sulfur, nitrogen optionally substituted with hydrogen or oxygen, oxygen, or sulfur. Suitable X₅ groups include oxygen, sulfur, nitrogen, carbonyl and carbon substituted with a halogen selected from fluorine, chlorine and bromine.

In one embodiment for compounds having formula (I), each of X₁, X₂, X₃, X₄, X₅ and X₆ is carbon or nitrogen, each of X₁, X₂, X₄, X₅ and X₆ is sp² or sp³ hybridized, X₃ is sp³ hybridized and L₁, L₃, L₄, Z₁, Z₃, Z₄, R₄₂, and R₄₄ are as described above. In an alternative embodiment, X₁, X₄ and X₅ are carbon, X₂ is carbonyl and X₃ and X₆ are nitrogen. In another embodiment, X₁, X₄ and X₆ are carbon, X₂ is carbonyl and X₃ and X₅ are nitrogen. In still another embodiment, X₁, X₄, X₅ and X₆ are carbon, X₂ is carbonyl and X₃ is nitrogen.

A further embodiment embraces compounds having formula (I), where X₁, X₂, X₃, X₄, X₅, and X₆ form a 6-membered heterocyclic or carbocyclic ring selected from a pyrazinone, pyrimidinone, 2-pyridone, 4-pyrone, 4-pyridone, pyridine-N-oxide, 1,4-quinone, benzene, uracil, piperidinone, dihydropyrimidone, tetrahydropyrimidinone, dehydropiperidinedione, dihydropyrazinone, dihydroisoxazinone, tetrahydrotriazinedione, tetrahydrotriazinone, piperidine, and piperazine and L₁, L₃, L₄, Z₁, Z₃, Z₄, R₄₂, and R₄₄ are as described above. In an alternative embodiment, X₁, X₂, X₃, X₄, X₅, and X₆ form a pyrazinone, pyrimidinone, 2-pyridone, 4-pyrone, 4-pyridone, pyridine-N-oxide, 1,4-quinone, benzene, or uracil ring. In still another embodiment, the ring is a pyrazinone, pyrimidinone, or 2-pyridone.

In an alternative embodiment for compounds having formula (I), X₁, X₂, X₃, X₄, and X₅ form a 5-membered heterocyclic or carbocyclic ring selected from a pyrazolinone, pyrrole, thiophene, pyrazole-N-oxide, 1-amino-pyrazole, 1,3,4-triazole, 2-amino-4-aryl-thiazole, 2-amino-5-aryl-thiazole, pyrrolidine, 2-amino-5-aryl-oxazole, 3-amino-pyrazole, 2-amino-4-aryl-asexual, tetrahydrofuran, cyclopentadienone, and N-hydroxypyrrolidine and L₁, L₃, L₄, Z₁, Z₃, Z₄, R₄₂, and R₄₄ are as described above. In another embodiment, X₁, X₂, X₃, X₄, and X₅ form a pyrazolinone, thiophene, pyrazole-N-oxide, 2-amino-5-aryl-thiazole, tetrahydrofuran, cyclopentadienone, or N-hydroxypyrrolidine ring. In yet another embodiment, the ring is a pyrazolinone, pyrazole-N-oxide, cyclopentadienone, or N-hydroxypyrrolidine. In a further embodiment, the ring is a pyrazolinone.

Yet another aspect of the invention embraces compounds that correspond to formula (II)

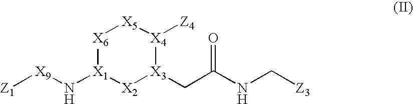

wherein:

each of X₁, X₂, X₃, X₄, X₅ and X₆ is carbon or nitrogen;

X₂ is a hydrogen bond acceptor;

X₉ is a direct bond or —(CH₂)ₘ— where m is 1 to 5;

R₄₂ and R₄₄ are as defined for compounds having formula (I); and

Z₁, Z₃, and Z₄ are as defined for compounds having formula (I).

In yet another embodiment for compounds having formula (II), each of X₁, X₂, X₃, X₄, X₅ and X₆ is carbon or nitrogen;

X₂ is a carbonyl;

X₉ is selected from the group consisting of a direct bond, methylene, and ethylene;

Z₁ is selected from the group consisting of C₁-C₈ alkyl; C₂-C₈ alkenyl, and C₂-C₈ alkynyl, the alkyl, alkenyl, or alkynyl being optionally substituted at any substitutable position with a halogen;

Z₃ comprises a phenyl, furanyl or thienyl ring, the phenyl, furanyl or thienyl ring being substituted with a derivatized amidine which, upon hydrolysis, oxidation, reduction or elimination yields an amidine group, and optionally further substituted with fluorine or hydroxy;

Z₄ comprises a phenyl or thienyl ring having two substituents, R₄₂ and R₄₄, and two ring atoms each of which is in the beta position relative to the ring atom of Z₄ through which Z₄ is covalently bonded to X, wherein one of R₄₂ and R₄₄ is covalently bonded to one of said beta positions and the other of R₄₂ and R₄₄ is covalently bonded to the other of said beta positions;

R₄₂ is amino; and

R₄₄ is selected from the group consisting of hydrocarbyl, substituted hydrocarbyl, heterocyclo, halogen, and an optionally substituted heteroatom selected from nitrogen, oxygen, sulfur and phosphorus.

In still another embodiment for compounds having formula (II), each of X₁, X₂, X₃, X₄, X₅ and X₆ is carbon or nitrogen;

X₂ is a carbonyl;

X₉ is a direct bond;

Z₁ is selected from the group consisting of cyclopropyl, isopropyl, methyl, ethyl, cyclobutyl, isobutyl, tert-butyl and sec-butyl optionally substituted at any substitutable position with fluorine, hydroxy, carboxy, or alkoxycarbonyl;

Z₃ is —R₃₀₀C(═NR₃₀₁)NR₃₀₂R₃₀₃, wherein R₃₀₀ is a phenyl ring, R₃₀₁, R₃₀₂, R₃₀₃ are independently selected from the group consisting of hydrogen, halogen, optionally substituted hydrocarbyl, and an optionally substituted heteroatom selected from the group consisting of oxygen, nitrogen, phosphorus and sulfur, provided at least one of R₃₀₁, R₃₀₂, R₃₀₃ is other than hydrogen;

Z₄ is a substituted phenyl ring; and

R₄₄ is selected from the group consisting of hydroxy, isobutylsulfonyl, trifluoromethyl, carboxamidobenzyl, carboxamidobutyl-2-yl, isobutyramido, isobutoxy, carboethoxy, carboxyl, amino, 3-aminomethylthiophene, benzylamine, phenethylamine, isobutylamine, methoxyethylamide, 1-carboxylbenzylamide, p-fluorobenzylamide, cyclobutylamide, m-fluorobenzylamide, 1-methylbenzylamide, sec-butylamide, benzylacylamine, isobutylamide, sec-pentylamine, cyclopentylacylamine, 1-carboxyl-2-methylbutylamide, isobutylacylamine, isobutylsulfoxyl, 2-cyclohexylamide, methoxy, sulfonamide, isobutylsulfonamide, aminoacyltrifluoromethyl, and carbmethoxy.

A further embodiment provides compounds having formula (II) that are represented by formula (IIa)

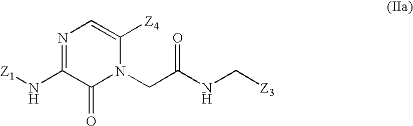

wherein:

each of Z₁, Z₃, Z₄, R₄₂ and R₄₄ are as defined for compounds having formulas (I) and (II).

In another embodiment for compounds having formula (IIa), Z₁ is selected from the group consisting of cyclopropyl, isopropyl, methyl, ethyl, cyclobutyl, isobutyl, tert-butyl and sec-butyl optionally substituted at any substitutable position with fluorine, hydroxy, carboxy, or alkoxycarbonyl;

Z₃ is —R₃₀₀C(═NR₃₀₁)NR₃₀₂R₃₀₃, wherein R₃₀₀ is a phenyl ring, R₃₀₁, R₃₀₂, R₃₀₃ are independently selected from the group consisting of hydrogen, halogen, optionally substituted hydrocarbyl, and an optionally substituted heteroatom selected from the group consisting of oxygen, nitrogen, phosphorus and sulfur, provided at least one of R₃₀₁, R₃₀₂, R₃₀₃ is other than hydrogen; and

Z₄ is a phenyl ring having formula (b)

wherein:

R₄₂ is amino;

R₄₄ is selected from the group consisting of hydroxy, isobutylsulfonyl, trifluoromethyl, carboxamidobenzyl, carboxamidobutyl-2-yl, isobutyramido, isobutoxy, carboethoxy, carboxyl, amino, 3-aminomethylthiophene, benzylamine, phenethylamine, isobutylamine, methoxyethylamide, 1-carboxylbenzylamide, p-fluorobenzylamide, cyclobutylamide, m-fluorobenzylamide, 1-methylbenzylamide, sec-butylamide, benzylacylamine, isobutylamide, sec-pentylamine, cyclopentylacylamine, 1-carboxyl-2-methylbutylamide, isobutylacylamine, isobutylsulfoxyl, 2-cyclohexylamide, methoxy, sulfonamide, isobutylsulfonamide, aminoacyltrifluoromethyl, and carbmethoxy; and

R₄₁, R₄₃ and R₄₅ are independently selected from the group consisting of hydrogen, halogen, hydrocarbyl, substituted hydrocarbyl, and an optionally substituted heteroatom selected from the group consisting of oxygen, nitrogen, and sulfur.

In yet another embodiment for compounds having formula (IIa), Z₁ is selected from the group consisting of cyclopropyl, isopropyl, methyl, ethyl, cyclobutyl, isobutyl, and sec-butyl optionally substituted at any substitutable position with fluorine, hydroxy, carboxy, or alkoxycarbonyl;

Z₃ may be any of the benzamidine derivatives illustrated in Table 1 or 3 below; and

Z₄ R₄₂ and R₄₄ are as described for any of the embodiments involving compounds having formula (IIa).

Yet a further embodiment provides compounds having formula (II) that are represented by formula (IIb)

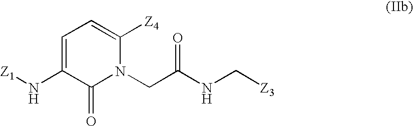

wherein:

each of Z₁, Z₃, Z₄, R₄₂ and R₄₄ are as defined for compounds having formulas (I) and (II).

In an alternative embodiment for compounds having formula (IIb), Z₁ is selected from the group consisting of cyclopropyl, isopropyl, methyl, ethyl, cyclobutyl, isobutyl, tert-butyl and sec-butyl optionally substituted at any substitutable position with fluorine, hydroxy, carboxy, or alkoxycarbonyl;

Z₃ is —R₃₀₀C(═NR₃₀₁)NR₃₀₂R₃₀₃, wherein R₃₀₀ is a phenyl ring, R₃₀₁, R₃₀₂, R₃₀₃ are independently selected from the group consisting of hydrogen, halogen, optionally substituted hydrocarbyl, and an optionally substituted heteroatom selected from the group consisting of oxygen, nitrogen, phosphorus and sulfur, provided at least one of R₃₀₁, R₃₀₂, R₃₀₃ is other than hydrogen; and

Z₄ is a phenyl ring having formula (b)

wherein:

R₄₂ is amino;

R₄₄ is selected from the group consisting of hydroxy, isobutylsulfonyl, trifluoromethyl, carboxamidobenzyl, carboxamidobutyl-2-yl, isobutyramido, isobutoxy, carboethoxy, carboxyl, amino, 3-aminomethylthiophene, benzylamine, phenethylamine, isobutylamine, methoxyethylamide, 1-carboxylbenzylamide, p-fluorobenzylamide, cyclobutylamide, m-fluorobenzylamide, 1-methylbenzylamide, sec-butylamide, benzylacylamine, isobutylamide, sec-pentylamine, cyclopentylacylamine, 1-carboxyl-2-methylbutylamide, isobutylacylamine, isobutylsulfoxyl, 2-cyclohexylamide, methoxy, sulfonamide, isobutylsulfonamide, aminoacyltrifluoromethyl, and carbmethoxy; and

R₄₁, R₄₃ and R₄₅ are independently selected from the group consisting of hydrogen, halogen, hydrocarbyl, substituted hydrocarbyl, and an optionally substituted heteroatom selected from the group consisting of oxygen, nitrogen, and sulfur.

In another embodiment for compounds having formula (IIb), Z₁ is selected from the group consisting of cyclopropyl, isopropyl, methyl, ethyl, cyclobutyl, isobutyl, and sec-butyl optionally substituted at any substitutable position with fluorine, hydroxy, carboxy, or alkoxycarbonyl;

Z₃ may be any of the benzamidine derivatives illustrated in Table 1 or 3 below; and

Z₄, R₄₂ and R₄₄ are as described for any of the embodiments involving compounds having formula (IIb).

Still a further embodiment provides compounds having formula (II) that are represented by formula (IIc)

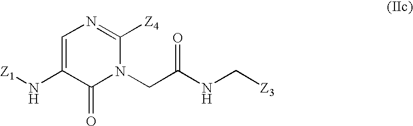

wherein:

each of Z₁, Z₃, Z₄, R₄₂ and R₄₄ are as defined for compounds having formulas (I) and (II).

In an alternative embodiment for compounds having formula (IIc), Z₁ is selected from the group consisting of cyclopropyl, isopropyl, methyl, ethyl, cyclobutyl, isobutyl, tert-butyl and sec-butyl optionally substituted at any substitutable position with fluorine, hydroxy, carboxy, or alkoxycarbonyl;

Z₃ is —R₃₀₀C(═NR₃₀₁)NR₃₀₂R₃₀₃ wherein R₃₀₀ is a phenyl ring, R₃₀₁, R₃₀₂, R₃₀₃ are independently selected from the group consisting of hydrogen, halogen, optionally substituted hydrocarbyl, and an optionally substituted heteroatom selected from the group consisting of oxygen, nitrogen, phosphorus and sulfur, provided at least one of R₃₀₁, R₃₀₂, R₃₀₃ is other than hydrogen; and

Z₄ is a phenyl ring having formula (b)

wherein:

R₄₂ is amino;

R₄₄ is selected from the group consisting of hydroxy, isobutylsulfonyl, trifluoromethyl, carboxamidobenzyl, carboxamidobutyl-2-yl, isobutyramido, isobutoxy, carboethoxy, carboxyl, amino, 3-aminomethylthiophene, benzylamine, phenethylamine, isobutylamine, methoxyethylamide, 1-carboxylbenzylamide, p-fluorobenzylamide, cyclobutylamide, m-fluorobenzylamide, 1-methylbenzylamide, sec-butylamide, benzylacylamine, isobutylamide, sec-pentylamine, cyclopentylacylamine, 1-carboxyl-2-methylbutylamide, isobutylacylamine, isobutylsulfoxyl, 2-cyclohexylamide, methoxy, sulfonamide, isobutylsulfonamide, aminoacyltrifluoromethyl, and carbmethoxy; and

R₄₁, R₄₃ and R₄₅ are independently selected from the group consisting of hydrogen, halogen, hydrocarbyl, substituted hydrocarbyl, and an optionally substituted heteroatom selected from the group consisting of oxygen, nitrogen, and sulfur.

In another embodiment for compounds having formula (IIc), Z₁ is selected from the group consisting of cyclopropyl, isopropyl, methyl, ethyl, cyclobutyl, isobutyl, and sec-butyl optionally substituted at any substitutable position with fluorine, hydroxy, carboxy, or alkoxycarbonyl;

Z₃ may be any of the benzamidine derivatives illustrated in Table 1 or 3 below; and

Z₄, R₄₂ and R₄₄ are as described for any of the embodiments involving compounds having formula (IIc).

A further aspect of the invention embraces compounds having formula (III)

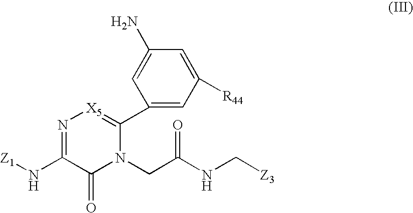

wherein:

each of Z₁, Z₃, X₅ and R₄₄ are as defined for compounds having formulas (I) and (II).

In another embodiment for compounds having formula (III), X₅ is CH, C(Cl) or C(F);

Z₁ is isopropyl, cyclopropyl, cyclobutyl or cycylopentyl optionally substituted by fluorine, hydroxy, carboxy, or alkoxycarbonyl;

Z₃ is —R₃₀₀C(═NR₃₀₁)NR₃₀₂R₃₀₃, wherein R₃₀₀ is a 6-membered carbocyclic aromatic ring, R₃₀₁, R₃₀₂, R₃₀₃ are independently selected from the group consisting of hydrogen, halogen, optionally substituted hydrocarbyl, and an optionally substituted heteroatom selected from the group consisting of oxygen, nitrogen, phosphorus and sulfur, provided at least one of R₃₀₁, R₃₀₂, R₃₀₃ is other than hydrogen; and

R₄₄ is selected from the group consisting of hydroxy, isobutylsulfonyl, trifluoromethyl, carboxamidobenzyl, carboxamidobutyl-2-yl, isobutyramido, isobutoxy, carboethoxy, carboxyl, amino, 3-aminomethylthiophene, benzylamine, phenethylamine, isobutylamine, methoxyethylamide, 1-carboxylbenzylamide, p-fluorobenzylamide, cyclobutylamide, m-fluorobenzylamide, 1-methylbenzylamide, sec-butylamide, benzylacylamine, isobutylamide, sec-pentylamine, cyclopentylacylamine, 1-carboxyl-2-methylbutylamide, isobutylacylamine, isobutylsulfoxyl, 2-cyclohexylamide, methoxy, sulfonamide, isobutylsulfonamide, aminoacyltrifluoromethyl, and carbmethoxy.

In another embodiment for compounds having formula (III), X₅ is CH, and Z₁, Z₃, and R₄₄ are any of the groups detailed in Table 1 below.

TABLE 1

| Z₁ | R₄₄ | Z₃ |
|---|---|---|
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsulfonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, 1- methylbenzylamide, sec- butylamide, benzylacylamine, isobutylamide, sec- pentylamine, or cyclopentylacylamine | 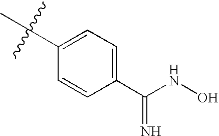 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsulfonamide, carboethoxy, aminoacyltrifluoro- ethyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, 1- methylbenzylamide, sec- butylamide, benzylacylamine, isobutylamide, sec- pentylamine, or cyclopentyl acyl amine | 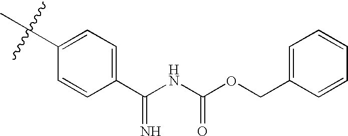 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsulfonamide, carboethoxy, aminoacyltrifluoro- ethyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, 1- methylbenzylamide, sec- butylamide, benzylacylamine, isobutylamide, sec- pentylamine, or cyclopentyl acyl amine | 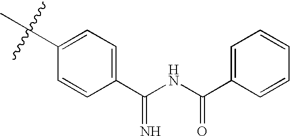 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 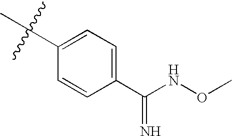 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 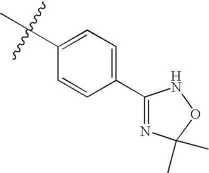 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 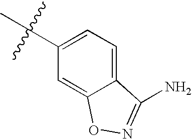 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 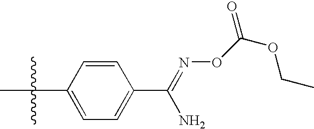 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 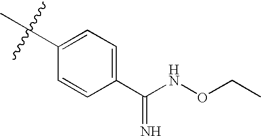 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 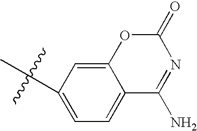 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 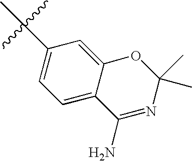 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 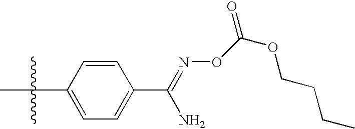 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 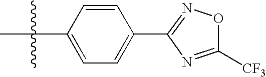 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 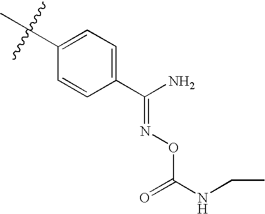 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 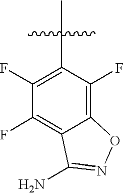 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 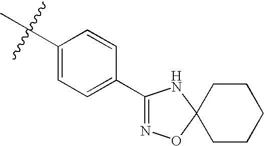 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 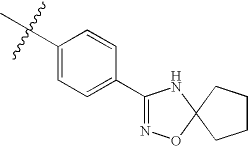 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 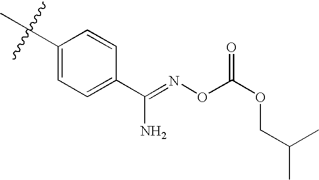 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 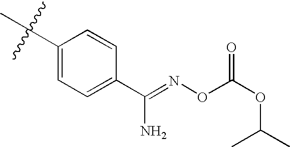 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 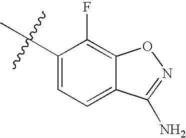 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 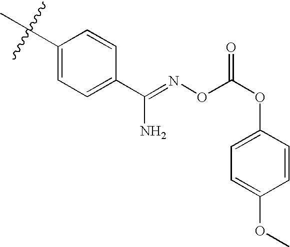 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 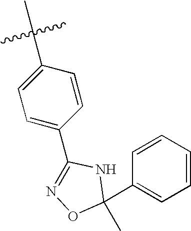 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 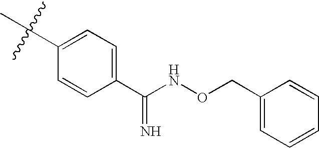 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 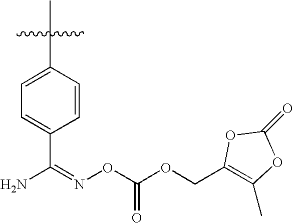 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 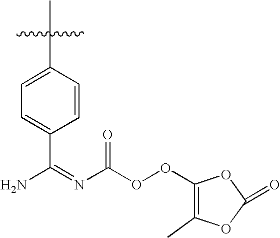 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 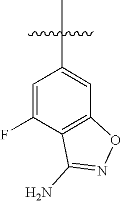 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 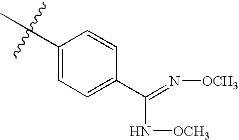 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 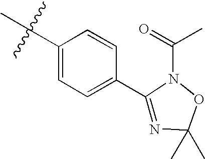 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 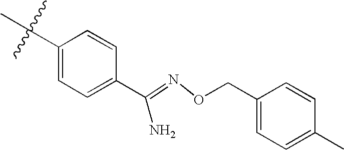 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 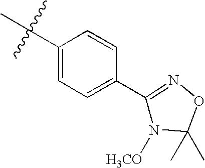 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 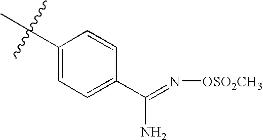 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 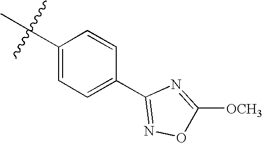 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 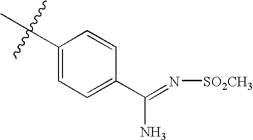 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 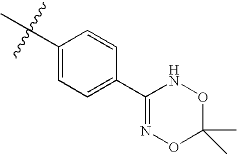 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 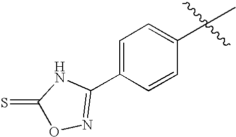 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 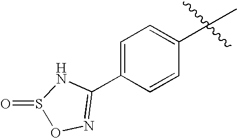 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 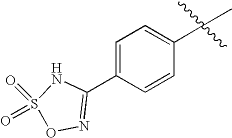 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 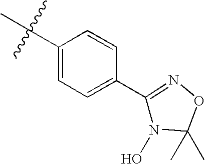 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 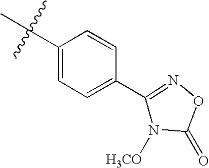 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 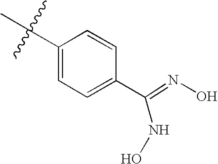 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 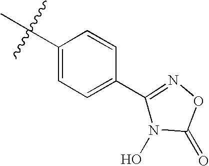 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 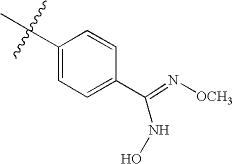 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 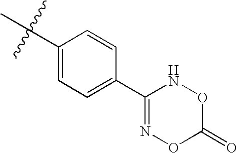 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 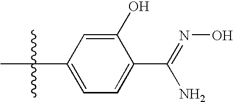 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 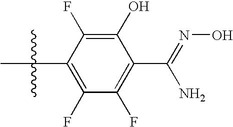 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 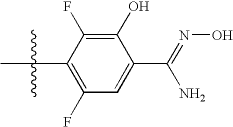 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 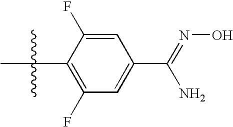 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 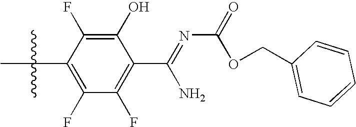 | |
| alkyl, substituted alkyl, phenyl, substituted phenyl, cycloalkyl, or substituted cycloalkyl | hydroxy, 1-carboxyl-2- methylbutylamide, isobutylsulfonyl, isobutylacylamine, trifluoromethyl, isobutylsulfoxyl, carboxamidobenzyl, 2- | |
| # cyclohexylamide, carboxamidobutyl-2-yl, methoxyl, isobutyramido, sulfonamide, isobutoxy, isobutylsultonamide, carboethoxy, aminoacyltrifluoro- methyl, carboxyl, carbmethoxy, amino, 3- aminomethylthiophene, benzylamine, phenethylamine, isobutylamine, | | |
| # methoxyethylamide, 1- carboxylbenzylamide, p- fluorobenzylamide, cyclobutylamide, m- fluorobenzylamide, 1- methylbenzylamide, sec- butylamide, benzylacylamine, isobutylamide, sec- pentylamine, or cyclopentylacylamine | 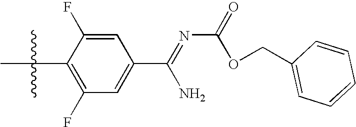 | |

For convenience, each of the substituents identified for R⁴⁴ in Table 1 is set forth below.

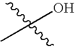

hydroxy

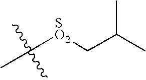

isobutylsulfonyl

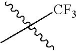

trifluoromethyl

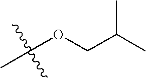

isobutoxy

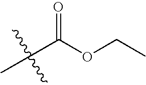

carboethoxy

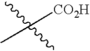

carboxyl

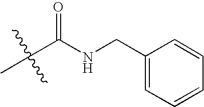

carboxamidobenzyl

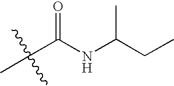

carboxamidobutyl-2-yl

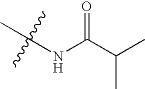

isobutyramido

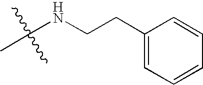

phenethylamine

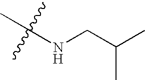

isobutylamine

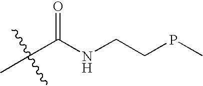

methoxyethylamide

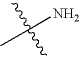

amino

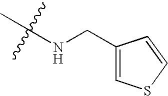

3-aminomethylthiophene

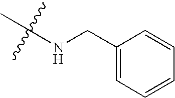

benzylamine

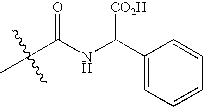

1-carboxylbenzylamide
R or S ISOMERS

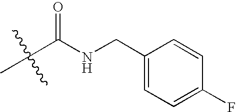

p-fluorobenzylamide

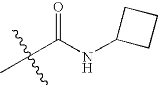

cyclobutylamide

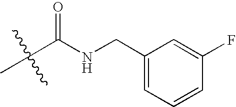

m-fluorobenzylamide

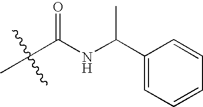

1-methylbenzylamide
RACEMIC or R or S

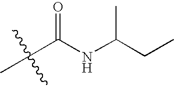

sec-butylamide
R or S ISOMERS

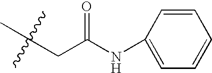

benzylacylamine

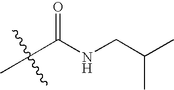

isobutylamide

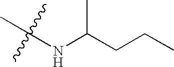

sec-pentylamine

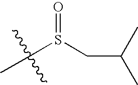

isobutylsulfoxyl

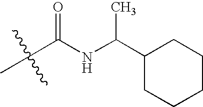

2-cyclohexylamide
R or S ISOMERS

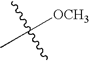

methoxy

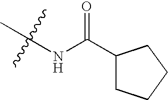

cyclopentylacylamine

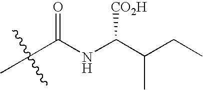

1-carboxyl-2-methylbutylamide

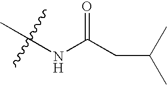

isobutylacylamine

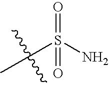

sulfonamide

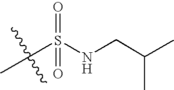

isobutylsulfonamide

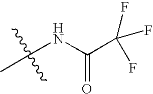

aminoacyltrifluoromethyl

A further embodiment provides compounds having formula (III) that are represented by formula (IIIa)

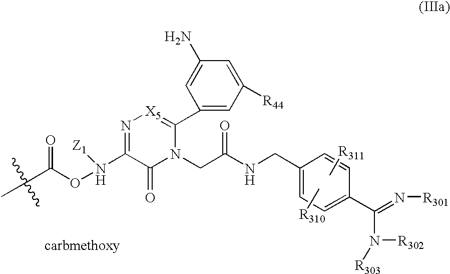

wherein:

each of Z₁, X₅ and R₄₄ are as defined for any embodiment of compounds having formula III;

R₃₀₁, R₃₀₂, and R₃₀₃ are independently selected from the group consisting of:

(i) hydrogen, —C(═O)Rₐ, —C(═O)ORₐ, —S(═O)ORₐ, —S(═O)SRₐ, —S(═O)₂ORₐ, —S(═O)₂SRₐ and alkenyl, wherein Rₐ is selected from the group consisting of hydrocarbyl, substituted hydrocarbyl, and heterocyclo, provided, however, that the carbon atom of R₃₀₁, R₃₀₂, and R₃₀₃ directly bonded to the amidine is sp² hybridized when R₃₀₁, R₃₀₂, and R₃₀₃ is alkenyl,

(ii) hydrogen, optionally substituted hydrocarbyl and aryl, provided, however, the carbon atom of R₃₀₁, R₃₀₂, and R₃₀₃ directly bonded to the amidine is sp³ hybridized when R₃₀₁, R₃₀₂, and R₃₀₃ is optionally substituted hydrocarbyl,

(iii) hydrogen, —OR_{b}, —SR_{b}, —NR_{b}, or —N(R_{b})₂, wherein each R_{b} is independently optionally substituted hydrocarbyl, and heterocyclo, and

(iv) hydrogen, substituted hydrocarbyl wherein the carbon bonded to the amidine group is substituted with —OR_{c}, —SR_{c}, —NR_{c}, or —N(R_{c})₂, wherein each R_{c} is independently —C(O)R_{d}, —C(O)NR_{d}, —C(O)OR_{d}, —C(O)N(R_{d})₂ and each R_{d} is independently hydrocarbyl, substituted hydrocarbyl or heterocyclo, and substituted alkyl with the carbon atom beta to the point of attachment to the amidine group being an unsaturated electron withdrawing group, provided, however, at least one of R₃₀₁, R₃₀₂, and R₃₀₃ is other than hydrogen; and

R₃₁₀ and R₃₁₁ are independently selected from the group consisting of hydrogen, fluorine, hydroxy, alkoxy, and carboxy, provided at least one of R₃₁₀ and R₃₁₁ is other than fluorine and hydrogen.

Yet a further aspect of the invention embraces compounds having formula (IV)

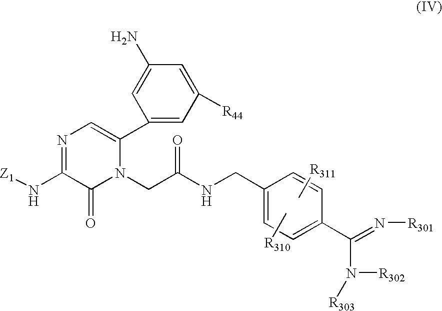

wherein:

each of Z₁, R₄₄, R₃₀₁, R₃₀₂, R₃₀₃, R₃₁₀ and R₃₁₁ are as defined for any embodiment of compounds having formula (IIIa).

Another embodiment provides compounds having formula (IV) that are represented by formula (IVa)

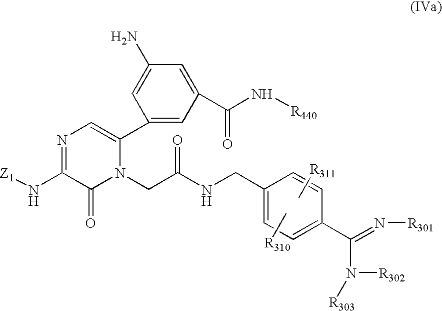

wherein:

each of Z₁, R₃₀₁, R₃₀₂, R₃₀₃, R₃₁₀ and R₃₁₁ are as defined for any embodiment of compounds having formula (IIIa); and

R₄₄₀ is C₁-C₆ alkyl, aryl, aralkyl, carboxy, or carboxyalkyl, wherein the alkyl, aryl, aralkyl, carboxy, or carboxyalkyl is optionally further substituted by fluorine.

Still a further embodiment provides compounds having formula (IV) that are represented by formula (IVb)

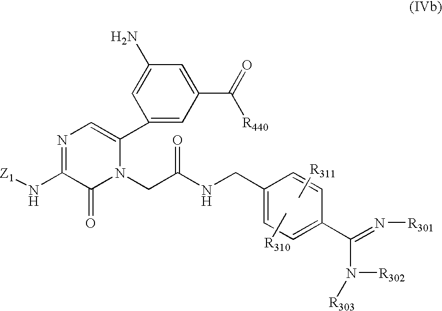

wherein:

each of Z₁, R₃₀₁, R₃₀₂, R₃₀₃, R₃₁₀ and R₃₁₁ are as defined for any embodiment of compounds having formula (IIIa); and

R₄₄₀ is C₁-C₆ alkyl, aryl, aralkyl, carboxy, or carboxyalkyl, wherein the alkyl, aryl, aralkyl, carboxy, or carboxyalkyl is optionally further substituted by fluorine.

In yet a further embodiment, compounds having any one of formulas (IV), (IVa) or (IVb) are selected from the compounds in Table 2 below:

TABLE 2

| | |
|---|---|
| (a) | 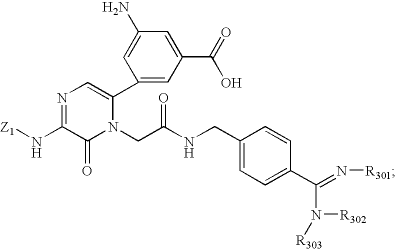 |
| (b) | 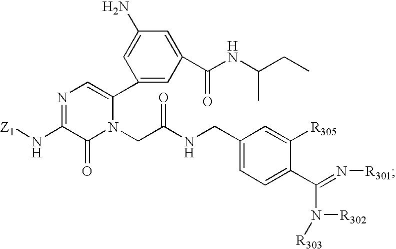 |
| (c) | 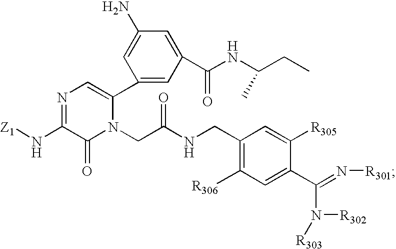 |
| (d) | 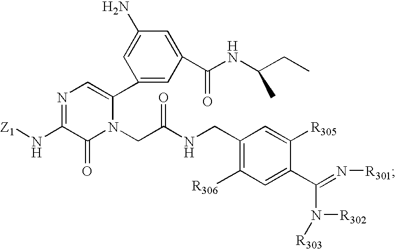 |
| (e) | 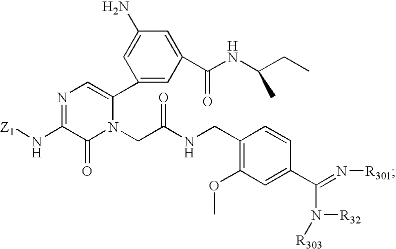 |
| (f) | 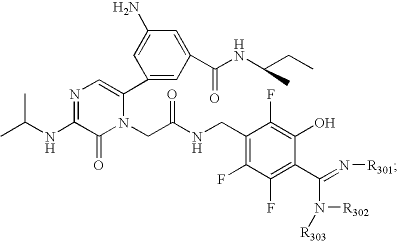 |
| (g) | 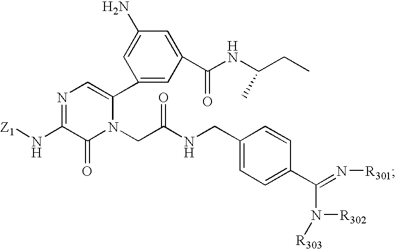 |
| (h) | 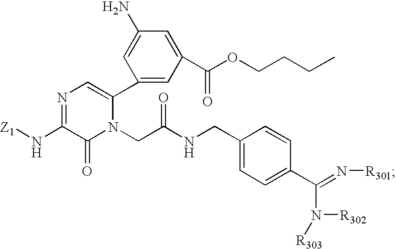 |
| (i) | 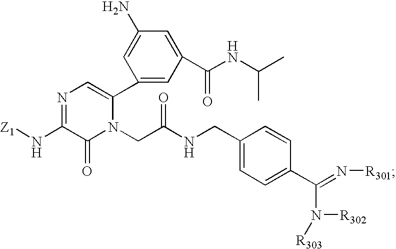 |
| (j) | 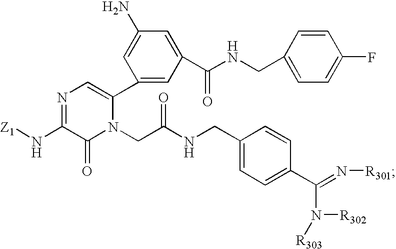 |
| (k) | 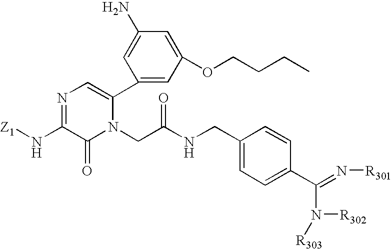 |
| (l) | 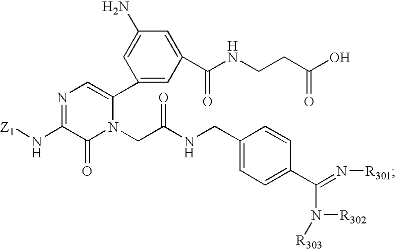 |
| (m) | 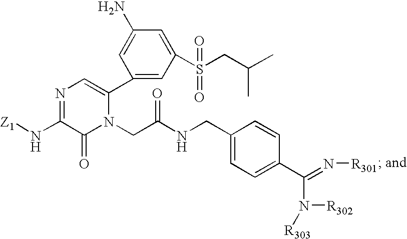 |
| (n) | 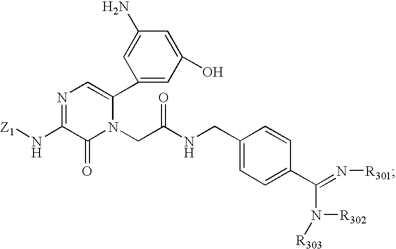 |

wherein:

Z₁ is isopropyl or cyclopropyl optionally substituted with fluorine, hydroxy, carboxy, or alkoxycarbonyl;

R₃₀₁, R₃₀₂, and R₃₀₃ are independently selected from the group consisting of:

(i) hydrogen, —C(═O)Rₐ, —C(═O)ORₐ, —S(═O)ORₐ, —S(═O)SRₐ, —S(═O)₂ORₐ, —S(═O)₂SRₐ and alkenyl, wherein Rₐ is selected from the group consisting of hydrocarbyl, substituted hydrocarbyl, and heterocyclo, provided, however, that the carbon atom of R₃₀₁, R₃₀₂, and R₃₀₃ directly bonded to the amidine is sp² hybridized when R₃₀₁, R₃₀₂, and R₃₀₃ is alkenyl,

(ii) hydrogen, optionally substituted hydrocarbyl and aryl, provided, however, the carbon atom of R₃₀₁, R₃₀₂, and R₃₀₃ directly bonded to the amidine is sp³ hybridized when R₃₀₁, R₃₀₂, and R₃₀₃ is optionally substituted hydrocarbyl,

(iii) hydrogen, —OR_{b}, —SR_{b}, —NR_{b}, or —N(R_{b})₂, wherein each R_{b} is independently optionally substituted hydrocarbyl, and heterocyclo, and

(iv) hydrogen, substituted hydrocarbyl wherein the carbon bonded to the amidine group is substituted with —OR_{c}, —SR_{c}, —NR_{c}, or —N(R_{c})₂, wherein each R_{c} is independently —C(O)R_{d}, —C(O)NR_{d}, —C(O)OR_{d}, —C(O)N(R_{d})₂ and each R_{d} is independently hydrocarbyl, substituted hydrocarbyl or heterocyclo, and substituted alkyl with the carbon atom beta to the point of attachment to the amidine group being an unsaturated electron withdrawing group, provided, however, at least one of R₃₀₁, R₃₀₂, and R₃₀₃ is other than hydrogen;

R₃₀₅, when present, is hydroxy or hydrogen; and

R₃₀₆, when present, is hydroxy or hydrogen, provided if R₃₀₅ is hydroxy then R₃₀₆ is hydrogen and if R₃₀₅ is hydrogen then R₃₀₆ is hydroxy.

Yet a further aspect of the invention embraces compounds having formula (V)

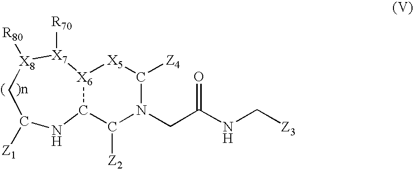

wherein

X₅ is nitrogen, CH, C(F), C(Cl), or C(Br);

X₆ is carbon or nitrogen, provided the dashed line represents a double bond when X₆ is carbon and the dashed line represents a single bond when X₆ is nitrogen;

X₇ and X₈ are independently carbon, nitrogen, oxygen or sulfur;

Z₁ is selected from the group consisting of C₁-C₈ alkyl, C₂-C₈ alkenyl, and C₂-C₈ alkynyl, the alkyl, alkenyl, or alkynyl being optionally substituted at any substitutable position with a halogen;

Z₂ is a hydrogen bond acceptor covalently or datively bonded to the carbon gamma to X₅.

Z₃ comprises a substituted phenyl, thienyl, or furanyl ring, the phenyl, thienyl or furanyl ring being substituted with a derivatized amidine group and optionally substituted at any substitutable position with fluorine, hydroxy, carboxy, alkoxycarbonyl, or hydrocarbyloxy;

Z₄ comprises a 5- or 6-membered heteroaryl or aryl ring, the ring atoms of Z₄ being Z₄₀, Z₄₁, Z₄₂, Z₄₄ and Z₄₅ when Z₄ is a 5-membered ring and Z₄₀, Z₄₁, Z₄₂, Z₄₃, Z₄₄ and Z₄₅ when Z₄ is a 6-membered ring, Z₄₀, Z₄₁, Z₄₂, Z₄₃, Z₄₄ and Z₄₅, being carbon, nitrogen, oxygen or sulfur, Z₄₀ being the ring atom through which Z₄ is attached to the heterocyclic core ring, Z₄₁ and Z₄₅ each being in an alpha position relative to Z₄₀, Z₄₂ and Z₄₄ each being in a beta position relative to Z₄₀, Z₄₃ being in the gamma position relative to Z₄₀ when Z₄ is a 6-membered ring, Z₄ having a substituent R₄₂ covalently attached to Z₄₂, and a second substituent bonded to one of Z₄₁, Z₄₃, Z₄₄, or Z₄₅, the substituent being R₄₁ when bonded to Z₄₁, the substituent being R₄₃ when bonded to Z₄₃, the substituent being R₄₄ when bonded to Z₄₄, and the substituent being R₄₅ when bonded to Z₄₅;

R₄₂ is amino;

R₄₁, R₄₃, R₄₄ and R₄₅ are independently hydrogen, hydrocarbyl, substituted hydrocarbyl, heterocyclo, halogen, or a substituted or unsubstituted heteroatom selected from nitrogen, oxygen, sulfur and phosphorus, provided at least one of R₄₁, R₄₃, R₄₄ or R₄₅ is other than hydrogen;

R₇₀ and R₈₀ are independently selected from the group consisting of hydrogen, halogen, amino, hydrocarbyl, substituted hydrocarbyl, aryl, wherein aryl is phenyl optionally substituted by hydroxy, amino, C₁-C₈ alkyl, or halogen provided that R₇₀ is not present when X₇ is a bond and R₈₀ is not present when X₈ is a bond; or R₇₀ and R₈₀, along with the ring atoms to which each is attached, form a 5- or 6-membered saturated ring; and

n is 0 to 2.

Still a further embodiment provides compounds having formula (V) that are represented by formula (Va)

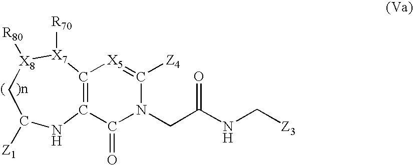

wherein each of X₅, X₇, X₈, Z₁, Z₃, Z₄, R₇₀, R₈₀ and n are as defined for compounds having formula (V).

In another embodiment, the compound represented by any of formulas (I)-(V) is selected from the group of compounds listed in Table 3 below. Certain compounds listed in Table 3 are pharmaceutically acceptable salts of compounds having any of formulas (I)-(V). By way of example, compound 78 has 1.6 molecules of C(O)OHCF₃ salt per molecule of compound 78 and 0.3 molecules of OH₂ per molecule of compound 78. By way of further example, compound 80 has 1 molecule of C(O)OHCF₃ salt per molecule of compound 80.

TABLE 3

| Compound No. | Compound |
|---|---|
| 1 | 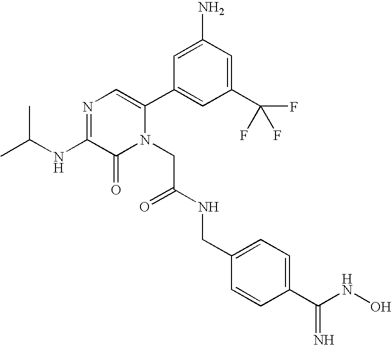 |
| 2 | 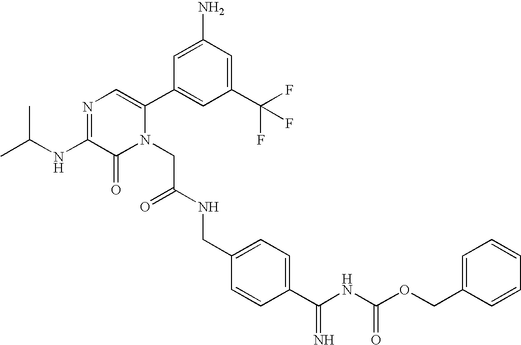 |
| 3 | 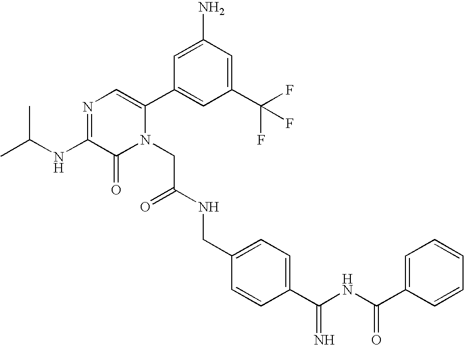 |
| 4 | 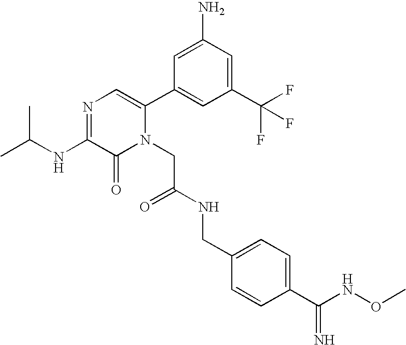 |
| 5 | 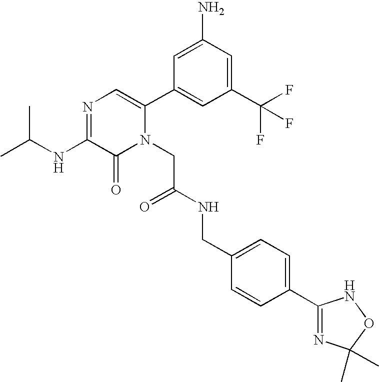 |
| 6 | 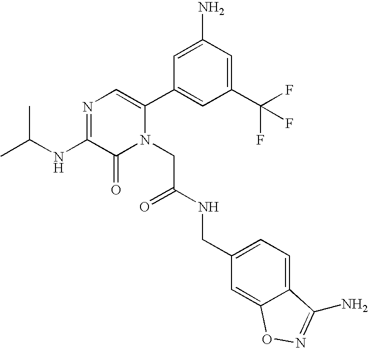 |
| 7 | 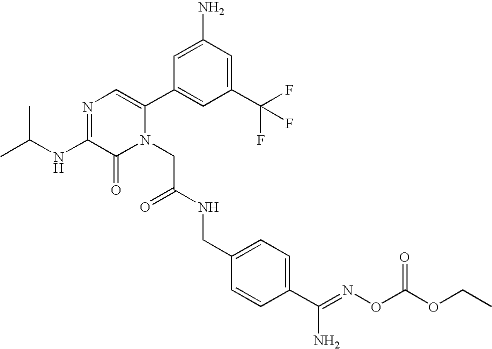 |
| 8 | 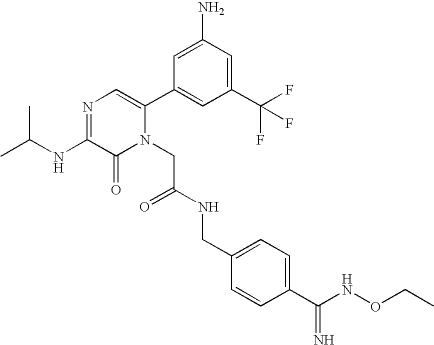 |
| 9 | 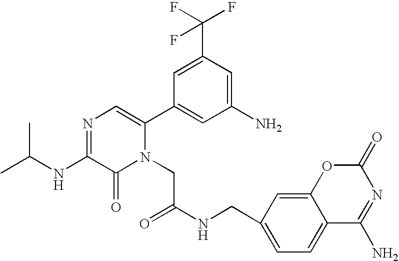 |
| 10 | 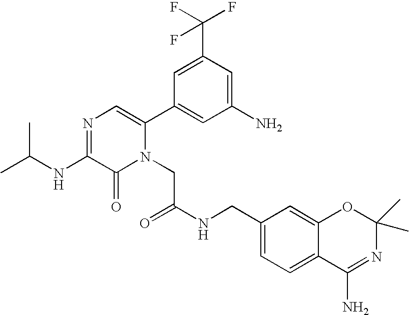 |
| 11 | 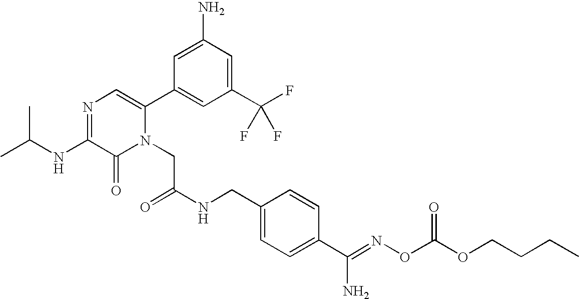 |
| 12 | 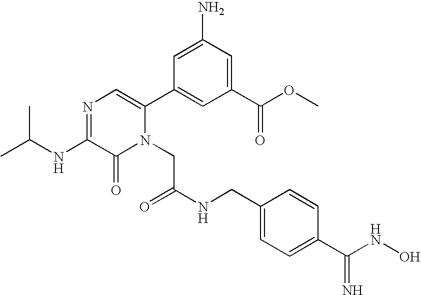 |
| 13 | 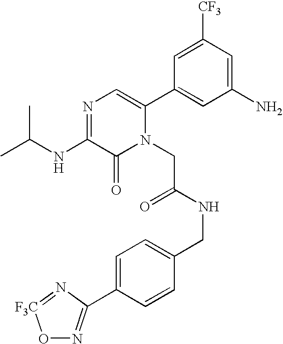 |
| 14 | 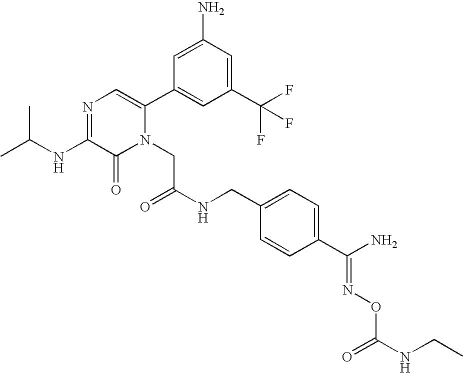 |
| 15 | 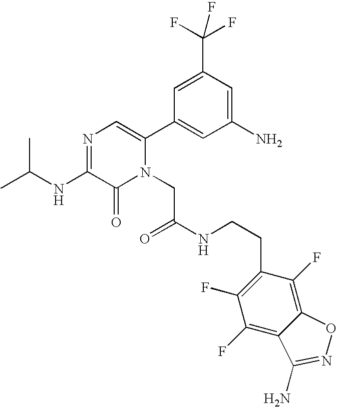 |
| 16 | 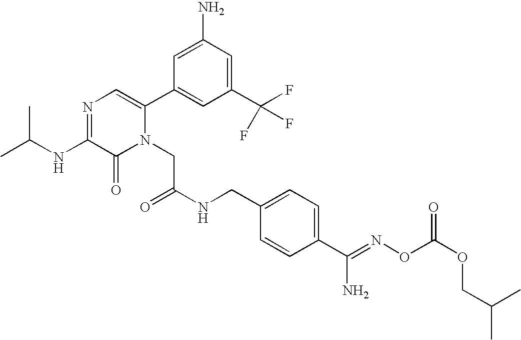 |
| 17 | 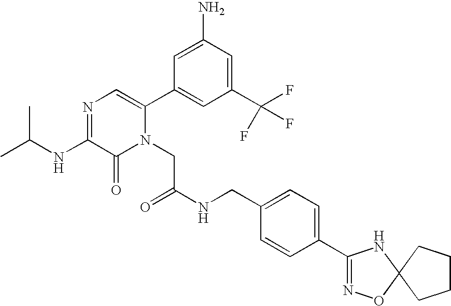 |
| 18 | 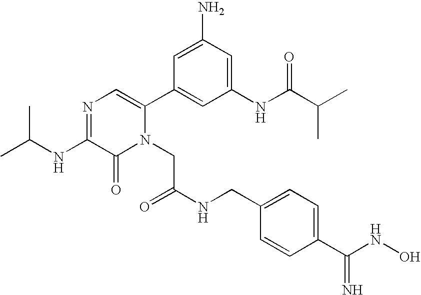 |
| 19 | 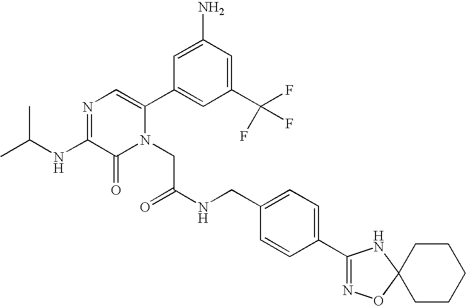 |
| 20 | 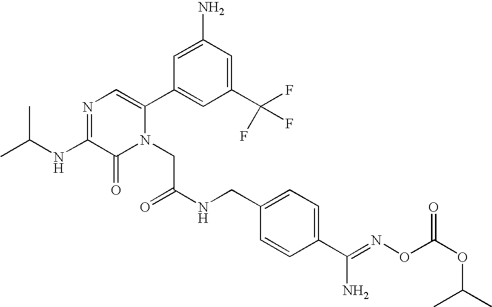 |
| 21 | 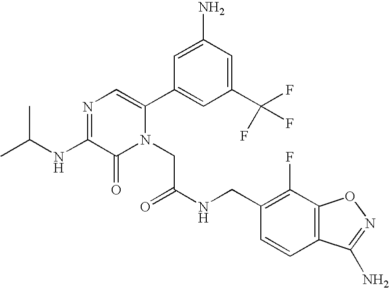 |
| 22 | 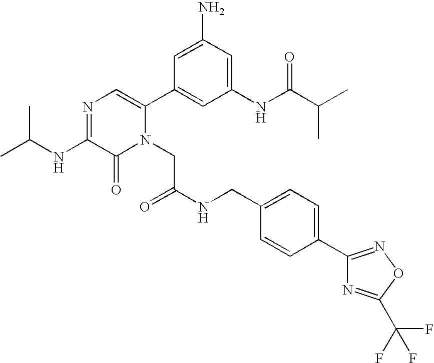 |
| 23 | 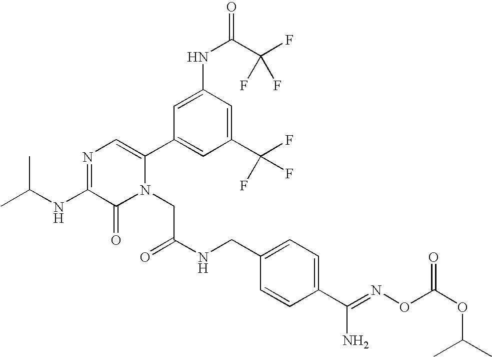 |
| 24 | 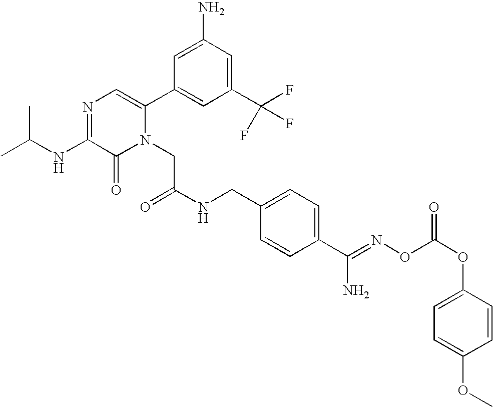 |
| 25 | 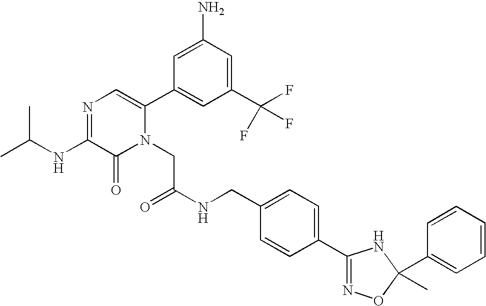 |
| 26 | 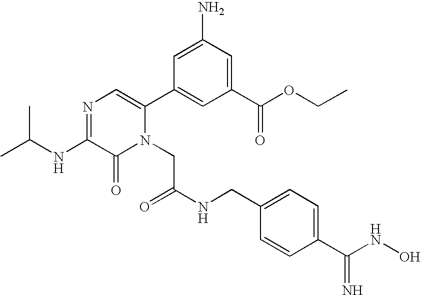 |
| 27 | 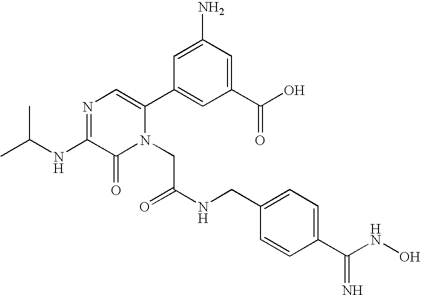 |
| 28 | 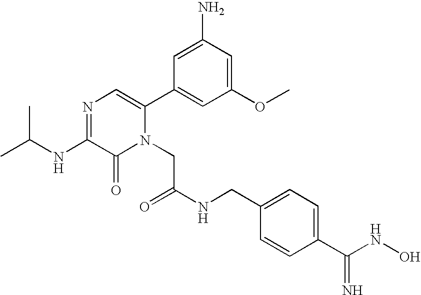 |
| 29 | 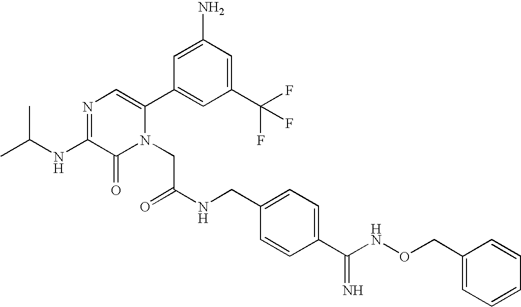 |
| 30 | 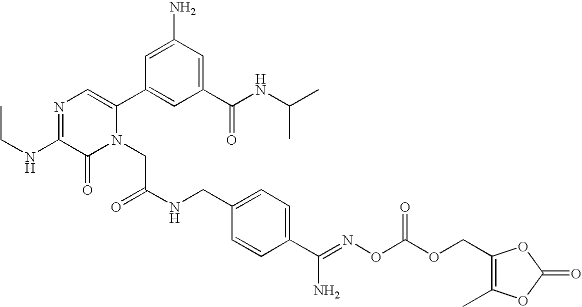 |
| 31 | 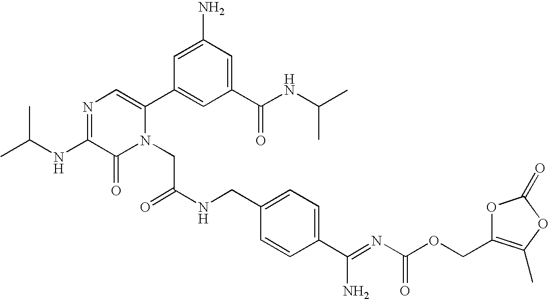 |
| 32 | 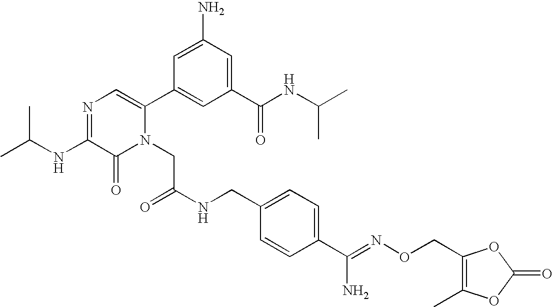 |
| 33 | 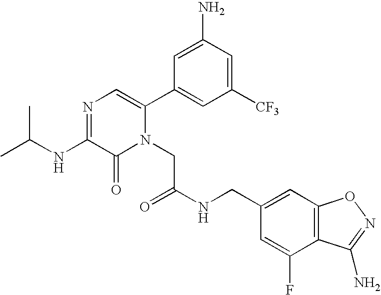 |
| 34 | 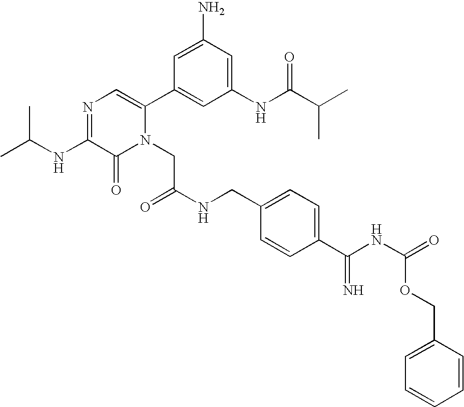 |
| 35 | 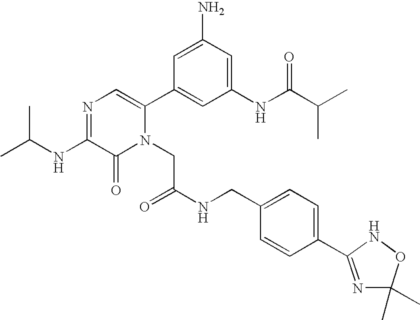 |
| 36 | 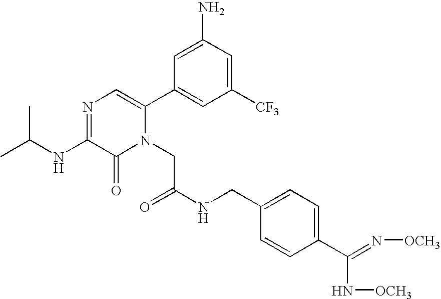 |
| 37 | 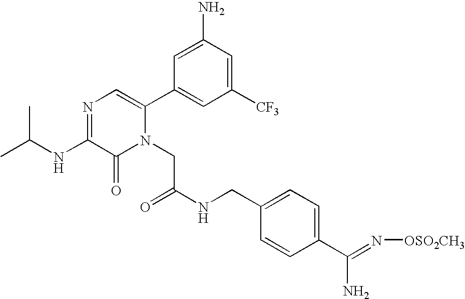 |
| 38 | 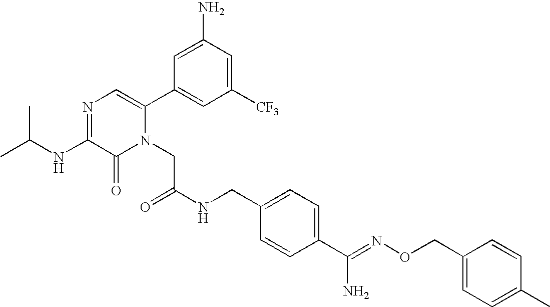 |
| 39 | 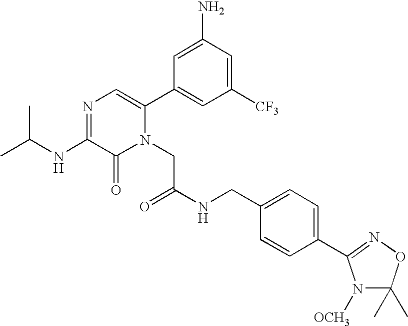 |
| 40 | 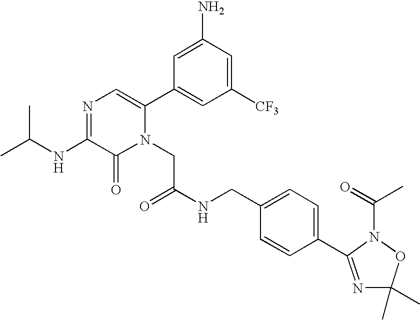 |
| 41 | 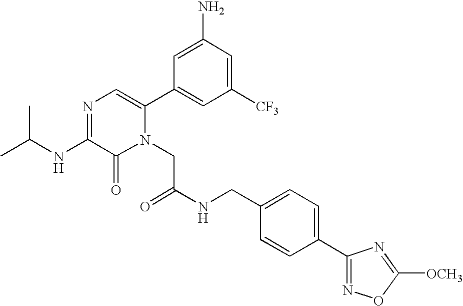 |
| 42 | 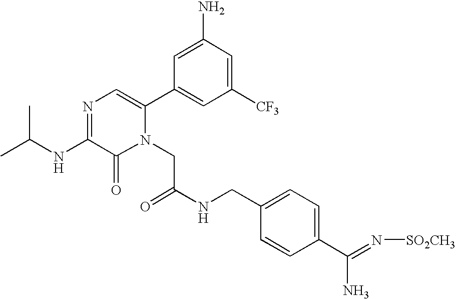 |
| 43 | 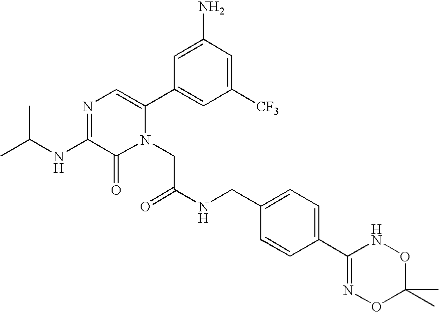 |
| 44 | 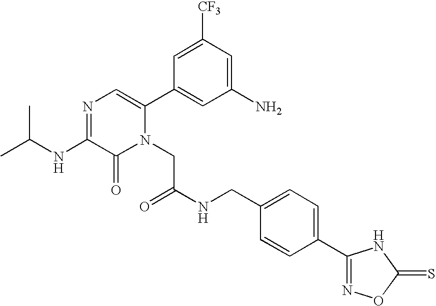 |
| 45 | 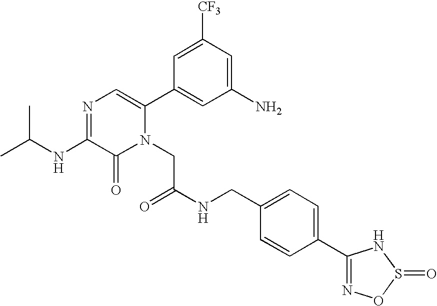 |
| 46 | 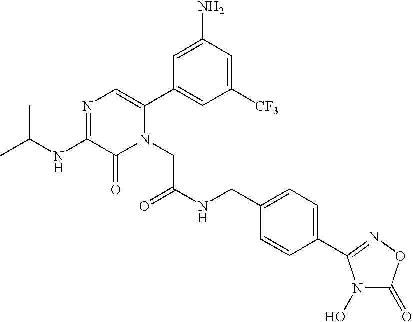 |
| 47 | 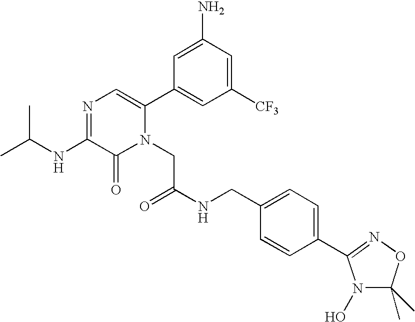 |
| 48 | 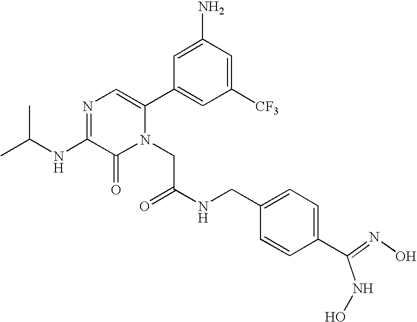 |
| 49 | 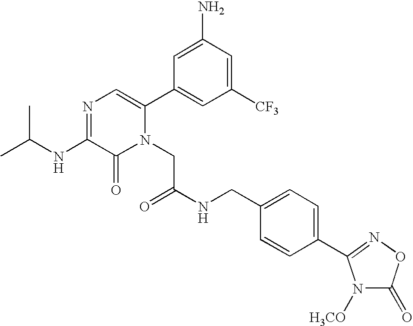 |
| 50 | 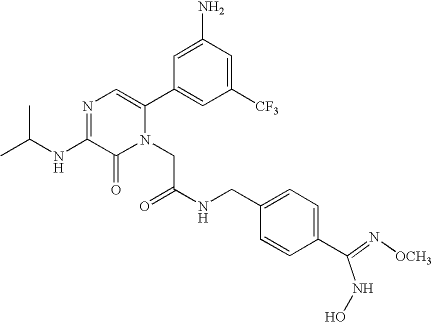 |
| 51 | 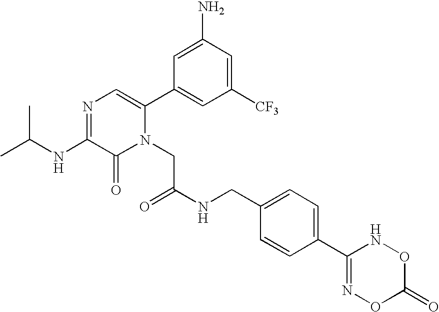 |
| 52 | 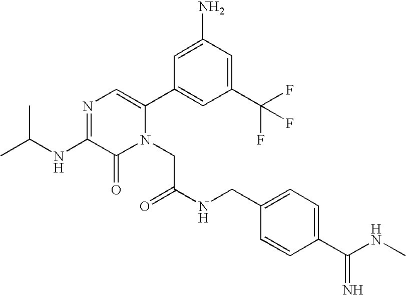 |
| 53 | 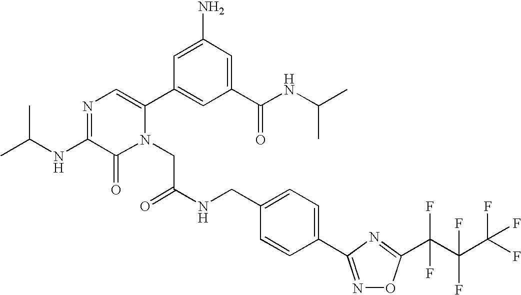 |
| 54 | 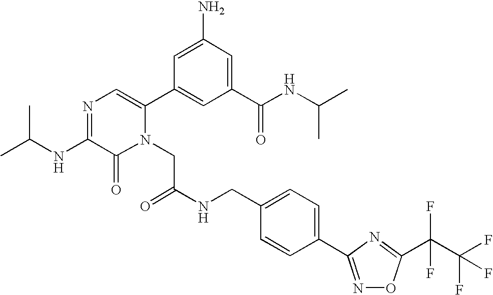 |
| 55 | 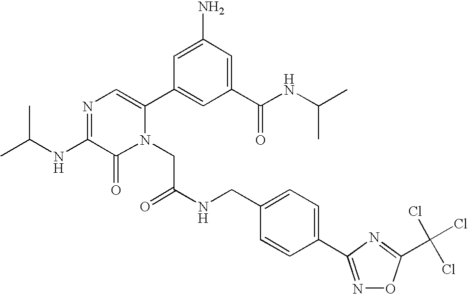 |
| 56 | 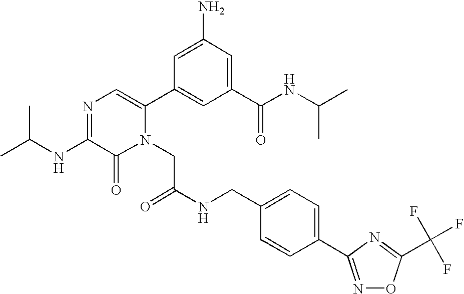 |
| 57 | 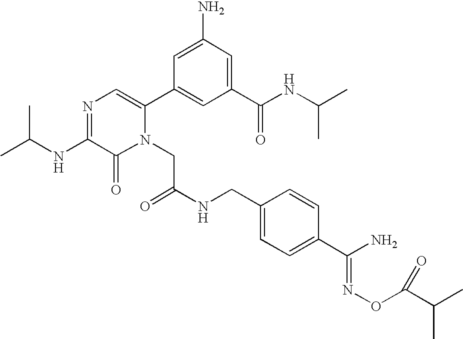 |
| 58 | 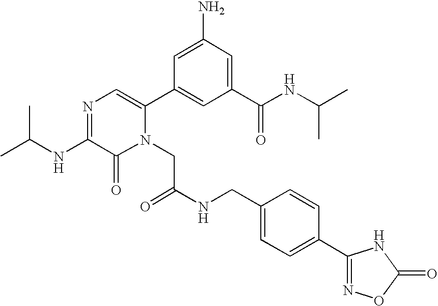 |
| 59 | 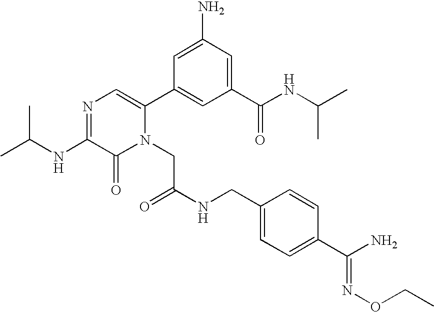 |
| 60 | 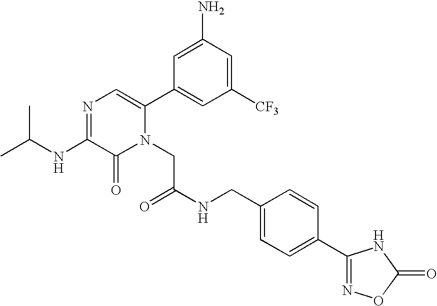 |
| 61 | 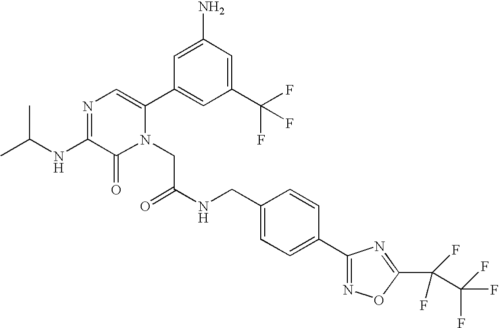 |
| 62 | 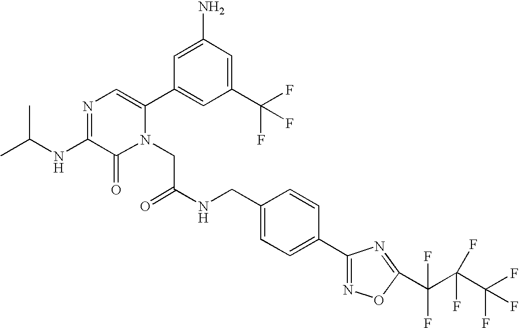 |
| 63 | 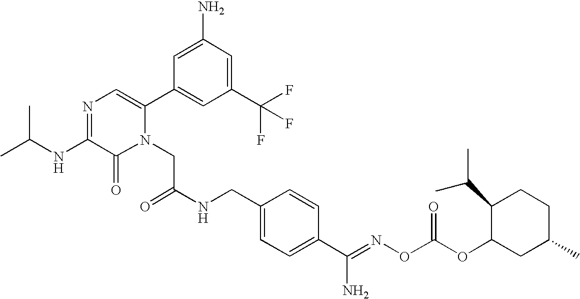 |
| 64 | 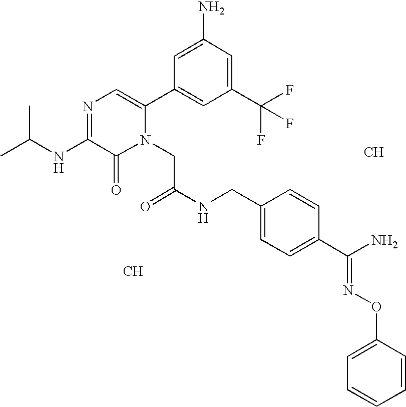 |
| 65 | 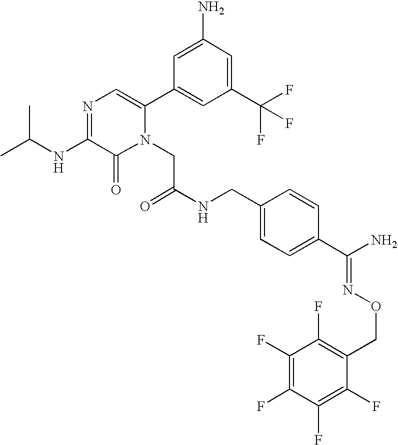 |
| 66 | 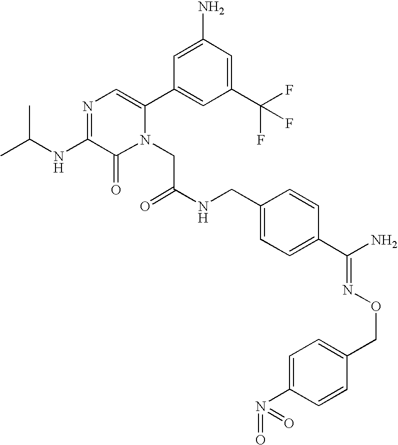 |
| 67 | 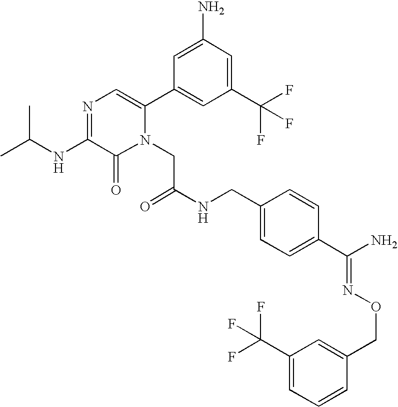 |
| 68 | 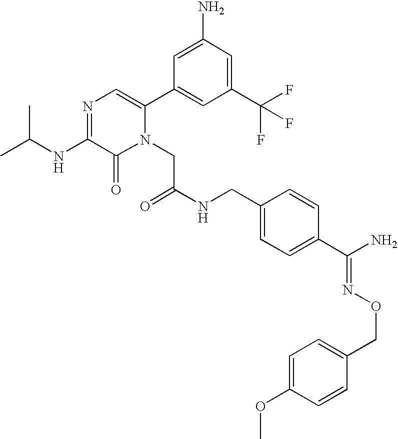 |
| 69 | 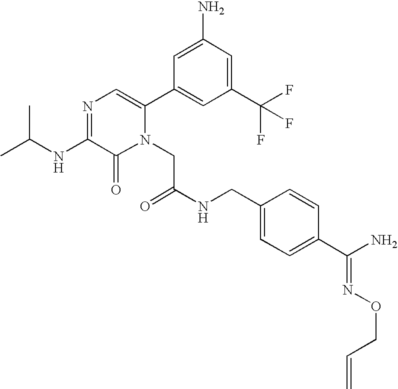 |
| 70 | 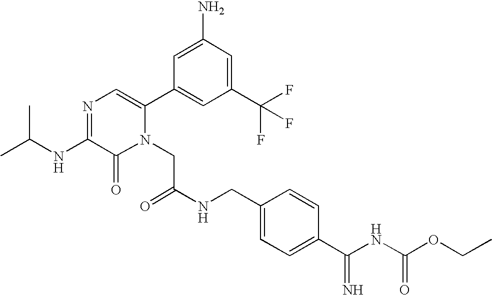 |
| 71 | 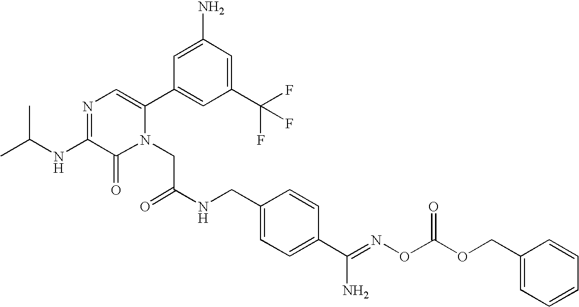 |
| 72 | 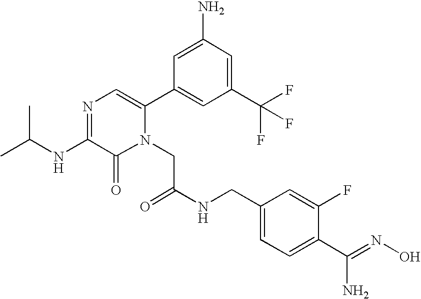 |
| 73 | 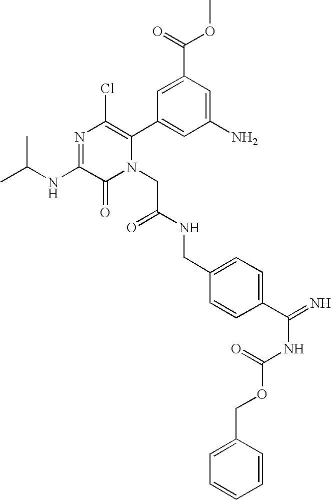 |
| 74 | 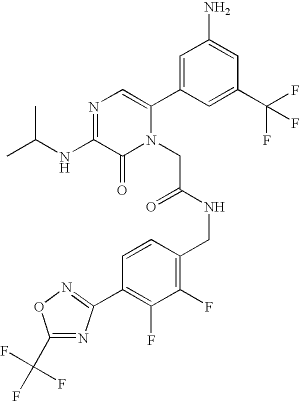 |
| 75 | 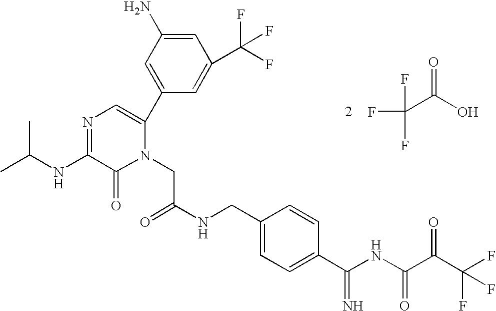 |
| 76 | 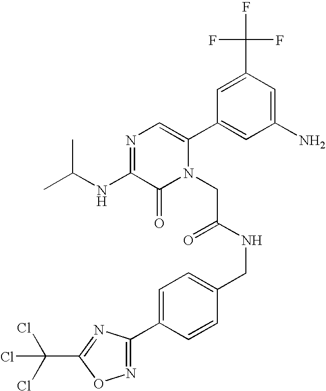 |
| 77 | 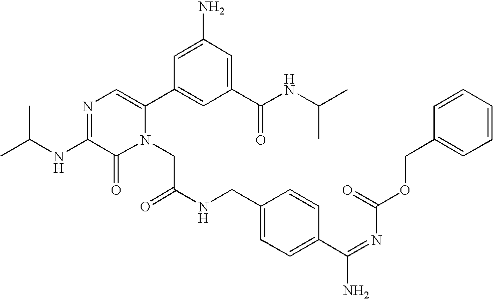 |
| 78 | 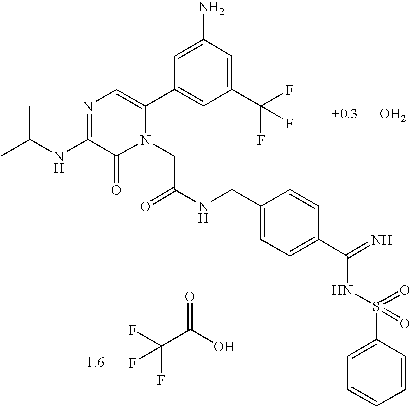 |
| 79 | 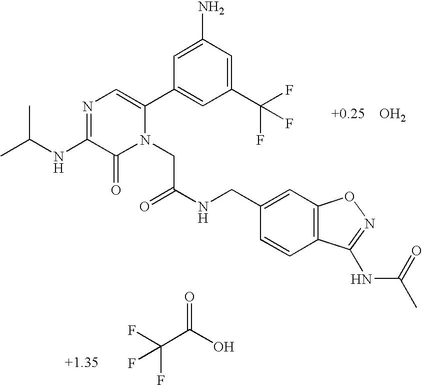 |
| 80 | 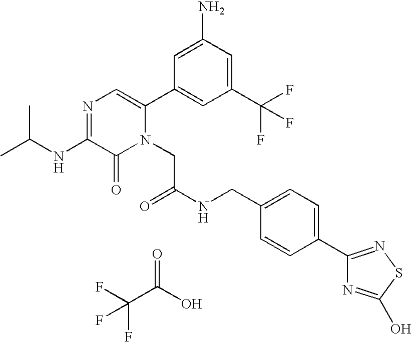 |
| 81 | 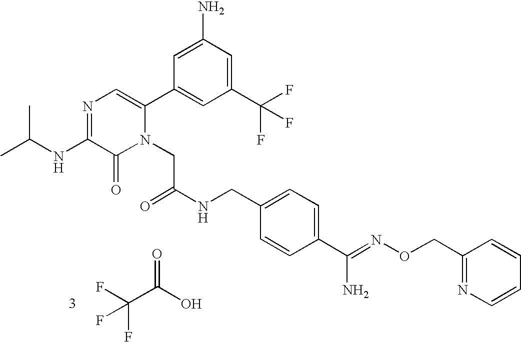 |
| 82 | 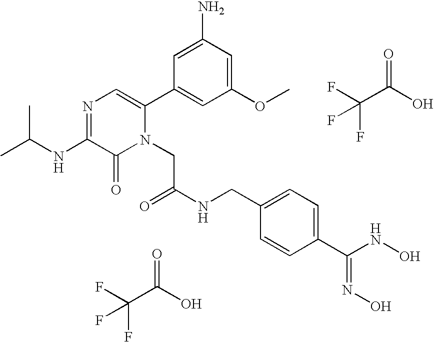 |
| 83 | 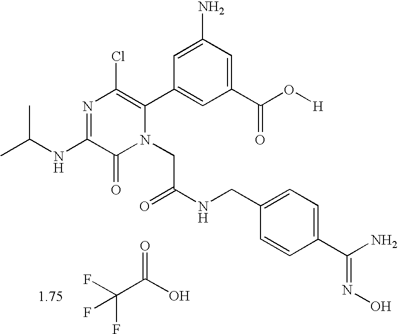 |
| 84 | 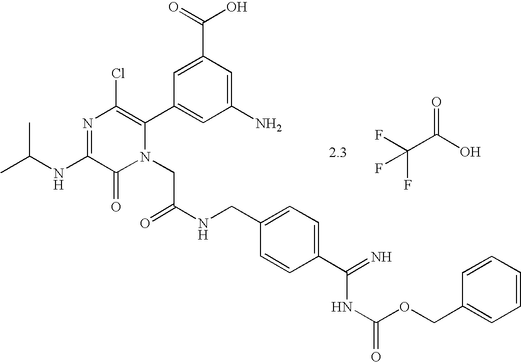 |
| 85 | 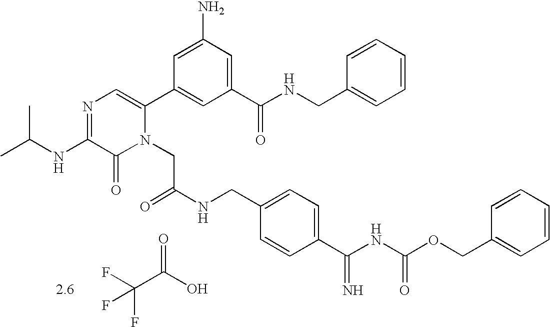 |
| 86 | 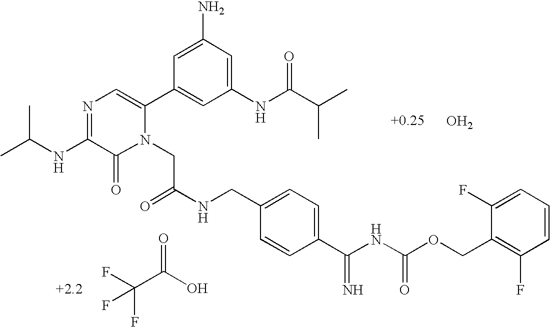 |
| 87 | 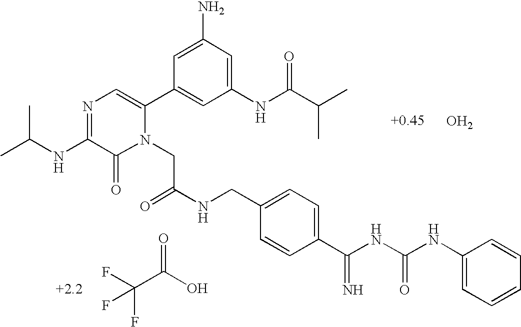 |
| 88 | 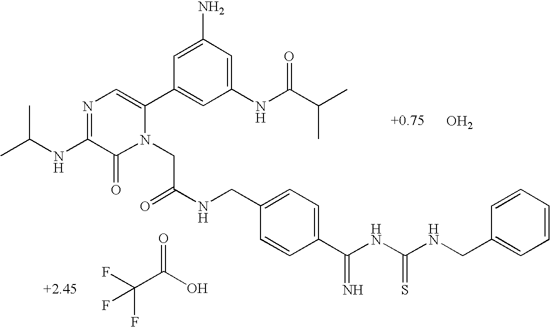 |
| 89 | 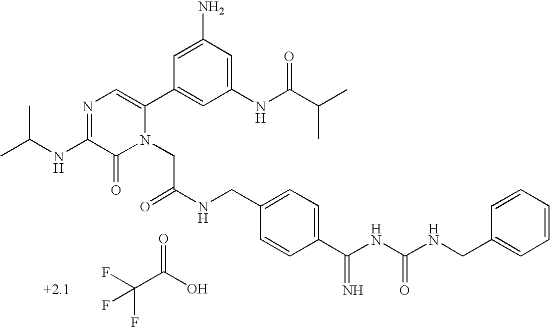 |
| 90 | 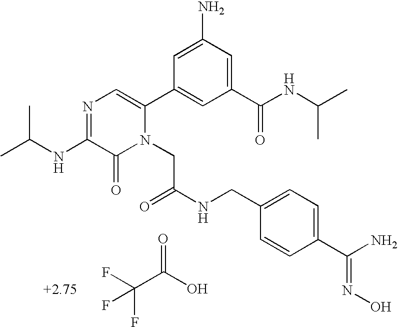 |
| 91 | 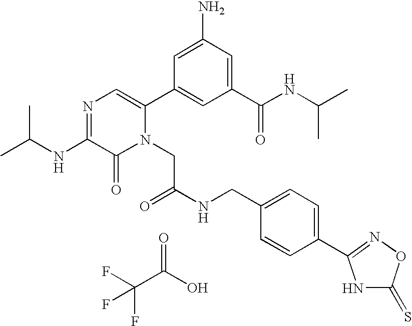 |
| 92 | 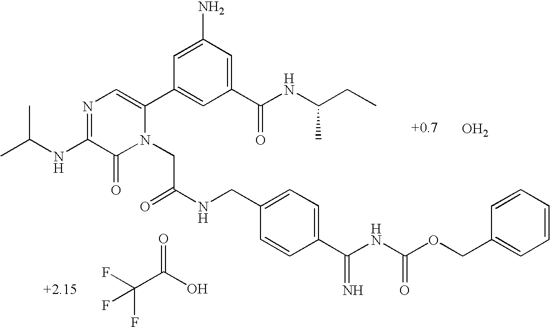 |
| 93 | 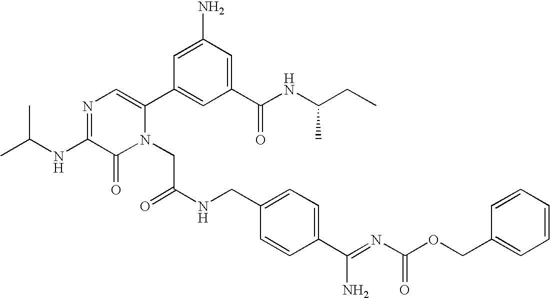 |
| 94 | 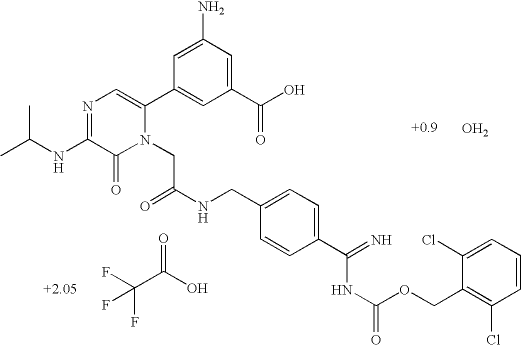 |
| 95 | 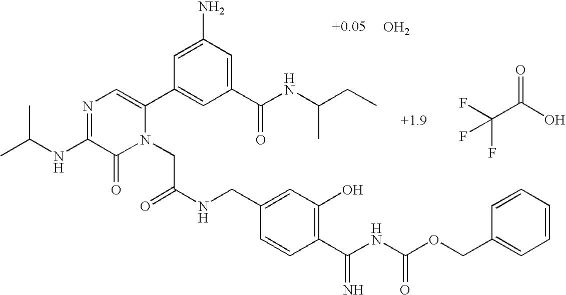 |
| 96 | 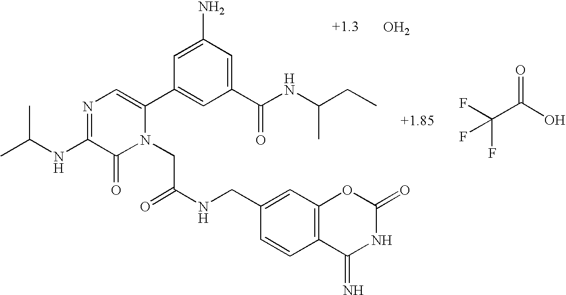 |
| 97 | 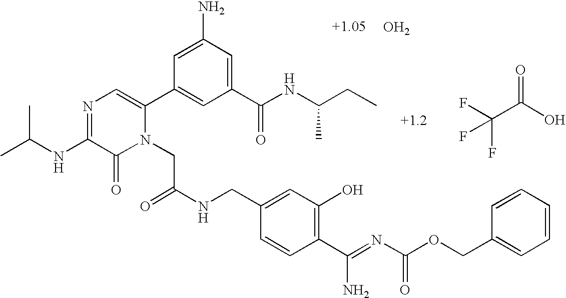 |
| 98 | 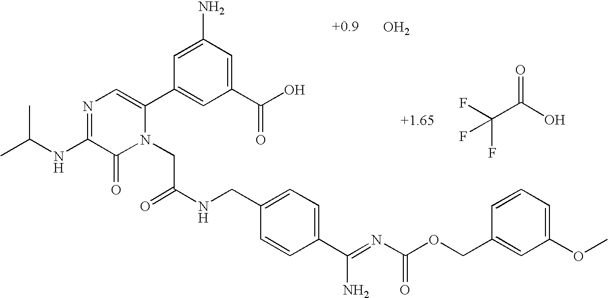 |
| 99 | 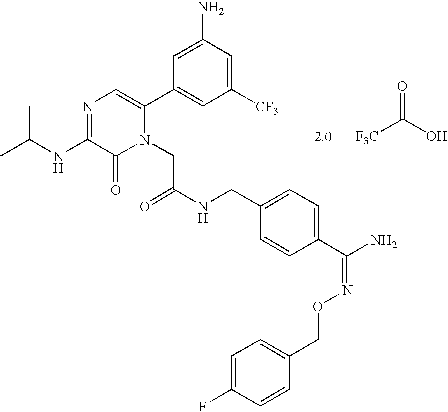 |
| 100 | 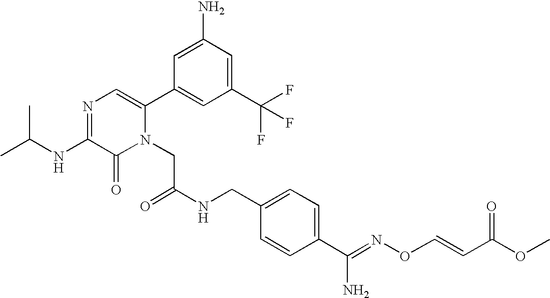 |
| 101 | 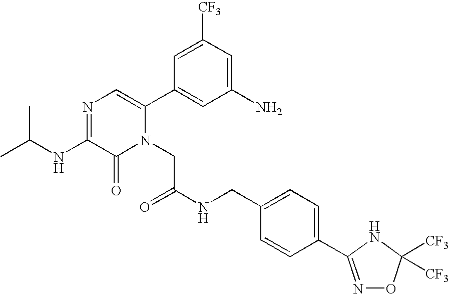 |
| 102 | 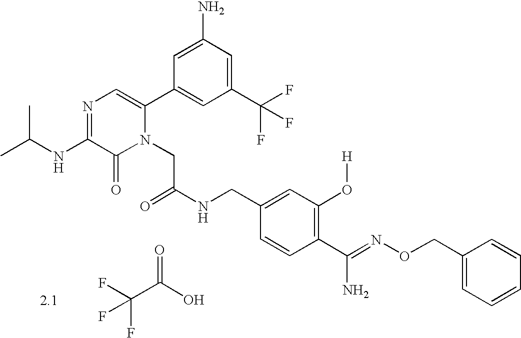 |
| 103 | 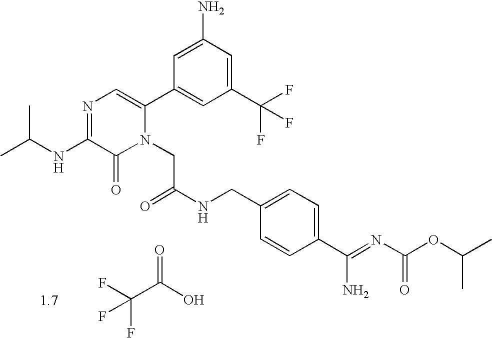 |
| 104 | 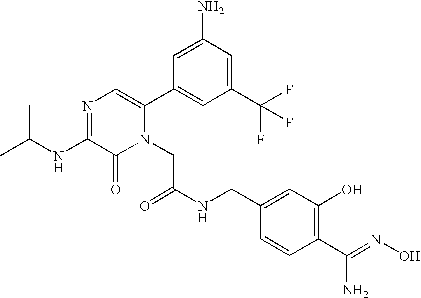 |
| 105 | 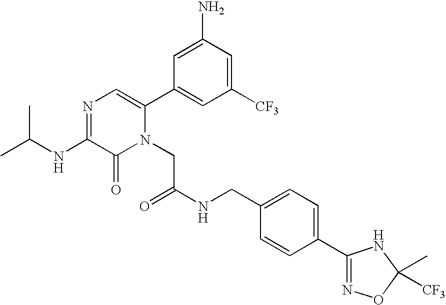 |
| 106 | 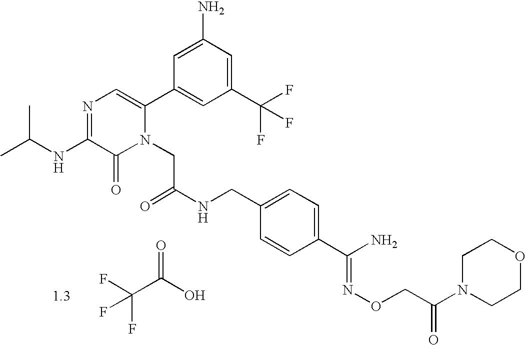 |
| 107 | 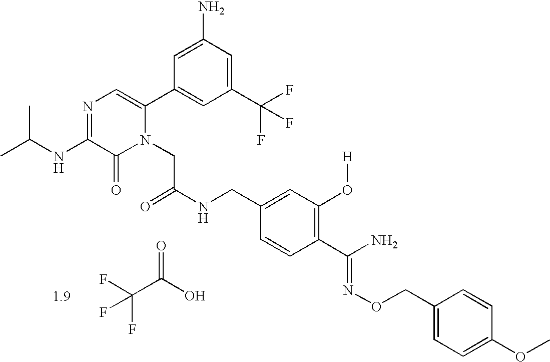 |
| 108 | 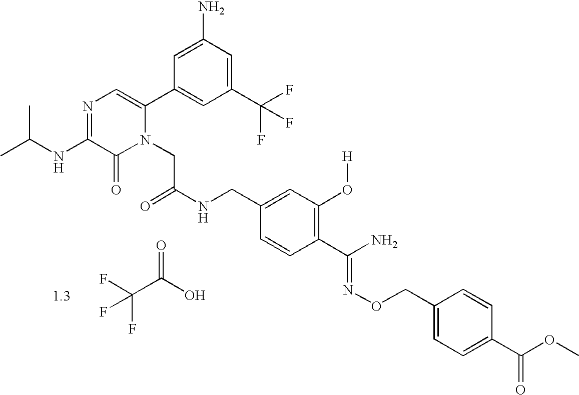 |
| 109 | 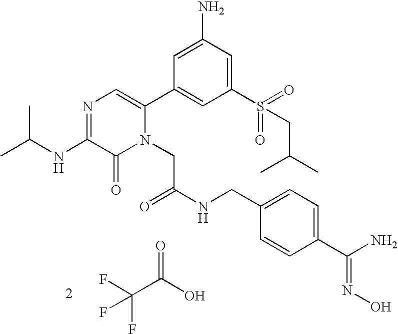 |
| 110 | 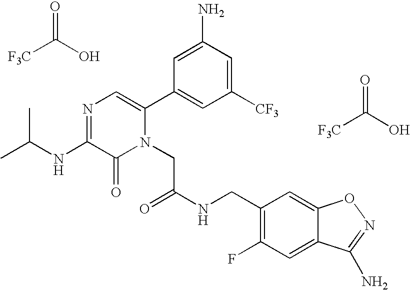 |
| 111 | 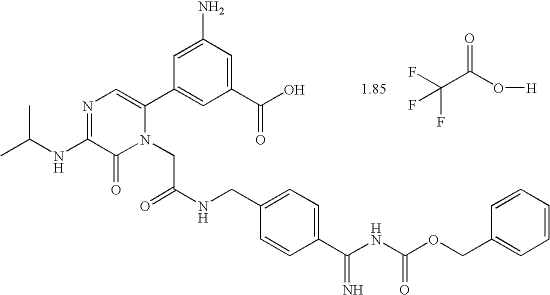 |
| 112 | 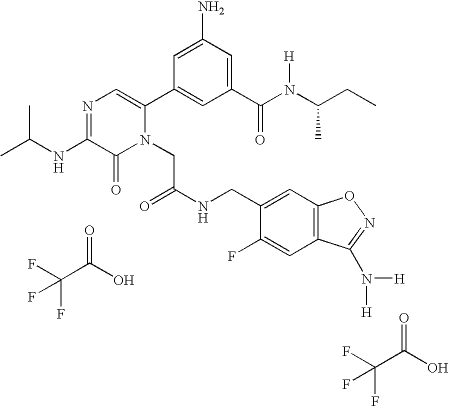 |
| 113 | 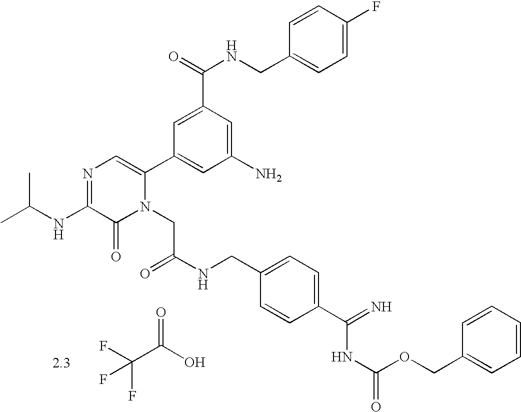 |
| 114 | 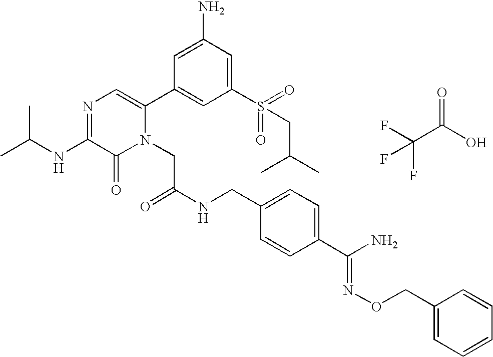 |
| 115 | 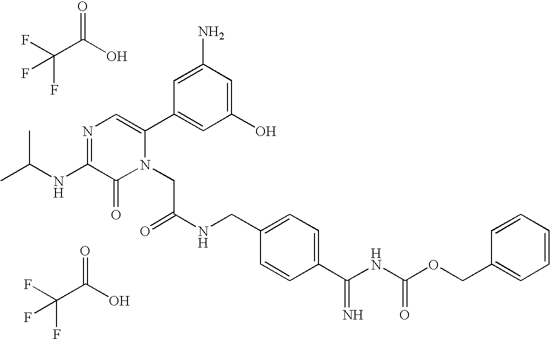 |
| 116 | 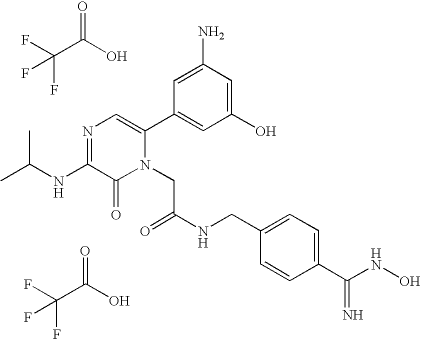 |
| 117 | 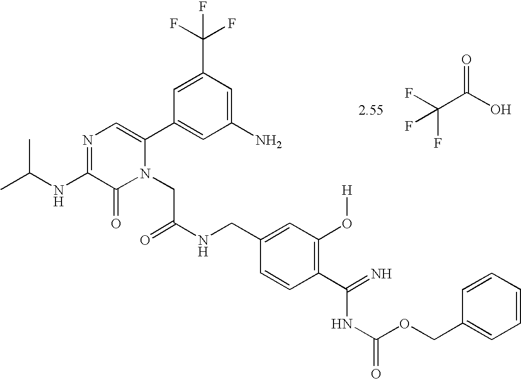 |
| 118 | 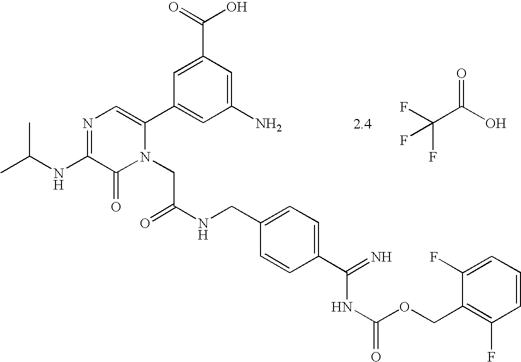 |
| 119 | 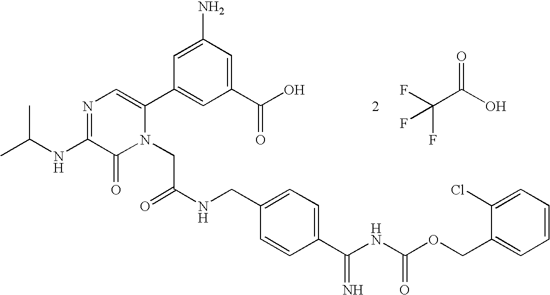 |
| 120 | 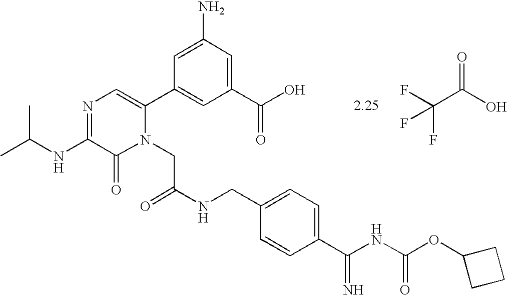 |
| 121 | 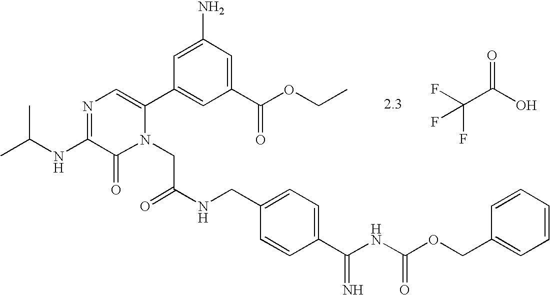 |
| 122 | 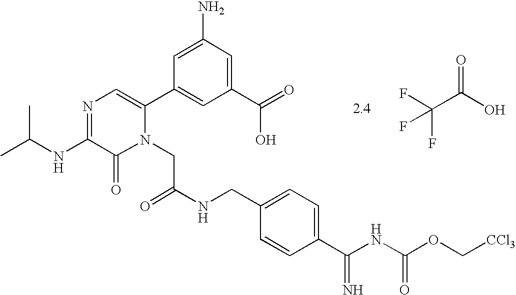 |
| 123 | 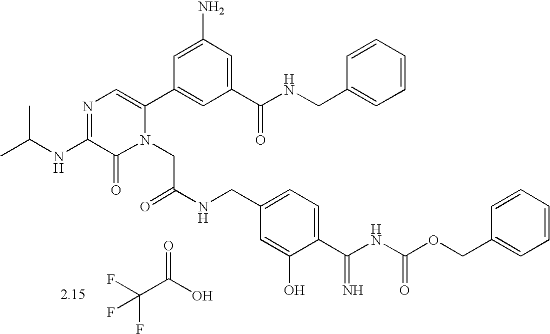 |
| 124 | 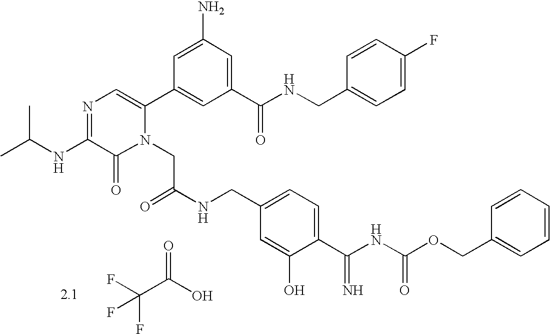 |
| 125 | 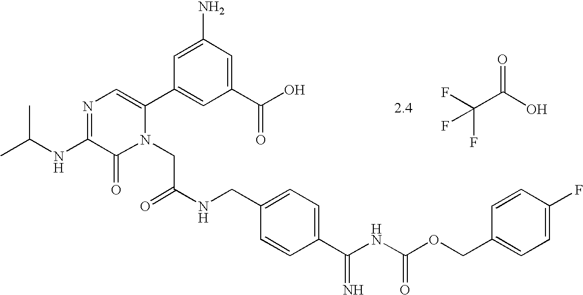 |
| 126 | 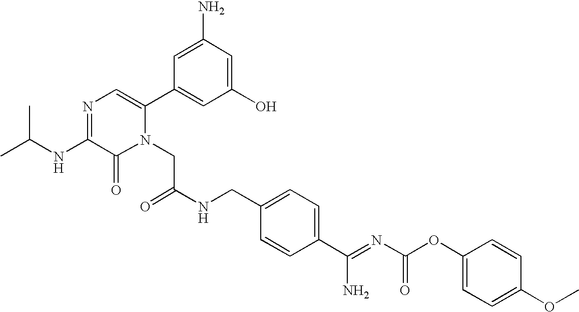 |
| 127 | 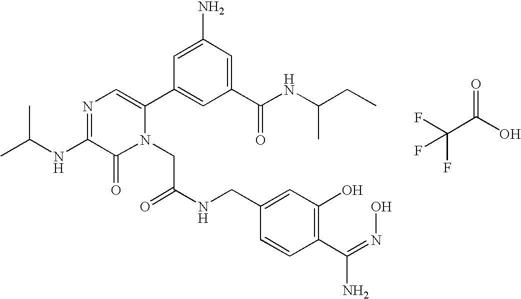 |
| 128 | 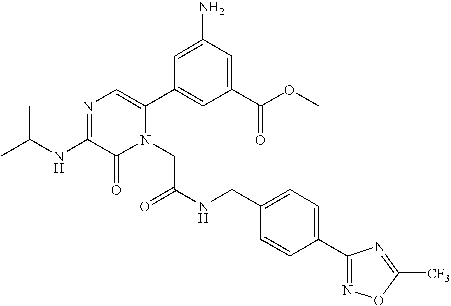 |
| 129 | 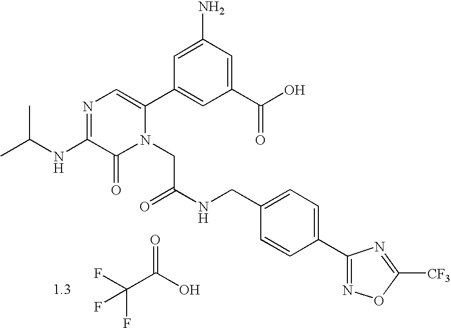 |
| 130 | 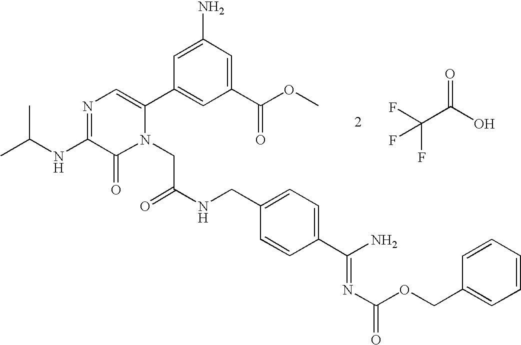 |
| 131 | 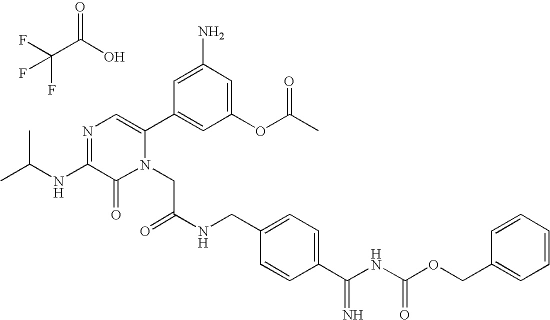 |
| 132 | 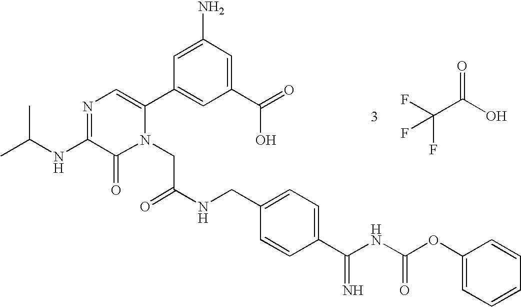 |
| 133 | 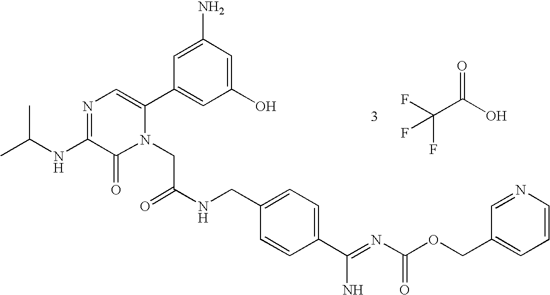 |
| 134 | 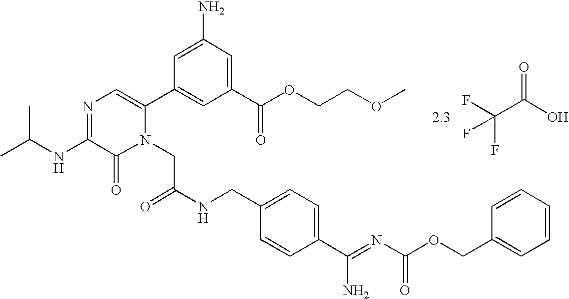 |
| 135 | 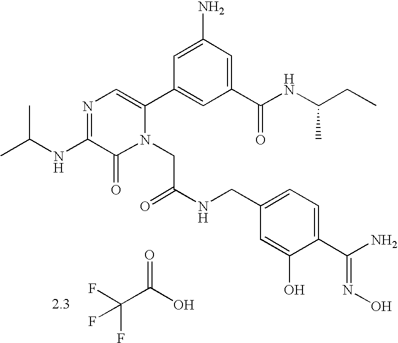 |
| 136 | 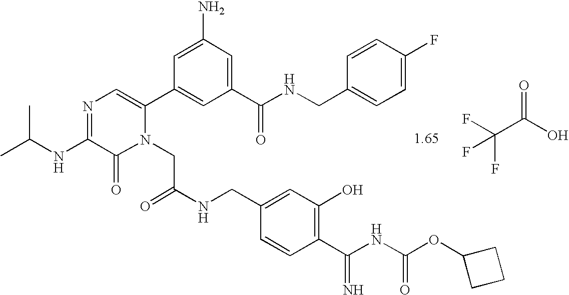 |
| 137 | 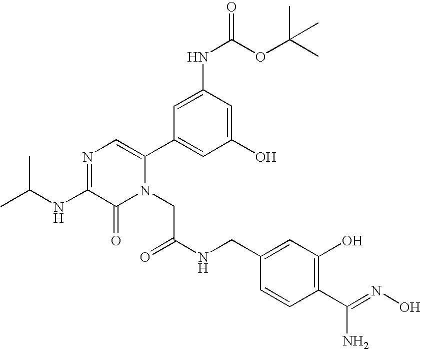 |
| 138 | 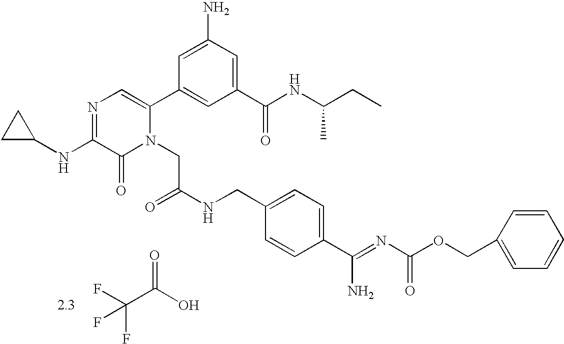 |
| 139 | 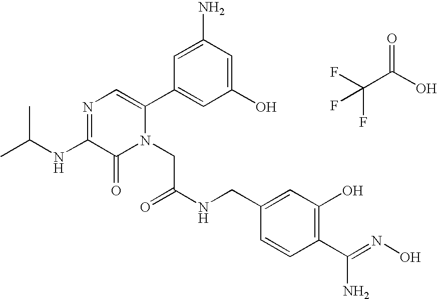 |
| 140 | 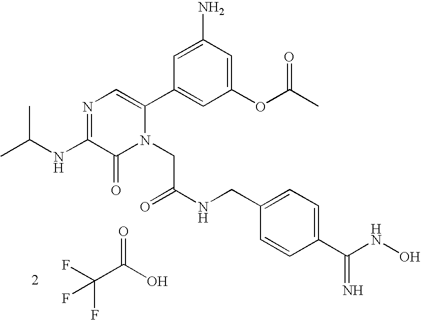 |
| 141 | 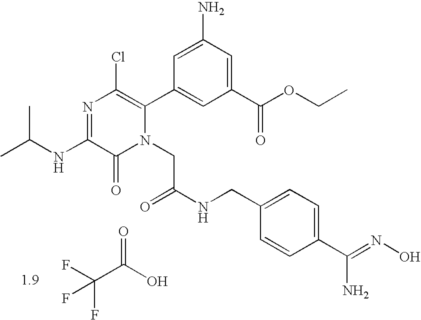 |
| 142 | 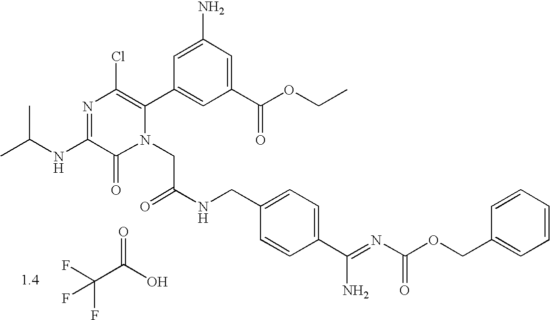 |
| 143 | 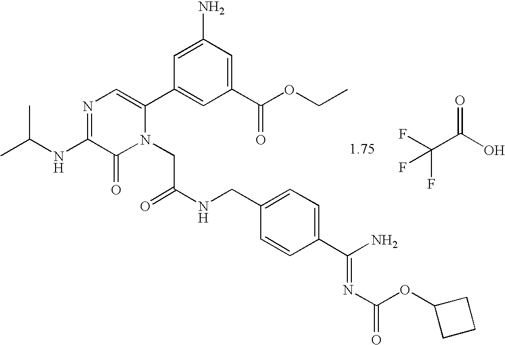 |
| 144 | 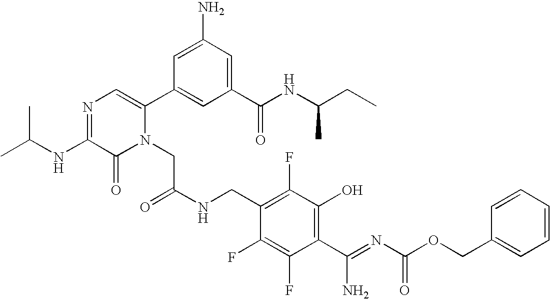 |
| 145 | 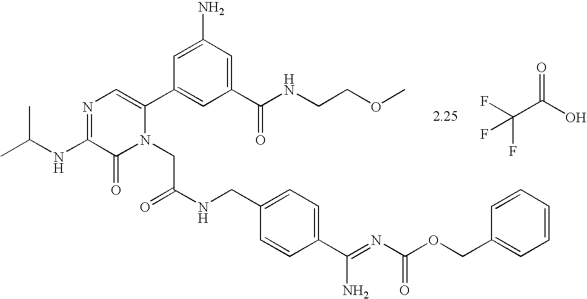 |
| 146 | 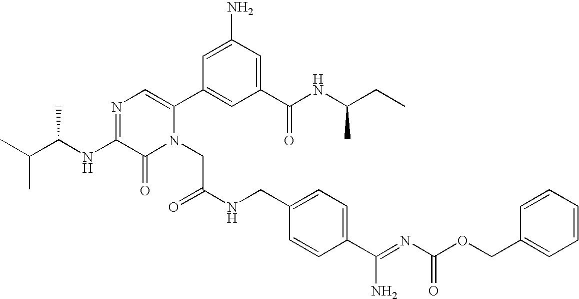 |
| 147 | 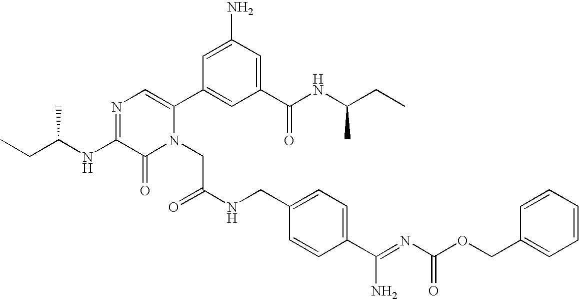 |
| 148 | 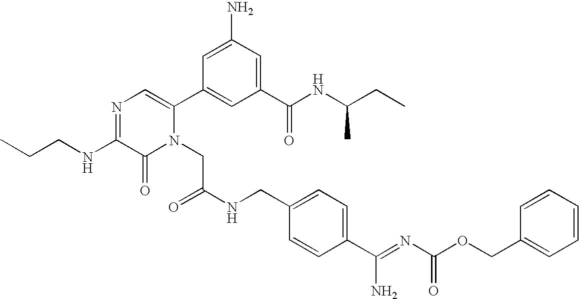 |
| 149 | 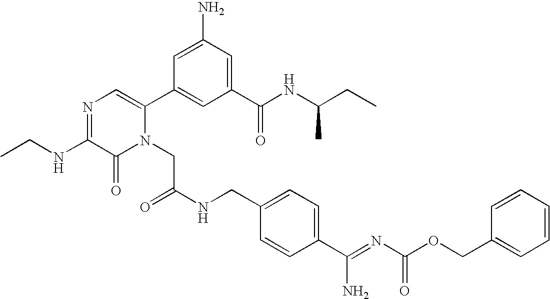 |
| 150 | 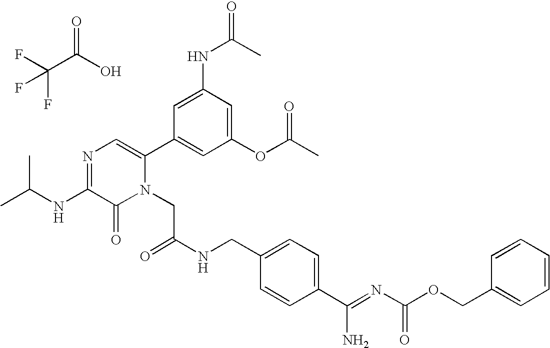 |
| 151 | 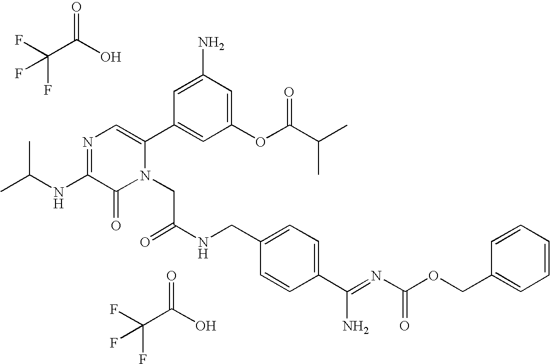 |
| 152 | 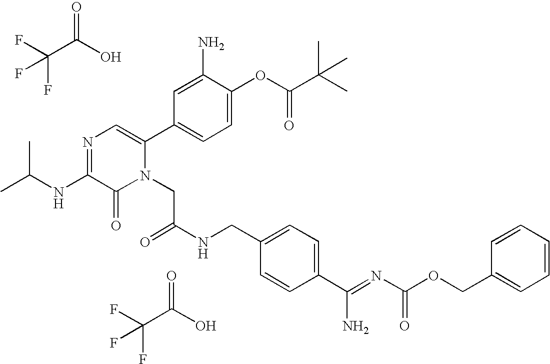 |
| 153 | 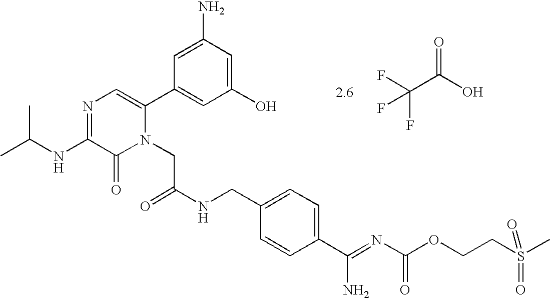 |
| 154 | 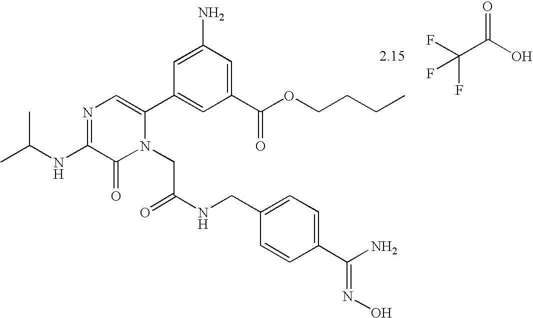 |
| 155 | 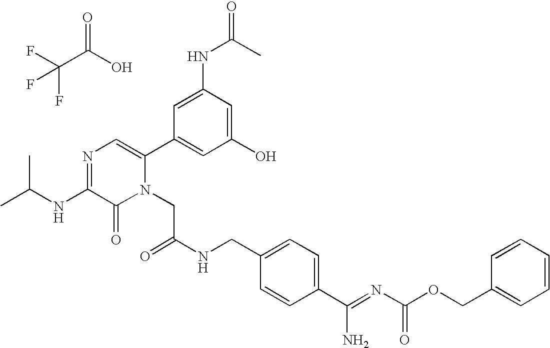 |
| 156 | 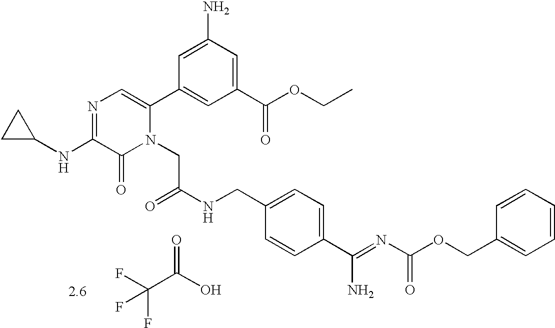 |
| 157 | 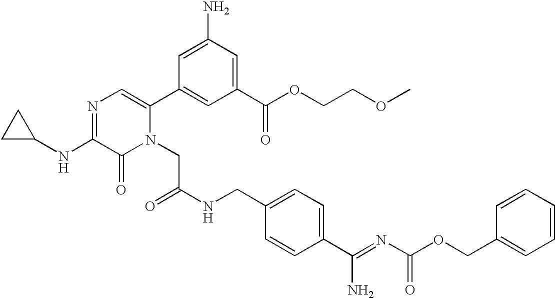 |
| 158 | 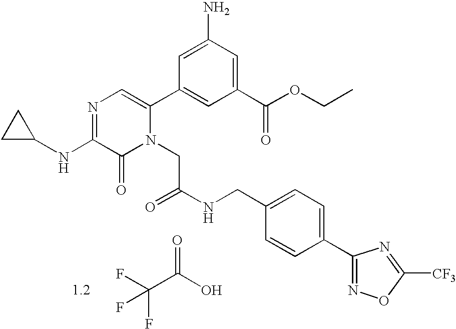 |
| 159 | 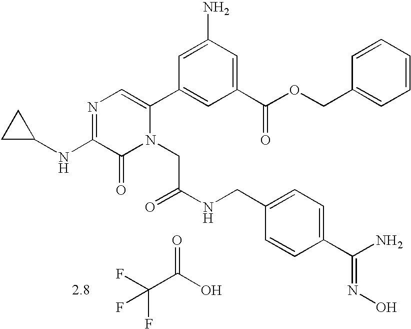 |
| 160 | 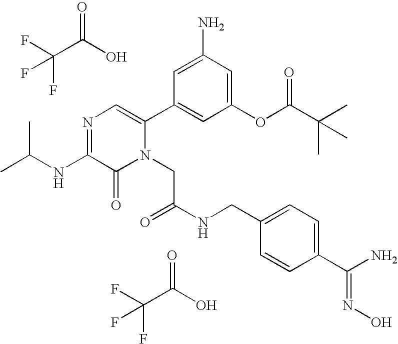 |
| 161 | 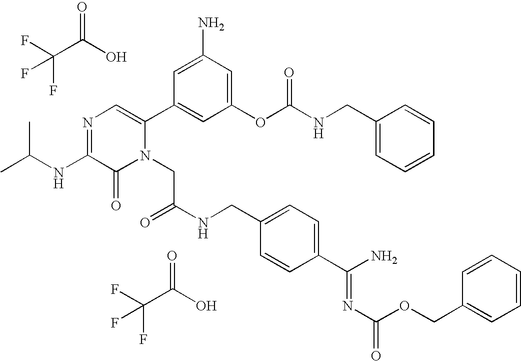 |
| 162 | 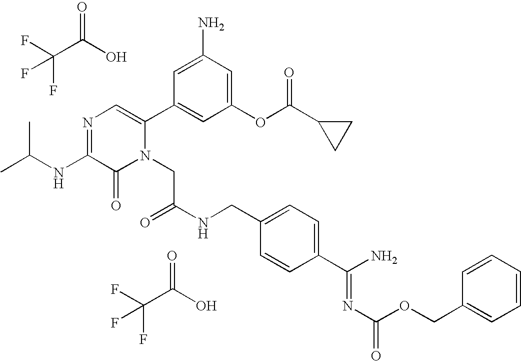 |
| 163 | 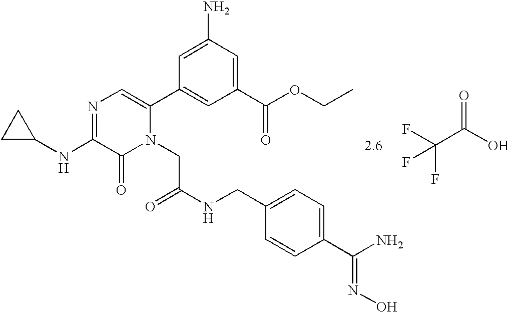 |
| 164 | 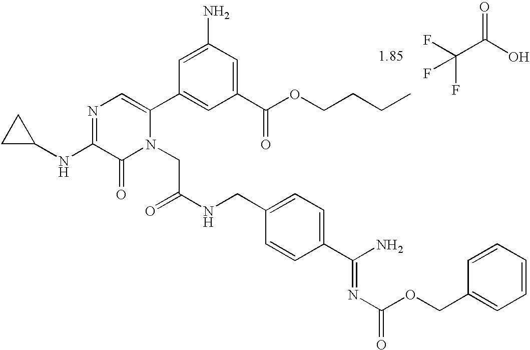 |
| 165 | 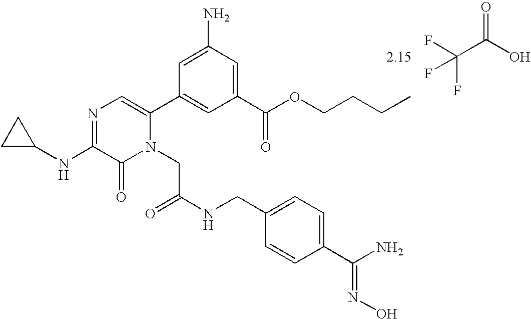 |
| 166 | 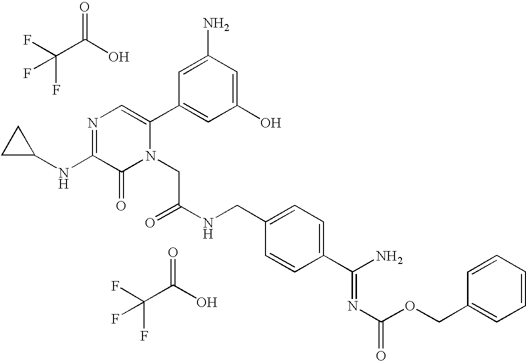 |
| 167 | 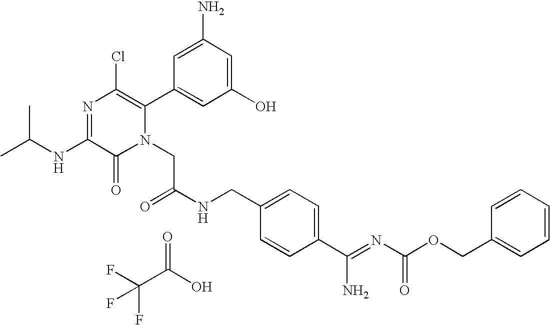 |
| 168 | 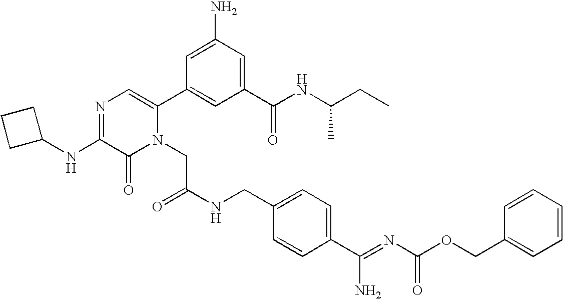 |
| 169 | 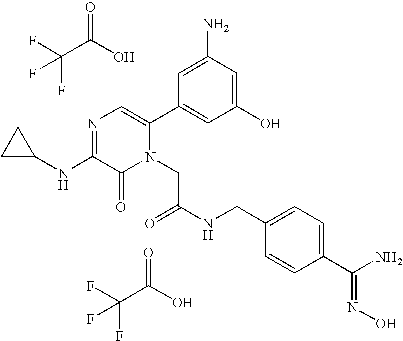 |
| 170 | 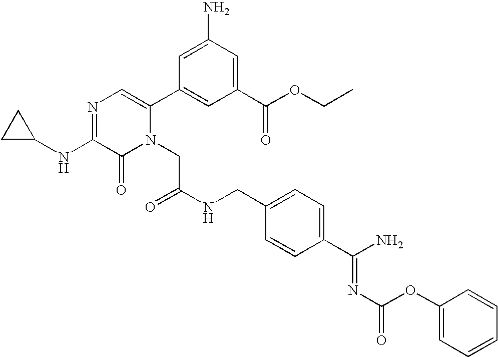 |
| 171 | 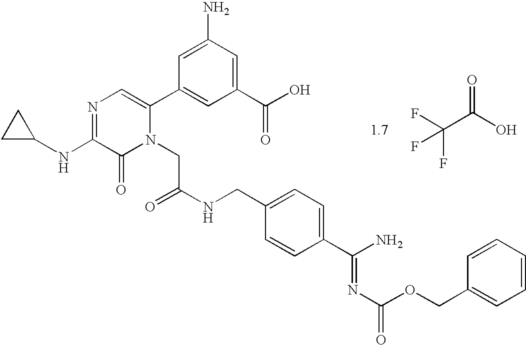 |
| 172 | 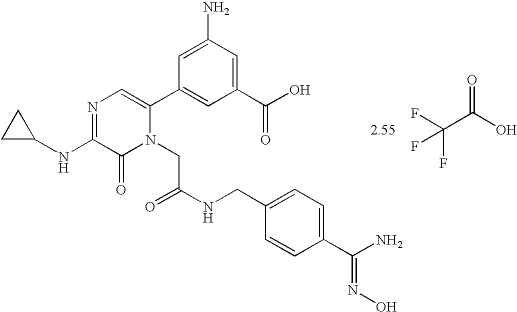 |
| 173 | 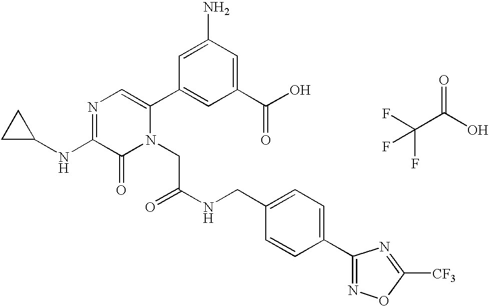 |
| 174 | 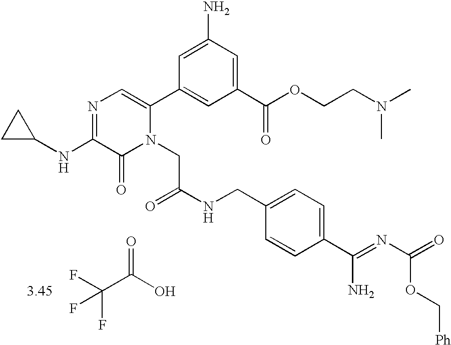 |
| 175 | 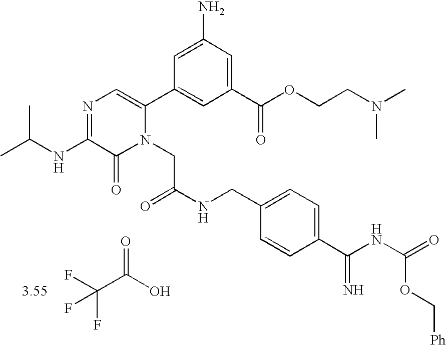 |
| 176 | 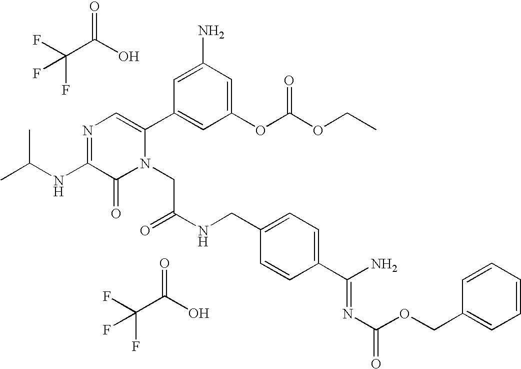 |
| 177 | 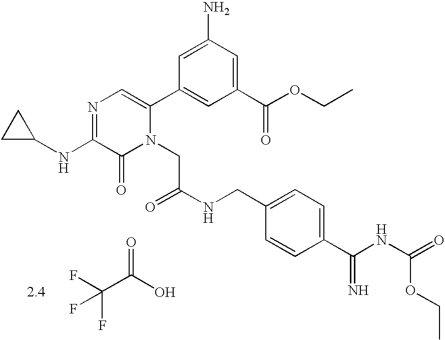 |
| 178 | 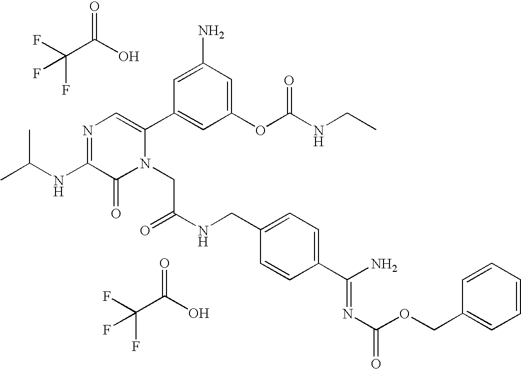 |
| 179 | 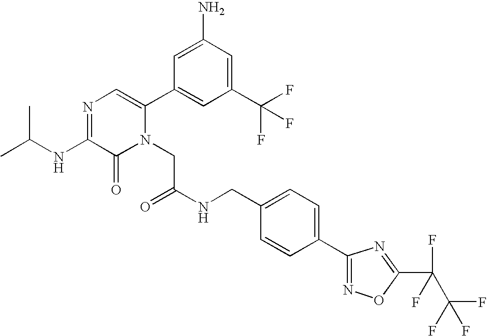 |
| 180 | 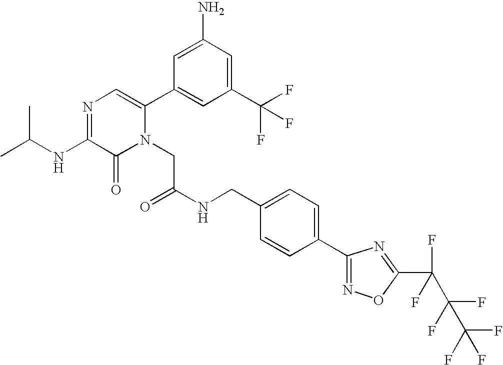 |
| 181 | 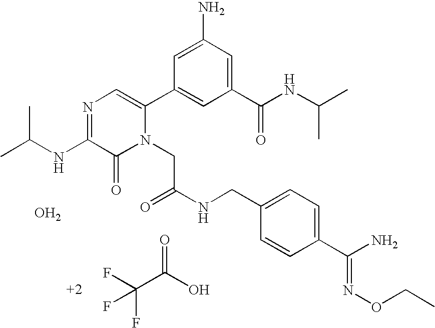 |
| 182 | 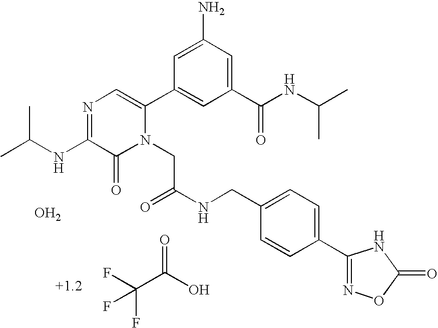 |
| 183 | 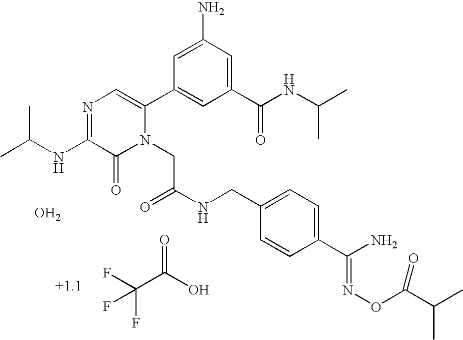 |
| 184 | 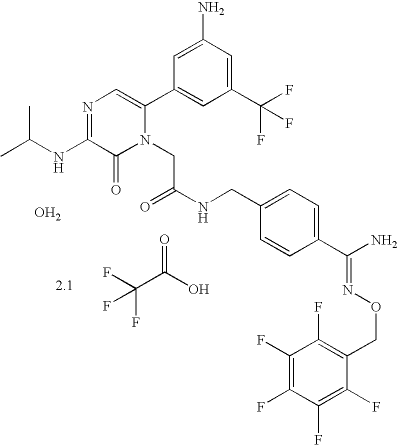 |
| 185 | 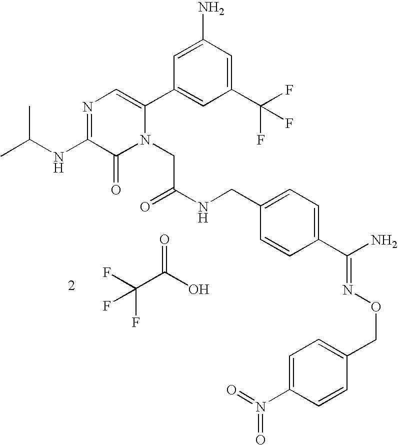 |
| 186 | 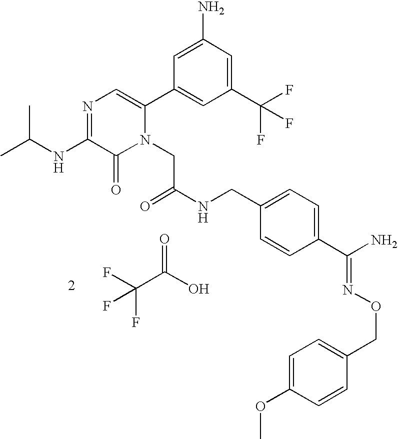 |
| 187 | 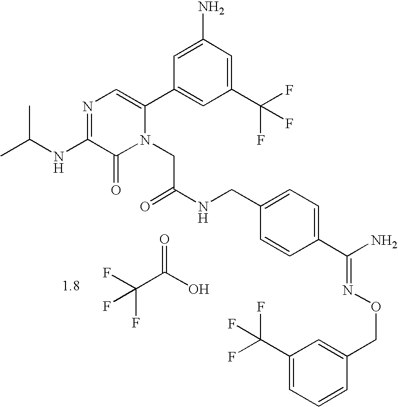 |
| 188 | 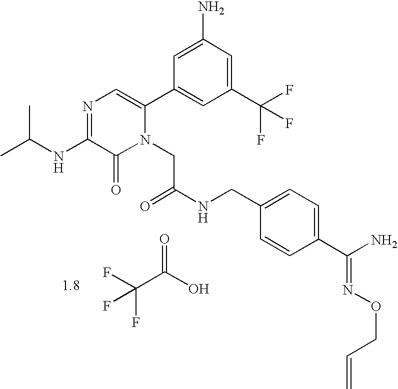 |
| 189 | 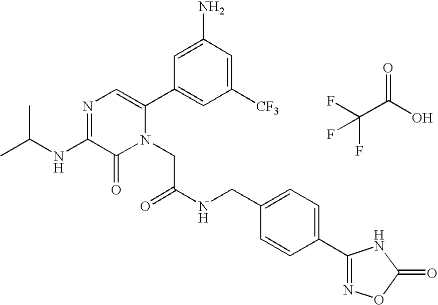 |
| 190 | 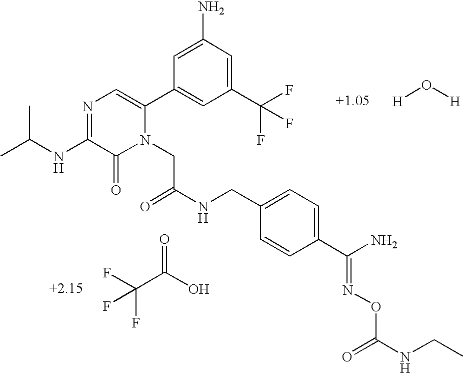 |
| 191 | 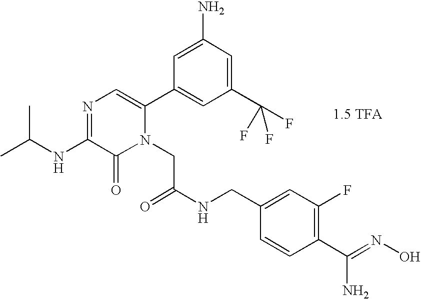 |
| 192 | 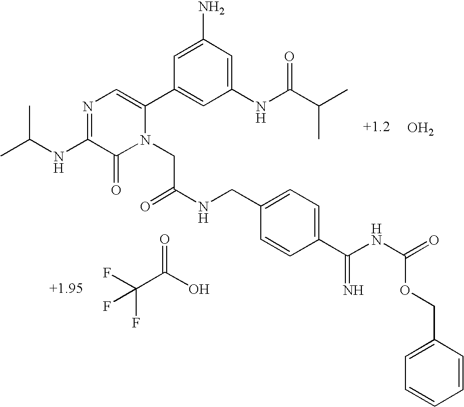 |
| 193 | 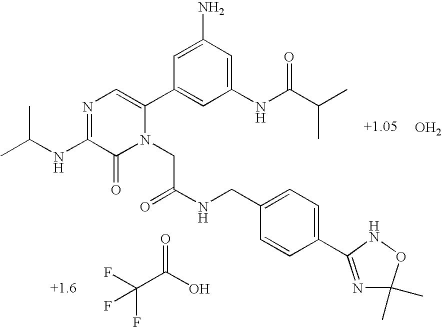 |
| 194 | 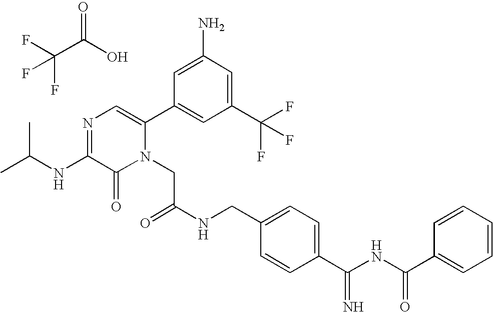 |
| 195 | 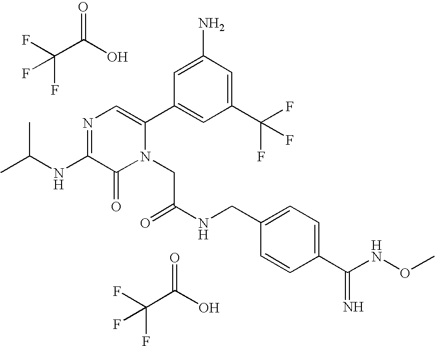 |
| 196 | 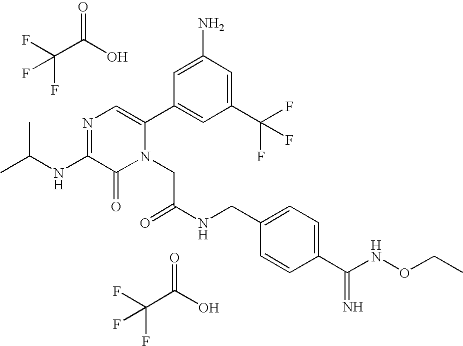 |
| 197 | 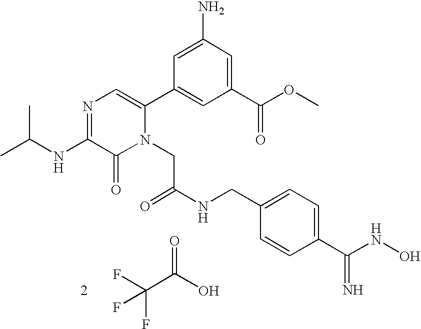 |
| 198 | 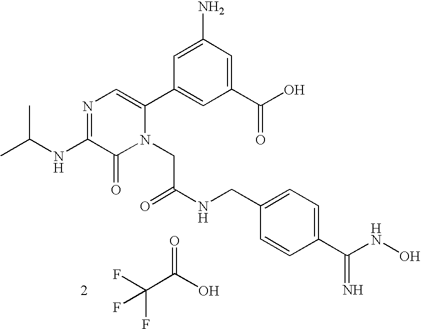 |
| 199 | 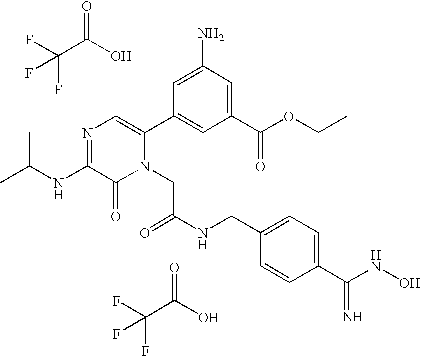 |
| 200 | 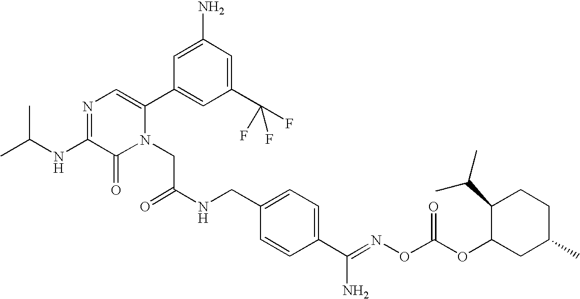 |
| 201 | 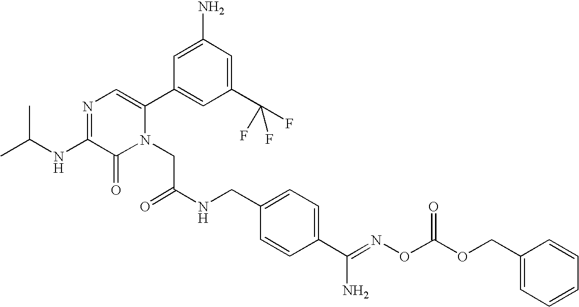 |
| 202 | 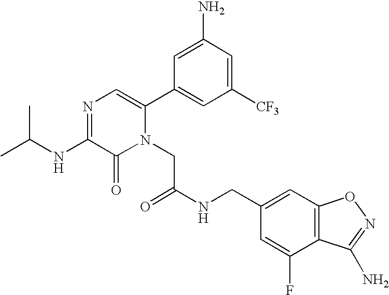 |
| 203 | 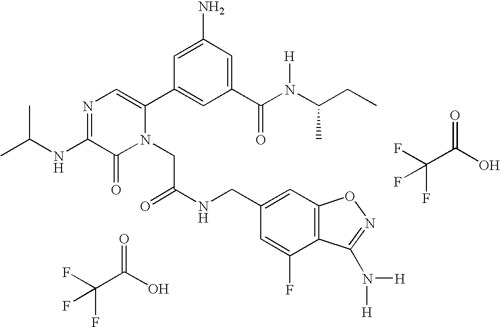 |
| 204 | 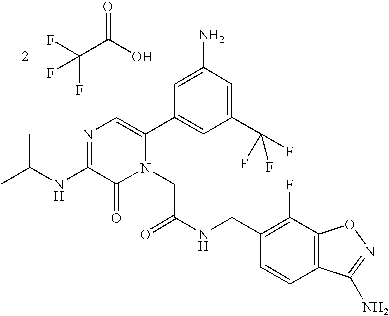 |
| 205 | 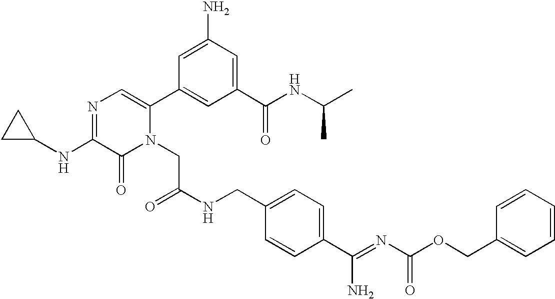 |
| 206 | 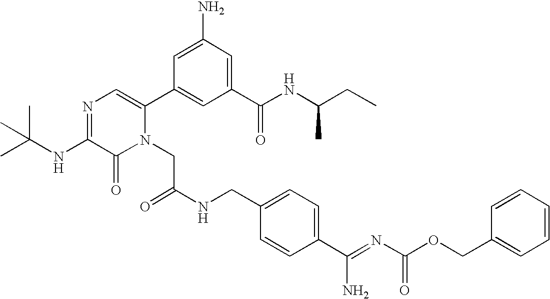 |
| 207 | 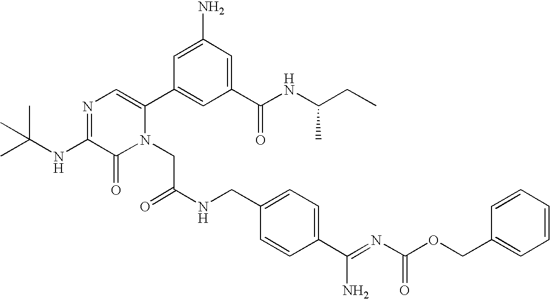 |
| 208 | 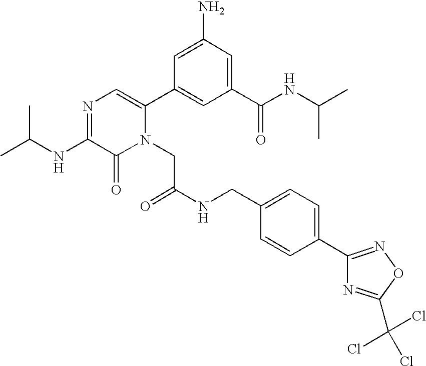 |
| 209 | 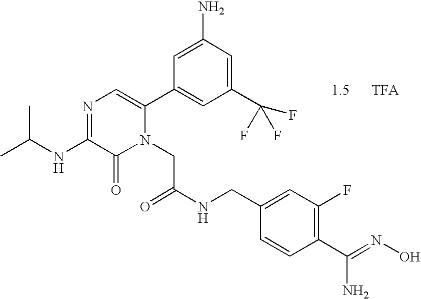 |

Any compound corresponding to any of formulas (I)-(V), having one or more prodrug moieties as part of the molecule, can be converted under physiological conditions to the biologically active drug by a number of chemical and biological mechanisms. In general terms, these prodrug conversion mechanisms are hydrolysis, reduction, oxidation, and elimination. For illustrative purposes, the following paragraphs detail prodrugs in which the prodrug moiety is covalently bonded to the amidine group on Z₃ as depicted embodiments for each of formulas (I)-(V) above.

In one embodiment, conversion of the prodrug to the biologically active drug can be accomplished by hydrolysis of the prodrug moiety provided the prodrug moiety is chemically or enzymatically hydrolyzable with water. The reaction with water typically results in removal of the prodrug moiety and liberation of the biologically active drug. By way of example, a hydrolyzable prodrug derivative at the amidine group may be a carbonyl derivative such as N-acyl. Hydrolysis results in freeing the amidine group of the drug by removal of the acyl as carbon acid. Other suitable hydrolyzable prodrug derivatives include carbonyl, thiocarbonyl, imine, enamine, and oxygenated sulfur.

Yet another aspect of the invention provides, conversion of the prodrug to the biologically active drug by reduction of the prodrug moiety. Typically in this embodiment, the prodrug moiety is reducible under physiological conditions in the presence of a reducing enzymatic process. The reduction preferably results in removal of the prodrug moiety and liberation of the biologically active drug. An example of a reducible prodrug derivative at the amidine group is an oxygen containing group in which an oxygen is directly attached to the amidine. Reduction results in freeing the amidine group of the drug by removal of oxygen as water or an alcohol. Generally speaking, other suitable reducible prodrug derivatives include a nitrogen containing group, and a sulfur containing group, provided both nitrogen and sulfur are each preferably in their most reduced state.

In another embodiment, conversion of the prodrug to the biologically active drug can also be accomplished by oxidation of the prodrug moiety. Typically in this embodiment, the prodrug moiety is oxidizable under physiological conditions in the presence of an oxidative enzymatic process. The oxidation preferably results in removal of the prodrug moiety and liberation of the biologically active drug. An example of an oxidizable prodrug derivative at the amidine group is a hydrocarbyl containing unsaturation in the carbon beta to the carbon directly connected to the amidine group. Oxidation results in forming an oxygenated intermediate that breaks down, thereby freeing the amidine group of the drug with concurrent hydrolysis of the oxygenated hydrocarbyl residue. Other suitable oxidizable prodrug derivatives of the amidine include saturated hydrocarbyl, unsaturated substituted hydrocarbyl, aryl, and aralkyl.

A further aspect of the invention encompasses conversion of the prodrug to the biologically active drug by elimination of the prodrug moiety. Generally speaking, in this embodiment the prodrug moiety is removed under physiological conditions with a chemical or biological reaction. The elimination results in removal of the prodrug moiety and liberation of the biologically active drug. By way of example, an eliminateable prodrug derivative at the amidine group is a hydrocarbyl containing an unsaturated electron withdrawing group bonded to the carbon beta to the carbon directly connected to the amidine. More specifically, for illustration purposes and exemplification, the hydrocarbyl group could have a cyano group beta to the carbon directly bonded to the amidino group. Elimination results in the freeing of the amidine group of the drug with concurrent removal of the unsaturated hydrocarbyl residue derived from the prodrug moiety. Other suitable eliminateable prodrug derivatives of the amidine include a hydrocarbyl substituted at the beta carbon with carbonyl, alkoxycarbonyl, amidocarbonyl, nitro, or sulfonyl or an alkyl group substituted with oxygen, nitrogen or sulfur at the carbon directly bonded to the amidine group.

Any compound of the present invention corresponding to formulas (I)-(V) may undergo any combination of the above detailed mechanisms to convert the prodrug to the biologically active compound. For example, a particular compound may undergo hydrolysis, oxidation, elimination, and reduction to convert the prodrug to the biologically active compound. Equally, a particular compound may undergo only one of these mechanisms to convert the prodrug to the biologically active compound.

The compounds of the present invention can exist in tautomeric, geometric or stereoisomeric forms. The present invention contemplates all such compounds, including cis- and trans-geometric isomers, E- and Z-geometric isomers, R- and S-enantiomers, diastereomers, d-isomers, l-isomers, the racemic mixtures thereof and other mixtures thereof, as falling within the scope of compounds having any of formulas (I)-(V). Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“sis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures or R and S forms for each stereocenter present.

Also included in the family of compounds having any of formulas (I)-(V) are the pharmaceutically-acceptable salts thereof. The term “pharmaceutically-acceptable salt” embraces salts commonly used to form alkali metal salts and to form addition salts of free acids or free bases. The nature of the salt is not critical, provided that it is pharmaceutically acceptable. Suitable pharmaceutically-acceptable acid addition salts of the compounds may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethylsulfonic, benzenesulfonic, sulfanilic, stearic, cyclohexylaminosulfonic, algenic, and galacturonic acid. Suitable pharmaceutically-acceptable base addition salts of the compounds include metallic salts made from aluminum, calcium, lithium, magnesium, potassium, sodium and zinc or organic salts made from N,N′-dibenzylethyleneldiamine, choline, chloroprocaine, diethanolamine, ethylenediamine, meglumine (N-methylglucamine) and procain. All of these salts may be prepared by conventional means from the corresponding compound by reacting, for example, the appropriate acid or base with the selected compound of any of formulas (I)-(V).

The present invention also comprises a pharmaceutical composition comprising a therapeutically-effective amount of the compound of the invention in association with at least one pharmaceutically-acceptable carrier, adjuvant or diluent. Pharmaceutical compositions of the present invention can comprise the active compounds of formulas (I)-(V) in association with one or more non-toxic, pharmaceutically-acceptable carriers and/or diluents and/or adjuvants (collectively referred to herein as “carrier” materials) and, if desired, other active ingredients. The active compounds of the present invention may be administered by any suitable route, preferably in the form of a pharmaceutical composition adapted to such a route, and in a dose effective for the treatment intended.

The active compounds and composition may, for example, be administered orally, intravascularly, intraperitoneally, subcutaneously, intramuscularly, oculary, or topically. For treating ocular build up of fibrin, the compounds may be administered intraocularly or topically as well as orally or parenterally.

The compounds can be administered in the form of a depot injection or implant preparation which may be formulated in such a manner as to permit a sustained release of the active ingredient. The active ingredient can be compressed into pellets or small cylinders and implanted subcutaneously or intramusculary as depot injections or implants. Implants may employ inert materials such as biodegradable polymers or synthetic silicones, for example, silastic, silicone rubber or other silicon containing polymers.

Moreover, the compounds can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxy-propyl-methacrylamide-phenol, polyhydroxyethyl-aspartamide-phenol, or ployethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphitpathic block copolymers of hydrogels.

For oral administration, the pharmaceutical composition may be in the form of, for example, tablets, capsules (each of which includes sustained release or timed release formulations), pills, powders, granules, elixers, tinctures, suspensions, liquids including syrups, and emulsions. The pharmaceutical composition is preferably made in the form of a dosage unit containing a particular amount of the active ingredient. Examples of such dosage units are tablets or capsules. The active ingredient may also be administered by injection as a composition wherein, for example, saline, dextrose or water may be used as a suitable carrier.

The amount of therapeutically active compounds which are administered and the dosage regimen for treating a disease condition with the compounds and/or compositions of this invention depends on a variety of factors, including the age, weight, sex and medical condition of the subject, the severity of the disease, the route and frequency of administration, and the particular compound employed, and thus may vary widely.

The pharmaceutical compositions may contain active ingredients in the range of about 0.1 to 2000 mg, and preferably in the range of about 0.5 to 500 mg. A daily dose of about 0.01 to 100 mg/kg body weight, and preferably between about 0.5 and about 20 mg/kg body weight, may be appropriate. The daily dose can be administered in one to four doses per day.

The compounds may be formulated in topical ointment or cream, or as a suppository, containing the active ingredients in a total amount of, for example, 0.075 to 30% w/w, preferably 0.2 to 20% w/w and most preferably 0.4 to 15% w/w. When formulated in an ointment, the active ingredients may be employed with either paraffinic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane.

The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as diisoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

For therapeutic purposes, the active compounds of the present invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

As a further embodiment, compounds having formula (I)-(V) or a pharmaceutically-acceptable salt thereof comprise a treatment and prophylaxis for thrombotic events resulting from coronary artery disease, cerebrovascular disease and other coagulation cascade related disorders in a subject. The treatment comprises administering to the subject having such disorder a therapeutically-effective amount of compounds having formulas (I)-(V) or a pharmaceutically-acceptable salt thereof.

In another aspect of the invention, the compounds or a pharmaceutically-acceptable salt thereof can also be used whenever inhibition of blood coagulation is required such as to prevent coagulation of stored whole blood and to prevent coagulation in other biological samples for testing or storage. Thus coagulation inhibitors of the present invention can be added to or contacted with stored whole blood and any medium containing or suspected of containing plasma coagulation factors and in which it is desired that blood coagulation be inhibited, e.g. when contacting the mammal's blood with material selected from the group consisting of vascular grafts, stents, orthopedic prothesis, cardiac prosthesis, and extracorporeal circulation systems.

Compounds of Formula (I)-(V) are capable of inhibiting activity of serine proteases related to the coagulation cascade. Thus, these compounds could be used in the manufacture of a medicament as a method for the prophylactic or therapeutic treatment of diseases mediated by coagulation cascade serine proteases, such as inhibiting the formation of blood platelet aggregates, inhibiting the formation of fibrin, inhibiting thrombus formation, and inhibiting embolus formation in a mammal, in blood, in blood products, and in mammalian organs. The compounds also can be used for treating or preventing unstable angina, refractory angina, myocardial infarction, transient ischemic attacks, atrial fibrillation, thrombotic stroke, embolic stroke, deep vein thrombosis, disseminated intravascular coagulation, ocular build up of fibrin, and reocclusion or restenosis of recanalized vessels in a mammal. Moreover, the compounds also can be used to study the mechanism of action of coagulation cascade serine proteases to enable the design of better inhibitors and development of better assay methods. The compounds would be also useful in prevention of cerebral vascular accident (CVA) or stroke.

In practicing the methods of the present invention for the treatment and prevention of a variety of thrombotic conditions including coronary artery and cerebrovascular disease, the compounds and pharmaceutical compositions are administered alone or in combination with one another, or in combination with other therapeutics or in vivo diagnostic agents. In another aspect, the compounds can also be co-administered with suitable anti-platelet agreggation agents, including, but not limited to aspirin, ticlopidine, or clopidrogel, fibrinogen receptor antagonists (e.g. to treat or prevent unstable angina or to prevent reocculsion after angioplasty and restenosis), anti-coagulants such as aspirin, warfarin or heparins, thrombolytic agents such as plasminogen activators or streptokinase to achieve synergistic effects in the treatment of various pathologies, lipid lowering agents including antihypercholesterolemics (e.g. HMG CoA reductase inhibitors such as mevastatin, lovastatin, simvastatin, pravastatin, and fluvastatin, HMG CoA synthatase inhibitors, etc.), anti-diabetic drugs, or other cardiovascular agents (e.g. loop diuretics, thiazide type diuretics, nitrates, aldosterone antagonistics (e.g. spironolactone and epoxymexlerenone), angiotensin converting enzyme (e.g. ACE) inhibitors, angiotensin II receptor antagonists, beta-blockers, antiarrythmics, anti-hypertension agents, and calcium channel blockers to treat or prevent atheriosclerosis. By way of example, patients suffering from coronary artery disease, and patients subjected to angioplasty procedures, would benefit from coadministration of fibrinogen receptor antagonists and coagulation cascade inhibitors of the present invention. Also, coagulation cascade inhibitors could enhance the efficiency of tissue plasminogen activator-mediated thrombolytic reperfusion.

Typical doses of compounds of the present invention with other suitable anti-platelet agents, anticoagulation agents, cardiovascular therapeutic agents, or thrombolytic agents may be the same as those doses of coagulation cascade inhibitors administered without coadministration of additional anti-platelet agents, anticoagulation agents, cardiovascular therapeutic agents, or thrombolytic agents, or may be substantially less than those doses of coagulation cascade inhibitors administered without coadministration of additional anti-platelet agents, anticoagulation agents, cardiovascular therapeutic agents, or thrombolytic agents, depending on a patient's therapeutic needs.

The present methods preferably employ prodrug compounds that when converted to the biologically active compound selectively inhibit human TF-VIIA over the inhibition of both human Thrombin II and human factor Xa. Preferably, the compounds have a human TF-VIIA IC₅₀ of less than 0.5 mM and also have a selectivity ratio of TF-VIIA inhibition over both human Thrombin II and human factor Xa inhibition of at least 10, and more preferably at least 100. Even more preferably, the compounds have a human TF-VIIA IC₅₀of less than 0.1 mM and also have a selectivity ratio of TF-VIIA inhibition over both human Thrombin II and human factor Xa inhibition of at least 1000, and most preferably at least 10,000.

All mentioned references are incorporated by reference as if here written.

Although this invention has been described with respect to specific embodiments, the details of these embodiments are not to be construed as limitations. The following examples are provided to illustrate the present invention and are not intended to limit the scope thereof. Without further elaboration, it is believed that one skilled in the art can, using the preceding descriptions, utilize the present invention to its fullest extent. Therefore, the following preferred specific embodiments are to be construed as merely illustrative and not limitative of the remainder of the disclosure in any way whatsoever. Compounds containing multiple variations of the structural modifications illustrated in the schemes or the following Examples are also contemplated. Those skilled in the art will readily understand that known variations of the conditions and processes of the following preparative procedures can be used to prepare these compounds.

One skilled in the art may use these generic methods to prepare the following specific examples, which have been or may be properly characterized by ¹H NMR, mass spectrometry, elemental composition, and similar procedures. These compounds also may be formed in vivo. The following examples contain detailed descriptions of the methods of preparation of compounds having each of formulas (I)-(V). These detailed descriptions fall within the scope and are presented for illustrative purposes only and are not intended as a restriction on the scope of the invention. All parts are by weight and temperatures are Degrees centigrade unless otherwise indicated.

### GENERAL SYNTHETIC PROCEDURES AND SPECIFIC EXAMPLES

The compounds of the present invention can be synthesized, for example, according to the following procedures and Schemes given below.

Abbreviations used in the schemes and tables include: “AA” represents amino acids, “AcCN” represents acetonitrile, “AcOH” represents acetic acid, “BINAP” represents 2,2′-bis(diphenylphosphino)-1,1′-binaphthyl, “BnOH” represents benzyl alcohol, “BnCHO” represents 2-phenylethanal, “BnSO₂Cl” represents benzylsulfonyl chloride, “Boc” represents tert-butyloxycarbonyl, “BOP” represents benzotriazol-1-yl-oxy-tris-(dimethylamino), “bu” represents butyl, “dba” represents dibenzylidene-acetone, “DCC” represents 1,3-dicyclohexylcarbodiimide, “DCM” represents dichloromethane or methylene chloride, “DIBAH” or “DIBAL” represents diisobutylaluminum hydride, “DIEA” represents diisopropyl ethylamine, “DMF” represents dimethylformamide, “DMSO” represents dimethylsulfoxide, “DPPA” represents diphenylphosphoryl azide, “EDC” or “EDCI” represents 1-[3-(dimethylamino)propyl]-3-ethylcarbodiimide hydrochloride, “Et₂O” represents diethyl ether, “Ex. No.” represents Example Number, “¹FNMR” represents fluorine NMR, “Fmoc” represents 9-fluorenylmethoxycarbonyl, “¹HNMR (MeOD)” represents proton NMR taken in deuterated methanol, “HOBt” represents hydroxybenzoltriazole, “LDA” represents lithium diisopropylamide, “MW” represents molecular weight, “NMM” represents N-methylmorpholine, “NMR” represents nuclear amgnetic resonance, “Ph” represents phenyl or aryl, “PHTH” represents a phthaloyl group, “pnZ” represents 4-nitrobenzyloxy-carbonyl, “PTC” represents a phase transfer catalyst , “py” represents pyridine, “R” represents a hydrocarbyl or a substituted hydrocarbyl, “RNH₂” represents a primary organic amine, “SEM” represents 2-(trimethylsilyl)ethoxy-methyl chloride, “p-TsOH” represents paratoluenesulfonic acid, “TBAF” represents tetrabutylammonium fluoride, “TBTU” represents 2-(1H-benzotriozole-1-yl)-1,1,3,3-tetramethyl uronium tetrafluoroborate, “TEA” represents triethylamine, “TFA” represents trifluoroacetic acid, “THF” represents tetrahydrofuran, “TMS” represents trimethylsilyl, “TMSCN” represents trimethylsilyl cyanide, and “Cbz” or “Z” represents benzyloxycarbonyl, “X” represents a halogen and typically is a bromine or chlorine. As used in the schemes and examples, Z₁, Z₃, Z₄, R₄₄, R₈₀, R₃₀₁, R₃₀₂, R₃₀₃, R₃₀₄, R₃₀₅, R₃₀₆, R₃₀₇, R₃₀₈, R₃₀₉, R₃₁₀, R₃₁₁, and X₇, along with any other variable depicted, encompasses every group described for each particular variable for each embodiment of compounds having formulas (I)-(V) as detailed herein.

A specific synthetic process, useful in the preparation of many of the heterocyclic compounds of the present invention, is the arylation or heteroarylation of an intermediate compound characterized by having a suitable leaving group on a sp² hybridized carbon of a heterocyclic ring. In the product of the reaction, the leaving group is replaced by an aryl group or a heteroaryl group. Suitable leaving groups for the reaction include chloro, bromo, iodo, methylthio, triflates and other similar groups. The heterocyclic ring with the leaving group will typically have an acetic acid group or a derivative thereof bonded to a ring atom alpha to the bromo and a substituted or unsubstituted amino group bonded to a ring atom that is both beta to the carbon having the acetic acid group and gamma to the carbon ring atom substituted with bromo. The aryl group that is reacted at the sp² hybridized carbon is generally an aryl boronic acid or an ester of the aryl boronic acid; similarly, heteroaryl boronic acids or esters of these boronic acids can be used in the same manner as aryl boronates. The aryl and heteroaryl boronates may be substituted or unsubstituted. The aryl or heteroaryl becomes bonded to the sp² hybridized carbon at the point at which the boron was attached to the aryl or heteroaryl ring. Aryl and heteroaryl organosn compounds can also be used instead of the corresponding boronates.

Suitable reaction conditions for carrying out this transformation include:

1. Pd[P(phenyl)₃]₄, 2M Na₂CO₃, 60-75° C., dimethoxyethane (DME), H₂O, N₂;

2. Pd[P(phenyl)₃]₄, Cs₂CO₃, dioxane, 100° C.;

3. Pd[P(phenyl)₃]₄, Cu(I)-2-thiophenecarboxylate, 70-75° C., anhydrous THF, argon; and

4. Z₄ [Sn(n-butyl)₃], Pd[P(phenyl)₃]₄, LiCl, anhydrous dioxane, 85° C., argon or N₂.

The organo palladium (Pd[P(phenyl)₃]₄) compound is used catalytically in a ratio of 1 to 40 mole percent. The carbonate base is normally used in an excess of 1.2 to 2 molar equivalents. Suitable solvents include dimethoxyethane (DME), dioxane, 1-propanol, and tetrahydrofuran. The temperature of the reaction is normally in the range of from about 50 to 100° C. Cu(I)-2-thiophenecarboxylate (Cu(I)-TC) is normally used in a mole percent of 110-150.

Scheme 1 and Example 1 show specific applications of this specific synthetic process. Procedures for preparing the intermediate heterocyclic or cycloalkenyl ring compounds having a suitable leaving group on sp² hybridized carbon and useful as suitable intermediates in this specific synthetic process are given in the schemes and examples listed above.

Scheme 8 illustrates a general synthetic process for substitution at a nitrogen of the heterocyclic ring. The synthetic process applies whether the ring is 5- or 6-membered.

The compounds of the present invention may be synthesized in accordance with one or more of the following schemes:

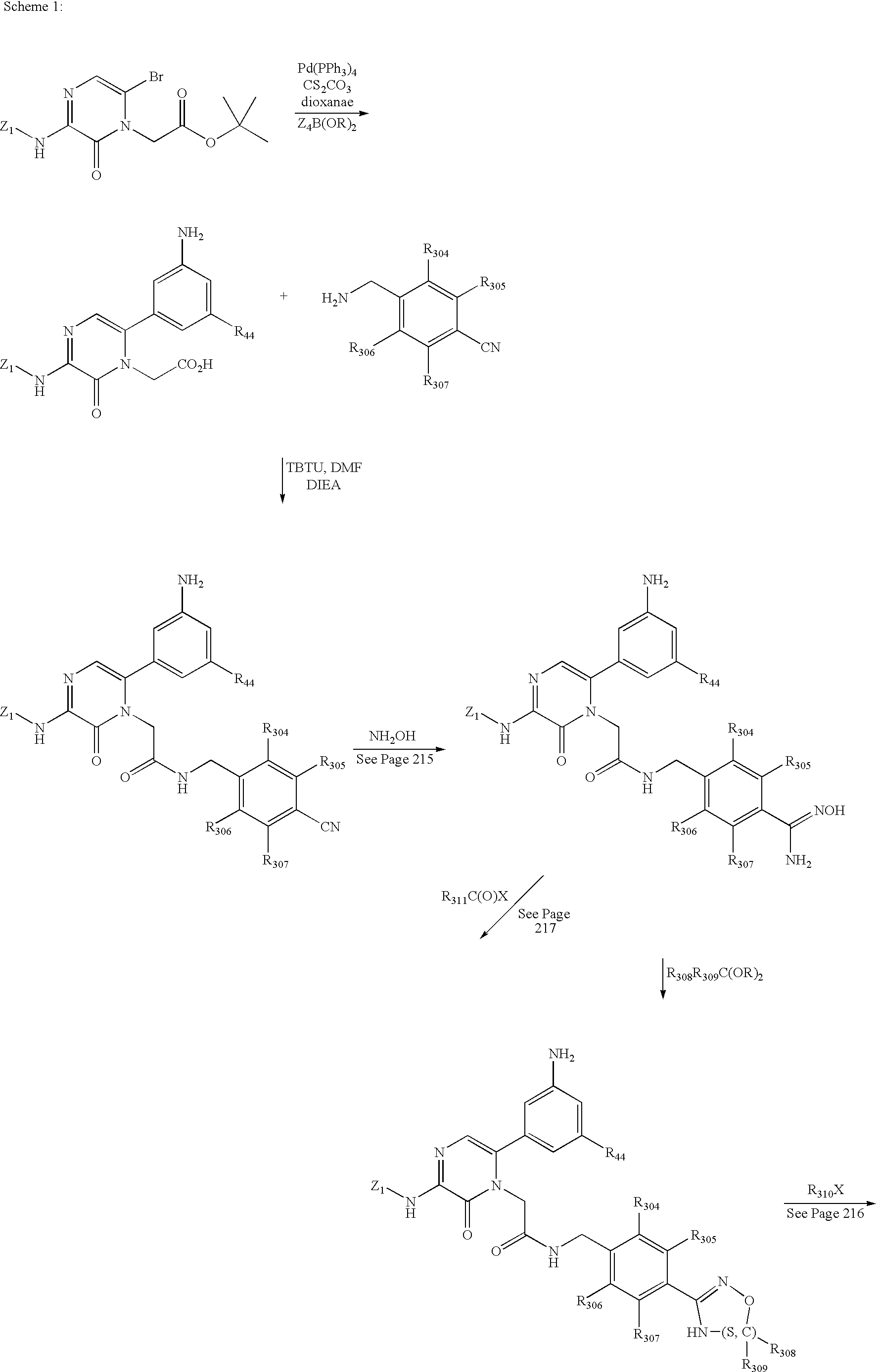

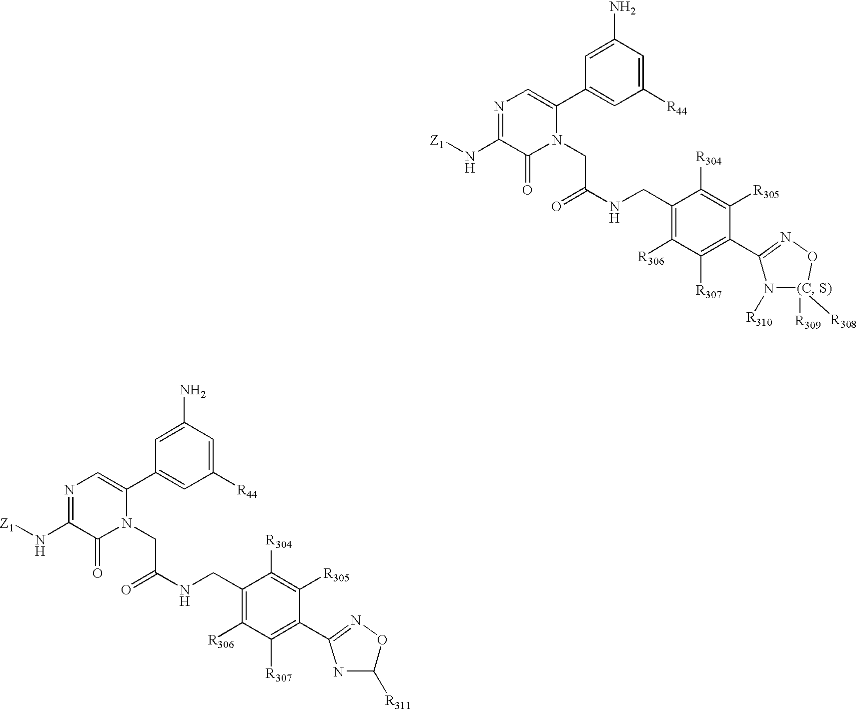

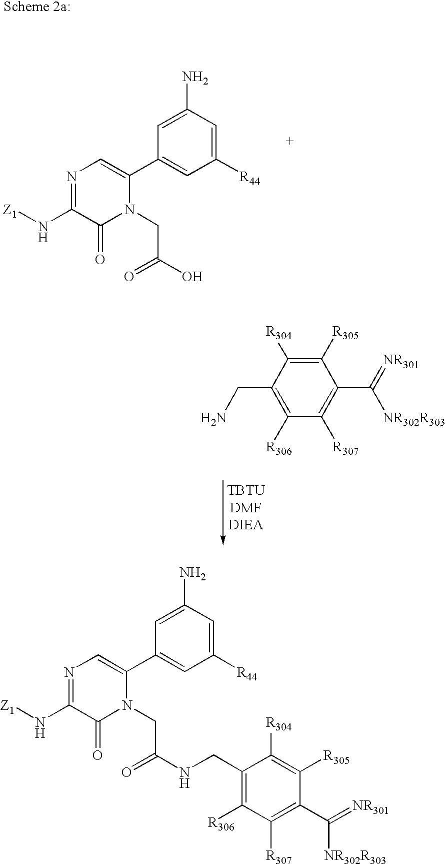

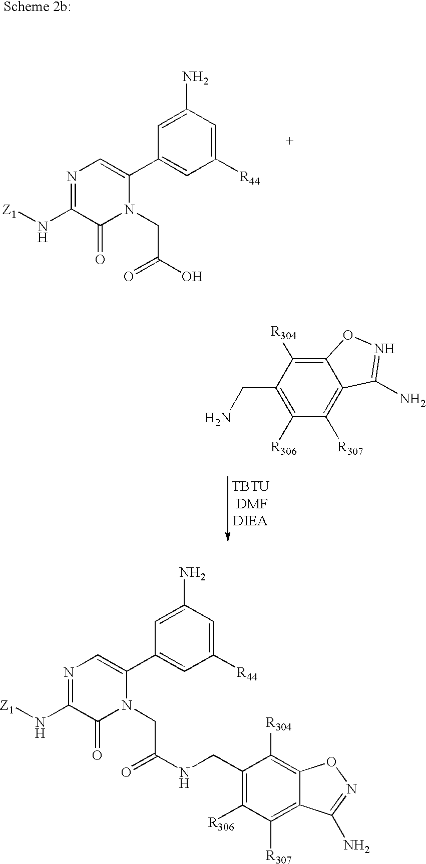

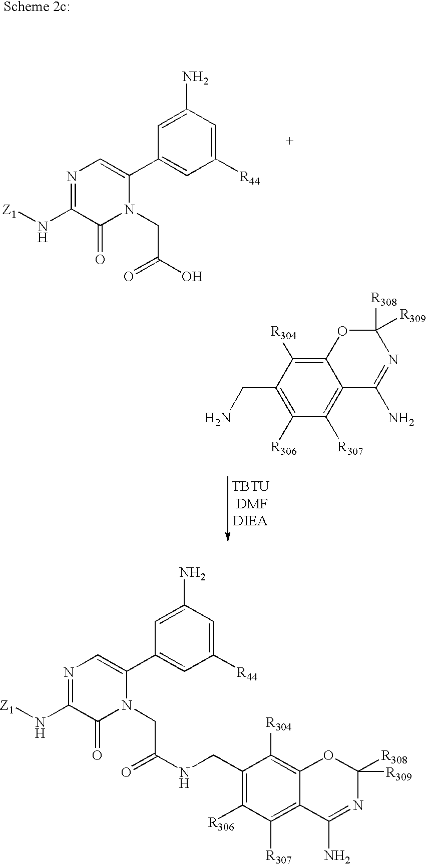

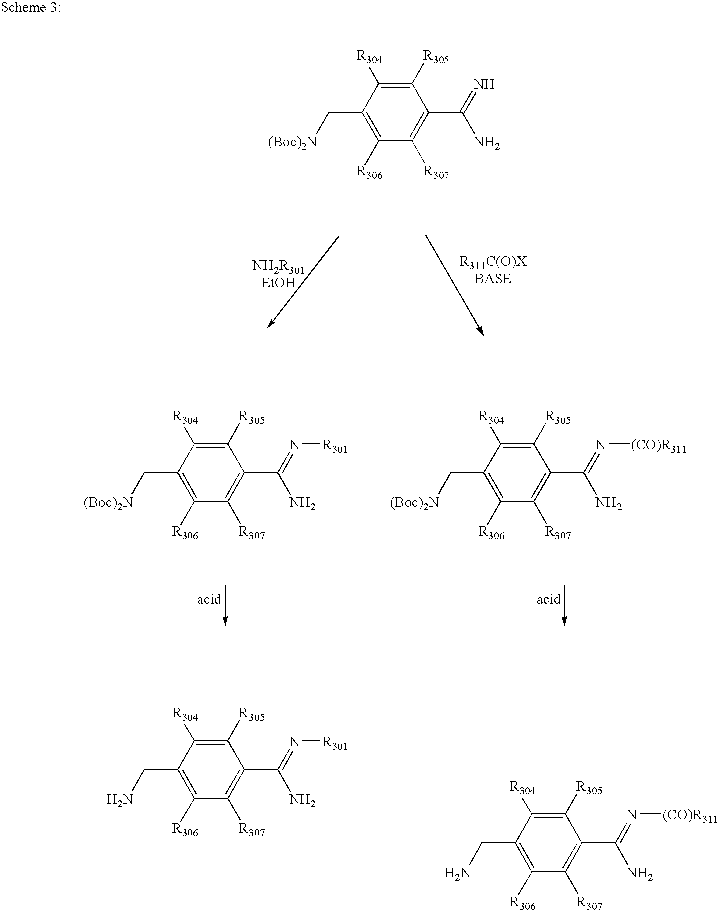

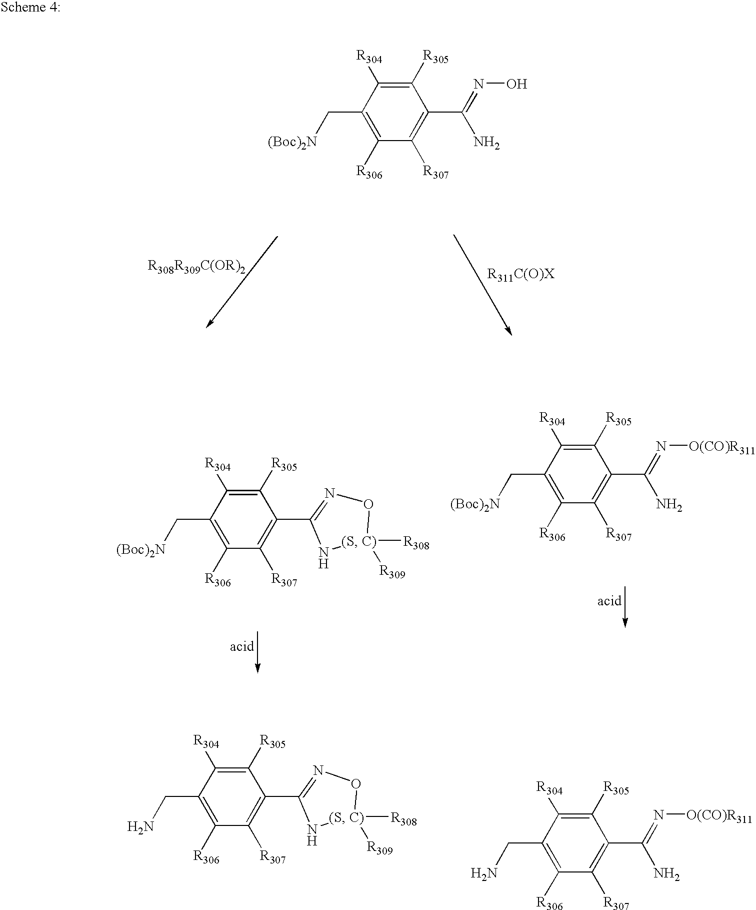

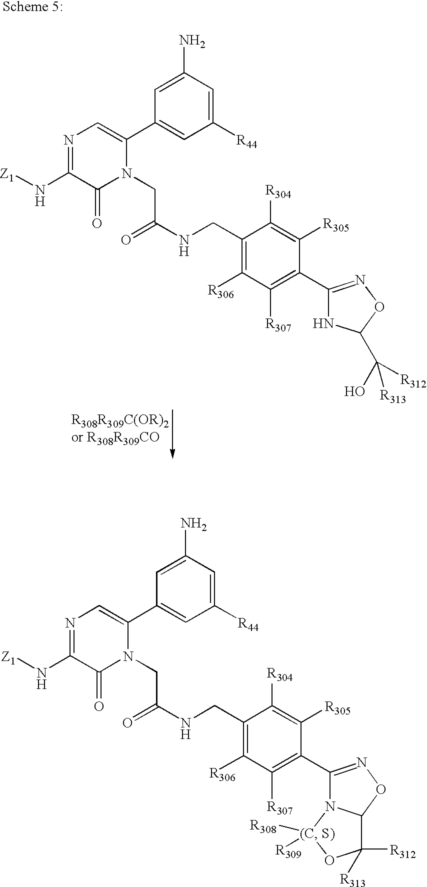

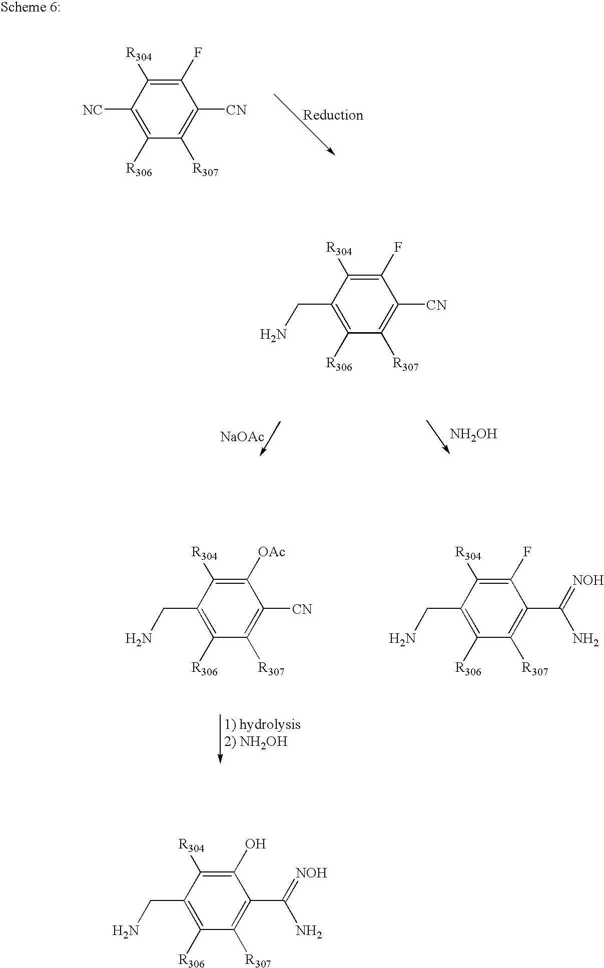

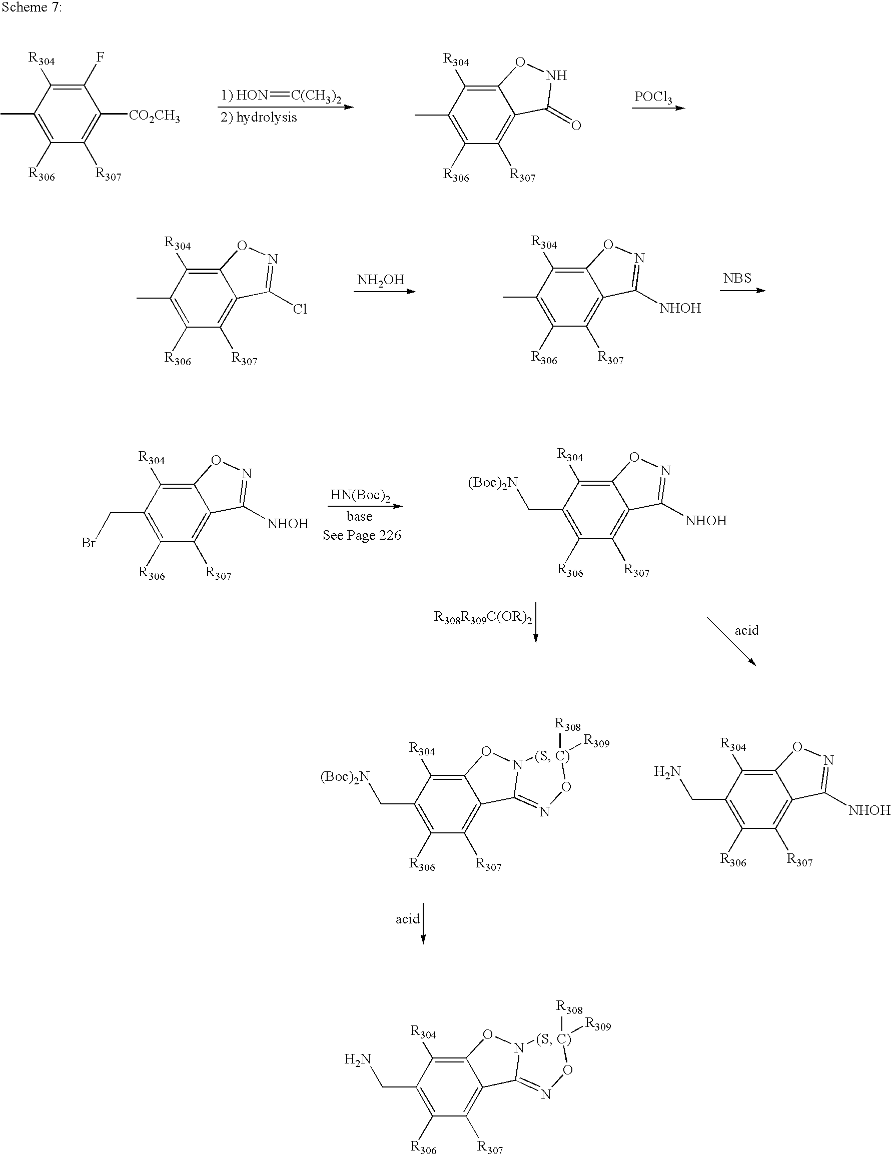

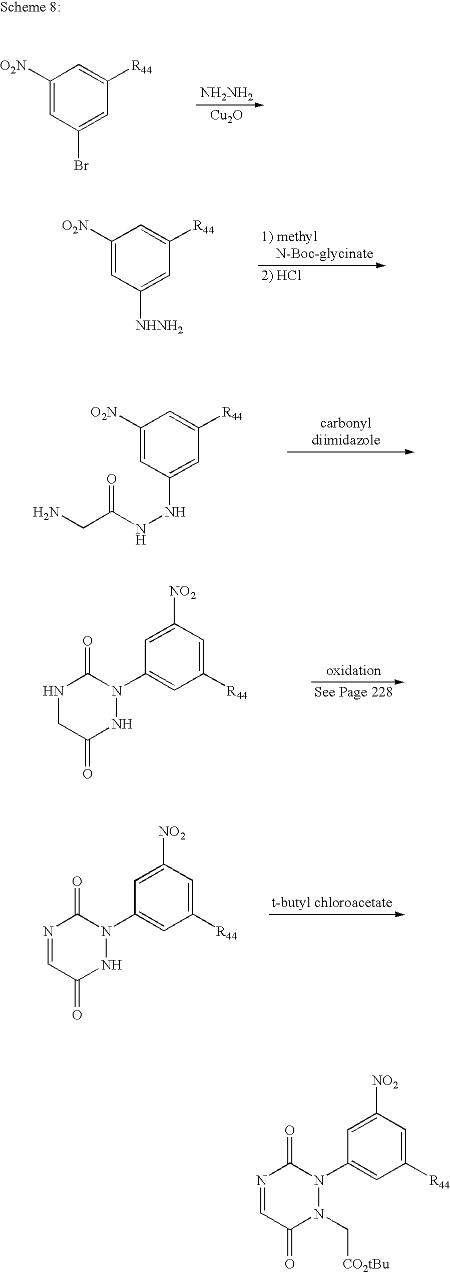

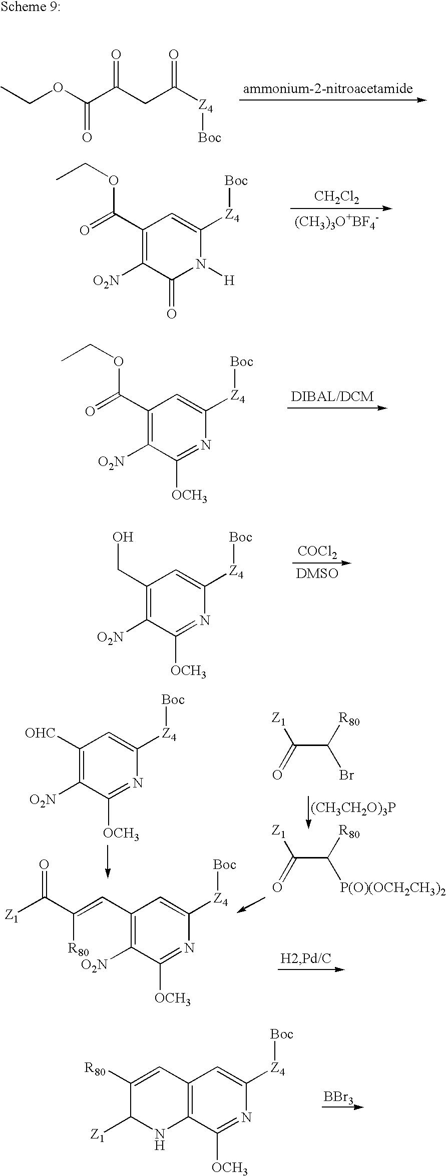

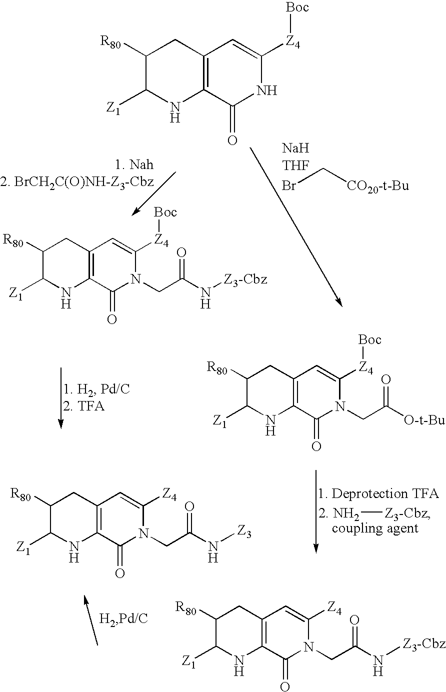

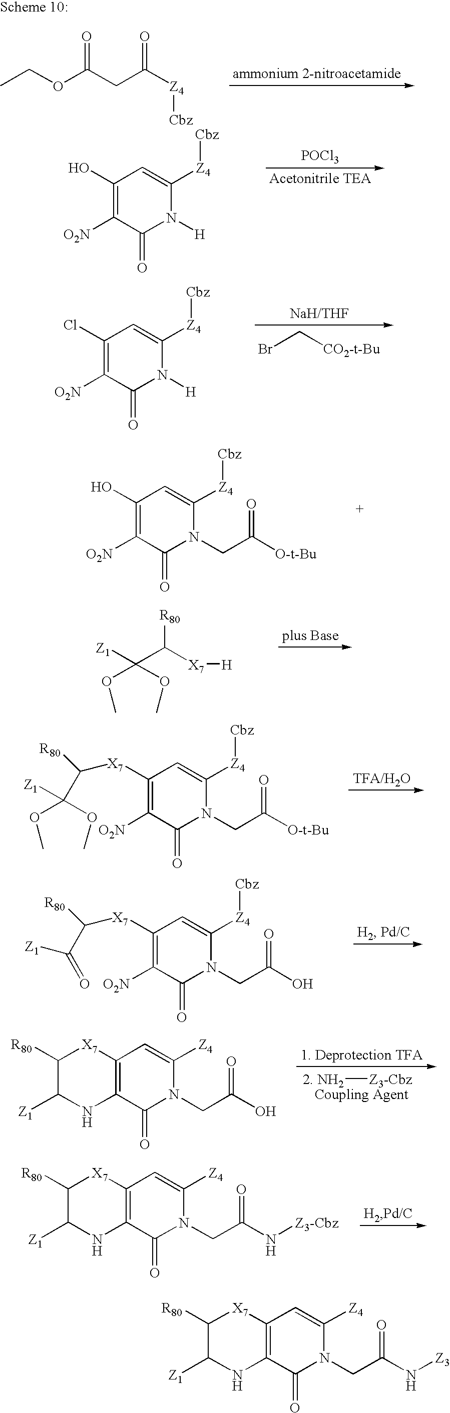

### EXAMPLES

### Example 1

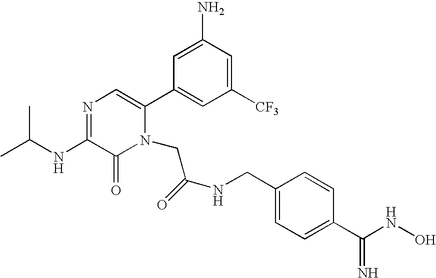

### N-isopropylaminopyrazine

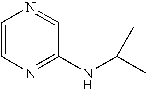

### Example 1a

A solution of 2-chloropyrazine (750 g, 6.55 mo) and isopopylamine (2 L, 23.45 mol) was heated to 130° C. in a pressure reaction flask under 100 psi nitrogen with stirring for 24 hours. The reaction mixture was allowed to cool and diluted with 1 L methanol followed by 4 L of ethyl acetate and 4 L of water. The organic layer was separated and the aqueous solution was extracted with 2L of ethyl acetate. The combined organic solutions were washed with 2L of water, dried over magnesium sulfate, filtered, and concentrated. The crude product afforded a melting point of 46.3° C.

### 2-(N-isopropylamino)-3,5-dibromopyrazine

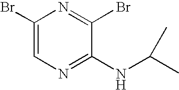

### Example 1b

To a solution of N-isopropylaminopyrazine (514 g, 3.747 mol) in 5.14 L dimethyl sulfoxide and 202 mL water over 30 minutes with the temperature being kept between 40-50° C. by the addition rate was added N-bromosuccinimide (1.866 kg, 11.24 mol). After the addition was completed the reaction mixture was allowed to cool followed by stirring at ambient temperature for 24 hours. An aqueous workup afforded the product as a black oil.

### 5-Bromo-2-(N-isopropylamino)-3-hydroxypyrazine

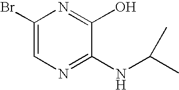

### Example 1c

To a suspension of 2-(N-isopropylamino)-3,5-dibromopyrazine in water was added potassium hydroxide in water. The resulting suspension was heated to reflux for approximately 18 hours to afford the 5-Bromo-2-(N-isopropylamino)-3-hydroxypyrazine.

### 6-Bromo-1-t-butoxycarbonylmethyl-3-(N-isopropylamino)pyrazinone

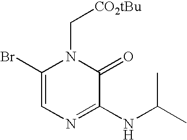

### Example 1d

To 3.3 L tetrahydrofuran was added 5-bromo-2-(N-isopropylamino)-3-hydroxypyrazine (600 g, 2.585 mol) and potassium t-butoxide (365.8 g, 3.1 mol). The resulting suspension was heated at 60° C. for 1 hour. A solution of tert-butyl bromoacetate (605.15 g, 3.1 mol) was then added to the mixture. This mixture was heated at 60° C. for four hours and then allowed to stand at room temperature overnight. The mixture was then diluted with 2.5 L of water and 2.5 L of ethyl acetate. The organic layer was washed with 2.5 L brine. The organic solution was dried over magnesium sulfate, filtered, concentrated, triturated with hexanes and filtered to afford 573 g of an off-white solid.

### 3-(4,4,5,5tetramethyl-1,3,2-dioxaborolan-2-yl)-5-(trifluoromethyl)aniline

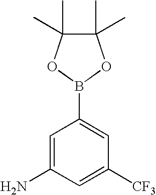

### Example 1e

To a 100 ml, 3 neck, round bottom flask, under nitrogen, magnetic stirrer, cold water condenser, heating mantel, and thermocouple was added 3-amino-5-bromobenzotrifluoride (1.0 g, 4.17 mmol), N,N-dimethyl formamide (50 ml, 645.7 mmol), Bis(pinacolato) diboron (1.06 g, 4.17 mmol) and stirred for one hour while nitrogen was bubbled through the reaction mixture. To the reaction mixture potassium acetate (1.23 g, 12.50 mmol) was added with continued stirring for 30 minutes. To the reaction mixture [1,1′-Bis(diphenylphosphino)-ferrocene], dichloropalladium(II) complex with dichloromethane(1:1) (0.102 g, 0.125 mmol) was added. The nitrogen purge was discontinued. The reaction was slowly warmed to 84° C. and maintained at that temperature for 16 hours. TLC in 50 ethyl acetate/50 hexane, developed in iodine and LCMS indicated the presence of product. The reaction mixture was allowed to cool to room temperature. The reaction mixture was diluted with ethyl acetate and washed with 3×100 ml of brine. The organic layer was dried over magnesium sulfate, filtered through a silica plug, and concentrated to give 2.04 g of a black oil. ¹H NMR indicated desired product and N,N-dimethyl formamide. The crude black oil was dissolved in diethyl ether and was washed with 3×100 ml of water, dried over magnesium sulfate, filtered, and concentrated to give 1.05 g (88%) of a brown oil.

LCMS (0-95 acetonitrile, in 10 min): 6.98 min.

Mass spec: M+H=288.

NMR (400 MHz, CDCl₃): ¹H δ 1.325 ppm (12 H, s), 3.797 ppm (2 H, broad s), 6.966 ppm (1 H, s), 7.252 ppm (1 H, s), 7.428 ppm (1 H, s); ¹⁹F −63.190 ppm (3 F, s).

### Tert Butyl[6-[3-amino-5-(trifluoromethyl)phenyl]-3-isopropylamino)-2-oxopyrazin-1(2H)-yl]acetate

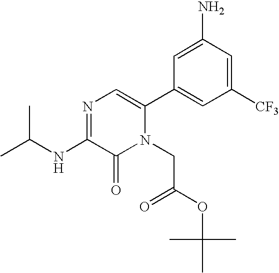

### Example 1f

To a 250 ml, 3 neck, round bottom flask, under nitrogen, magnetic stirrer, cold water condenser, and heating mantel was added the 3-(4,4,5,5 tetramethyl-1,3,2-dioxaborolan-2-yl)-5-(trifluoromethyl) aniline from Example 1a, (2.0 g, 6.97 mmol), tert butyl [6-bromo-3-(isopropylamino)-2-oxopyrazin-1(2H)-yl]acetate (2.16 g, 6.25 mmol), and dioxane (100 ml, 1.17 mol). To the reaction mixture Cesium carbonate (2.44 g, 7.5 mmol) and tetrakis(triphenylphosphine)palladium(0) (1.0 g, 0.865 mmol) were added and warmed to reflux for 15 hours. TLC in 50 ethyl acetate/50 hexane, developed in iodine and MS indicated the presence of product. The reaction mixture was allowed to cool to room temperature, filtered, and washed with 2×100 ml of ethyl acetate. The organic layers were combined, dried over magnesium sulfate, filtered and concentrated to give 5.24 g of a reddish black oil. The crude product was then chromatographed on silica, eluded with 5% ethanol/95% dichloromethane, 25 ml fractions. Desired product was isolated in fractions 40-45. 1.53 g (57%) of a pale orange whitish solid was recovered.

Mass spec: M+H 427.

NMR (400 MHz, CDCl₃): ¹H δ 1.265 ppm (6 H, d), 1.413 ppm (9 H, s), 3.988 ppm (2 H,broad s), 4.178 ppm (1 H, m), 4.349 ppm (2 H,s), 6.105 ppm (1 H, broad s), 6.743 (1 H, s), 6.772 ppm (1 H, s), 6.898 ppm (1 H, s), 6.921 ppm (1 H, s); ¹⁹F δ 63.418 ppm (3 F, s)

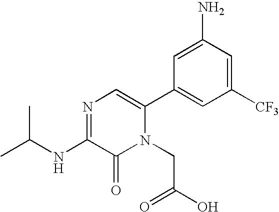

### Example 1g

2.6 g (6.1 mmol) of the product of Example 1f was hydrolyzed with stirring in 15 mL TFA for 90 minutes. The solvent was evaporated and the residue was redissolved in EtOAc, which was then evaporated to dryness. Yield: 2.9 g (6.0 mmol; 98%) solid form.

Mass spec: M+H=371.4

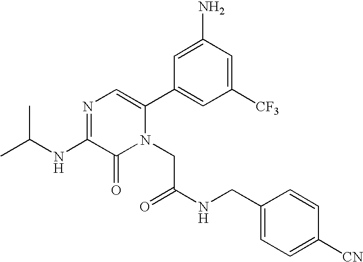

### Example 1h

3.32 g (10 mmol) N,N-di-Boc-4-aminobenzonitrile was deprotected in 50 mL CH₂Cl₂/TFA (4:1) for 30 minutes and the solvent was evaporated thoroughly to dryness. 2.9 g (6.0 mmol) of the product of Example 1g was coupled with the 4-aminobenzonitrile in 40 mL DMF in the presence of 2.25 g (7 mmol) TBTU and 3.5 mL (20 mmol) DIPEA with stirring for 12 hours. The DMF was evaporated and the product was precipitated by addition of 200 mL water and filtered. Yield: 2.2 g (4.5 mmol; 76%) white solid.

Mass spec: M+H=485.3

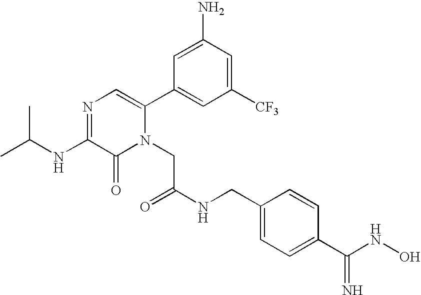

### Example 1

2.2 g (4.5 mmol) of the product of Example 1h was dissolved in 50 mL EtOH. 1.44 g (20 mmol) hydroxylamine HCl and 4.4 mL (25 mmol) DIPEA were added to the solution and the mixture was refluxed for 3 hours. The EtOH was then evaporated and the product was precipitated by addition of 200 mL water. The product was filtered and dried. The crude product (1.1 g) was purified on preparative HPLC using a gradient of acetonitrile (10-50% AcN in 30 minutes), yielding the title product at 33% AcN, 1.1 g (67%) white solid.

Mass spec: M+H=518.3

¹H NMR: 300 MHz, CD₃OD: δ 7.66-7.61 (d, 2H), 7.47-7.42 (d, 2H), 7.02 (s, 1H), 6.90-6.85 (d, 2H), 6.68 (s, 1H), 4.52 (s, 2H), 4.46 (s, 2H), 4.10-4.00 (m, 1H) and 1.40-1.34 (m, 6H).

¹⁹F NMR: 300 MHz, CD₃OD: δ −64.9 (s, 3F) and −75.5 (s, 6F).

Elemental analysis:

| | | | |
|---|---|---|---|
| Found | C: 43.60 | H: 3.69 | N: 12.94 |
| Calculated | C: 43.38 | H: 3.87 | N: 12.47 |

### Example 2

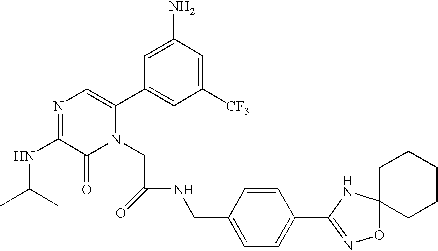

The product of Example 1 (258 mg; 0.0005 mole) and cyclohexanone dimethyl ketal (360 mg; 0.0025 mole) were dissolved in 4.0 ml of a 1:1 mixture of acetic acid and 1,2-dichloroethane. The mixture was gently refluxed for 0.5 hours. HPLC [0 to 95% MeCN/H₂O * TFA over 6 minutes] indicated that the N-hydroxylbenzamidine was consumed (“* TFA” represents “containing 0.05% TFA”). The reaction mixture was then concentrated. The residue was triturated with acetone and the solid was filtered and washed with Et₂O and suction dried to yield 233 mg of a solid.

Calculated for C₃₀H₃₄N₇F₃O₃+1.0 HOAc +0.5 H₂O +0.25 Acetone;

Elemental:

| | | | |
|---|---|---|---|
| Theory: | C: 57.74; | H: 5.99; | N: 14.39 |
| Found: | C: 57.68; | H: 5.87; | N: 14.15. |

### Example 3

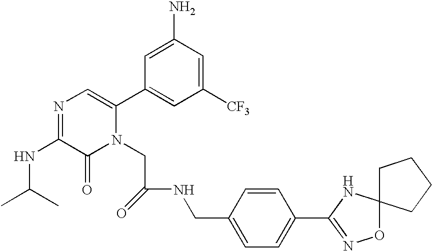

The product of Example 1 (258 mg; 0.0005 mole) and cyclopentanone dimethyl ketal (325 mg; 0.0025 mole) were dissolved in 4.0 ml of a 1:1 mixture of acetic acid and 1,2-dichloroethane. The mixture was gently refluxed for 0.5 hours and worked up as described above to yield 103 mg of a solid.

Calculated for C₂₉H₃₂N₇F₃O₃+0.8 HOAc+1.0 Acetone;

Elemental:

| | | | |
|---|---|---|---|
| Theory: | C: 58.51; | H: 6.02; | N: 14.22 |
| Found: | C: 58.90; | H: 5.73; | N: 13.80. |

### Example 4

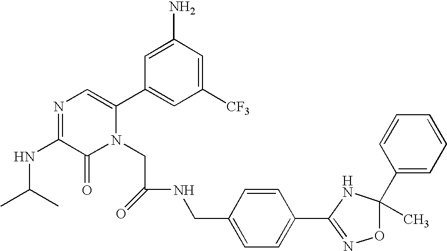

N-hydroxylbenzamidine (258 mg; 0.0005 mole) and (1,1-dimethoxyethyl)benzene (415 mg; 0.0025 mole) were dissolved in 4.0 ml of a 1:1 mixture of acetic acid and 1,2-dichloroethane. The mixture was gently refluxed for 0.5 hours and concentrated. The residue was chromatographed over silica, eluting with a MeOH/CH₂Cl₂ system, to yield 59 mg of a solid.

Calculated for C₃₂H₃₂N₇F₃O₃+0.75 H₂O;

Elemental:

| | | | |
|---|---|---|---|
| Theory: | C: 60.70; | H: 5.33; | N: 15.49 |
| Found: | C: 60.73; | H: 5.02; | N: 15.02. |

### Example 5

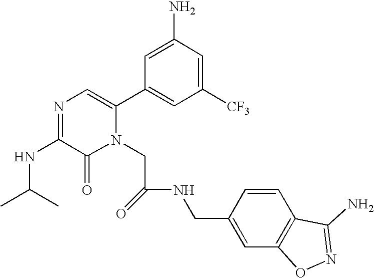

Carboxylic acid (1.1 g; 0.0025 mole), benzylamine HCl (0.5 g; 0.0025 mole), diisopropylethyl amine (DIEA) (1.6 g; 0.0125 mole) and TBTU (0.882 g; 0.11275 mole) were dissolved in 15 ml of DMF and stirred for 18 hours. The reaction mixture was poured into 50 ml of water and the solid was filtered to yield 1.0 g.

Mass Spec: M+H=516.

¹HMR(MeOD): δ ppm 1.25 (d, 6H); 4.09 (sep, 1H); 4.48 (s, 1H); 4.51 (s, 1H); 6.71 (s, 1H); 6.84 (s, 1H); 6.86 (s, 1H); 6.96 (s, 1H); 7.13 (d, 1H); 7.26 (s, 1H); 7.67 (d, 1H).

¹⁹FMR (MeOD): δ ppm −64.83.

### Example 6

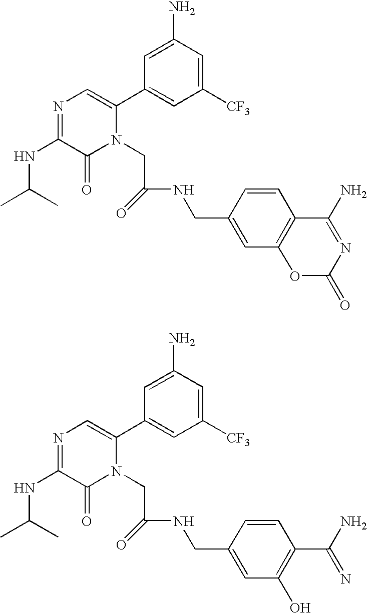

### Example 6a

The product of Example 5 (1.0 g; 0.0019 mole) and ammonium formate (244 mg; 0.0038 mole) were dissolved in MeOH (20 ml) and N₂ gas was bubbled through the mixture. Pd Black (100 mg) was suspended in MeOH (3 ml) and added to the above mixture. Starting material was still present after 0.5 hours. An additional portion of ammonium acetate (244 mg) was added. After 0.5 hours, the remaining starting material was consumed. The reaction mixture was filtered and concentrated to yield 1.2 g of a solid.

Mass Spec: M+H=518.

¹H NMR(MeOD): δ ppm 1.25 (d, 6H); 4.09 (sep, 1H); 4.32 (s, 1H); 4.49 (s, 1H); 6.71 (s, 1H); 6.77 (d, 1H); 6.85 (s, 2H); 6.86 (s, 1H); 6.96 (s, 1H); 7.56 (d, 1H).

¹⁹F NMR (MeOD): δ ppm −64.80.

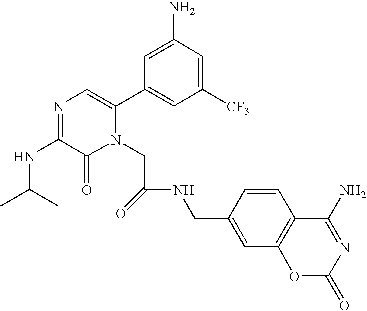

### Example 6

The product of Example 6a (517 mg; 0.001 mole) and triethylamine (303 mg; 0.003 mole) were dissolved in DMF (5.0 ml). To the mixture, 1,1′-carbonyldiimidazole (648 mg; 0.004 mole) was added and the mixture was stirred for 18 hours. The mixture was poured into 50 ml of water and the solid was filtered. The solid was triturated with MeOH and filtered to yield 211 mg of a solid.

Calculated for C₂₅H₂₄N₇F₃O₄+3.5 H₂O

Elemental:

| | | | |
|---|---|---|---|
| Theory: | C: 49.50; | H: 5.15; | N: 16.16 |
| Found: | C: 49.46; | H: 5.06; | N: 15.96. |

Mass Spec: M+H=544.

¹HMR(DMSO): δ ppm 1.15 (d, 6H); 4.07 (sep, 1H); 4.32 (s, 1H); 4.34 (s, 1H); 6.66 (s; 1H); 6.74 (broad s, 2H); 6.85 (s, 1H); 7.07 (s, 1H); 7.11 (d, 1H); 6.96 (s, 1H); 7.97 (d, 1H).

¹⁹FMR (DMSO): δ ppm −62.47.

### Example 7

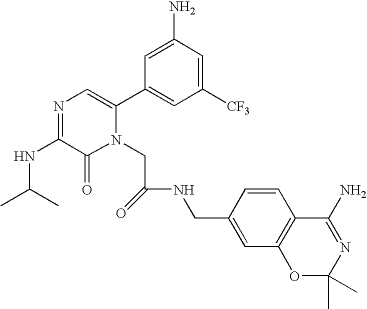

The product of Example 6a (517 mg; 0.001 mole) and 2,2-dimethoxypropane (10 ml) were dissolved in 10.0 ml of a 1:1 mixture of acetic acid and 1,2-dichloroethane and refluxed for 3 days. HPLC indicated two new components. The reaction mixture was concentrated and purified by RPLC (0 to 60% MeCN/H₂O *THF over 6 minutes). Retention time of the product was 3.79 minutes yielding 60 mg of a glass.

Calculated for C₂₇H₃₀N₇F₃O₃+2.5 TFA +1.0 H₂O;

Elemental:

| | | | |
|---|---|---|---|
| Theory: | C: 44.66; | H: 4.04; | N: 11.39 |
| Found: | C: 44.48; | H: 4.71; | N: 9.45. |

Mass Spec: M+H=558.

¹HMR(MeOD): δ ppm 1.36 (d, 6H); 1.66 (s, 6H); 4.05 (sep, 1H); 4.45 (s, 1H); 4.55 (s, 1H); 6.65 (s, 1H); 6.86 (broad s, 2H); 6.97 (s, 1H); 7.02 (s, 1H); 7.10 (d, 1H); 7.87 (d, 1H).

¹⁹FMR (MeOD): δ ppm −64.85 and −77.42.

### Example 8

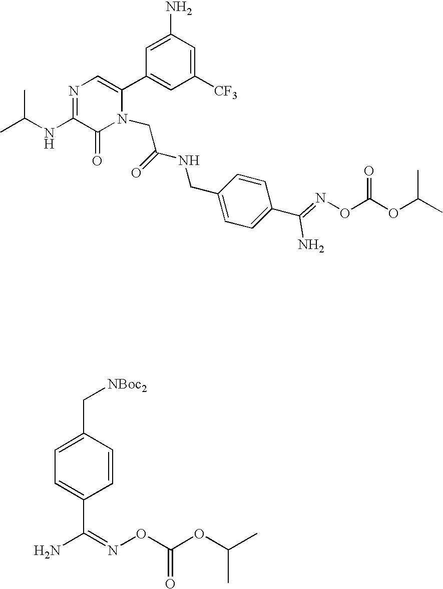

### Example 8a

To a 250 mL RBF was added NaH (60%, 0.54 g, 14 mmol) in dry THF (30 mL). The dibocaminobenzyl-4-hydroxamidine (5.0 g, 13.7 mmol) was then added to the slurry at 0° C. The reaction was stirred for 0.5 hrs. To the reaction was added isopropyl chloroformate (1 M solution in Toluene, 13.7 mL). The reaction was stirred for three hours and then quenched with 50 ml of water and extracted with ethyl acetate (3×50 mL). The organics were dried over magnesium sulfate and then concentrated. The resulting solid was purified on silica using 40% ethyl acetate:60% hexane to afford Example 8a 4.29 g (69%) as a white solid.

C₂₂H₃₂N₃O₇; M.W.450.22

¹H NMR (CDCl₃, 300 MHz) δ1.31(d, 6H), δ 1.45 (s, 18H) δ 4.75 (s, 2H), δ 4.95(q, 1H), δ 5.15(bs, 2H), δ 5.35(s, 1H), δ 3.80(d, 2H), δ 7.60(d, 2H).

¹³C NMR (CDCl₃, 75 MHz) δ21.73, 27.94, 49.10, 72.49, 82.70, 126.67, 127.29, 129.69, 141.58, 152.38, 153.27, 155.90.

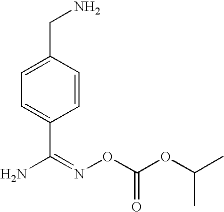

### Example 8b

To a 250 ml RBF was added Example 8a (2.29 g, 5.1 mmol) in 4 N HCl in 50 ml of dioxane. The reaction was stirred for 4 hours. The reaction, monitored by mass spectrometry, was then concentrated in vacuo to afford 1.34 g of the dihydrochloride salt. Because of the hydroscopic nature of the intermediate, the amine was carried onto the next reaction without further manipulations.

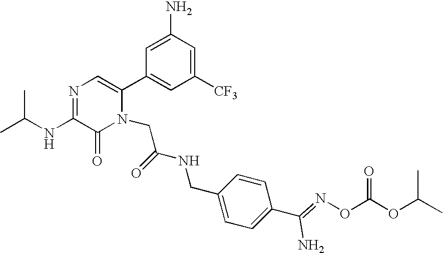

### Example 8

To a 250 ml RBF was added Example 8b (1.34 g, 4.15 mmol) and the product of Example 1g (2.29 g, 4.73 mmol) in 50 ml of DMF. To the solution was added DIEA (14.41 g, 20 ml) and TBTU (1.82 g, 5.7 mmol). The reaction was stirred over night. The reaction was then poured into ethyl acetate and washed with 10% KHSO4, then brine. The organics were dried over magnesium sulfate and concentrated. The resulting solid was purified on silica 20% ramped to 100% ethyl acetate:hexane to afford Example 8 (1.44 g), 58% yield.

C₂₈H₃₂F₃N₇O₅; M.W.603.59; Calculated C 55.72 H 5.34 N16.24 F 9.44, found C 54.95 H 5.38 N 15.80 F 9.31;

¹H NMR (DMSO-d₆, 300 MHz) δ1.24(d, 6H), 1.3(d, 6H) 4.14 (q, 2H), 4.36 (dd, 4H), 4.89(q, 1H), 4.89(q, 1H), 5.83(bs, 2H), 6.74(s, 1H), 6.88(m, 6H), 7.29(d, 2H), 6.77(d, 2H), 8.66(t, 1H).

¹³C NMR (CDCl₃, 75 MHz) δ 21.12, 21.44, 41.16, 41.83, 47.43, 48.09, 70.97, 11.93, 117.54, 120.80, 121.91, 125.52, 126.20, 126.45, 127.11, 129.25, 129.48, 129.66, 133.79, 141.00, 148.81, 149.26, 150.57, 152.55, 155.61, 166.01.

### Example 9

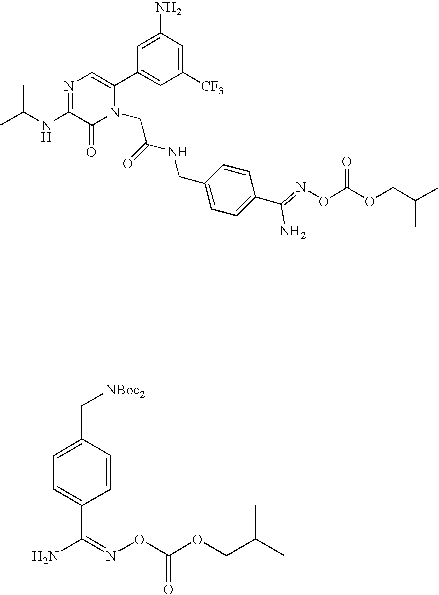

### Example 9a

Following the method detailed in, Example-8a dibocaminobenzyl-4-hydroxamidine (6.0 g, 16.4 mmol), NaH (60%, 0.69 g, 18.1 mmol), isobutyl chloroformate (2.19 g, 16.4 mmol) afforded 3.8 g in 50% yield.

C₂₃H₃₅N₃O₇; M.W.465.54

¹H NMR (CDCl₃, 300 MHz) δ 0.91(d, 6H), δ 1.45 (s, 18H), δ 1.69 (bs, 1H), δ 2.05(q, 1H), δ 4.10(d, 2H), δ 4.78(s, 2H), δ 5.09(bs, 2H), δ 7.32(d, 2H), δ 7.63(d, 2H).

¹³C NMR (CDCl₃, 75 MHz) δ18.89, 27.87, 28.02, 49.17, 74.46, 82.75, 126.71, 127.46, 129.80, 141.75, 152.49, 153.99, 155.97.

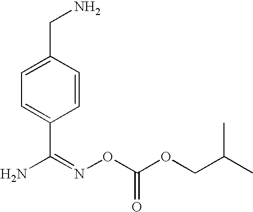

### Example 9b

Following the method detailed in Example 8b, the product of Example 9a (1.05 g, 2.25 mmol) in 4 N HCl/dioxane afforded 0.759 g of the dihydrochloride.

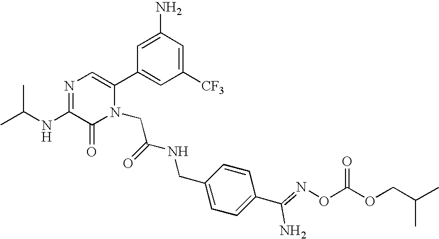

Example 9

To a 250 RBF was added the product of Example 1g (1.0 g, 2.27 mmol), HOBt (0.922 g, 6.82 mmol) and EDCI (2.03 g, 6.82 mmol) in 75 ml of DMF. To the solution was added Example 9b (0.759 g, 2.25 mmol) and DIEA (1.45 g, 11.35 mmol). The reaction was stirred over night. To the reaction was added 5% citric acid and ethyl acetate. The organics were dried over magnesium sulfate and concentrated in vacuo. The resulting solid was purified on silica using 20% to 100% ethyl acetate:hexane to afford

### Example 9

(0.30 g, 21% yield) C₂₉H₃₄F₃N₇O₅; M.W.617. 62;

Calculated C 56.40 H 5.55 N 15.88 F 9.23, found C 56.20 H 5.58 N 14.90 F 8.60;

¹H NMR (CDCl₃, 300 MHz) δ 1.24(d, 6H), 1.3(d, 6H), 4.14 (q, 21H), 4.36 (dd, 4H), 4.89(q, 1H), 4.89(q, 1H) 5.83(bs, 2H), 6.74(s, 1H), 6.88(m, 6H), 7.29(d, 2H), 6.77(d, 2H), 8.66(t, 1H).

¹³C NMR (CDCl₃, 75 MHz) δ 21.12, 21.44, 41.16, 41.83, 47.43, 48.09, 70.97, 11.93, 117.54, 120.80, 121.91, 125.52, 126.20, 126.45, 127.11, 129.25, 129.48, 129.66, 133.79, 141.00, 148.81, 149.26, 150.57, 152.55, 155.61, 166.01.

### Example 10

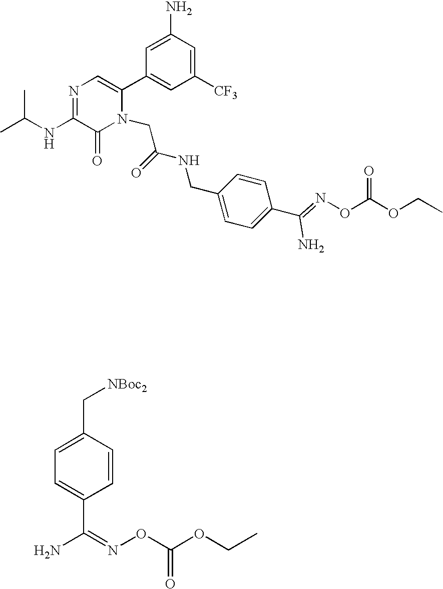

### Example 10a

Prepared by the method of Example 8a. dibocaminobenzyl-4-hydroxamidine (2.55 g, 6.98 mmol), NaH (60%, 0.28 g, 7.25 mmol), ethyl chloroformate(0.832 g, 7.67 mmol) afforded 1.16 g in 53.78% yield.

C₂₁H₃₁N₃O₇; M.W.437.49

¹H NMR (CDCl₃, 300 MHz) δ 1.41(t, 3H), δ 1.55 (s, 18H) δ 4.38(q, 2H), δ 4.85(s, 2H), δ 5.19(bs, 2H), δ 7.32(d, 2H), δ 7.63(d, 2H).

¹³C NMR (CDCl₃, 75 MHz) δ14.59, 28.26, 49.43, 64.90, 83.15, 127.03, 127.69, 129.80, 141.75, 152.50, 153.90, 155.92.

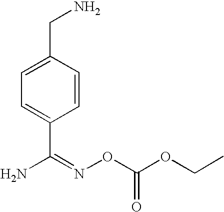

### Example 10b

Prepared by the method of Example 8b. The product of Example 10a (0.55 g, 1.24 mmol) in 4 N HCl/dioxane afforded 0.42 g of the dihydrochloride.

### Example 10c

To a 100 RBF was added the product of Example 1g (1.22 g, 2.77 mmol), HOBt (1.125 g, 8.31 mmol), EDCI (2.5 g, 8.31 mmol) in 45 ml of DMF. To the solution was added the product of Example 10b (0.74 g, 2.77 mmol) and DIEA (1.79 g, 13.85 mmol). The reaction was stirred over night. To the reaction was added 5% citric acid and ethyl acetate. The organics were dried over MgSO₄ and concentrated in vacuo. The resulting solid was purified on silica using 20% to 100% ethyl acetate:hexane to afford Example 10 (0.40 g, 25% yield) C₂₇H₃₀F₃N₇O₅+0.55 H₂O M.W.589.57;

Calculated C 55.00 H 5.13 N 16.63, found C 54.14 H 5.23 N 16.36;

¹H NMR (CDCl₃, 300 MHz) δ1.18(d, 6H), δ1.23(t, 2H), δ 1.31 (t, 2H), δ 4.09 (m, 2H), δ 4.24(m, 3H), δ 4.43(s, 1H), δ 5.45(bs, 2H), δ 6.20(s, 1H), δ 6.71(m, 2H), δ 6.82(d, 1H), δ 6.99(d, 1H), δ 7.30(t, 2H), δ 7.62(m, 2H).

¹³C NMR (CDCl₃, 75 MHz) δ14.23, 22.29, 41.55, 42.83, 43.11, 48.94, 64.67, 11.271, 115.58, 116.79, 118.94, 1121.95, 126.36, 126.92, 127.61, 128.67, 141.15, 147.63, 154.32, 156.66, 167.02.

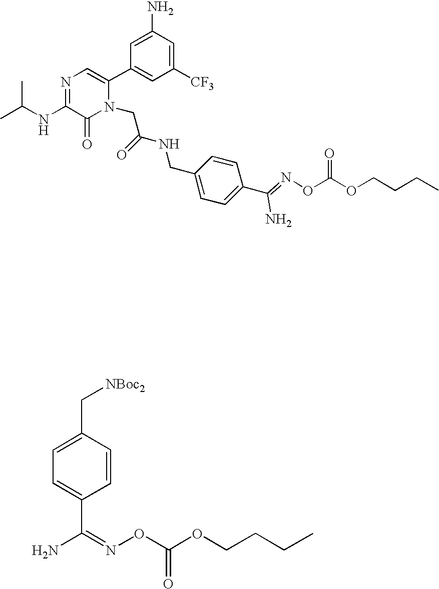

### Example 11

### Example 11a

To a 50 mL RBF was added the dibocaminobenzyl-4-hydroxamidine (1.0 g, 2.736 mmol) and DIEA (0.523 g, 4.11 mmol). The reaction was stirred for one hour and n-butyl chloroformate (0.467 g, 3.42 mmol) was added at room temperature. The reaction was then stirred over night. To the reaction was added ethyl acetate and 5% citric acid. The organics were back washed with brine then dried over MgSO₄. After the organics were concentrated, the resulting oil was purified on silica using 10%-50% ethylacetate:hexanes. This afforded Example 11a (0.43 g) in 34% yield.

C₂₃H₃₅N₃O₇; M.W.465.54 Mass Spec: M+H(466.5), M+Na⁺ (488.5)

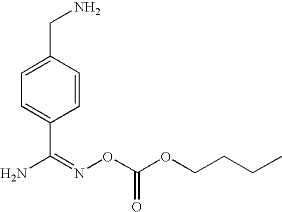

### Example 11b

By following the method of Example 8b, the product of Example 11a (0.43 g, 0.91 mmol) in 4 N HCl/dioxane afforded 0.31 g of the dihydrochloride.

### Example 11c

To a 100 RBF was added the product of Example 1g (0.402 g, 0.9136 mmol), HOBT (0.370 g, 2.74 mmol) and EDCI (0.814 g, 2.74 mmol) in 25 ml of DMF. To the solution was added the product of Example 11b (0.31 g, 0.9136 mmol) and DIEA (0.59 g, 4.575 mmol). The reaction was stirred over night. To the reaction was then added 5% citric acid and ethyl acetate. The organics were dried over MgSO₄ and concentrated in vacuo. The resulting solid was purified on silica using 20% to 100% ethyl acetate:hexane to afford Example 11c (0.063 g, 11% yield) C₂₉H₃₄F₃N₇O₅; M.W.617.62;

Calculated C 56.4.00 H 5.55 N 15.88, found C 56.45 H 5.48 N 15.58;

¹H NMR (D₂O, 300 MHz) δ0.98(t, 3H), δ1.23(d, 6H), δ1.45 (q, 2H), δ 1.70 (qu, 2H), δ 4.28(t, 2H), δ 4.43(s, 2H), δ 4.51(s, 2H), δ 6.75(s, 1H), δ 6.88(d, 2H), δ 6.98(s, 1H), δ 7.35(d, 2H), δ 7.45(m, 2H), δ 7.89(d, 2H)

### Example 12

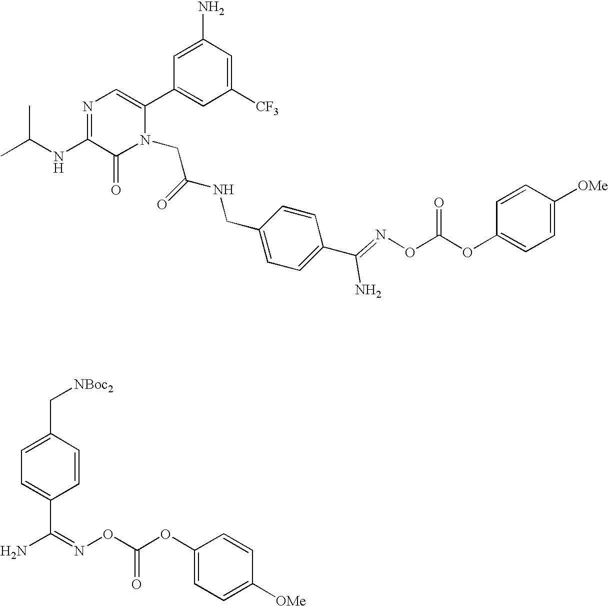

### Example 12a

Synthesized by the method of Example 8a. Dibocaminobenzyl-4-hydroxamidine (2.3 g, 6.9 mmol), NaH (60%, 0.31 g, 7.7 mmol), p-methoxyphenyl chloroformate (1.21 g, 6.5 mmol) afforded 2.1 g in 62% yield.

C₂₆H₃₃N₃O₈; M.W.515.56; M.S. M+H⁺ (516.5), M+Na⁺ (538.5);

¹H NMR (CDCl₃, 300 MHz) δ 1.45(s, 18H), δ 3.83 (s, 3H) δ 4.85 (s, 2H), δ 5.21(bs, 2H), δ 6.90(d, 2H), δ 7.19(d, 2H), δ 7.39(d, 2H), δ 7.70(d, 2H)

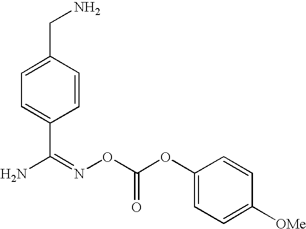

### Example 12b

Synthesized by the method of Example 8b, the product of Example 12a (1.05 g, 1.94 mmol) in 4 N HCl/dioxane afforded 0.746 g of the dihydrochloride.

### Example 12c

To a 250 ml RBF was added the product of Example 12b (0.746 g, 1.94 mmol) and the product of Example 1g (1.94 mmol) in 40 ml of DMF. To the solution was added DIEA (1.98 g, 2.75 mL) and TBTU (0.685 g, 2.1 mmol). The reaction was stirred over night. The reaction was then poured into ethyl acetate and washed with 10% KHSO4, then brine. The organics were dried over MgSO4 and concentrated. The resulting solid was purified on silica 20% ramped to 100% ethyl acetate:hexane to afford Example 12c (0.71 g, 56% yield. C₃₂H₃₂F₃N₇O₆+0.7EA; M.W.667.68;

Calculated C 57.57 H 4.83 N 14.69 F 8.54, found C 57.30 H 5.15 N 13.43 F 7.66;

¹H NMR (CDCl₃, 300 MHz) δ1.29(d, 6H), δ3.29(q, 2H), δ3.76 (s, 3H), δ 4.09 (m, 2H), δ 3.80 (s, 2H), δ 4.49(s, 2H), δ6.66(s, 1H), δ 6.79(s, 1H), δ 6.83(s, 1H), δ 6.90(d, 1H), δ6.96(s, 1H), δ 7.12(d, 2H), δ 7.26(d, 2H), δ 7.65(dd, 2H).

### Example 13

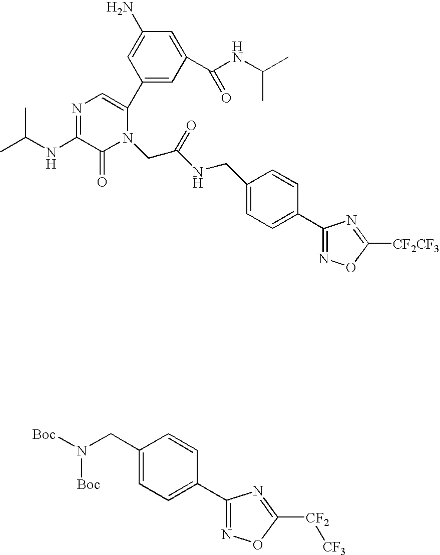

### Example 13a

To a solution of dibocaminobenzyl-4-hydroxamidine (0.51 g, 1.39 mmol) in 20 ml of dichloromethane was added pyridine (0.25 ml, 3.06 mmol) and pentafluoropropionic anhydride (0.29 ml, 1.46 mmol) at 0° C. The reaction mixture was allowed to warm to room temperature and stirred for 2 hrs. The reaction mixture was then diluted with water. The layers were separated and the aqeous layer extracted with dichlormethane (2×). The organic extracts were washed with brine (1×). The organic fractions were dried (Na₂SO₄) and the solvent removed in vacuo to give a white solid, which after chromatography (silica, 10% ethyl acetate/hexanes to 30% ethyl acetae/hexanes) gave Example 13a as a white solid (0.60 g). m/z+1=494.

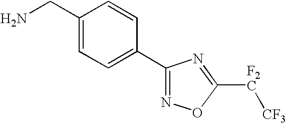

### Example 13b

To a round bottom containing the product of Example 13a (0.60 g, 1.20 mmol) was added 4.0 N HCl in dioxane (20 mL) at room temperature. After stirring for 3 hrs at room temperature the precipitate was filtered and dried on high vacuum to give a white powder Example 13b (0.349 g). m/z+1=294

### Example 13c

To a solution of the corresponding acid (yellow solid, Mass Spec M+H=418) (prepared analogously to Example 1a-1g) (0.25 g, 0.64 mmol) in 15 ml of DMF was added TBTU (0.20 g, 0.64 mmol) at 0° C. After 5 min, the product of Example 13b (0.19 g, 0.64 mmol) and DIEA (0.45 mL, 2.56 mmol) were added. The reaction was stirred for 1 hr and then diluted with water and ethyl acetate. The layers were separated and the organic layer was washed with saturated sodium bicarbonate and dried (Na₂SO₄). The solvent was removed to give a semi-solid, which after chromatography (silica, ethyl acetate) gave Example 13c (0.18 g) as a yellow solid. m/z+1=663

### Example 14

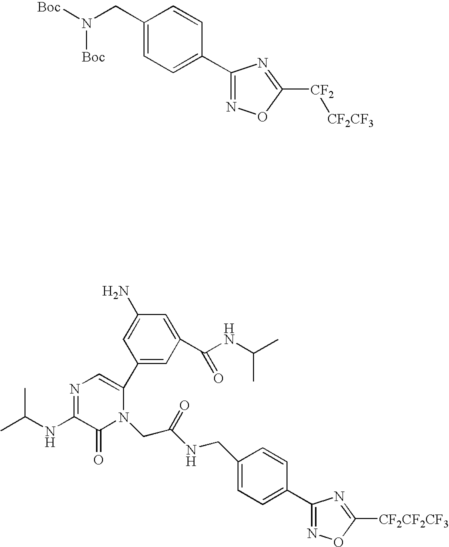

### Example 14a

To a solution of dibocaminobenzyl-4-hydroxamidine (0.75 g, 2.05 mmol) in 20 ml dichloromethane was added pyridine (0.38 mL, 4.51 mmol) and heptafluoropropionic anhydride (0.53 mL, 2.15 mmol) at 0° C. The reaction mixture was allowed to warm to room temperature and stirred for 2 hrs. The reaction mixture was then diluted with water. The layers were separated and the aqueous layer extracted with dichloromethane (2×). The organic extracts were washed with brine (1×). The organic fractions were dried (Na₂SO₄) and the solvent removed in vacuo to give a white solid, which after chromatography (silica, 10% ethyl acetate/hexanes) gave Example 14a as a white solid (0.92 g). m/z+1=544.

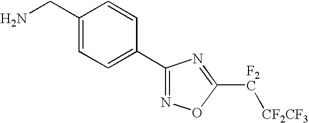

### Example 14b

To a solution of the product of Example 14a (0.92 g, 1.70 mmol) was added 4.0 N HCl in 20 ml of dioxane at room temperature. After stirring for 3 hrs at room temperature the precipitate was filtered and dried on high vacuum to give a white powder Example 14b (0.552 g). m/z+1=344

### Example 14c

To a solution of corresponding acid as used in Example 13 (0.25 g, 0.64 mmol) in 15 ml of DMF was added TBTU (0.20 g, 0.64 mmol) at 0° C. After 5 min, the product of Example 14b (0.22 g, 0.64 mmol) and DIEA (0.45 mL, 2.56 mmol) were added. The reaction was stirred for 1 hr and then diluted with water and ethyl acetate. The layers were separated and the organic layer washed with saturated sodium bicarbonate and dried (Na₂SO₄). The solvent was removed to give a semi-solid, which after chromatography (silica, ethyl acetate) gave Example 14c (0.18 g) as a yellow solid. m/z+1=713

### Example 15

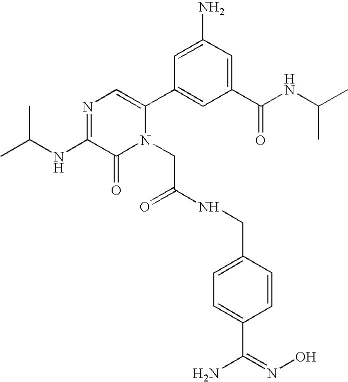

Prepared analogously to Example 1.

### Example 16

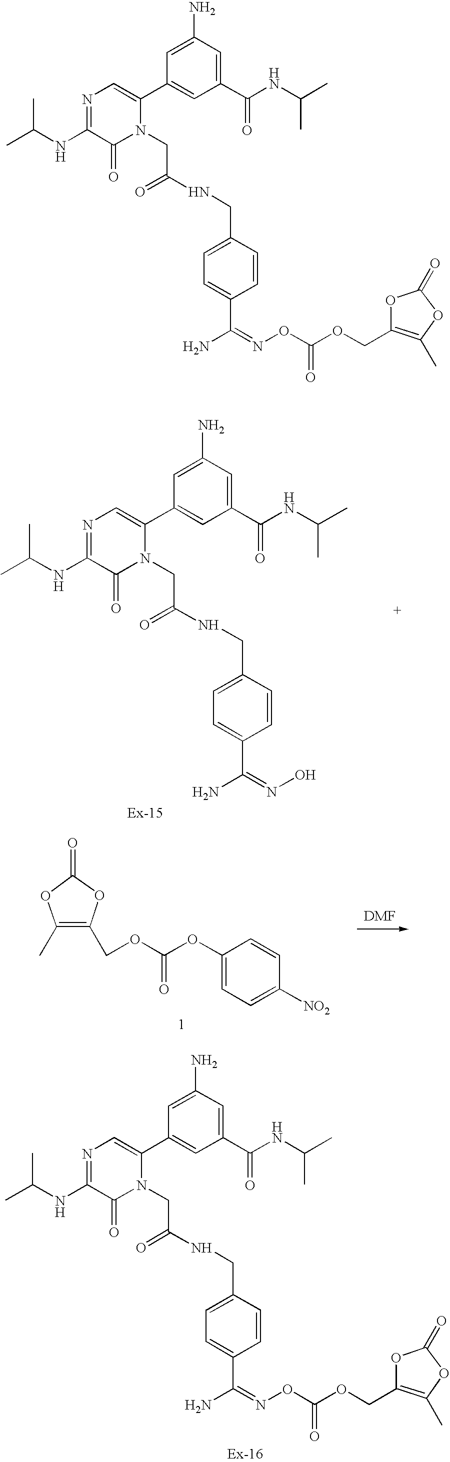

The product of Example 15 (87.3 mg, 0.163 mmol) and Compound 1 (prepared as in U.S. Pat. No. 5,466,811) (53.0 mg, 0.180 mmol) were stirred overnight at room temperature in 2 ml of DMF. The bright yellow crude reaction mixture was purified by reverse phase HPLC and lyophilized to afford a colorless solid Example 16 (11.0 mg, 0.012 mmol) as a TFA salt. HPLC/MS calc. Mass Spec M+H: 691.2762. Found: 691.29.

### Example 17

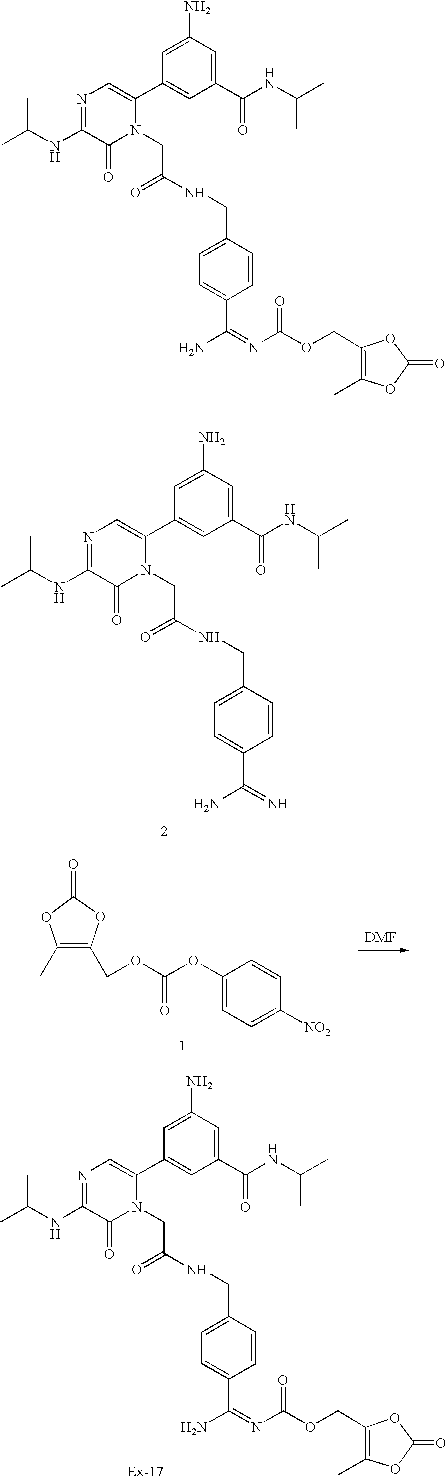

Compound 2 (prepared by catalytic hydrogenation of Example 15) (50.0 mg, 0.96 mmol) and 1 (31.0 mg, 0.106 mmol) were stirred at room temperature for 1 hour in DMF (1 mL). The crude reaction mixture was purified by reverse phase HPLC and lyophilized to afford a light yellow solid Example 17 (33.0 mg, 0.031 mmol) as a TFA salt. ¹H-NMR (300 MHz, CD₃OD): δ 1.25 (d, J=6.6 Hz, 6H), 1.40 (d, J=6.4 Hz, 6H), 2.30 (s, 3H), 4.15 (m, 2H), 4.51 (s, 2H), 4.69 (s, 2H), 5.28 (s, 2H), 6.73 (s, 1H), 6.87 (s, 1H), 7.06 (s, 1H), 7.22 (s, 1H), 7.47 (d, J=7.9 Hz, 2H), 7.80 (d, J=8.26 Hz, 2H); HRMS calculated for C₃₃H₃₈N₈O₈ Mass Spec M+H: 675.2813. Found: 675.2885.

### Example 18

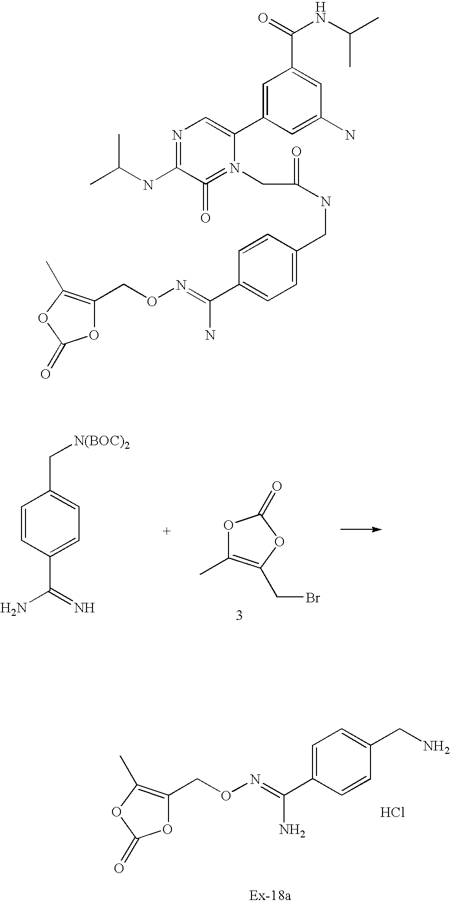

### Example 18a

Dibocaminobenzyl-4-hydroxamidine (50.0 mg, 0.14 mmol), compound 3 (prepared as in U.S. Pat. No. 5,466,811) (30.0 mg, 0.16 mmol) and KHCO₃ (16.0 mg, 0.16 mmol) were dissolved in 0.5 ml of DMF and 5 ml of acetonitrile at 60° C. for 5 hours. The crude reaction mixture was concentrated under a stream of N₂. To the resulting orange oil was added 4 N HCl (1 mL) in 1 ml of dioxane and stirred at room temperature for 5 hours. The crude reaction mixture was concentrated under a stream of N₂ to afford Example 18a as an orange solid.

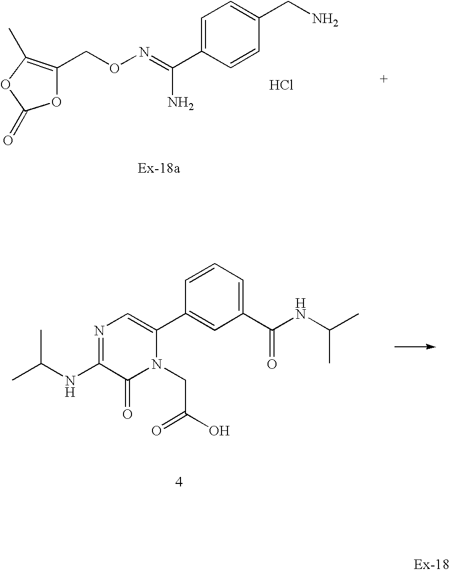

### Example 18b

To the product of Example 18a was added compound (4) (yellow solid, Mass spec M+H=418) (20.0 mg, 0.052 mmol), NMM (21 mg, 0.21 mmol), HOBT (8.4 mg, 0.062 mmol), and DMF (0.75 mL). EDC (12.0 mg, 0.062 mmol) was then added and the reaction mixture was stirred at room temperature for 2 hours. The crude reaction mixture was diluted with methanol, purified by reverse phase HPLC, and lyophilized to afford a yellow solid, Example 18b (8.0 mg, 0.0092 mmol) as a TFA salt. HPLC/MS calc. Mass spec M+H: 647.2863. Found: 647.43.

### Example 19

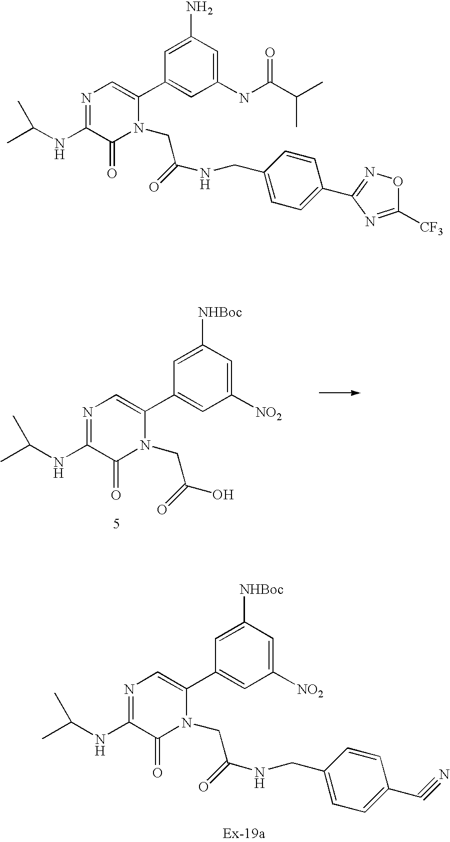

### Example 19a

To compound 5 (prepared analogously to Example 1a-1 g) (1.5 g, 3.36 mmol) was added HOBT (0.45 g, 3.36 mmol) and EDC (0.71 g, 3.7 mmol) in 12 ml of DMF. The mixture was stirred at RT for 30 min. The amine HCl salt (0.62 g, 3.7 mmol) in DMF (8 ml) and NMM (0.67 g, 6.7 mmol) was then added to the mixture which was kept stirring overnight. Water was then added to the mixture and filtered to yield 2 g solid. MS confirmed the product. MS (ES, m/z) 562.23 (M+H).

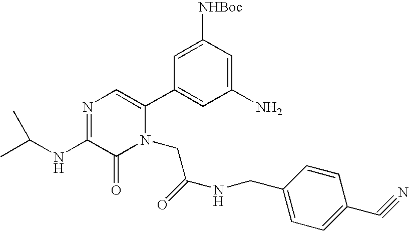

### Example 19b

To the product of Example 19a (1.6 g) in THF (20 ml) was added Pd/C (10% 0.5 g). The mixture was set on hydrogenation shake at 25 psi for 3 hr, then filtered and concentrated to yield 1.5 g solid without purification. MS (ES, m/z) 532.26 (M+H)

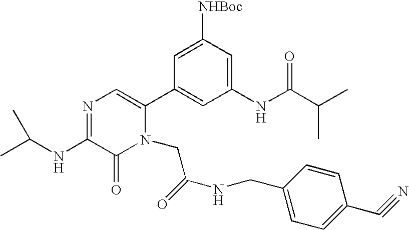

### Example 19c

To the product of Example 19b (1.7 g, 3.22 mmol) in CH₂Cl₂ (15 ml) was added NMM (0.37 g, 3.5 mmol) and isobutryl chloride (0.37 g, 3.5 mmol). The mixture was kept stirring at room temperature for 1 hr. The mixture was then washed with 40 ml of 10% citric acid, 40 ml of saturated NaHCO₃ and 40 ml of water. Combined CH₂Cl₂ was dried with MgSO₄, filtered and concentrated to yield 2 g crude products without purification. MS (ES, m/z) 602.30 (M+H)

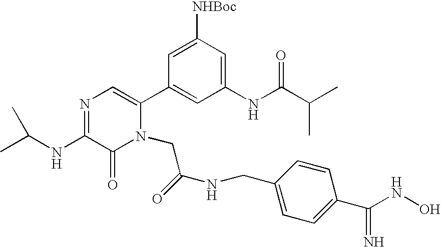

### Example 19d

To the product of Example 19c (0.85 g, 1.4 mmol) in 4 ml of ethanol was added NH₂OH (225 mg, 3.15 mmol) and K₂CO₃ (450 mg, 3.15 mmol) at room temperature. The mixture was heated at 80° C. overnight, filtered and concentrated to yield 0.7 g solid without purification. MS (ES, m/z) 635.32 (M+H).

### Example 19

To the product of Example 19d (0.24 g, 0.38 mmol) in 2 ml pyridine was added trifloroacetic anhydrate (99 mg, 0.47 mmol). The mixture was heated to 65° C. for overnight, then concentrated and added with CH₂Cl₂/TFA (1 ml/2 ml). The mixture was stirred at room temperature for 1 hr, then purified on RP-HPLC to yield 85 mg white solid.

mp 221-224° C.

HRMS calculated for C₂₉H₃₁F₃N₈O₄ Mass Spec M+H: 613.2493. Found: 613.2506.

Anal. Calculated for C₂₉H₃₁F₃N₈O₄+1CF3COOH, 2H₂O: C, 48.82; H, 4.75; N, 14.69.

Found: C, 48.87; H, 4.47; N, 14.32.

¹H NMR (d₄-DMSO) δ 1.08 (6H, d, J=6.7 Hz, CH₃), 1.25 (6H, d, J=6.4 Hz, CH₃), 2.59 (1H, m, CH), ?4.09 (1H, m, CH), 4.39 (2H, d, J=5.9 Hz, CH₂), 4.45 (2H, s, CH₂), 6.42 (1H, s, CH), 6.68 (1H, s, CH), 6.94 (1H, s, CH), 7.23(1H, s, CH), 7.44 (2H, d, J=8.3 Hz, 2CH), 8.01 (2H, d, J=8.3 Hz, 2CH), 8.65 (1H, s, NH), 9.80 (1H, s, NH).

### Example 20

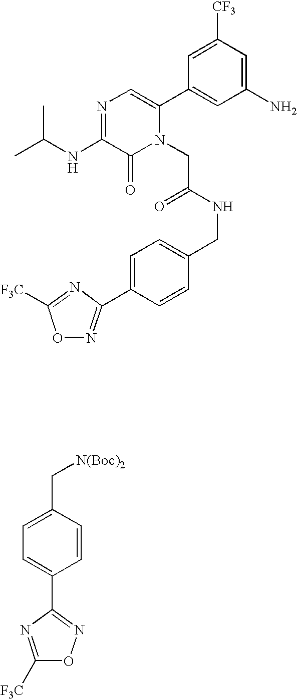

### Example 20a

To a mixture of dibocaminobenxyl-4-hydroxamidine (8.2 mmol, 3.0 g) and pyridine (31.5 mmol, 2.55 ml) was added trifluoroacetic acid anhydride (18.1 mmol, 2.55 ml) while cooling in a water bath. The reaction was stirred at room temperature for 2 hours. The reaction was then concentrated in vacuo and the residue was mixed with ethyl acetate (100 ml), washed with 1 N sodium hydrogen sulfate (3×25 ml), saturated sodium bicarbonate (2×25 ml) and brine (25 ml), dried over magnesium sulfate, filtered, and concentrated in vacuo to give 3.49 g of Example 20a as an off-white solid.

¹H NMR (CDCl₃) δ 1.51 (s, 18H), 4.89 (s, 2H), 7.79 (d, J=8.7 Hz, 2H), 8.14 (d, J=8.4 Hz, 2H).

¹⁹F NMR (CDCl₃) δ −65.06 (s, 3F).

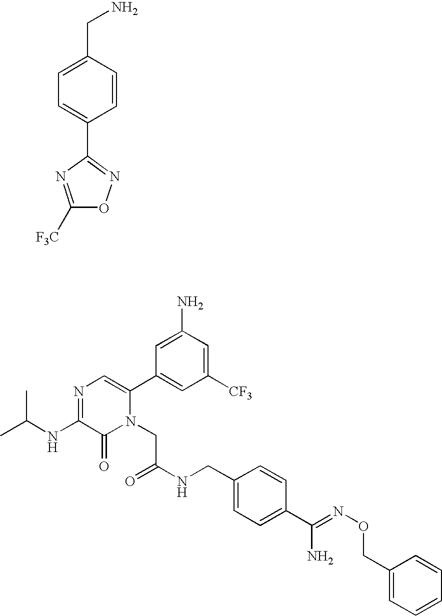

### Example 20b

A mixture of the product of Example 20a (7.5 mmol, 3.34 g) in 20 ml of dioxane was stirred with 4 N hydrogen chloride in 40 ml of dioxane at ambient temperature for 1 hour. The reaction was concentrated in vacuo to give 2.46 g of Example 20b as an off-white solid.

¹H NMR (d₆-DMSO) δ 4.17 (s, 2H), 7.78 (d, J=8.7 Hz, 2H), 8.14 (dd, J=1.8, 6.6 Hz, 2H), 8.66 (br s, 2H).

¹⁹F NMR (d₆-DMSO) δ −65.06 (s, 3F)

MS (ES) M+H m/z 244.

### Example 20c

To the product of Example 1g (0.97 mmol, 0.43 g), N-methylmorpholine (1.0 mmol, 0.11 mL), N-cyclohexylcarbodiimide-N′-methylpolystyrene (PS-DCC) (4.25 mmol, 2.5 g), and HOBT (1.06 mmol, 0.144 g) in 15 ml of DCM, cooled in an ice bath, was added a warm solution of Example 20b (0.88 mmol, 0.247 g,) in DMF (5 ml) and N-methylmorpholine (4.0 mmol, 0.44 mL). The reaction was slowly allowed to warm to room temperature and stirred for 16 hr. The reaction was filtered and the solids washed with DCM and DMF. The combined filtrate and washes were concentrated in vacuo and purification by reverse phase HPLC (30-70% acetonitrile/water) followed by lyophilization yielding 169 mg (27% yield) of Example 20 as an off-white solid.

mp: 196-198° C.

¹H NMR (d₆-DMSO) δ 1.29 (s, J=6.6 Hz, 6H), 4.07-4.20 (m, 1H), 4.39 (s, 2H), 4.41 (s, 2H), 6.77, (s, 1H), 6.82 (s, 2H), 7.45 (d, J=8.4 Hz, 2H), 8.03 (d, J=8.4 Hz, 2H), 8.74 (t, J=5.8 Hz, 1H).

¹⁹F NMR (d₆-DMSO) δ −61.98 (s, 3F), −65.08 (s, 3F)

HRMS calculated for C₂₆H₂₄F₆N₇O₃₃ (M+H): 596.1839. Found: 596.1809.

Anal. calculated for C₂₆H₂₃ F₆N₇O₃+0.9 TFA, 0.15 H₂O: C, 47.64; H, 3.48; N, 13.99.

Found: C, 47.68; H, 3.54; N, 13.84.

### Example 21

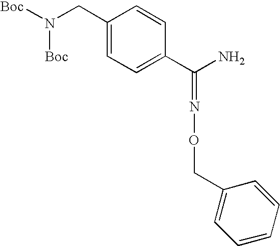

### Example 21a

A solution of di(tert-butyl) 4-[amino(imino)methyl]benzylimidodicarbonate (2.34 g, 5.72 mmol), diisopropyl ethyl amine (2.22 g, 17.2 mmol), and O-benzylhydroxylamine hydrochloride (1.83 g, 11.4 mmol) in 200 ml of ethanol was refluxed in a 500 ml round bottom flask for 12 hours. The reaction mixture was then allowed to cool and concentrated to give 9.71 g of crude product. The crude product was chromatographed on silica to give 1.54 g of a white crystalline product, 59% yield.

LC (0-60%, acetonitrile/water, in 8 min): 5.91 min.

MS M+H 456

NMR (400 MHz, CDCl₃): ¹H 1.433 ppm (18H,s), 4.763 ppm (2H,s), 5.135 ppm (2H,s), 7.349 ppm (7H,m), 7.602 ppm (2H,m).

### 4-(aminomethyl)-N′-(benzyloxy)benzenecarboximidamide Hydrochloride

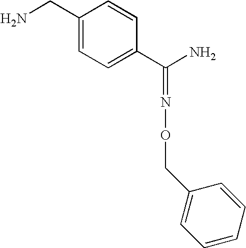

### Example 21b

The product from Example 21a (0.40 g, 0.878 mmol) was dissolved in 4 M hydrochloride acid in dioxane (50 ml, 200 mmol) and stirred 2 hours. The reaction mixture was concentrated, redissolved in ethyl acetate and concentrated to give 0.33 g of a white powder, 4-(aminomethyl)-N′-(benzyloxy)benzenecarboximidamide hydrochloride.

LC (0-60%, acetonitrile/water, in 8 min): 2.13 min.

MS M+H 256

NMR (400 MHz, CDCl₃) ¹H 4.109 ppm (2 H,s), 5.008 ppm (2 H,s), 7.348 ppm (3 H,m), 7.448 ppm (6 H,m).

### Example 21

A solution of the product from Example 1g (0.35 g, 0.58 mmol), the product from Example 21b (0.33 g, 1.0 mmol), benzotriazol-1-yl tetramethyluronium tetrafluoroborate (1.1 g, 3.43 mmol), diisopropyl ethyl amine (0.84 g, 6.5 mmol), and in 50 ml of N,N-dimethyl formamide and stirred 3 hours. To the reaction mixture was added 20 ml of 10% Potassium hydrogen sulfate. A precipitate formed and was filtered off. The precipitate was concentrated and then dissolved in acetonitrile and water. The product was purified by HPLC to 300 mg of 1 N-(4-{(Z)-amino[(benzyloxy)imino]methyl}benzyl)-2-[6-[3-amino-5-(trifluoromethyl)phenyl]-3-(isopropylamino)-2-oxopyrazin-1(2H)-yl]acetamide trifluoroacetate a whitish yellow solid, 71% yield.

LC (0-60%, acetonitrile/water, in 8 min): 4.39 min.

MS M+H 608

NMR (400 MHz, CD₃OD): ¹H 1.345 ppm (6 H,d), 4.046 ppm (1 H,m), 4.421 ppm (2 H,broad d), 4.511 ppm (2 H,s), 5.085 ppm (2 H,s), 6.662 ppm (1 H,s), 6.869 ppm (2 H,d), 7.001 ppm (1 H,s), 7.384 ppm (5 H,m), 7.517 ppm (4 H,m).

¹⁹F −64.848 ppm (3 F,s), −77.509 ppm (3 F,s).

Elemental Analysis

| | | | |
|---|---|---|---|
| Found: | C: 50.61 | H: 4.25 | N: 11.89 |
| Calc. | C: 51.99 | H: 4.29 | N: 12.41 |

### Example 22

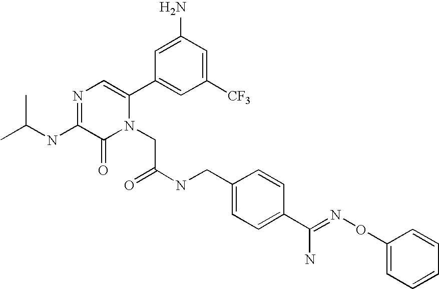

### Example 22a

A solution of di(tert-butyl) 4-[amino(imino)methyl]benzylimidodicarbonate (2.35 g, 5.74 mmol), diisopropyl ethyl amine (2.23 g, 17.2 mmol), and O-phenylhydroxylamine hydrochloride (1.67 g, 11.4 mmol) in 100 ml of ethanol was refluxed in a 500 ml round bottom flask for 36 hours. The reaction mixture was allowed to cool and concentrated to give 5.8 g of crude product. The crude product was chromatographed on silica to give 0.65 g of a white crystalline product, 25% yield.

LC (0-60, acetonitrile/water, in 8 min): 5.31 min.

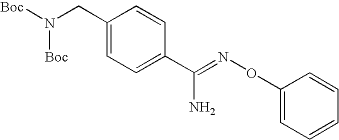

MS M+H 442

NMR (400 MHz, CDCl₃): ¹H 1.447 ppm (18H,s), 4.796 ppm (2 H,s), 6.908 ppm (1 H,m), 7.289 ppm (6 H,m), 7.699 ppm (2 H,m).

### 4-(aminomethyl)-N′-phenoxybenzenecarboximidamide Hydrochloride

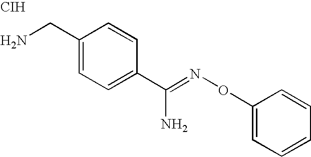

### Example 22b

The product from Example 22a (0.650 g, 1.47 mmol) was dissolved in 4 M hydrochloride acid in dioxane (25 ml, 100 mmol) and stirred for 8 hours. The reaction mixture was concentrated, redissolved in ethyl acetate and concentrated to give 0.463 g of a white powder, 4-(aminomethyl)-N′-(phenyloxy)benzenecarboximidamide hydrochloride.

LC (0-60%, acetonitrile/water, in 8 min): 2.622 min.

MS M+H 242

### Example 22c

A solution of the product from Example 1g (0.98 g, 2.21 mmol), the product from Example 22b (0.463 g, 1.47 mmol), benzotriazol-1-yl tetramethyluronium tetrafluoroborate (1.76 g, 5.48 mmol), diisopropyl ethyl amine (1.41 g, 10.9 mmol), and in 50 ml of N,N-dimethyl formamide and stirred 6 hours. A precipitate formed and was filtered off. The precipitate dissolved in acetonitrile and water. The product was purified by HPLC to 400 mg of 1 N-(4-{(Z)-amino[(phenyloxy)imino]methyl}benzyl)-2-[6-[3-amino-5-(trifluoromethyl)phenyl]-3-(isopropylamino)-2-oxopyrazin-1(2H)-yl]acetamide trifluoroacetate a whitish yellow solid, 45% yield.

LC (0-60%, acetonitrile/water, in 8 min): 5.36 min.

MS M+H 594

NMR (400 MHz, CD₃OD): ¹H 1.323 ppm (6 H,d), 4.020 ppm (1 H,m), 4.332 ppm (2 H,broad d), 4.444 ppm (2 H,s), 6.622 ppm (1 H,s), 6.877 ppm (2 H,d), 6.957 ppm (1 H,s), 7.022 ppm (1 H,s), 7.236 ppm (6 H,m), 7.618 ppm (1 H,m) 7.700 ppm (2 H,d).

¹⁹F −63.841 ppm (3 F,s), −76.861 ppm (3 F,s).

Elemental Analysis

| | | | |
|---|---|---|---|
| Found | C: 48.98 | H: 3.91 | N: 11.79 |
| Calculated | C: 49.70 | H: 3.93 | N: 11.93 |

### Example 23

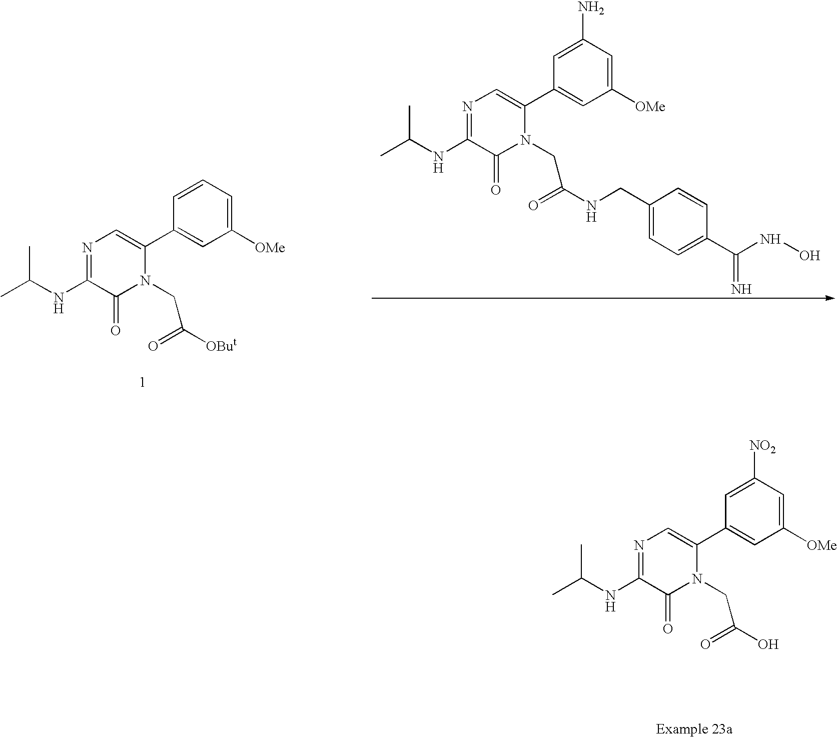

### Example 23a

0.234 g (0.56 mmol) of compound 1 was hydrolyzed with 10 mL TFA for 2 hours stirring at room temperature. TFA was evaporated to dryness to yield 0.19 g (0.52 mmol) of Example 23a as a white solid.

Mass Spec: M+H =363.2.

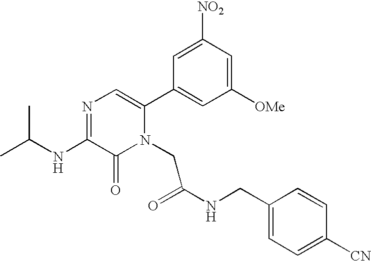

### Example 23b

Example 23a was dissolved in 15 mL DMF and coupled with 0.332 g (1 mmol) 4-aminobenzonitrile in the presence of 0.256 g (0.8 mmol) TBTU and 0.525 mL DIPEA for 16 hours. Adding 200 mL H₂O, the product precipitated and it was filtered and dried to yield 0.205 g (0.43 mmol; 76%) of Example 23b as a yellow solid.

Mass Spec: M+H =477.3.

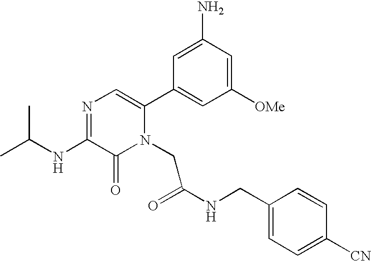

### Example 23c

The product of Example 23b was dissolved in 20 mL MeOH and reduced with 0.126 g HCOONH₄ in the presence of 0.05 g Pd black stirring under N₂ for 15 minutes. The catalyst was filtered off and the solvent was evaporated to afford Example 23c.

### Example 23d

The clear oily residue was dissolved in 10 mL EtOH and refluxed with H₂N—OH xHCl in the presence of 1 mL DIPEA for 4 hours. The solvent was evaporated and the residue was purified on preparative HPLC using a gradient of acetonitrile (10-50% AcN in 30 minutes), yielding the title product at 38% AcN, 0.092 g (23%) as a white hygroscopic solid.

Mass Spec: M+H=480.4.

¹H NMR: 400 MHz, CD₃OD: 7.68-7.62 (d, 2H), 7.48-7.42 (d, 2H), 6.84-6.80 (s, 2H), 6.57-6.50 (m, 2H), 4.62-4.56 (s, 2H), 4.50-4.42 (m, 2H), 4.08-3.98 (m, 1H), 3.75-3.70 (m, 3H) and 1.42-1.34 (m, 6H).

Elemental analysis:

| | | | |
|---|---|---|---|
| Found | C: 45.32 | H: 4.63 | N: 12.71 |
| Calculated | C: 45.58 | H: 4.47 | N: 13.10 |

### Example 24

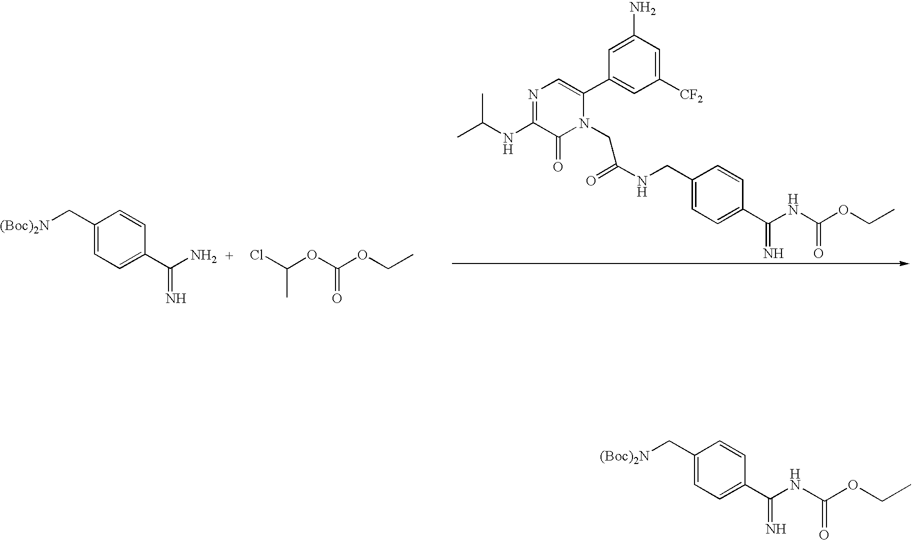

### Example 24a

1 g (2.4 mmol) of 4-(N,N-diBoc-amino)-benzylamidine acetate was dissolved in 10 mL DMF and it was reacted with 0.67 mL (5 mmol) 1-chloroethyl ethyl carbonate in the presence of 7.5 mL (7.5 mmol) 1 N NaOH for 16 hours. Then DMF was evaporated and the residue was dissolved in 100 mL EtOAc. It was washed with brine, dried over MgSO₄, filtered and the solvent was evaporated to afford Example 24a. Yield: 0.91 g (2.1 mmol; 90%) oil.

Mass Spec: M+H=422.2.

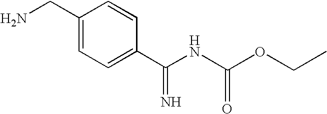

### Example 24b

The product of Example 24a was dissolved in 40 mL CH₂Cl₂ and it was deprotected with 10 mL TFA stirring for 30 minutes to afford Example 24b.

Mass spec: M+H=222.3.

Example 24

The product of Example 24b was coupled with 0.484 g (1 mmol) of the product of Example 1g in the presence of 0.875 mL (5 mmol) DIPEA and 0.385 g (1.2 mmol) TBTU with stirring for 3 hours in 30 mL DMF. The solvent was evaporated and the residue was purified on preparative HPLC using a gradient of acetonitrile (10-50% AcN in 30 minutes), yielding the title product at 34% AcN, 0.46 g (57%) as a light brown solid.

Mass Spec: M+H =574.3.

¹H NMR: 400 MHz, CD₃OD: 7.78-7.73 (d, 2H), 7.51-7.44 (d, 2H), 7.02 (s, 1H), 6.90-6.84 (d, 2H), 6.68 (s, 1H), 4.54-4.38 (m, 6H), 4.10-4.00 (m, 1H) and 1.42-1.32 (m, 9H).

¹⁹F NMR: 400 MHz, CD₃OD: −64.9 (s, 3F) and −77.5 (s, 6F)

Elemental analysis:

| | | | |
|---|---|---|---|
| Found | C: 45.58 | H: 4.10 | N: 12.41 |
| Calculated | C: 45.43 | H: 4.18 | N: 11.96 |

### Example 25

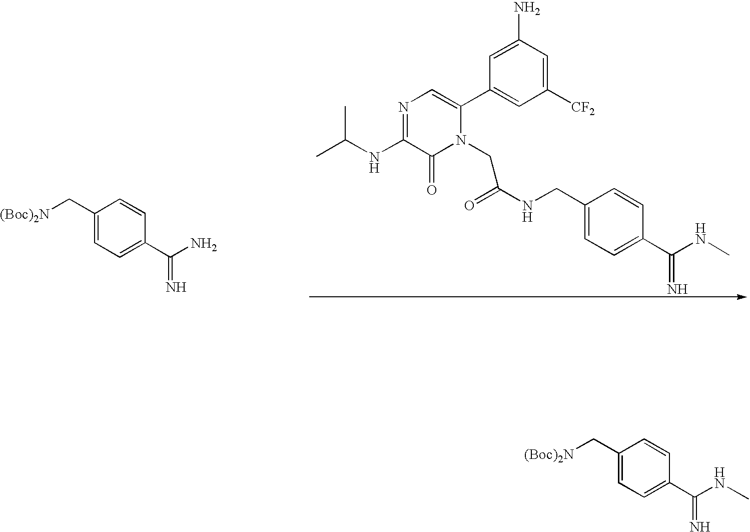

### Example 25a

2.5 g (6.1 mmol) 4-(N,N-diBoc-amino)-benzylamidine acetate was dissolved in 30 mL MeOH and it was refluxed with 1.35 g (20 mmol) methylamine xHCl in the presence of 5.25 mL (30 mmol) DIPEA for 12 hours. MeOH was evaporated and the residue was dissolved in 100 mL EtOAc and it was washed with brine, dried over MgSO₄ and the solvent was evaporated to yield 1.5 g (4.2 mmol; 67%) of Example 25a as an oil.

Mass Spec: M+H=364.3.

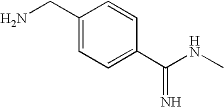

### Example 25b

The product of Example 25a was dissolved in 40 mL CH₂Cl₂ and it was deprotected with 10 mL TFA stirring for 30 minutes to afford Example 25b.

### Example 25

The product of Example 25b was coupled with 0.484 g (1 mmol) of the product of Example 1g in the presence of 1.75 mL (20 mmol) DIPEA and 0.353 g (1.1 mmol) TBTU with stirring for 3 hours in 25 mL DMF. The solvent was evaporated and the residue was purified on preparative HPLC using a gradient of acetonitrile (10-50% AcN in 30 minutes), yielding the title product at 32% AcN, 0.27 g (36%) as a white solid.

Mass Spec: M+H=516.2.

¹H NMR: 400 MHz, CD₃OD: 7.69-7.62 (d, 2H), 7.47-7.42 (d, 2H), 7.00 (s, 1H), 6.88-6.82 (d, 2H), 6.68 (s, 1H), 4.52 (s, 2H), 4.46 (m, 2H), 4.10-4.00 (m, 1H), 3.32-3.28(m, 3H) and 1.38-1.28 (m, 6H).

¹⁹F NMR: 400 MHz, CD₃OD: −64.9 (s, 3F) and −77.5 (s, 6F)

Elemental analysis:

| | | | |
|---|---|---|---|
| Found | C: 45.62 | H: 4.11 | N: 13.16 |
| Calculated | C: 45.74 | H: 4.24 | N: 12.87 |

### Example 26

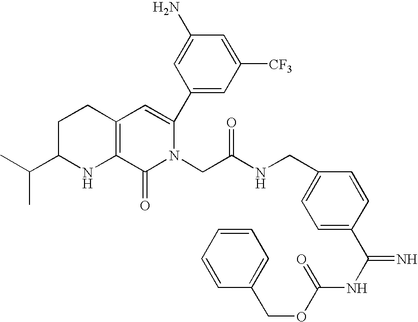

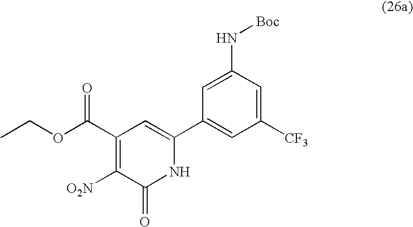

To a slurry of 500 mmol of the ammonium salt of 2-nitroacetamide in 400 grams of water is added 600 mmol of ethyl 2,4-dioxo-4-(3-(t-butoxycarbonylamino)-5-trifluoromethylphenyl) butanoate (prepared by standard methods from diethyl oxalate and 1-acetyl-3-(t-butoxycarbonylamino)-5-trifluoromethylbenzene). A solution of piperidinium acetate (prepared by adding 72 mL of piperidine to 42 mL of acetic acid in 200 mL of water) is then added. The resulting reaction mixture is stirred at 40° C. for about 24 hours. The reaction product 26a is then separated, dried and used in the next step.

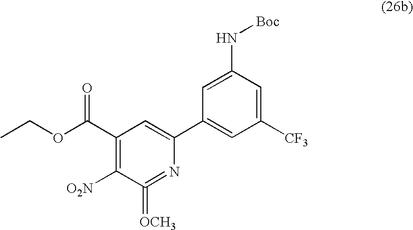

A solution of the pyridone 26a from Step A (400 mmol) in 500 mL of methylene chloride is treated with 500 mmol of solid trimethyloxonium tetrafluoroborate and the mixture stirred at 40° C. until the reaction is complete as monitored by liquid chromatography. The reaction mixture is concentrated about 70% and chromatographed on silica gel to afford the methoxy pyridine 26b.

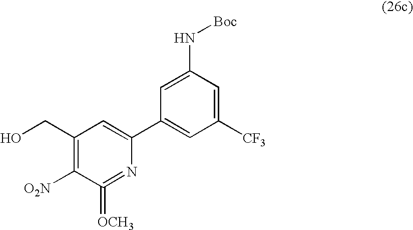

To a solution of the pyridine 26b from Step B (350 mmol) in 1000 mL of methylene chloride at −70° C. is added with 700 mmol of DIBAL (1 molar in hexane) using a dropping funnel. The resulting solution is stirred for 1 hour and then warmed to room temperature over an additional hour. The reaction mixture is quenched by the careful addition of saturated sodium potassium tartrate. After stirring for 30 additional minutes, the solid is filtered and washed with 500 mL methylene chloride. The filtrate is washed twice with 500 mL of saturated sodium potassium tartrate and then 500 mL of brine. The solution is concentrated and then chromatographed to afford the desired alcohol 26c.

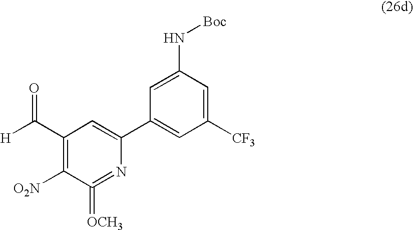

To a solution of phosgene (350 mmol) in 1000 mL of methylene chloride at −70° C. is added 700 mmol of DMSO in 100 mL methylene chloride using a dropping funnel. Then, the resulting solution is treated with the pyridone alcohol 26c from Step C (300 mmol) in 500 mL of methylene chloride, stirred for an additional 15-30 minutes, treated with 225 mL of triethylamine, and then warmed to room temperature over an additional 1.5 hours. The reaction mixture is quenched by the addition of 1000 mL water and the two phases separated. The aqueous is extracted twice with 1000 mL of methylene chloride and the combined organic extracts is washed with 500 mL of brine. The methylene chloride solution is dried over MgSO₄, concentrated, and then chromatographed to afford the desired aldehyde 26d.

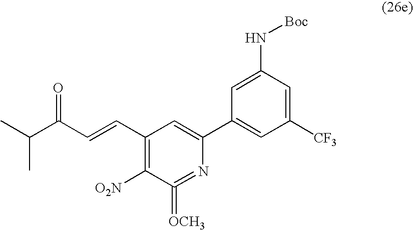

To a solution of diethyl 2-(3-methyl-2-oxo-butyl) phosphonate (250 mmol; obtainable through a standard Arbuzov reaction between 1-bromo-3-methyl-2-butanone and triethyl phosphite) in 1000 mL of THF at 0° C. is added 250 mmol of NaH. Then, the resulting solution is stirred until hydrogen evolution ceased and then treated with the pyridine aldehyde 26d from Step D (250 mmol) in 800 mL of THF. The solution is heated at 50° C. for 180 minutes, cooled, and evaporated. The residue is redissolved in 2000 mL of ethyl acetate and quenched to a pH of 7 with saturated ammonium chloride. The organic phase is washed with brine, dried over MgSO₄, concentrated, and then chromatographed to afford the desired nitro ketone 26e.

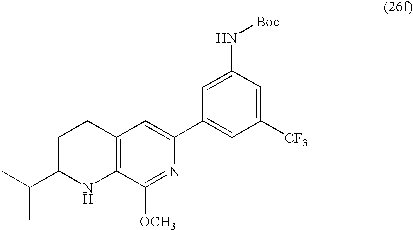

To a solution of nitro ketone 26e from Step E (225 mmol) in 1000 mL of ethyl acetate is added 20 grams of 10% Pd/C. Hydrogen gas is added until uptake of hydrogen stopped. The reaction mixture is filtered through Celite and the filtrate evaporated. The residue is then chromatographed to afford the desired bicyclic methoxy pyridine 26f.

Step G:

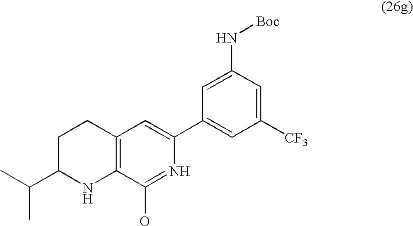

To a solution of methoxy pyridine 26f from Step F (200 mmol) in 1000 mL of dichloroethane at ambient temperature is added 400 mmol of boron tribromide in 400 mL methylene chloride. After stirring for about two hours, the reaction mixture is quenched to a pH of 8 with saturated sodium bicarbonate. The mixture was diluted with 2000 mL of ethyl acetate and 200 mL of THF. The aqueous phase is discarded and the organic solution washed with 200 mL water followed by 200 mL of brine. The reaction mixture evaporated to afford the desired bicyclic pyridone 26 g.

Step H:

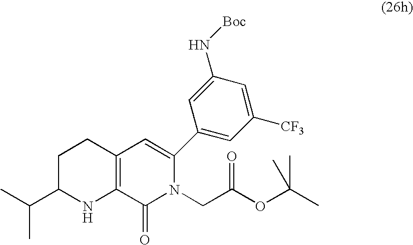

The bicyclic pyridone 26 g from Step G (150 mmol) is alkylated with tert-butyl bromoacetate using the procedure of Example 1d to afford the desired bicyclic pyridone acetate 26h.

Step I:

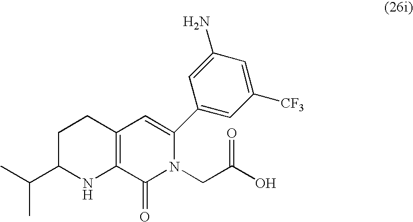

The bicyclic pyridone acetate 26 h from Step H (100 mmol) is deprotected with trifluoroacetic acid as described in Example 1g to afford the desired bicyclic pyridone acetic acid 26i.

A solution of compound bicyclic pyridone acetic acid 26i (50 mmol) in DMF (250 mL) is treated with N-hydroxybenzotriazole (60 mmol) and EDC hydrochloride (60 mmol). The mixture is stirred at room temperature for 30 min and treated with 4-(N-Cbz-amidinobenzylamine (50 mmol). The resulting mixture is allowed to stir overnight. Typical aqueous workup is followed by chromatographic purification to afford pure Example 26 product.

### Example 27x

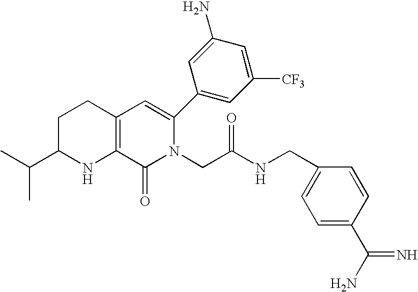

Compound of Example 26 (10 mmol) and 10% Pd on activated carbon (0.100 g) are mixed with 100 mL methanol. The mixture is stirred for 2 hours under an atmosphere of hydrogen that is introduced through a rubber balloon. After filtering off the catalyst and removing the methanol, the remaining residue is obtained as Example 27.

### Example 28x

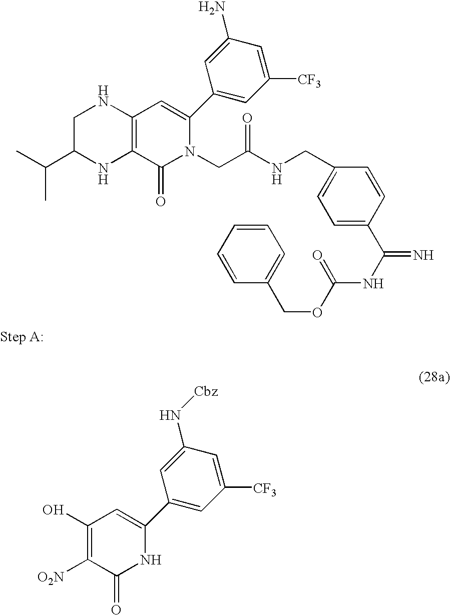

Step A:

To a slurry of 500 mmol of the ammonium salt of 2-nitroacetamide in 400 grams of water is added 600 mmol of ethyl 3-oxo-3-(3-(t-butoxycarbonylamino)-5-trifluoromethylphenyl) propanoate (prepared by standard methods from diethyl carbonate and 1-acetyl-3-(t-butoxycarbonylamino)-5-trifluoromethylbenzene). A solution of piperidinium acetate (prepared by adding 36 mL of piperidine to 21 mL of acetic acid in 100 mL of water) is then added. The resulting reaction mixture is stirred at 40° C. for about 24 hours. The reaction product 28a is then separated, dried and used in the next step.

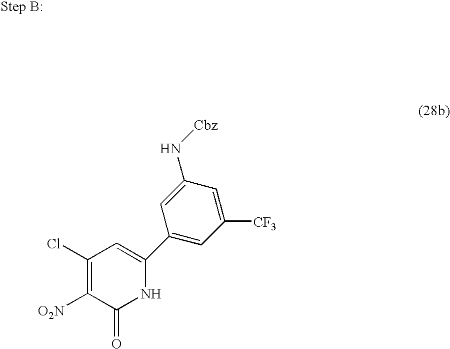

A solution of the pyridone 28a from Step A (400 mmol) in 2000 mL of acetonitrile is treated with 1.6 moles of phosphorusoxychloride and 1.5 moles N-benzyl-N,N,N-triethylammonium chloride. The mixture is stirred at 40° C. and then heated at reflux until the reaction is complete as monitored by liquid chromatography. The reaction mixture is concentrated to remove solvent, and the residue is slurried with water (1000 mL). The product is separated to afford the chloro pyridone 28b.

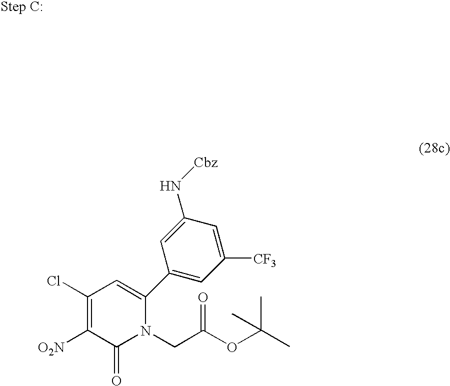

The chloro pyridone 28b from Step B (350 mmol) is alkylated with tert-butyl bromoacetate using the procedure of Example 1d to afford the desired bicyclic pyridone acetate 28c.

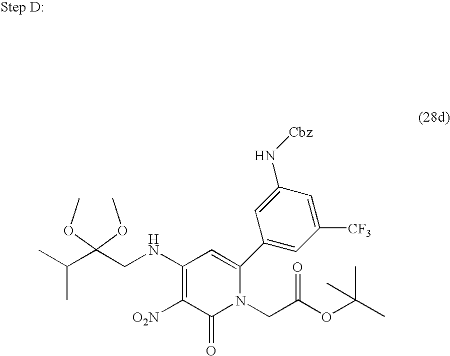

To a solution of bicyclic pyridone acetate 28c from Step C (300 mmol) in 1500 mL of ethanol is added 2,2-dimethoxy-3-methylbutanamine (300 mmol) and 600 mmol of triethylamine. The solution is stirred at 70° C. for 16 hours or until the reaction is complete. The reaction mixture is cooled and evaporated to remove all of the ethanol. The residue is partitioned between ethyl acetate and water, and the organic phase is washed with brine, dried over MgSO₄, concentrated, and then chromatographed to afford the desired nitro ketal 28d.

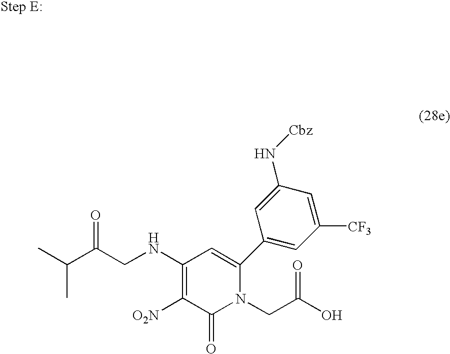

The nitro ketal 28d from Step D (250 mmol) is hydrolyzed and the tert-butyl ester removed by stirring with trifluoroacetic acid (50 mL), water (200 mL) and THF (500 mL) until completion as monitored by chromatography. The reaction mixture is concentrated at ambient temperature to give the trifluroacetic acid salt of unpurified nitro ketone 28e and used as is in the next step.

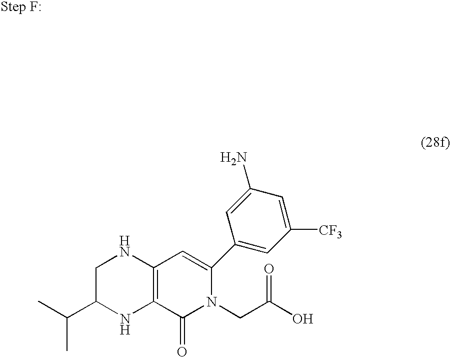

To the nitro ketone 28e from Step E (225 mmol) in 1000 mL of ethyl acetate is added 20 grams of 10% Pd/C. Hydrogen gas is added until uptake of hydrogen stopped. The reaction mixture is filtered through Celite and the filtrate evaporated. The residue is then chromatographed to afford the desired bicyclic pyridone acetic acid 28f.

A solution of compound bicyclic pyridone acetic acid 28f (50 mmol) in DMF (250 mL) is treated with N-hydroxybenzotriazole (60 mmol) and EDC hydrochloride (60 mmol). The mixture is stirred at room temperature for 30 min and treated with 4-(N-Cbz-amidinobenzylamine (50 mmol). The resulting mixture is allowed to stir overnight. Typical aqueous workup is followed by chromatographic purification to afford pure Example 28 product.

### Example 29x

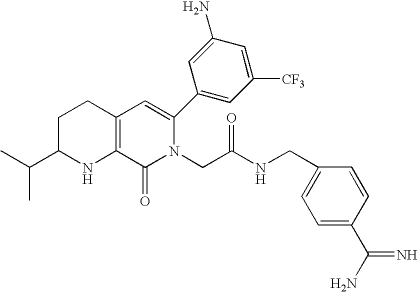

Compound of Example 28 (10 mmol) and 10% Pd on activated carbon (0.100 g) are mixed with 100 mL methanol. The mixture is stirred for 2 hours under an atmosphere of hydrogen that is introduced through a rubber balloon. After filtering off the catalyst and removing the methanol, the remaining residue is obtained as Example 29.

Using these methods and ordinary skill in the art of synthetic numerous novel compounds of the present invention have been or can be prepared.

### Metabolic Stability Assay

### Hepatic S9 Fraction Incubation

In order to determine the metabolic stability of each of the tested compounds, the following assay conditions were used: 100 mM-phosphate buffer, 1.0 mM NADPH, 3.3 mM magnesium chloride, 2.0 mg/mL protein from Hepatic S9 Fraction and 1.0 μM of substrate.

The assay was performed on a 96-well conical shaped plate with a volume of 200 μL, after addition of methanol. For each species, a set of samples was prepared as shown below (volumes are in μL).

| | Inactivated S9 | Active S9 |
|---|---|---|
| | Fraction | Fraction |
| Solution Added | Protein | Protein |
| 2X Cofactor Buffer | 40 | 40 |
| 10X Inactivated S9 | 10 | 0 |
| Fraction | | |
| 10X Active S9 Fraction | 0 | 10 |
| Protein | | |
| 2X Substrate | 50 | 50 |
| MeOH (with Internal | 100 | 100 |
| Standard) | | |

The plate was sealed and incubated in a Thermal Mixer at 37° C. for 30 minutes at 400 rpm. 100 μL of methanol was added to each well and the plate was mixed for several minutes and then covered with an aluminum seal. Finally, the plate was centrifuged at 1000 rpm for 10 minutes.

The assay was analyzed using liquid chromatography and positive ion electrospray mass spectrometry. The chromatography column used was Agilent Zorbax SB-C18 (3.0×150 mm, 5 μm) with a flow rate of 0.5 mL/min.

Calculations were performed to determine the percent remaining, which is calculated by dividing the Peak Area Ratio of the analyte measured in the active protein versus the Peak Area Ratio of the analyte measured in the inactivated protein sample. The Peak Area Ratio is defined as the peak area of the analyte divided by the peak area of the internal standard. Analyte is defined as the substrate of a specific measurable metabolite.

The results of the assay are summarized in Table 1 below.

TABLE 1

| Comp. No. | BA | S9 Rat | S9 Hum. | % Conv. | BA Drug |
|---|---|---|---|---|---|
| 1 | | 11% | 2% | | |
| 2 | | | | | |
| 3 | | | | | |
| 4 | 17% | 0% | 0% | | |
| 5 | 5% | 13% | 0.50% | | |
| 6 | 7% | | | | |
| 7 | | 54% | 5% | 22% | 0.60% |
| 8 | 20% | 3% | 1% | 0.30% | 0.40% |
| 9 | 1% | 0% | 0% | 9% | |
| 10 | | | | | |
| 11 | | 44% | 40% | | |
| 12 | 3% | | | 11% | 0.50% |
| 13 | 68% | 2% | 4% | 6% | 3% |
| 14 | 1% | 1% | 1% | 2.40% | 0% |
| 15 | 4% | 25% | 39% | | |
| 16 | | 0% | 0% | | |
| 17 | | | | | |
| 18 | | 36% | 5% | | |
| 19 | | | | | 1.60% |
| 20 | 2% | LR | LR | | 1% |
| 21 | | | | | |
| 22 | | | | | |
| 23 | | NR | NR | | |
| 24 | | 1% | 1% | | |
| 25 | | 13% | 3% | | |
| 29 | | 14% | 17% | | |

“BA” represents bioavailability of prodrug after oral adminstration,
“S9 rat” and “S9 human” represent respectively rat and human liver S9 fractions.

### Bioavailability

Test System:

Healthy male rats [Crl:CD(SD)BR] were obtained from Charles River Breeding Laboratory (Canada). The rats did not receive any drug treatments prior to the initiation of the study. The animals weighed 250 to 320 g and were individually identified by labeling on each metabolism cage. The rats were housed in individual metabolism cages during dosing and sample collections. The animals were acclimated to a diet of Purina Rodent Chow #5002 (Ralston Purina, St. Louis, Mo.) for at least 5 days and were fasted for 15-20 hours prior to the administration of the compound. Food was available from 4 hours after dose administration and ad libitum throughout the remainder of the study.

Doses:

Each animal received the prodrug orally at an equivalent dose of 10 mg of free base of active moiety per kg of body weight or active moiety orally at an equivalent dose of 10 mg equivalent free base per kg body weight. For intravenous (IV) study, the animals received 1 mg free base/kg body weight. Sufficient amount of the test article was dissolved in appropriate vehicle (See table below) such that the final concentration of dose solution was 2.0 mg free base/mL and 0.5 mg free base/mL for oral and IV doses, respectively. The dose volume was 5 mL/kg and 2 mL/kg for oral and IV doses, respectively

Sample Collection and Analysis:

Blood samples were collected from the jugular vein at specified time intervals. Concentrations of test article and/or prodrug were analyzed using a LC-MS/MS procedure.

Phamacokinetic Analysis:

Model independent pharmacokinetic parameters (Cmax, Tmax, AUC, T1/2, CL, Vss) were obtained using Watson computer program. The bioavailability (BA) was calculated as follows:

$$\% BA = \frac{{[ AUC ]}_{oral} / Oral Dose}{{[ AUC ]}_{IV} / IV dose} \times 100$$

The percentages of conversion of prodrug to active moiety was calculated using the following equation:

$$\% Conversion = \frac{{[ AUC ]}_{Active Moiety} After IV Dose of Prodrug}{{[ AUC ]}_{Active Moiety} After IV Dose of Active Moiety} \times 100$$

“AUC” represents area under the curve.

The results are shown in Table 1 in Example 30.

Table for vehicles
IG Formulations:

1. 10% EtOH/10% Tween 80/80% Capmul MCM
2. 10% EtOH/10% PEG 200/80% H20
3. 10% EtOH/10% PEG 400/80% H2O
4. 10% Tween 80/90% Capmul MCM
5. 100% PEG 400
6. 10% EtOH/5% Tween 80/85% Capmul MCM

IV Formulations:

1. 10% PEG 400/10% EtOH/80% Saline
2. 30% PEG 400/70% Saline
3. 35% PEG 400/65% Saline
4. 40% PEG 400/60% Saline
5. 10% PEG 400/10% EtOH/80% Saline
6. 30% PEG 400/5% Tween 80/65% Saline
7. 100% Saline

### Example 27

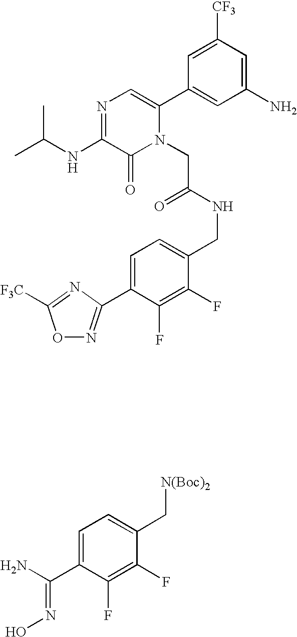

### Example 27a

A mixture of di-(tert-butyl) 4-cyano-2, 3-difluorobenzylimidodicarbonate (0.5 g, 1.4 mmol), hydroxylamine hydrochloride (0.28 g, 4.1 mmol), and triethylamine (0.57 ml, 4.1 mmol) in ethanol was heated to reflux for 1 hour. The reaction was concentrated in vacuo and the residue mixed with ethyl acetate, washed with 1N potassium hydrogen sulfate, saturated sodium bicarbonate, brine, dried over magnesium sulfate, filtered, and evaporated in vacuo to give 0.5 g of Ex-27a (89% yield). LCMS (M+H) m/z 402. ¹HNMR (300 MHz, CDCl₃) δ 1.50 (s, 18 H), 4.92 (s, 2H), 5.56 (br s, 2H), 7.05-7.14 (m, 1H), 7.42-7.53 (m, 1H). ¹⁹FNMR (282 MHz, CDCl₃) δ −142.53 to −142.32 (m, 1F), −141.3 to −141.0 (m, 1F). LCMS (ES+) m/z M+H 402.

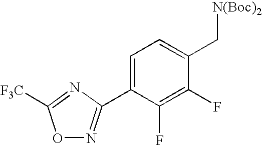

### Example 27b

To a stirred solution of Ex. 27a (0.45 g, 1.1 mmol) in pyridine (0.35 ml) and dichloromethane (0.5 ml) was added trifluoroacetic acid anhydride (0.35 ml, 2.5 mmol) while cooling in a water bath and stirring was continued at ambient temperature for 20 minutes. The reaction was concentrated in vacuo and the residue dissolved in ethyl acetate and washed with 1N sodium hydrogen sulfate, brine, dried over magnesium sulfate, filtered, and concentrated in vacuo to gave 0.43 g as an off-white solid. ¹HNMR (300 MHz, CDCl₃) δ 1.52 (s, 18H), 4.99 (s, 2H), 7.19-7.26 (m, 1H), 7.82-7.90 (m, 1H). ¹⁹FNMR (282 MHz, CDCl₃) δ −141.8 to −141.6 (m, 1F), −133.2 to −132.9 (m, 1F), −65.6 (s, 3F).

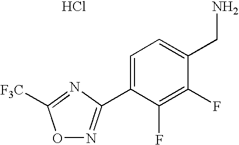

### Example 27c

¹HNMR (300 MHz, DMSO-d₆) δ 4.25 (s, 2H), 7.68-7.75 (m, 1H), 7.97-8.05 (m, 1H), 8.73 (br s, 3H). ¹⁹FNMR (282 MHz, DMSO-d₆) δ −139.5 to −139.4 (m, 1F), −134.7 to −134.6 (m, 1F), −65.0 (s, 3F)

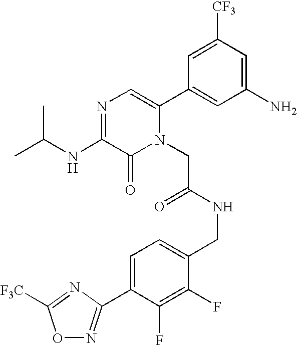

### Example 27

¹HNMR (300 MHz, DMSO-d₆) δ 1.23 (d, J=6.3 Hz, 6H), 4.05-4.20 (m, 1H), 4.41 (s, 2H), 4.46 (d, J=5.7 Hz, 2H), 5.81 (s, 2H), 6.72 (s, 1H), 6.80 (s, 1H), 6.89-6.95 (m, 2H), 7.33 (t, J=6.7 Hz, 1H), 7.82-7.90 (m, 1H), 8.78 (t, J=5.8 Hz, 1H). ¹⁹FNMR (282 MHz, DMSO-d₆) δ −142.7 to −142.5(m, 1F), −135.4 to −135.2 (m, 1F), −65.0 (s, 3F), −62.0 (s, 3F). HRMS (ES) calcd for C₂₆H₂₂N₇O₃F₈ (M+H): 632.1651. Found: 632.1674. Anal. Calcd for C₂₆H₂₁N₇O₃F₈+0.15 CH₄OH: C, 49.36; H, 3.42; N, 15.40. Found: C, 49.47; H, 3.33; N, 15.27.

### Example 28

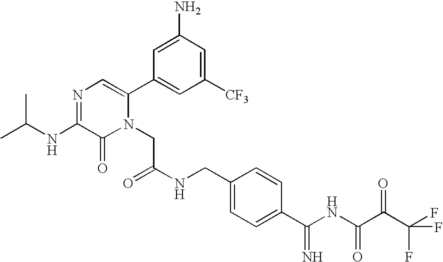

### Example 28

MH⁺=626.2

¹HNMR: 400 MHz, CD₃OD: 7.86-7.80 (d, 2H), 7.30-7.40 (d, 2H), 7.00 (s, 1H), 6.84-6.76 (d, 2H), 6.62 (s, 1H), 4.50 (s, 2H), 4.40 (s, 2H) 4.08-4.00 (m, 1H) and 1.40-1.32 (m, 6H).

¹⁹FNMR: 400 MHz, CD₃OD: −66.0 (s, 3F) and −84.5 (s, 3F)

Elemental analysis: C₂₇H₂₅N₇O₄F₆ + 2xTFA + 1.5xH₂O

| | | | |
|---|---|---|---|
| Found | C: 42.08 | H: 3.46 | N: 11.43 |
| Calc. | C: 42.28 | H: 3.43 | N: 11.13 |

### Example 29

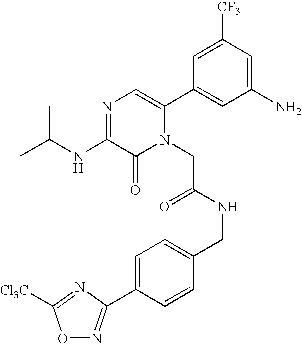

### Example 29

¹HNMR (300 MHz, DMSO-d₆) δ 1.23 (d, J=6.3 Hz, 6H), 4.08-4.20 (m, 1H), 4.39-4.48 (m, 4H), 5.84 (s, 2H), 6.73 (s, 1H), 6.80-6.85 (m, 2H), 6.91-6.96 (m, 2H), 7.46 (d, J=8.4 Hz, 2H), 8.02 (d, J=8.4 Hz, 2H), 8.72 (t, J=5.7 Hz, 1H). ¹⁹FNMR (282 MHz, DMSO-d₆) δ −61.92 (s). HRMS (ES) calcd for C₂₆H₂₄N₇O₃F₃Cl₃ (M+H): 644.0953. Found: 644.0984. Anal. Calcd for C₂₆H₂₃N₇O₃F₃Cl₃: C, 48.43; H, 3.59; N, 15.20; Cl, 16.49. Found: C, 48.60; H, 3.56; N, 15.07; Cl, 16.28.

### Example 30

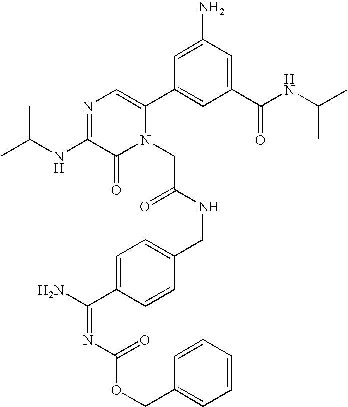

### Example 30

¹HNMR (300 MHz, DMSO-d₆) δ 1.15 (d, J=6.6 Hz, 6H), 1.23 (d, J=6.6 Hz, 6H), 4.03-4.20 (m, 2H), 4.36 (d, J=5.4 Hz, 2H), 4.44 (s, 2H), 5.15 (s, 2H), 5.50 (br s, 2H), 6.68 (s, 1H), 6.72 (s, 1H), 6.83 (d, J=8.1 Hz, 1H), 6.99 (s, 1H), 7.11 (s, 1H), 7.32 (d, J=8.1 Hz, 2H), 7.36-7.48 (m, 5H), 7.95 (d, J=8.1 Hz, 2H), 8.03 (d, J=7.5 Hz, 1H), 8.61 (t, J=5.4 Hz, 1H) 9.22 (br s, 2H). HRMS (ES) calcd for C₃₅H₄₁N₈O₅ (M+H): 653.3194. Found: 653.3240. Anal. Calcd for C₃₅H₄₀N₈O₅+0.1 water: C, 64.40; H, 6.18; N, 17.17. Found: C, 64.23; H, 6.16; N, 16.97.

### Example 31

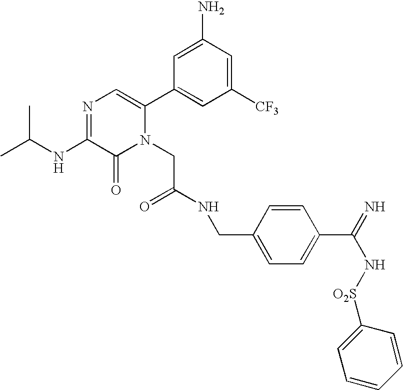

### 2-[6-[3-amino-5-(trifluoromethyl)phenyl]-3-(isopropylamino)-2-oxopyrazin-1(2H)-yl]-N-(4-{imino[(phenylsulfonyl)amino]methyl}benzyl)acetamide

### Example 31

HRMS calcd for C₃₀H₃₀F₃N₇O₄S₁ (M+H): 642.2105. Found: 642.2148.

Anal. Calcd for C₃₀H₃₀F₃N₇O₄S₁ + 1.6TFA + 0.3H2O:

| | | | |
|---|---|---|---|
| | C: 48.07; | H: 3.91; | N: 11.81. |
| Found: | C: 48.10; | H: 4.01; | N: 11.72. |

¹H NMR (DMSO-d₆, 300 MHz) δ 1.26 (d, 6H), 2.63 (s, 1H), 4.12 (m, 2H), 4.36 (m, 4H), 6.77 (s, 1H), 6.81 (bs, 2H), 6.96(s, 1H), 7.31(d, 2H), 7.57-7.66 (m, 3H), 7.83 (d, 2H), 7.96(d, 2H), 8.28(s, 1H), 8.69 (t, 1H), 9.10 (s, 1H).

### Example 32

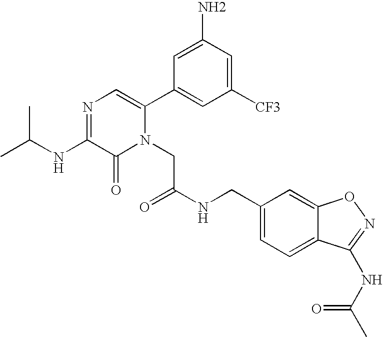

### N-{[3-(acetylamino)-1,2-benzisoxazol-6-yl]methyl}-2-[6-(3-amino-5-methylphenyl)-3-(isopropylamino)-2-oxopyrazin-1(2H)-yl]acetamide

### Example 32

HRMS calcd for C₂₆H₂₆F₃N₇O₄ (M+H): 558.2071 Found: 558.2088.

Anal. Calcd for C₂₆H₂₆F₃N₇O₄ + 1.35TFA + 0.25H2O:

| | | | |
|---|---|---|---|
| | C: 48.14; | H: 3.92; | N: 13.69. |
| Found: | C: 48.13; | H: 4.91; | N: 13.69. |

¹H NMR (DMSO-d₆, 300 MHz) δ 1.26 (d, 6H), 2.12 (s, 3H), 4.13 (m, 2H), 4.42 (bs, 3H), 6.80 (d, 2H), 7.11(d, 1H), 7.25 (s, 1H), 7.47 (bs, 1H), 7.75(d, 2H), 7.83(s, 1H), 8.17 (s, 1H), 8.72 (t, 1H), 10.45 (s, 1H).

### Example 33

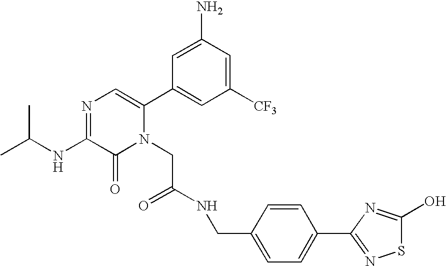

### 2-[6-[3-amino-5-(trifluoromethyl)phenyl]-3-(isopropylamino)-2-oxopyrazin-1(2H)-yl]-N-[4-(5-hydroxy-1,2,4-thiadiazol-3-yl)benzyl]acetamide

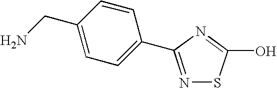

### Example 33a

Di(tert-butyl)4-[amino(imino)methyl]-benzylimidodicarbonate (2 g, 5 mmol) in 10 ml CH2Cl2 was added perchloromethylmercaptan (0.83 g, 4.5 mmol). The mixture was added NaOH/H2O (1 g/1.5 ml) under −8° C. and kept stirring for 7hrs, then quenched with water (50 ml) and extracted with CH2Cl2 (3×25 ml). The combined CH2Cl2 was then dried over MgSO4, concentrated and purified on silica gel column to yield 0.2 g solid. The solid in CH2Cl2 (2 ml) was then added TFA (1.5 ml) at RT for 1 hr, then concentrated and purified on RP-HPLC to yield 1.3 g solid (50%).

Cl₉H₂₅N₃O₅S M.W. 407.49.

### Example 33

HRMS calcd for C₂₅H₂₄F₃N₇O₃S₁(M+H): 560.1686. Found: 560.1709.

Anal. Calcd for C₂₅H₂₄F₃N₇O₃S₁ + 0.6TFA + 0.85H2O:

| | | | |
|---|---|---|---|
| | C: 48.91; | H: 4.12; | N: 15.24. |
| Found: | C: 48.93; | H: 4.19; | N: 15.19. |

¹H NMR (DMSO-d₆, 300 MHz) δ 1.26 (d, 6H), 4.13 (m, 3H), 4.39 (m, 4H), 5.79(s, 1H), 6.75 (s, 1H), 6.80 (d, 2H), 6.95 (s, 1H), 7.36 (d, 2H), 7.91(d, 2H), 8.68(t, 1H), 13.42 (s, 1H).

### Example 34

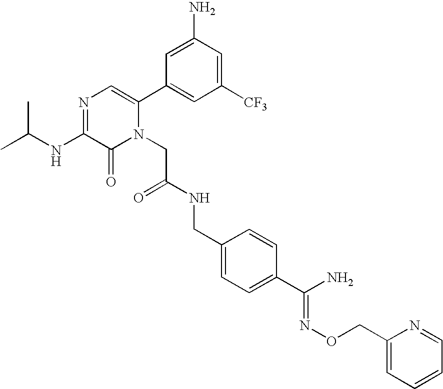

### N-(4-{(Z) -amino[(pyridin-2-ylmethoxy)imino]methyl}benzyl)-2-[6-[3-amino-5-(trifluoromethyl)phenyl]-3-(isopropylamino)-2-oxopyrazin-1(2H)-yl]acetamide

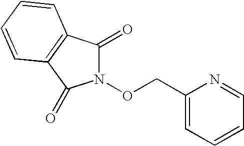

### 2-(pyridin-2-ylmethoxy)-1H-isoindole-1,3(2H)-dione

### Example 34a

To a round bottom flask containing 2-hydroxymethyl pyridine (5.0 g, 45.8 mmol), N-hydroxy phthalimide (18.7 mg, 114.5 mmol) and triphenylphosphine (33.7 g, 128 mmol) in 800 ml of tetrahydrofuran was added diisopropyl azodicarboxylate (25.3 ml, 128 mmol) dropwise. The solution was stirred overnight and then concentrated in vacuo. The residue was treated with 500 ml of ethyl acetate and extracted twice with 100 ml of 1M HCl. The combined aqueous layers were basified with 20 g of sodium bicarbonate and then treated with 150 ml of sodium bicarbonate (sat.) solution. The resulting precipitate was collected by vacuum filtration and dried over phosphorous pentoxide under high vacuum to give (7.2 g, 62% yield) of a white solid. MS-ESI (M+H)=255.

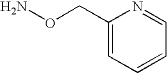

### 2-[(aminooxy)methyl]pyridine

### Example 34b

To the product from Ex. 34a) (7.2 g, 28.3 mmol) in 100 ml of methanol was added hydrazine (1.8 ml, 57.3 mmol) and the mixture was stirred over the weekend. The mixture was filtered and concentrated in vacuo. The residue was purified by chromatography (silica, 10-20% MeOH:CH₂Cl₂) to give (1.23 g, 35% yield) of a solid. ¹H NMR (400 MHz, CDCl₃): δ 8.56 (d, 1 H), 7.68-7.64 (m, 1 H), 7.34 (d, 1 H), 7.18 (t, 1 H), 5.59 (bs, 2 H), 4.79 (s, 2 H).

### Example 34

¹H NMR (400 MHz, CD₃OD): δ 8.72 (d, 2 H), 8.40-8.36 (m, 1 H), 7.96 (d, 1 H), 7.81 (t, 1 H), 7.54 (d, 2 H), 7.25 (d, 2 H), 7.0 (s, 1 H), 6.84 (d, 2 H), 6.63 (s, 1 H), 5.32 (s, 2 H), 4.51 (s, 2 H), 4.37 (d, 2 H), 4.03-4.00 (m, 1 H), 1.36 (d, 6 H); MS-ESI (M+H)=609; Analysis: C₃₀H₃₁F₃N₈O₃+3.0 TFA+1.1 H₂O calcd: C, 45.26; H, 3.73; N, 11.72; O, 15.4; found: C, 44.58; H, 3.59; N, 11.41; O, 14.93.

### Example 35

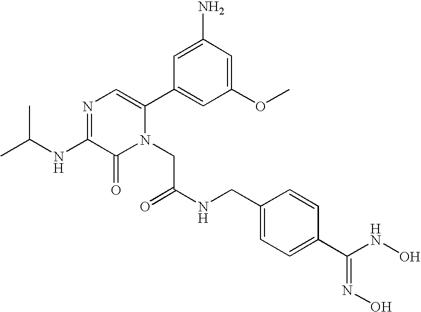

### Example 35a

This compound was isolated from the crude reaction mixture of example 23, on preparative HPLC using a gradient of acetonitrile (10-50% AcN in 30 minutes), yielding the title product at 28% AcN, 0.05 g (15%) as a hygroscopic solid.

MH⁺=496.2

¹HNMR: 400 MHz, CD₃OD: 7.68-7.60 (d, 2H), 7.50-7.42 (d, 2H), 6.64 (s, 1H), 6.48 (s, 1H), 6.42-6.36 (d, 2H), 4.59 (s, 2H), 4.50-4.42 (d, 2H), 4.10-3.98 (m, 1H), 3.70 (s, 3H) and 1.40-1.32 (m, 6H).

Elemental analysis: C₂₄H₂₉N₇O₅ + 2.5xTFA + 1.2xH₂O

| | | | |
|---|---|---|---|
| Found | C: 43.43 | H: 4.28 | N: 11.69 |
| Calc. | C: 43.42 | H: 4.26 | N: 12.22 |

### Example 36

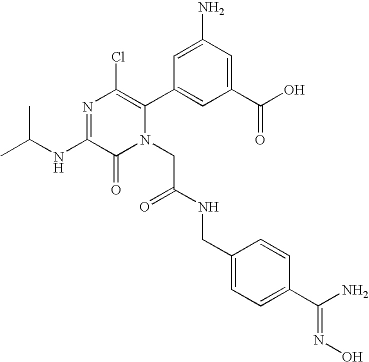

### 3-amino-5-[1-[2-({4-[(Z)-amino(hydroxyimino)methyl]benzyl}amino)-2-oxoethyl]-3-chloro-5-(isopropylamino)-6-oxo-1,6-dihydropyrazin-2-yl]benzoic Acid

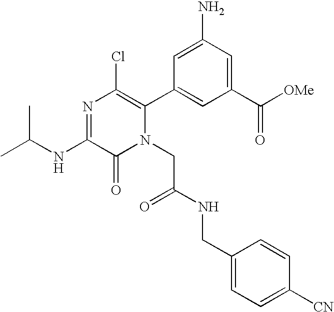

### Methyl 3-amino-5-[3-chloro-1-{2-[(4-cyanobenzyl)aminol-2-oxoethyl}-5-(isopropylamino)-6-oxo-1,6-dihydropyrazin-2-yl]benzoate

### Example 36a

¹H NMR (400 MHz, CD₃OD): δ 7.64 (d, 2 H), 7.40 (t, 1 H), 7.31 (d, 2 H), 7.17 (t, 1 H), 6.81 (t, 1 H), 4.44 (s, 2 H), 4.37 (s, 2 H), 4.18-4.14 (m, 1 H), 3.84 (s, 3 H), 1.26 (d, 6 H); MS-ESI (M+H)=509/510.

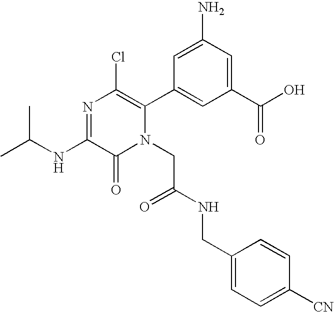

### 3-amino-5-[3-chloro-1-{2-[(4-cyanobenzyl)amino]-2-oxoethyl}-5-(isopropylamino)-6-oxo-1,6-dihydropyrazin-2-yl]benzoic Acid

### Example 36b

To a solution of Example 36a (0.60 g, 1.18 mmol) in THF:MeOH:H₂O (7:2:1) was added LiOH*H₂O (0.09 g, 2.36 mmol) at room temperature. The reaction was stirred at room temperature overnight and then acidified with acetic acid. The solvent was removed in vacuo to give a foam (0.58 g, 107%), which was used without further purification in the next step. MS-ESI (M+H)=496/497.

### Example 36c

To a solution of Example 36b (0.58 g, 1.10 mmol) in ethanol (15 mL) at room temperature was added K₂CO₃ (0.6 g, 4.84 mmol), DIEA (0.84 mL, 4.84 mmol) and H₂NOH*HCl (0.17 g, 2.42 mmol). The reaction mixture was heated to 75° C. for 2 hrs and then allowed to cool to room temperature. The solid was filtered and washed with ethanol. The filtrate was concentrated and acidified with TFA. The crude mixture was purified by RP-HPLC (CH₃CN:H₂O) to give after lypholization the desired product (0.28 g). ¹H NMR (400 MHz, CD₃OD): δ 7.62 (d, 2 H), 7.44 (t, 1 H), 7.40 (d, 2 H), 7.25 (t, 1 H), 6.86 (t, 1 H), 4.45 (s, 2 H), 4.41 (s, 2 H), 4.14-4.18 (m, 1 H), 1.26 (d, 6 H); MS-ESI (M+H)=528/529; Analysis: C₂₄H₂₆ClN₇O₅+1.75 TFA+0.8 H₂O calcd: C, 44.51; H, 3.98; N, 13.21; O, 20.05; found: C, 44.52; H, 4.08; N, 13.18; O, 19.98.

### Example 37

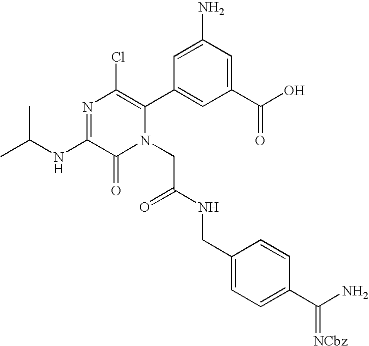

### Example 37

¹H NMR (400 MHz, CD₃OD): δ 7.72 (d, 2 H), 7.49-7.37 (m, 7 H), 7.22 (t, 1 H), 6.84 (t, 1 H), 5.39 (s, 2 H), 4.46 (s, 2 H), 4.40 (s, 2 H), 4.19-4.13 (m, 1 H), 1.27-1.25 (m, 6 H); MS-ESI (M+H)=646/647; Analysis: C₃₂H₃₂ClN₇O₆+2.15 TFA+1.15 H₂O calcd: C, 47.62; H, 4.05; N, 10.7; found: C, 47.61; H, 4.03; N, 10.72.

### Example 38

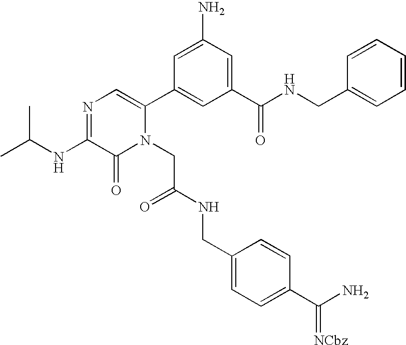

### Benzyl (1E)-amino{4-[({[6-(3-amino-5-{[(benzyl)amino]carbonyl}phenyl)-3-(isopropylamino)-2-oxopyrazin-1(2H)-yl]acetyl}amino)methyl]phenyl}methylidenecarbamate

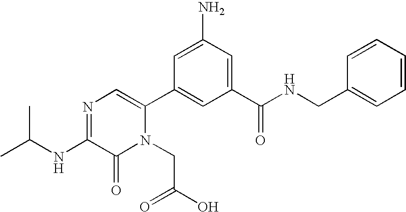

### [6-{3-amino-5-[(benzylamino)carbonyl]phenyl}-3-(isopropylamino)-2-oxopyrazin-1(2H)-yl]acetic Acid

### Example 38a

¹H NMR (400 MHz, CD₃OD): δ 7.34-7.19 (m, 7 H), 6.94 (t, 1 H), 6.63 (s, 1 H), 4.54 (s, 2 H), 4.52 (s, 2 H), 4.04-4.00 (m, 1 H), 1.39 (d, 2 H); MS-ESI (M+H)=436.

### Example 38b

¹H NMR (400 MHz, CD₃OD): δ 7.65 (d, 2 H) 7.51-7.19 (m, 13 H), 7.05 (t, 1 H), 6.86 (t, 1 H), 6.66 (s, 1 H), 5.41 (s, 2 H), 4.66 (s, 2 H), 4.51 (s, 2 H), 4.42-4.40 (m, 2 H), 4.07-4.03 (m, 1 H), 1.37 (d, 2 H); MS-ESI (M+H)=701; Analysis: C₃₉H₄₀N₈O₅+2.6 TFA+0.5 H₂O calcd: C, 52.75; H, 4.36; N, 11.13; found: C, 52.75; H, 4.38; N, 11.1.

### Example 39

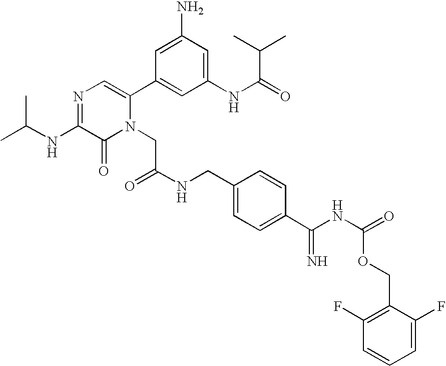

### 2,6-difluorobenzyl{4-[({[6-[3-amino-5-(isobutyrylamino)phenyl]-3-(isopropylamino)-2-oxopyrazin-1(2H)-yl]acetyl}amino)methyl]phenyl)(imino)methylcarbamate

### Example 39

Tert-butyl 3-[1-[2-({4-[amino(imino)methyl]benzyl}amino)-2-oxoethyl]-5-(isopropylamino)-6-oxo-1,6-dihydropyrazin-2-yl]-5-(isobutyrylamino)phenylcarbamate (0.2 g, 0.32 mmol) and 2,6-difluorobenzyl 4-nitrophenyl carbonate (120mg, 0.39 mmol) in THF (2 ml) was added NMM (40 mg, 0.40 mmol). The mixture was kept stirring for 3 hr. The mixture was concentrated and then TFA/CH2Cl2 was added. The mixture was kept stirring at RT for 1 hr, then concentrated and purified on RP-HPLC to yield 125 mg of solid (41%).

C₃₅H₃₈F₂N₈O₅ M.W. 688.72.

Anal. Calcd for C₃₅H₃₈F₂N₈O₅ + 2.2TFA + 0.25H20:

| | | | |
|---|---|---|---|
| | C: 50.12; | H: 4.34; | N: 11.86. |
| Found: | C: 50.20; | H: 4.46; | N: 11.68. |

¹H NMR (DMSO-d₆, 300 MHz) δ 1.07 (d, 6H), 1.23 (d, 6H) 2.10(s, 2H), 4.12 (m, 3H), 4.38 (d, 2H), 4.44 (s, 2H), 5.38 (s, 2H), 6.31 (s, 1H), 6.67 (bs, 1H), 6.81 (s, 1H), 7.12(s, 1H), 7.23 (t, 2H), 7.38 (d, 2H), 7.59(m, 1H), 7.79(d, 2H), 8.67 (t, 1H), 9.73 (s, 1H)

### Example 40

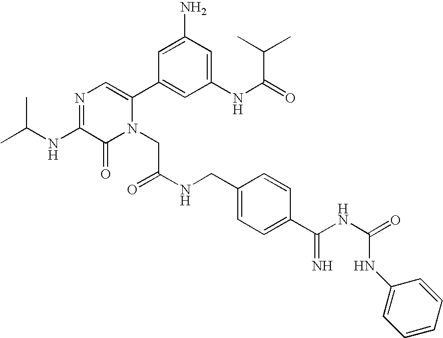

### N-{3-amino-5-[1-[2-({4-[[(anilinocarbonyl)amino](imino)methyl]benzyl}amino)-2-oxoethyl]-5-(isopropylamino)-6-oxo-1,6-dihydropyrazin-2-yl]phenyl}-2-methylpropanamide

### Example 40

Tert-butyl 3-[1-[2-({4-[amino(imino)methyl]benzyl}amino)-2-oxoethyl]-5-(isopropylamino)-6-oxo-1,6-dihydropyrazin-2-yl]-5-(isobutyrylamino)phenylcarbamate (0.18 g, 0.29 mmol) and isocyanatobenzene (42 mg, 0.35 mmol) in THF (2 ml) was added NMM (40 mg, 0.40 mmol). The mixture was kept stirring for 3 hr. The mixture was concentrated and then TFA/CH2Cl2 was added. The mixture was kept stirring at RT for 1 hr, then concentrated and purified on RP-HPLC to yield 85 mg of solid (33%).

HRMS calcd for C₃₄H₃₉N₉O₄ (M+H): 638.3198. Found: 638.3162.

Anal. Calcd for C₃₄H₃₉N₉O₄ + 2.2TFA + 0.45H2O:

| | | | |
|---|---|---|---|
| | C: 51.43; | H: 4.73; | N: 14.05. |
| Found: | C: 51.47; | H: 4.80; | N: 13.90. |

¹H NMR (DMSO-d₆, 300 MHz) δ 1.09 (d, 6H), 1.24 (d, 6H), 4.12 (m, 3H), 4.41 (d, 2H), 4.47 (s, 2H), 6.30 (s, 1H), 6.67 (s, 1H), 6.80 (s, 1H), 7.10(s, 1H), 7.17 (t, 1H), 7.42 (t, 2H), 7.48 (d, 2H), 7.58(d, 2H), 7.88 (d, 2H), 8.71(t, 1H), 9.72 (s, 1H).

### Example 41

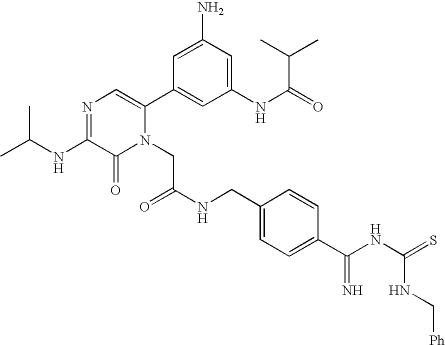

### N-{3-amino-5-[1-[2-({4-[{[(benzylamino)carbonothioyl]amino}(imino)methyl]benzyl}amino)-2-oxoethyl]-5-(isopropylamino)-6-oxo-1,6-dihydropyrazin-2-yl]phenyl}-2-methylpropanamide

### Example 41

Tert-butyl 3-[1-[2-({4-[amino(imino)methyl]benzyl}amino)-2-oxoethyl]-5-(isopropylamino)-6-oxo-1,6-dihydropyrazin-2-yl]-5-(isobutyrylamino)phenylcarbamate (0.18 g, 0.29 mmol) and (isothiocyanatomethyl)benzene (52 mg, 0.35 mmol) in THF (2 ml) was added NMM (40 mg, 0.40 mmol). The mixture was kept stirring for 3 hr. The mixture was concentrated and then TFA/CH2Cl2 was added. The mixture was kept stirring at RT for 1 hr, then concentrated and purified on RP-HPLC to yield 85 mg of solid (30%).

HRMS calcd for C₃H₄₁N₉O₃S₁ (M+H): 668.3126. Found: 668.3141.

Anal. Calcd for C₃₅H₄₁N₉O₃S₁ + 2.45TFA + 0.75H2O:

| | | | |
|---|---|---|---|
| | C: 49.88; | H: 4.71; | N: 13.12. |
| Found: | C: 49.92; | H: 4.75; | N: 13.01. |

¹H NMR (DMSO-d₆, 300 MHz) δ 1.08 (d, 6H), 1.24 (d, 6H), 4.12 (m, 2H), 4.36 (t, 2H), 4.44 (m, 2H), 4.63 (d, 1H), 4.77 (d, 1H), 6.36 (s, 1H), 6.68 (s, 1H), 6.87 (s, 1H), 7.17 (s, 1H), 7.33 (m, 6H), 7.89 (t, 2H), 8.65(t, 1H), 9.77 (s, 1H).

### Example 42

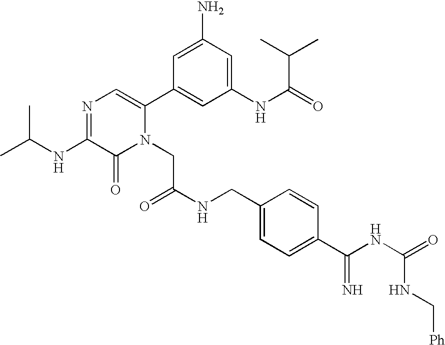

### N-{3-amino-5-[1-[2-({4-[{[(benzylamino)carbonyl]amino}(imino)methyl]benzyl}amino)-2-oxoethyl]-5-(isopropylamino)-6-oxo-1,6-dihydropyrazin-2-yl]phenyl}-2-methylpropanamide

### Example 42

Tert-butyl 3-[1-[2-({4-[amino(imino)methyl]benzyl}amino)-2-oxoethyl]-5-(isopropylamino)-6-oxo-1,6-dihydropyrazin-2-yl]-5-(isobutyrylamino)phenylcarbamate (0.18 g, 0.29 mmol) and (isocyanatomethyl)benzene (52 mg, 0.39 mmol) in THF (2 ml) was added NMM (40 mg, 0.40 mmol). The mixture was kept stirring for 3 hr. The mixture was concentrated and then TFA/CH2Cl2 was added. The mixture was kept stirring at RT for 1 hr, then concentrated and purified on RP-HPLC to yield 104 mg of solid (40%).

HRMS calcd for C₃₅H₄₁N₉O₄ (M+H): 652.3354. Found: 652.3368.

Anal. Calcd for C₃₅H₄₁N₉O₄ + 2.1TFA + 1.0H2O:

| | | | |
|---|---|---|---|
| | C: 52.83; | H: 4.87; | N: 14.14. |
| Found: | C: 53.07; | H: 5.04; | N: 13.35. |

¹H NMR (DMSO-d₆, 300 MHz) δ 1.08 (d, 6H), 1.23 (d, 6H), 1.89 (s, 1H), 4.09 (m, 2H), 4.27 (d, 1H), 4.40 (d, 2H), 4.45 (m, 3H), 6.30 (s, 1H), 6.67 (s, 1H), 6.81 (s, 1H), 7.11 (s, 1H), 7.31 (m, 6H), 7.48 (d, 2H), 7.78 (d, 2H), 7.93 (t, 1H), 8.71 (t, 1H), 9.72 (s, 1H), 10.65(s, 1H)

### Example 43

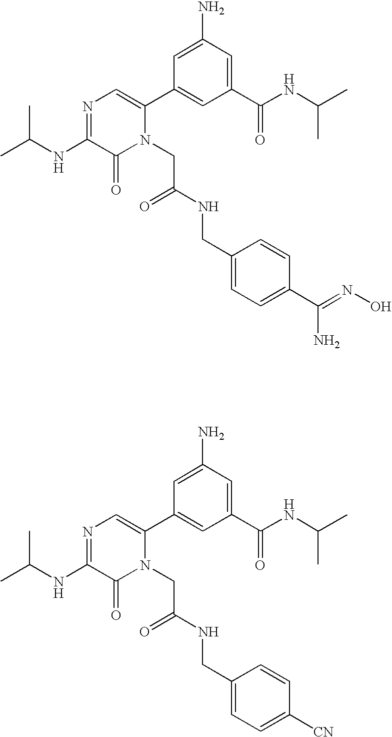

### Example 43a

LCMS (ES+) m/z M+H 502.

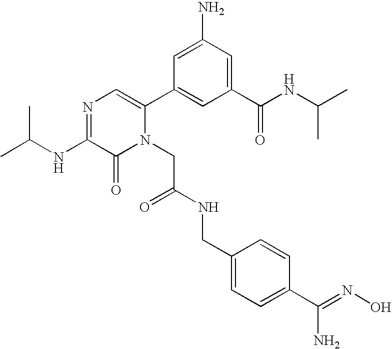

### Example 43

¹HNMR (300 MHz, DMSO-d₆) δ 1.14 (d, J=6.9 Hz, 6H), 1.25 (d, J=6.3 Hz, 6H), 4.02-4.18 (m, 2H), 4.36 (d, J=5.1 Hz, 2H), 4.44 (s, 2H), 6.72 (s, 1H), 6.73 (s, 1H), 7.04 (s, 1H), 7.16 (s, 1H), 7.39 (d, J=8.1 Hz, 2H)), 7.66 (d, J=8.1 Hz, 2H), 8.08 (d, J=7.8 Hz, 1H), 8.70 (t, J=5.4 Hz, 1H), 9.08 (br s, 2H), 11.20 (br s, 1H). HRMS (ES) calcd for C₂₇H₃₆N₈O₄ (M+H): 535.2776. Found: 535.2744. Anal. Calcd for C₂₇H₃₅N₈O₄+2.75 TFA+0.75 H₂O: C, 45.3; H, 4.47; N, 13.0. Found: C, 45.28; H, 4.48; N, 13.0.

### Example 44

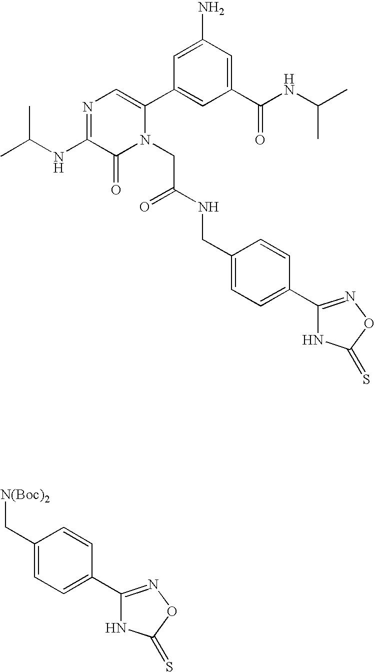

### Example 44a

LCMS (ES+) m/z M+H 408.

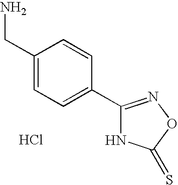

### Example 44b

Example 44a (0.5 g, 1.2 mmol) was stirred with 4 N hydrogen chloride in dioxane (6 ml) for 2 hours and concentrated in vacuo to give an off-white solid. Material was used without further purification. LCMS (ES+) m/z M+H 208.

### Example 44

¹HNMR (300 MHz, DMSO-d₆) δ 1.14 (d, J=6.6 Hz, 6H), 1.25 (d, J=6.6 Hz, 6H), 4.01-4.17 (m, 2H), 4.35 (d, J=4.8 Hz, 2H), 4.43 (s, 2H), 6.73 (s, 2H), 7.05 (s, 1H), 7.17 (s, 1H), 7.35 (d, J=8.4 Hz, 2H), 7.90 (d, J=8.1 Hz, 2H), 8.07 (d, J=7.8 Hz, 1H), 8.60-8.68 (m, 1H), 13.40 (br s, 1H). HRMS (ES) calcd for C₃₃H₃₆N₇O₆ (M+H): 626.2722. Found: 626.2723. Anal. Calcd for C₃₃H₃₅N₇O₆+2.05 TFA+0.75 H₂O: C, 51.04; H, 4.45; N, 11.23. Found: C, 51.09; H, 4.49; N, 11.16.

### Example 45

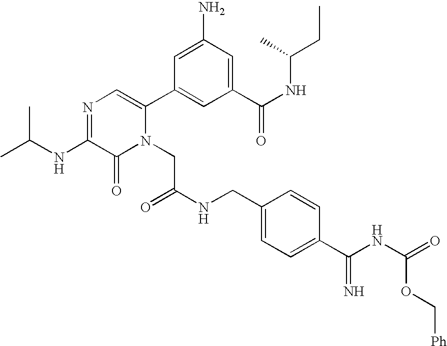

### Benzyl {4-[({[6-[3-amino-5-({[(1R)-1-methylpropyl]amino}carbonyl)phenyl]-3-(isopropylamino)-2-oxopyrazin-1(2H)-yl]acetyl}amino)methyl]phenyl}(imino)methylcarbamate

### Example 45

(0.72 g, 1.8 mmol), benzyl [4-(aminomethyl)phenyl] (imino)methylcarbamate (640 mg, 2 mmol), TBTU (642 mg, 2 mmol) and DIEA (1.8 g, 14 mmol) in 20 ml of DMF was kept stirring at RT for 2 hr. The mixture was added 10 ml EtOAc and 50 ml water, then extracted with EtOAc(3×20 ml). The combined EtOAc was then concentrated and purified on RP-HPLC to yield 520 mg white solid (31%).

HRMS calcd for C₃₆H₄₂N₈O₅ (M+H): 667.3351. Found: 667.3308.

Anal. Calcd for C₃₆H₄₂N₈O₅ + 2.15TFA + 0.7H2O:

| | | | |
|---|---|---|---|
| | C: 52.35; | H: 4.96; | N: 12.11. |
| Found: | C: 52.36; | H: 5.00; | N: 12.10. |

¹H NMR (DMSO-d₆, 300 MHz) δ 0.85 (t, 3H), 1.10 (d, 3H), 1.24 (m, 6H), 1.48 (m, 2H), 3.89 (m, 1H), 4.11 (m, 1H), 4.39 (m, 3H), 4.45 (bs, 1H), 5.37 (s, 2H), 6.71 (d, 2H), 7.01 (s, 1H), 7.13 (s, 6H), 7.39-7.49 (m, 6H), 7.77 (d, 2H), 8.00 (d, 1H), 8.71 (t, 1H), 10.49(bs, 1H)

### Example 46

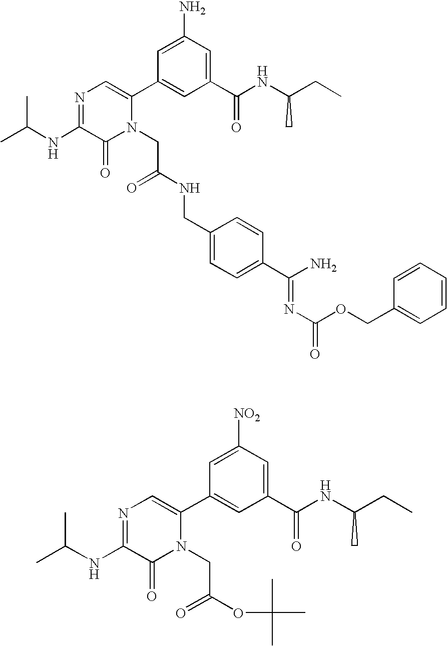

### Example 46a

LCMS (ES+) m/z M+H 488.

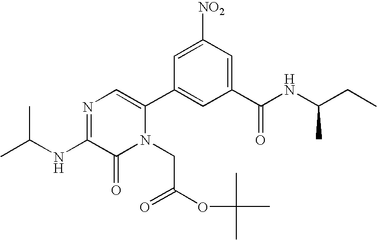

### Example 46b

LCMS (ES+) m/z M+H 488.

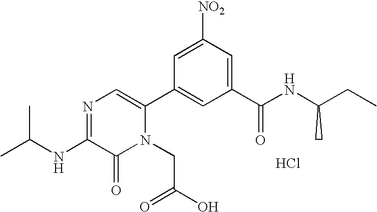

### Example 46c

Example 46a (1.02 g, 2.1 mmol) was stirred with 4N hydrogen chloride in dioxane for 18 hours, heated at 60° C. for 1 hour followed by the addition of 6N hydrogen chloride solution (1 ml) to the warm reaction and stirred for an additional 30 minutes. The reaction was concentrated in vacuo, dissolved in ethanol and again concentrated in vacuo to give 0.98 g (100% yield) of a yellow solid. LCMS (ES+) m/z M+H 432.

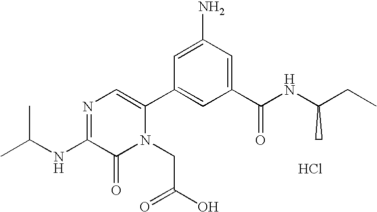

### Example 46d

A suspension of Example 46c (0.97 g, 2.1 mmol) with 10% palladium on carbon (1.0 g) in ethanol was shaken under 42 psi hydrogen for 1 hour. The reaction was filtered and concentrated in vacuo to give 0.81 g (89% yield) of a yellow solid. LCMS (ES+) m/z M+H 402.

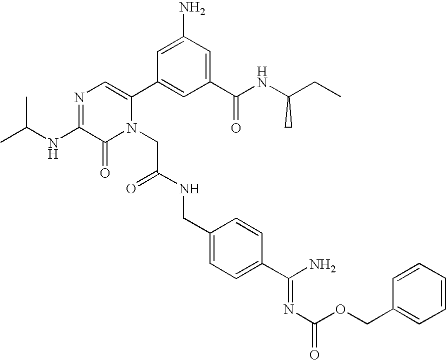

### Example 46

¹HNMR (300 MHz, DMSO-d₆) δ 0.85 (t, J=7.5 Hz, 3H), 1.10 (d, J=6.6 Hz, 3H), 1.22 (d,J=6.6 Hz, 6H), 1.40-1.58 (m, 2H), 3.82-3.98 (m, 1H), 4.01-4.19 (m, 1H), 4.34 (d, J=5.4 Hz, 2H), 4.42 (s, 2H), 5.13 (s, 2H), 5.45 (s, 2H), 6.67 (s, 1H), 6.71 (s, 1H), 6.81 (d, J=8.1 Hz, 1H), 6.98 (s, 1H), 7.10 (s, 1H), 7.31 (d, J=8.1 Hz, 2H), 7.33-7.46 (m, 5H), 7.94 (d, J=8.1 Hz, 2H), 8.60 (t, J=5.4 Hz, 1H), 9.14 (br s, 2H). HRMS (ES) calcd for C₃₆H₄₃N₈O₅ (M+H): 667.3351. Found: 667.3355. Anal. Calcd for C₃₆H₄₂N₈O₅+0.35 ethyl acetate+0.35 water: C, 63.99; H, 6.46; N, 16.22. Found: C, 64.02; H, 6.37; N, 16.26.

### Example 47

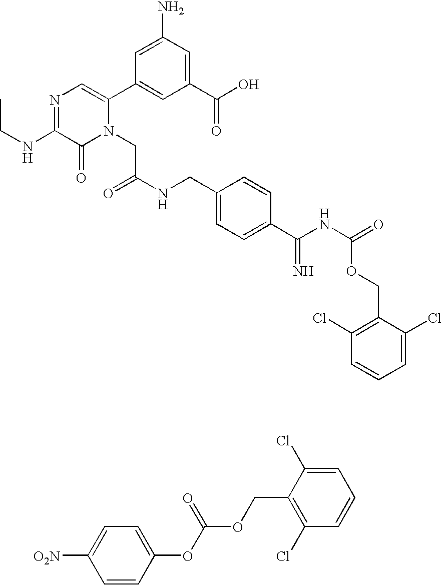

### Example 47a

To the (2,6-dichlorophenyl)methanol (5 g, 28.4 mmol) and NMM (6.85 g, 68.2 mmol) in 100 ml of CH2Cl2 at 0° C. was added 4-nitrophenyl chloridocarbonate (6.85 g, 34.1 mmol). The mixture was allowed to RT after 1 hr and stirred overnight. Then the mixture was recrystallized in CH2Cl2/Hexane and filtered. The liquid was then concentrated to yield 4.2 g solid (43%).

C₁₄H₉Cl₂N₁O₅ M.W. 342.13

### Example 47

Example 47a (0.3 g, 0.58 mmol) and 2,6-dichlorobenzyl4-nitrophenyl carbonate (200 mg, 0.58 mmol) in DMF (2 ml) was added NMM (240 mg, 2.4 mmol). The mixture was kept stirring overnight and purified on RP-HPLC to yield 120 mg of solid (22%).

C₃₂H₃₁Cl₂N₇O₆ M.W. 680.54.

Anal. Calcd for C₃₂H₃₁Cl₂N₇O₆ + 2.05TFA + 0.9H2O:

| | | | |
|---|---|---|---|
| | C: 46.59; | H: 3.77; | N: 10.53. |
| Found: | C: 46.60; | H: 3.81; | N: 10.50. |

¹H NMR (DMSO-d₆, 300 MHz) δ 1.24 (d, 6H), 4.11 (m, 3H), 4.39 (m, 4H), 5.54 (s, 2H), 6.75 (d, 2H), 7.11 (s, 1H), 7.28 (s, 1H), 7.38 (d, 2H), 7.54 (d, 1H), 7.62 (d, 2H), 7.79 (d, 2H), 8.66 (t, 1H), 10.26(bs, 1H).

### Example 48

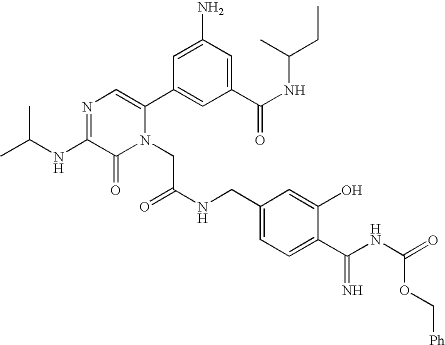

HRMS calcd for C₃₆H₄₂N₈O₆ (M+H): 683.3300. Found: 683.3326.

Anal. Calcd for C₃₆H₄₂N₈O₆+1.9TFA+0.05H2O:

C: 52.46; H: 4.99; N: 12.61.

Found: C: 52.53; H: 5.00; N: 12.38.

¹H NMR (DMSO-d₆, 300 MHz) δ 0.86 (t, 3H), 1.12 (d, 3H), 1.24 (m, 6H), 1.49 (m, 2H), 4.26 (m, 2H), 4.42 (m, 2H), 5.29 (s, 2H), 6.74 (bs, 3H), 6.83 (s, 1H), 7.04 (s, 1H), 7.15 (s, 1H), 7.38-7.52 (m, 6H), 7.66 (d, 1H), 8.06 (d, 1H), 8.64 (bs, 1H), 10.14(bs, 1H)

### Example 49

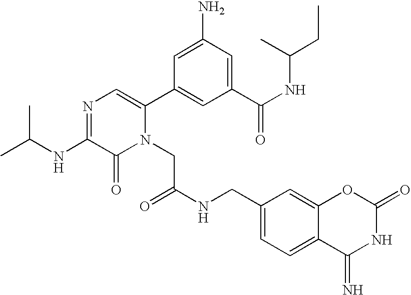

HRMS calcd for C₂₉H₃₄N₈O₅ (M+H): 575.2725. Found: 575.2757.

Anal. Calcd for C₂₉H₃₄N₈O₅ + 1.85TFA + 1.3H2O:

| | | | |
|---|---|---|---|
| | C: 48.54; | H: 4.79; | N: 13.85. |
| Found: | C: 48.58; | H: 4.86; | N: 13.73. |

¹H NMR (DMSO-d₆, 300 MHz) δ 0.87 (t, 3H), 1.12 (d, 3H), 1.26 (d, 6H), 1.49 (m, 2H), 3.90 (m, 1H), 4.39 (d, 3H), 4.45 (s, 2H), 6.75 (s, 2H), 7.07 (s, 1H), 7.13-7.18 (m, 2H), 8.02 (m, 2H), 8.73 (m, 2H).

### Example 50

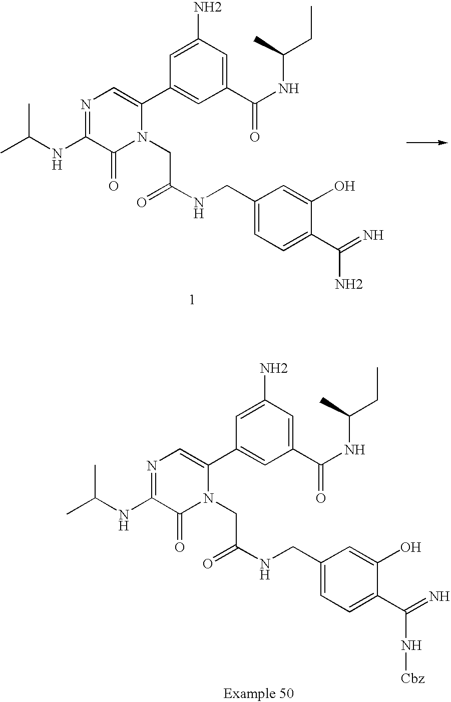

### Example 50

Intermediate 1 (0.3 g, 0.55 mmol) and benzyl 4-nitrophenyl carbonate (149 mg, 0.55 mmol) in DMF (2 ml) was added NMM (240 mg, 2.4 mmol). The mixture was kept stirring overnight and purified on RP-HPLC to yield 120 mg of solid (26%).

HRMS calcd for C₃₆H₄₂N₈O₆ (M+H): 683.3300. Found: 683.3278.

Anal. Calcd for C₃₆H₄₂N₈O₆ + 1.2TFA + 1.05H2O:

| | | | |
|---|---|---|---|
| | C: 55.00; | H: 5.44; | N: 13.36. |
| Found: | C: 54.99; | H: 5.41; | N: 13.43. |

¹H NMR (DMSO-d₆, 300 MHz) δ 0.86 (t, 3H), 1.12 (d, 3H), 1.24 (m, 6H), 1.48 (m, 2H), 3.88 (m, 2H), 4.11(m, 2H), 4.25 (d, 2H), 4.42 (m, 2H), 5.25 (s, 2H), 6.73 (bs, 3H), 6.80 (s, 1H), 7.01 (s, 1H), 7.13 (s, 1H), 7.38-7.46 (m, 4H), 7.73 (d, 1H), 8.00 (m, 2H), 8.61 (bs, 1H), 9.94(bs, 1H).

### Example 51

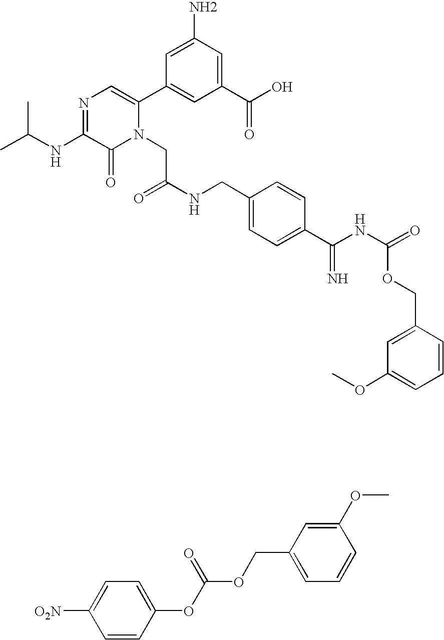

### Example 51a

To the (3-methoxyphenyl)methanol (5 g, 36 mmol) and NMM (6.85 g, 68.2 mmol) in 100 ml of CH2Cl2 at 0° C. was added 4-nitrophenyl chloridocarbonate(6.58 g, 32.6 mmol). The mixture was allowed to RT after 1 hr and stirred overnight. Then the mixture was recrystallized in CH2Cl2/Hexane and filtered. The liquid was then concentrated to yield 4.2 g solid (38.5%).

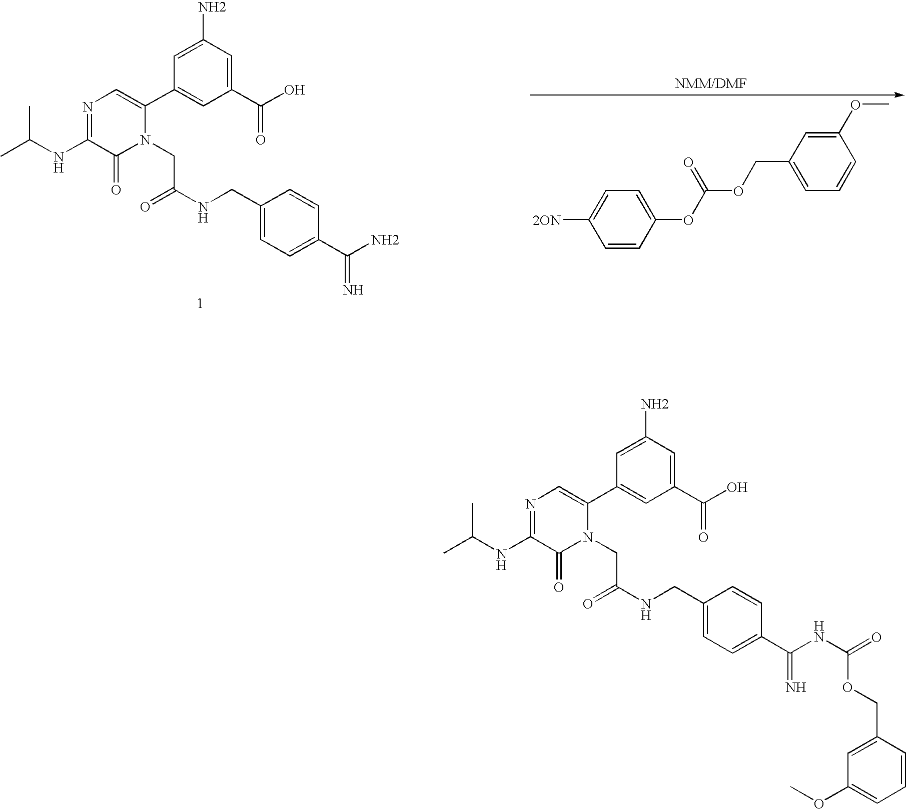

### Example 51

Intermediate 1 (0.3 g, 0.58 mmol) and (3-methoxybenzyl)-4-nitrophenyl carbonate (176 mg, 0.58 mmol) in DMF (2 ml) was added NMM (240 mg, 2.4 mmol). The mixture was kept stirring overnight and purified on RP-HPLC to yield 120 mg white solid (24%).

HRMS calcd for C₃₃H₃₅N₇O₇ (M+H): 642.2671. Found: 642.2704.

Anal. Calcd for C₃₃H₃₅N₇O₇ + 1.65TFA + 0.9H2O:

| | | | |
|---|---|---|---|
| | C: 51.53; | H: 4.58; | N: 11.58. |
| Found: | C: 51.54; | H: 4.66; | N: 11.50. |

¹H NMR (DMSO-d₆, 300 MHz) δ 1.24 (d, 6H), 3.80 (S, 3H), 4.11 (m, 3H), 4.39 (m, 5H), 5.32 (s, 2H), 6.71 (s, 1H), 6.76 (s, 1H), 6.98 (d, 2H), 7.06 (m, 3H), 7.11 (s, 1H), 7.27 (s, 1H), 7.40 (d, 2H), 7.79 (d, 2H), 8.69 (t, 2H).

### Example 52

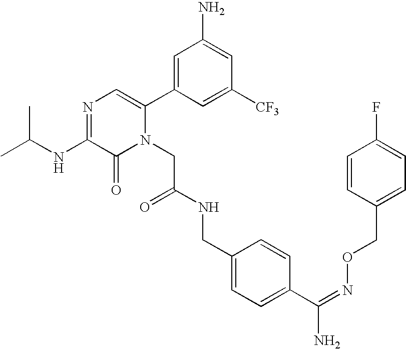

### N-[4-((E)-amino{[(4-fluorobenzyl)oxylimino}methyl)benzyl]-2-[6-[3-amino-5-(trifluoromethyl)phenyl]-3-(isopropylamino)-2-oxopyrazin-1(2H)-yl]acetamide Trifluoroacetate

### Example 52a

In a round bottom flask with a magnetic stirrer, under nitrogen, di(tert-butyl) imidodicarbonate (24.38 g, 112 mmol) was combined with tetrahydrofuran (150 ml) and sodium hydride (4.49 g, 112 mmol). The reaction foam and off-gas for 16 minutes. To the reaction mixture 4-(bromomethyl)benzonitrile (20.0 g, 102 mmol) and stirred for 18 hours. Reaction mixture was washed with sat KHSO₄ (2×100 ml), water (2×100 ml), dried over magnesium sulfate, filtered and concentrated to give 36.59 g (100% crude yield) of a white solid, di(tert-butyl) 4-cyanobenzylimidodicarbonate.

Mass Spectrometry (Electrospray): M+Na 355.1

NMR (400 MHz, CDCl₃) ¹H: 7.573 ppm (d, 2H), 7.352 ppm (d, 2H), 4.775 ppm (s, 2H), 1.410 ppm (s, 18H).

### Example 52b

In a round bottom flask with a magnetic stirrer, heating mantel, and a cold water condenser, under nitrogen, di(tert-butyl) 4-cyanobenzylimidodicarbonate (10.31 g, 31.02 mmol) was combined with ethanol, diisopropyl ethyl amine (27.02 ml, 155 mmol), and hydroxylamine (10.78 g, 155 mmol). The reaction mixture refluxed 2 hours. The reaction mixture was allowed to cold to room temperature, concentrated and then dissolved in ethyl acetate (200 ml). The ethyl acetate mixture was washed with NaHCO₃ (2×10 ml), dried over magnesium sulfate, filtered and concentrated to give 9.5 g (84% crude yield) of a white solid, di(tert-butyl) 4-[(Z) -amino(hydroxyimino)methyl] benzylimidodicarbonate.

Mass Spectrometry (Electrospray): M+H 366.1

NMR (400 MHz, DMSO) ¹H: 7.602 ppm (d, 2H), 7.193 ppm (d, 1H), 6.997 ppm (d, 1H), 4.341 ppm (s, 2H), 1.271 ppm (s, 18H).

### Example 52c

In a round bottom flask with a magnetic stirrer, under nitrogen, di(tert-butyl) 4-[(Z)-amino(hydroxyimino)methyl] benzylimidodicarbonate (10.9 g, 29.83 mmol) was combined with ammonium formate (4.76 g, 75.48 mmol) and methanol (50 ml). To the reaction palladium black (0.34 g, 3.19 mmol) was added and stirred 4 hours. The reaction mixture was filtered, and concentrated to give 11.27 g (100 crude yield) of a white solid, di(tert-butyl) 4-[amino(imino)methyl]benzylimido-dicarbonate.

Mass Spectrometry (Electrospray): M+H 350.1

NMR (400 MHz, CDCl₃) ¹H: 7.573 ppm (d, 2H), 7.286 ppm (d, 2H), 4.775 ppm (s, 2H), 1.445 ppm (s, 18H).

### Example 52d

In a round bottom flask with a magnetic stirrer, under nitrogen, heating mantel, cold water condenser di(tert-butyl) 4-[amino(imino)methyl]benzylimido-dicarbonate (2.31 g, 5.65 mmol), was combined with diisopropyl ethyl amine (2.19 g, 16.95 mmol), ethanol (50 ml), and 1-[(aminooxy)methyl]-4-fluorobenzene hydrochloride (2.0 g, 11.29 mmol). The reaction mixture refluxed for 6 hours. The reaction mixture was concentrated to give 4.74 g of an orangish oil. The crude product was chromatographed on silica with ethyl acetate and hexane. Elution began at 10% ethyl acetate and finished at 20% ethyl acetate. 1.4 g of a white solid, di(tert-butyl) 4-((E)-amino{[(4-fluorobenzyl)oxy]imino}methyl)benzylimido dicarbonate, (52% yield), was recovered.

Mass Spectrometry (Electrospray): M+H 474.3

NMR (400 MHz, CDCl₃) ¹H: 7.557 ppm (d, 2H), 7.374 ppm (dd, 2H), 7.259 ppm (d, 2H), 7.005 ppm (d, 2H), 5.051 ppm (s, 2H), 4,842

NMR (400 MHz, CDCl₃) ¹⁹F: −115.079 ppm (sextet, 1F)

### Example 52e

In a round bottom flask with a magnetic stirrer, under nitrogen di(tert-butyl) 4-((E)-amino{[(4-fluorobenzyl)oxy]imino}methyl)benzyl imidodicarbonate (0.45 g, 0.95 mmol) was dissolve in 4M HCl in dioxane (20 ml, 80 mmol) and stirred 3 hours. The reaction mixture was concentrated to give a white solid, (0.40 g, 100% crude yield), 4-(aminomethyl)-N′-[(4-fluorobenzyl)oxy]benzenecarbox imidamide hydrochloride.

Mass Spectrometry (Electrospray): M+H 274.1

NMR (400 MHz, CD₃OD) ¹H: 7.686 ppm (s, 4H), 7.592 ppm (dd, 2H), 7.160 ppm (t, 2H), 5.114 ppm (s, 2H), 4.225 ppm (s, 2H).

NMR (400 MHz, CD₃OD) ¹⁹F: −114.389 ppm (br s, 1F).

### Example 52f

In a round bottom flask with a magnetic stirrer, under nitrogen, heating mantel, thermocouple, and a cold water condenser was added 3-bromo-5-(trifluoromethyl)aniline (1.0 g, 4.17 mmol), dimethyl sulfoxide (50 ml), 4,4,4′,4′,5,5,5′,5′-octamethyl-2,2′-bi-1,3,2-dioxaborolane (1.06 g, 4.17 mmol), potassium acetate (1.23 g, 12.50 mmol). The reaction stirred 30 minutes while argon was bubbled through the solution. To the reaction mixture was added [1,1′-bis(diphenylphosphino)-ferrocene]dichloropalladium(ll), complex with dichloromethane (1:1) (0.102 g, 0.125 mmol). The reaction was heated to 84° C. and stirred for 18 hours. The reaction mixture was allowed to cool to room temperature and then brine (100 ml) and ethyl acetate (100 ml) was added and stirred 30 minutes. The organic layer was separated was further washed with brine (2×100 ml), dried over magnesium sulfate, filtered through silica, and concentrated to give 3.04 g (91% crude yield) of a black oil, 3-(4,4,5,5-tetramethyl-1,3,2-dioxaborolan-2-yl)-5-(trifluoromethyl)aniline.

Mass Spectrometry (Electrospray): M+H 288.1

NMR (400 MHz, CDCl₃) ¹H: 7.411 ppm (s, 1H), 7.244 ppm (s, 1H), 6.946 ppm (s, 1H), 3.730 ppm ( br s, 2H), 1.314 ppm (s, 12H).

NMR (400 MHz, CD₃OD)¹⁹ F: −63.141 ppm (s, 3F).

### Example 52g

In a round flask with a magnetic stirrer, under nitrogen, heating mantel, cold water condenser was added 3-(4,4,5,5-tetramethyl-1,3,2-dioxaborolan-2-yl)-5-(trifluoromethyl)aniline (5.0 g, 17.4 mmol), tert-butyl [6-bromo-3-(isopropylamino)-2-oxopyrazin-1(2H)-yl]acetate (5.42 g, 15.8 mmol), cesium carbonate (6.19 g, 19.0 mmol) and dioxane (100 ml). The reaction mixture stirred 30 minutes while argon was bubbled through the solution. To the reaction mixture was added tetrakis(triphenylphosphine)palladium(0) and reflux 15 hours. The reaction mixture was allowed to cool to room temperature and then was filter through celite/silica plug. The plug was washed ethyl acetate (100 ml). The organics were combined and concentrate to give 10.81 g reddish black oil. The oil was dissolved in dichloromethane and chromatograph on silica. The silica column was eluded with 1% ethanol and 99% dichloromethane. 6.2 g (93% yield) of a dark reddish brown oil, tert-butyl [6-[3-amino-5-(trifluoromethyl)phenyl]-3-(isopropylamino)-2-oxopyrazin-1(2H)-yl]acetate.

Mass Spectrometry (Electrospray): M+H 427.5

NMR (400 MHz, CDCl₃) ¹H: 6.921 ppm (d, 2H), 6.771 ppm (s, 1H), 6.743 ppm (s, 1H), 6.105 ppm (br s, 1H), 4.349 ppm (s, 2H), 4.178 ppm (m, 1H), 3.988 ppm (br s, 1H), 1.413 ppm (s, 9H), 1.265 ppm (d, 6H).

NMR (400 MHz, CD₃OD) ¹⁹F: −63.409 ppm (s, 3F)

### Example 52h

In a round bottom flask with a magnetic stirrer, under nitrogen was added tert-butyl [6-[3-amino-5-(trifluoromethyl)phenyl]-3-(isopropylamino)-2-oxopyrazin-1(2H)-yl]acetate (20.50 g, 48.08 mmol) and 4M HCl in dioxane (100 ml, 400 mmol). The reaction mixture stirred for 12 hour. The reaction mixture was concentrated to give 20.78 g (100% yield) of a white solid, [6-[3-amino-5-(trifluoromethyl)phenyl]-3-(isopropylamino)-2-oxopyrazin-1(2H)-yl]acetic acid dihydrochloride.

Elemental analysis: C−42.21, H−4.33, N−12.22, F−12.05

Mass Spectrometry (Electrospray): M+H 371.0

NMR (400 MHz, D₂O) ¹H: 7.653 ppm (s, 1H), 7.607 ppm (s, 1H), 7.443 ppm (s, 1H), 6.657 ppm (s, 1H), 4.341 ppm (s, 2H), 3.885 ppm (pentet, 1 H), 1.231 ppm (d, 6H).

NMR (400 MHz, D₂O) ¹⁹F: −63.311 ppm (s, 3F).

### Example 52

In a round bottom flask, with a magnetic stirrer, under nitrogen, was added the product from Ex-52e, 4-(aminomethyl)-N′-[(4-fluorobenzyl)oxy]benzenecarboximidamide hydrochloride (0.40 g, 1.16 mmol), the product from Ex-52h, [6-[3-amino-5-(trifluoromethyl)phenyl]-3-(isopropylamino)-2-oxopyrazin-1(2H)-yl]acetic acid dihydrochloride (0.77 g, 1.74 mmol), dimethyl formamide (50 ml), diisopropyl ethyl amine (1.12 g, 8.69 mmol), and O-(1H-benzotriazol-1-yl)-N,N,N′,N′-pentamethylene-uronium tetrafluoroborate (1.30 g, 4.06 mmol). The reaction stirred at room temperature for 18 hours. The reaction was concentrated to 3.89 g of a reddish oil. The oil was dissolved in acetonitrile (25 ml) and water (50 ml) and acidified with acetic acid (1 ml). The reaction mixture was chromatographed by HPLC, 10 to 50% acetonitrile over 30 minutes. 0.592 g (59% yield) of N-[4-((Z)-amino{[(4-fluorobenzyl)oxy]imino}methyl)benzyl]-2-[6-[3-amino-5-(trifluoromethyl)phenyl]-3-(isopropylamino)-2-oxopyrazin-1(2H)-yl]acetamide bis(trifluoroacetate).

Elemental analysis: C—49.63, H—3.86, N—11.14, F—20.37

NMR (400 MHz, CD₃OD) ¹H: 7.538 ppm (dt, 4H), 7.389 ppm (d, 2H), 7.137 ppm (t, 2H), 7.016 ppm (s, 1H), 6.873 ppm (d, 2H), 6.648 ppm (s, 1H), 5.070 ppm (s, 2H), 4.518 ppm (s, 2H), 4.421 ppm (s, 2H), 4.032 ppm (septet, 1H), 1.369 ppm (d, 6H).

NMR (400 MHz, CD₃OD) ¹⁹F: −64.925 ppm (s, 3F), −77.645 ppm (s, 6F), −115.076 ppm (s, 1F)

### Example 54

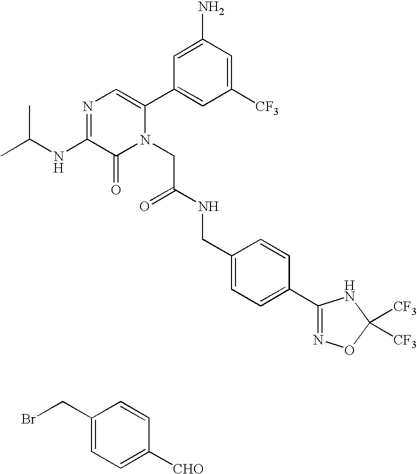

### 4-(bromomethyl)benzaldehyde

### Example 54a

To a solution of 4-(bromomethyl)benzonitrile (10 g, 50 mmol) in dichloromethane (30 mL) at 0° C. was added DibalH (56 mL, 55 mmol) dropwise. After addition the reaction mixture was heated to 45° C. for 4 hrs and then cooled to room temperature. The reaction was quenched with the addition of 10% H₂SO₄ and stirred overnight. The layers were separated and the aqueous layer extracted with dichloromethane (2×50 mL). The organic extracts were washed with brine and dried (Na₂SO₄). The solvent was removed in vacuo to give the product as a solid (7.12 g, 72%). ¹H NMR (400 MHz, CDCl₃): 9.99 (s, 1 H), 7.85 (d, 2 H), 7.54 (d, 2 H), 4.49 (s, 2 H).

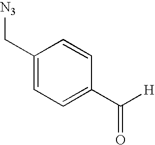

### 4-(azidomethyl)benzaldehyde

### Example 54b

To a solution of Example 54a (1.5 g, 0.75 mmol) in DMF at room temperature was added sodium azide (0.58 g, 8.9 mmol). The reaction was stirred at room temperature for 2 hrs and then diluted with water and ether. The layers were separated and the organic layer washed with water and brine. The organic extracts were dried (Na₂SO₄) and the solvent removed to give the desired product as an oil (1.02 g, 85%). ¹H NMR (400 MHz, CDCl₃) δ 10.0 (s, 1 H), 7.89 (d, 2 H), 7.48 (d, 2 H), 4.44 (s, 2 H).

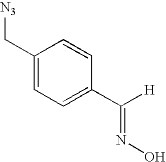

### 4-(azidomethyl)benzaldehyde Oxime

### Example 54c

To a solution of Example 54b (1.37 g, 8.55 mmol) in dichloromethane/ethanol was added H₂NOH*HCl (0.64 g, 9.21 mmol) and pyridine (0.83 mL, 10.21 mmol). After 3 hrs, the reaction was diluted with water and dicholoromethane. The layers were separated and the organic layer washed with brine and dried (Na₂SO₄). The solvent was removed in vacuo to give an oil, which after chromatography (silica, 10%-30% ethyl acetate:hexanes) gave the desired product (1.2 g, 80%). ¹H NMR (400 MHz, CDCl₃) δ 8.13 (s, 1 H), 7.73 (bs, 1 H), 7.59 (d, 2 H), 7.33 (d, 2 H), 4.35 (s, 2 H).

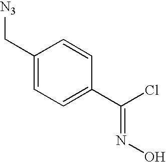

### 4-(azidomethyl)-N-hydroxybenzenecarboximidoyl Chloride

### Example 54d

To a solution of Example 54c (1.20 g, 6.80 mmol) in DMF (15 mL) at room temperature was added N-chlorosuccinimide. The reaction was heated to 40° C. for 3 hrs and then allowed to cool to room temperature. The reaction mixture was diluted with water and ether. The layers were separated and the organic layer washed with brine and dried (Na₂SO₄). The solvent was removed in vacuo to give an oil, which after chromatography (10-20% ethyl acetate:hexane) gave the desired product as an oil (0.98 g, 68%). ¹H NMR (400 MHz, CDCl₃): δ 8.51 (bs, 1 H), 7.85 (d, 2 H), 7.34 (d, 2 H), 4.37 (s, 2 H).

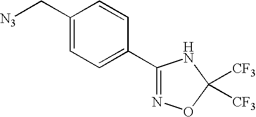

### 3-[4-(azidomethyl)phenyl]-5,5-bis(trifluoromethyl)-4,5-dihydro-1,2,4-oxadiazole

### Example 54e

To a solution of Example 54d (0.15 g, 0.71 mmol) in ether at 0° C. was bubbled in 1,1,1,3,3,3-hexafluoropropan-2-imine (Synquest) for 3 min. Triethylamine was added and the reaction was stirred at room temperature for 2 hrs. The reaction mixture was diluted with water and ether. The layers were separated and the organic layer washed with brine and dried (Na₂SO₄). The solvent was removed to give an oil, which after chromatography (silica, 5%-30% ethyl acetate:hexane) gave the desired product as an oil (0.11 g, 47%). ¹H NMR (400 MHz, CDCl₃): δ 7.71 (d, 2 H), 7.43 (d, 2 H), 5.43 (bs, 1 H) 4.42 (s, 2 H); ¹⁹F NMR (371 MHz, CDCl₃): δ −80.6 (s, 6 F); MS-ESI (M+H) 381.

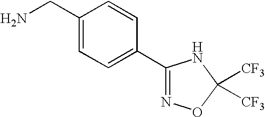

### 4-[5,5-bis(trifluoromethyl)-4,5-dihydro-1,2,4-oxadiazol-3-yl]benzylamine

### Example 54f

To a solution of Example 54e (0.83 g, 2.41 mmol) in THF:H₂O (9 mL:1 mL) was added triphenylphosphine (0.77 g, 2.88). The reaction was stirred at 40° C. for 2 hrs and then the solvent removed in vacuo to give an oil, which after chromatography (silica, 30%-50% MeOH/DCM) gave the desired product as a white solid (0.62 g, 83%). ¹H NMR (400 MHz, CD₃OD) δ 7.70 (d, 2 H), 7.46 (d, 2 H), 3.86 (s, 2 H).

### Example 54

¹H NMR (400 MHz, CD₃OD): δ 7.67 (d, 2 H), 7.36 (d, 2 H), 6.96 (s, 1 H), 6.84 (d, 2 H), 4.50 (s, 1 H), 4.41 (s, 1 H), 4.10-4.07 (m, 1 H), 1.24 (d, 6 H); ¹⁹F NMR (371 MHz, CD₃OD): δ −64.84 (s, 3 F), −82.36 (s, 6 F); MS-ESI (M+H)=666.

### Example 55

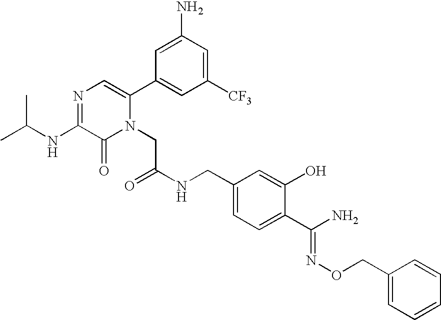

### Example 55

m/z(M+H) ⁺624

Analysis: C₃₁H₃₂F₃N₇O₄+2.1 TFA+0.25 H₂O calcd: C, 48.73; H, 4.02; N, 11.30; found: C, 48.81; H, 4.16; N, 11.04.

HRMS calcd: 624.2541; Found: 624.2523

¹H NMR(400 MHz, DMSO): 1.22(6H, d), 4.07(1H, m), 4.19(2H, m), 4.31(2H, s), 5.01(2H, s), 6.67-6.77(5H, m), 6.91(1H, s) 7.29-7.44(5H, m), 7.51(1H, d), 8.56(1H, t).

### Example 56

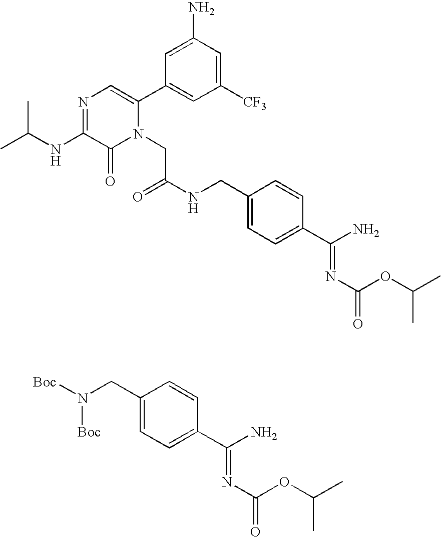

### Example 56a

A solution of di(tert-butyl)-4-[amino(imino)methyl]benzylimidodicarbonate (5.0 g, 12.7 mmol) in 225 ml of tetrahydrofuran and 25 ml of water at 0° C. was added sodium carbonate (6.75 g, 63.7 mmol) and 1M isopropyl chloroformate in tetrahydrofuran (28 ml, 28 mmol). The reaction mixture was stirred for 4 hours while warming to room temperature. The mixture was treated with water and extracted with ethyl acetate. The organic layer was dried over sodium sulfate, filtered and evaporated in vacuo to give an oil. The oil was purified by silica gel chromatography with 10-50% EA/Hex to give ˜2.0 g of a white solid. m/z(M+H) ⁺436

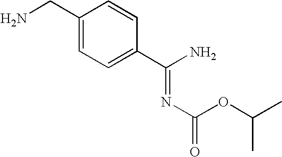

### Example 56b

The product from Example 56a (˜2.0 g, 12.7 mmol) at 0° C. was dissolved in 4M hydrogen chloride in dioxane (20 ml, 80 mmol) and stirred for 2 hours while warming to room temperature. The mixture was diluted with 150 ml of ethyl ether and the resulting precipitate was collected by vacuum filtration to give 1.37 g (40% yield over two steps) of a white solid. m/z(M+H) ⁺236

### Example 56

m/z(M+H)⁺588

Analysis: C₂₈H₃₂F₃N₇O₄+1.7 TFA+0.45 H₂O calcd: C, 47.77; H, 4.42; N, 12.42; found: C, 47.78; H, 4.49; N, 12.31.

HRMS calcd: 588.2541; Found: 588.2558

¹H NMR(400 MHz, DMSO): 1.20(6H, d), 1.33(2H, d), 4.09(1H, m), 4.37(4H, s), 5.06(1H, s), 6.71(1H, s), 6.77(1H, s), 6.80(1H, s), 6.90(1H, s), 7.38(2H, d), 7.72(2H, d), 8.75(1H, t).

### Example 57

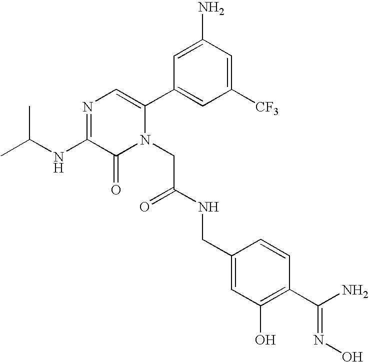

### N-{4-[(Z)-amino(hydroxyimino)methyl]-3-hydroxybenzyl}-2-[6-[3-amino-5-(trifluoromethyl)phenyl]-3-(isopropylamino)-2-oxopyrazin-1(2H)-yl]acetamide

### Example 57

To a 100 mL RBF was added free amidine (0.50 g, 0.98 mmol) in ethanol (75 mL). To the reaction was added hydroxyl amine hydrochloride (0.173 g, 2.5 mmol) and triethyl amine (0.50 mL). The reaction was heated to reflux for 16 hours. By L.C. and M.S. the starting material was consumed and desired product was observed. The reaction was concentrated in vacu and water was added (200 mL). The solid was filtered and dried in a dessicator in the presence of P2O5 to afford Ex-16 (0.410 g) as a tan solid in 76% yield.

M.S. 533.51 (MH+534.7)

Elemental-Isolated as a hydrate with 1.25 waters

| | | | |
|---|---|---|---|
| Calculated: | C 54.03 | H 4.91 | N 18.38 |
| Found: | C 51.84 | H 5.01 | N 17.55 |

### Example 58

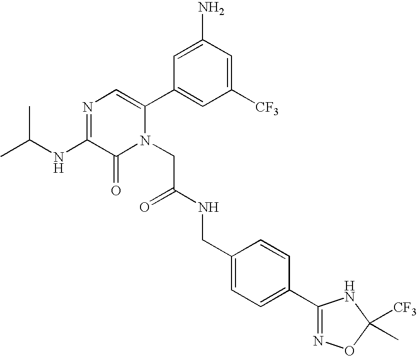

### 2-[6-[3-amino-5-(trifluoromethyl)phenyl]-3-(isopropylamino)-2-oxopyrazin-1(2H)-yl]-N-{4-[5-methyl-5-(trifluoromethyl)-4,5-dihydro-1,2,4-oxadiazol-3-yl]benzyl}acetamide

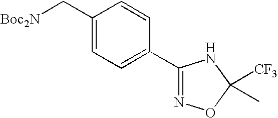

### Di(tert-butyl) 4-[5-methyl-5-(trifluoromethyl)-4,5-dihydro-1,2,4-oxadiazol-3-yl]benzylimidodicarbonate

### Example 58a

¹H NMR (400 MHz, CDCl₃): δ 7.62 (d, 2 H), 7.33 (d, 2 H), 4.83 (s, 1 H), 4.78 (s, 2 H), 1.77 (s, 3 H), 1.44 (s, 18 H); MS-ESI (M+H)=460.

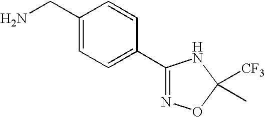

### 4-[5-methyl-5-(trifluoromethyl)-4,5-dihydro-1,2,4-oxadiazol-3-yl]benzylamine

### Example 58b

To a round bottom flask containing Example 58a (1.44 g, 3.00 mmol) was added 4N HCl in dioxane (25 mL). After 2 hrs the reaction was filtered and the solid collected and washed with ether. The solid was dried under high vacuum and used without further purification (0.88 g, 96%). ¹H NMR (400 MHz, d⁶-DMSO): δ 8.49 (bs, 3 H), 7.70 (d, 2 H), 7.57 (d, 2 H), 4.75 (bs, 1 H), 4.04 (q, 2 H), 1.64 (s, 3 H); MS-ESI (M+H)=260.

### Example 58

¹H NMR (400 MHz, CD₃OD): δ 7.62 (d, 2 H), 7.32 (d, 2 H), 6.96 (s, 1 H), 6.84 (d, 2 H), 6.71 (s, 1 H), 4.50 (s, 2 H), 4.39 (s, 2 H), 4.11 (m, 1 H), 1.68 (s, 3 H), 1.26 (d, 6 H); ¹⁹F NMR (371 MHz, CD₃OD): δ −64.8 (s, 3 F), −88.3 (s, 3 F); MS-ESI (M+H)=612.

### Example 59

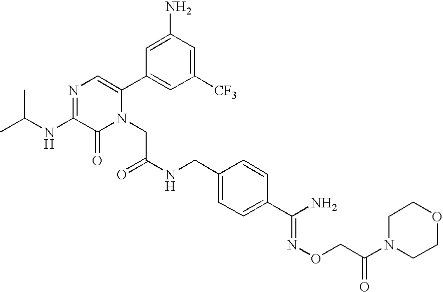

### Example 59

m/z(M+H)⁺645

Analysis: C₃₀H₃₅F₃N₈O₅+1.3 TFA+1.8 H₂O calcd: C, 47.29; H, 4.71; N, 13.13; found: C, 47.23; H, 4.58; N, 13.30.

HRMS calcd: 645.2755; Found: 645.2764

¹H NMR(400 MHz, DMSO): 1.22(6H, d), 3.43(2H, br s), 3.53(6H, br s), 4.08(1H, m), 4.28(2H, m), 4.34(2H, s), 4.62(2H, s), 6.74(1H, s), 6.77(1H, s), 6.79(1H, s), 6.92(1H, s), 7.20(2H, d), 7.56(2H, d), 8.61(1H, t).

### Example 60

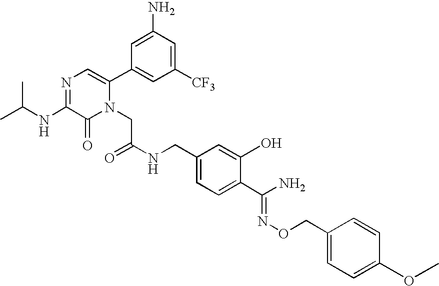

### Example 60

m/z(M+H) ⁺654

Analysis: C₃₂H₃₄F₃N₇O₅+1.9 TFA+0.95 H₂O calcd: C, 48.45; H, 4.29; N, 11.05; found: C, 48.47; H, 4.30; N, 10.97.

HRMS calcd: 654.2646; Found: 654.2665

¹H NMR(400 MHz, DMSO): 1.22(6H, d), 3.74(3H, s), 4.07(1H, m), 4.20(2H, m), 4.32(2H, s), 4.93(2H, s), 6.67-6.76(5H, m), 6.91(1H, s), 6.93(2H, d), 7.36(2H, d), 7.48(1H, d), 8.57(1H, t).

### Example 61

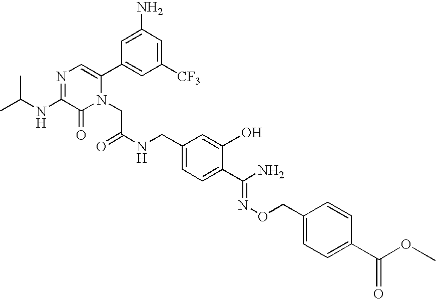

### Example 61

m/z(M+H)+682

Analysis: C₃₃H₃₄F₃N₇O₆+1.3 TFA+0.45 H₂O calcd: C, 51.03; H, 4.35; N, 11.70; found: C, 51.08; H, 4.44; N, 11.60.

HRMS calcd: 682.2595; Found: 682.2609

¹H NMR(400 MHz, DMSO): 1.21(6H, d), 3.84(3H, s), 4.07(1H, m), 4.19(2H, d), 4.31(2H, s), 5.11(2H, s), 6.67(1H, s), 6.72(1H, s), 6.93(2H, d), 6.75(2H, m), 6.84(1H, br s), 6.91(1H, s), 7.54(1H, m), 7.56(1H, d), 7.95(1H, d), 8.54(1H, t).

### Example 62

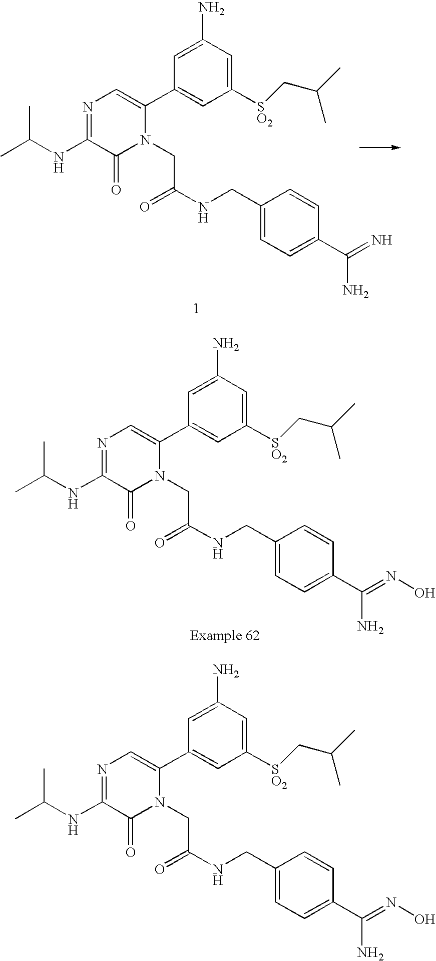

### N-{4-((Z)-amino(hydroxyimino)methyl]benzyl}-2-(6-[3-amino-5-(isobutylsulfonyl)phenyl]-3-(isopropylamino)-2-oxopyrazin-1(2H)-yl]acetamide

### Example 62

To a 25 mL RBF was added free amidine 1 (0.66 g, 0.887 mmol) in ethanol (10 mL) and DIEA (0.5 mL). To the solution was added hydroxyl amine (0.69 g, 1.0 mmol). The reaction stirred at reflux for 24 hours. By LCMS the starting material was only 50% consumed. The solution was diluted with 100 mL of 1N HCl and purified by reverse phase chromatography to afford Ex-18 (0.40 g) in 79% yield.

M.S. 569.3(MH+570.5)

NMR (400MHZ, CDCL3): 1H 1.08 ppm (6H, d), 4.01 ppm (1H, s), 4.2 ppm(1H, q), 4.25 ppm (2H, d), 4.36 ppm (2H, s), 6.75 ppm (2H, s), 6.79 ppm (2H, d), 6.9 ppm (1H, d), 6.98 ppm (1H, s), 7.58 (1H, d) 8.48 (1H, t).

Elemental-Isolated as a hydrate with 2 TFAs and
1 water as the hydrate

| | | | |
|---|---|---|---|
| Calculated: | C 56.93 | H 6.19 | N 17.21 |
| Found: | C 45.68 | H 4.78 | N 12.15 |

### Example 63

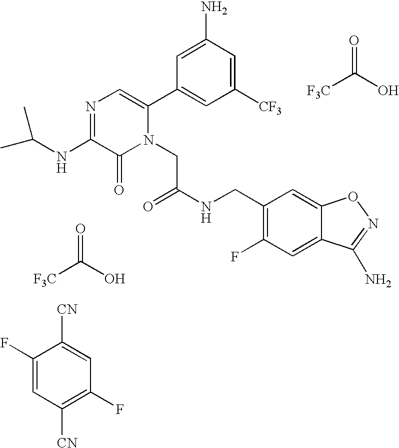

### 2,5-difluoroterephthalonitrile

### Example 63a

A solution of 1,4-dibromo-2,5-difluorobenzene (24.3 g, 89.6 mmol) and CuCN (24.06 g, 269 mmol) in DMF (150 mL) was heated to 150° C. while stirring for 16 hours. The reaction mixture was allowed to cool to room temperature and poured into a 15% NH₄OH solution (150 mL), diluted with CH₂Cl₂ (150 mL), filtered and transferred to a separatory funnel. The organic layer was separated and the aqueous solution was extracted with CH₂Cl₂. The combined organic solutions were dried over Na₂SO₄, filtered and concentrated. The crude product was purified by sublimation in a Kugelrhor apparatus to give Example 63a as a yellow solid.

¹H-NMR, 300 MHz, CDCl₃ δ 7.54 (t, J=6.00 Hz, 2H)

¹⁹F-NMR, 282 MHz, CDCl₃ δ −108.99 (t, J=6.00 Hz, 2F)

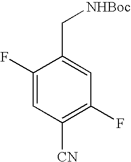

### Tert-butyl 4-cyano-2,5-difluorobenzylcarbamate

### Example 63b

To Example 63a, 2,5-difluoroterephthalonitrile (5.08 g, 30.9 mmol) in EtOH (150 mL) was added di-tert butylpyrocarbonate (7.11 mL, 30.9 mmol) and PtO₂.XH₂O (250 mg). The solution was purged with Argon and hydrogenated in a Fischer-Porter bottle at 60 psi while stirring for 16 hours. The reaction mixture was filtered through celite and concentrated to give Example 63b.

¹H-NMR, 300 MHz, CDCl₃ δ 7.20-7.30 (m, 2 H), 5.00 (bs, 1 H), 4.38 (d, J=6.2 Hz, 2 H), 1.45 (s, 9 H)

¹⁹F-NMR, 282 MHz, CDCl₃ δ −122.13 (mult), −111.614 (mult)

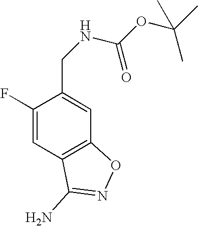

### Tert-butyl (3-amino-5-fluoro-1,2-benzisoxazol-6 -yl)methylcarbamate

### Example 63c

A solution of acetohydroxamic acid (2.20 g, 29.2 mmol) and Potassium tert-butoxide (3.44 g, 29.2 mmol) in DMF (75 mL) stirred at ambient temperature for 0.5 hour followed by addition of Example 63b, tert-butyl 4-cyano-2,5-difluorobenzylcarbamate in DMF (20 mL). The reaction stirred for 16 hours at ambient temperature. The reaction mixture was diluted with brine (20 mL) and ethyl acetate (20 mL). The organic layer was separated and the aqueous layer extracted with ethyl acetate (3×20 mL). The organic layers were combined dried over magnesium sulfate, filtered and concentrated to give Example 63c.

LCMS (RP, 15-90% gradient acetonitrile in 0.1% ammonium acetate over 6 min): retention time=3.38; (M+H)⁺=282.

¹H-NMR, 300 MHz, CDCl3 δ 7.40 (d, J5.44 Hz, 1 H), 7.16 (d, J=8.66 Hz, 1 H) 4.62 (d, J=5.84, 2 H), 1.46 (s, 9 H).

¹⁹F-NMR, 282 MHz, CDCl δ −126.191 (apparent dd, J=5.64, J=7.34 F)

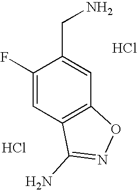

### 6-(aminomethyl)-5-fluoro-1,2-benzisoxazol-3-amine dihydrochloride

### Example 63d

A solution of Example 63c, tert-butyl (3-amino-5-fluoro-1,2-benzisoxazol-6-yl)methylcarbamate and 4 N HCl in dioxane were stirred at room temperature for 16 hours. The reaction was concentrated to give Ex-13d as a white solid.

LCMS (RP, 15-90% gradient acetonitrile in 0.1% ammonium acetate over 14 min): retention time=0.83; (M+H)⁺=182.

¹H-NMR, 300 MHz, DMSO-d₆ δ 8.65 (bs, 3 H), 7.74 (d, J=9.46 Hz, 1 H), 7.17 (d, J=6.64 Hz, 1 H), 5.35 (bs, 3 H), 4.09-4.17 (m, 2 H), ¹⁹F-NMR, 282 MHz, DMSO-d₆ δ −125.181 (apparent dd, J=8.71 Hz, J=5.81 1F).

### Example 63

The carboxylic acid, [3-amino-5-(trifluoromethyl)phenyl]-3-(isopropylamino)-2-oxopyrazin-1(2H)-yl]acetic acid, (727 mg, 1.64 mmol) the benzyl amine, 6-(aminomethyl)-5-fluoro-1,2-benzisoxazol-3-amine dihydrochloride, Example 63d (959 mg, 3.77 mmol) and HOBt-H₂O (332 mg, 2.46 mmol) were placed in a flask. DMF (4 mL) and CH₂Cl₂ (60 mL) were added. To this gently stirred solution was added polymeric DCC resin (7.94 g, loading 1.4 mmol/g, 11 mmol) and triethylamine was added to pH=9. The resulting mixture was stirred over night followed by gentle heating to 35° C. for 1 hour. Polymeric tris-amine resin (2.46 g, loading 2.46 mmol/g, 1 mmol) was added and stirred for 1 hour. The resins were filtered and washed with CH₂Cl₂ (200 mL). The filtrate was concentrated and the residue was purified by prep HPLC (RP, 5-90% gradient, acetonitrile in 0.1% TFA) to give Example 63.

LCMS (RP, 15-90% gradient acetonitrile in 0.1% ammonium acetate over 14 min): retention time=6.61; (M+H)⁺=534.

### Example 64

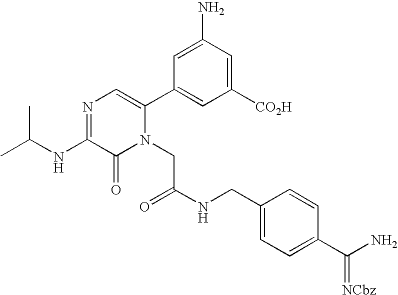

### Example 64a

m/z(M+H)⁺626

### Example 64

To the product from Example 64a (0.62 g, 0.99 mmol) in 9.3 ml of tetrahydrofuran and 2.5 ml of 2-propanol was added 2.5 N sodium hydroxide (1.2 ml, 3 mmol) and the mixture was stirred overnight. The reaction was quenched with 0.23 ml of trifluoroacetic acid and concentrated to a small volume. The mixture was treated with methyl sulfoxide until dissolution was complete. The solution was purified by reverse phase chromatography with 5-50% CH₃CN/H₂O to give 115 mg (14% yield) of a white solid. m/z(M+H)⁺612

Analysis: C₃₂H₃₃N₇O₆+1.85 TFA+1.15 H₂O calcd: C, 50.85; H, 4.44; N, 11.63; found: C, 50.82; H, 4.40; N, 11.65.

HRMS calcd: 612.2565; Found: 612.2578

¹H NMR(400 MHz, DMSO): 1.21(6H, d), 4.09(1H, m), 4.35-4.39(4H, m), 5.34(2H, s), 6.69(1H, s), 6.75(1H, s), 7.09 (1H, s), 7.26(1H, s), 7.37-7.50(7H, m), 7.75(2H, d), 8.68 (1H, t).

### Example 65

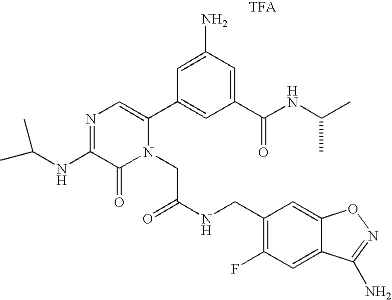

### Example 65

The carboxylic acid, [3-(isopropylamino)-6-[3-({[(1S)-1-methylpropyl]amino}carbonyl)-5-aminophenyl]-2-oxopyrazin-1(2H)-yl]acetic acid (tan solid, M+H =475) (260 mg, 0.55 mmol), the benzyl amine, 6-(aminomethyl)-5-fluoro-1,2-benzisoxazol-3-amine dihydrochloride (139 mg, 0.55 mmol) and HOBt-H₂O (93 mg, 0.61 mmol), were dissolved in DMF (8 mL) and CH₂Cl₂ (50 mL). To this gently stirred mixture was added polymeric DCC resin (2.60 g, loading 1.4 mmol/g, 3.64 mmol) and triethylamine was added to pH=9. The reaction was stirred for 72 h at room temperature (for convenience). The resin was filtered and the filtrate was concentrated. Purification by prep HPLC (RP, acetonitrile gradient in 0.1% TFA) afforded 220 mg of Example 65: (71%) as a colorless amorphous solid.

LCMS (RP, 15-90% acetonitrile in TFA over 14 min): retention time=3.51 min; (M+H)⁺=565.

### Example 66

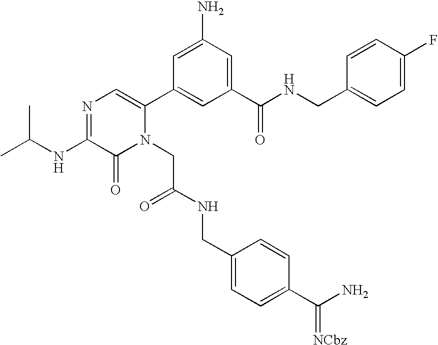

### Benzyl (1E)-amino{4-[({[6-(3-amino-5-{[(4-fluorobenzyl)amino]carbonyl}phenyl)-3-(isopropylamino)-2-oxopyrazin-1(2H)-yl]acetyl}amino)methyl]phenyl}methylidenecarbamate

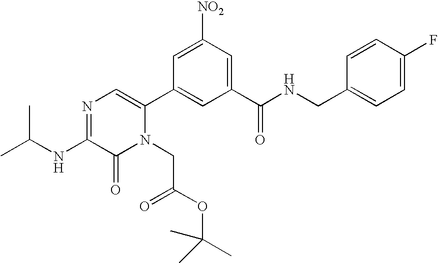

### Tert-butyl [6-(3-{[(4-fluorobenzyl)amino]carbonyl}-5-nitrophenyl)-3-(isopropylamino)-2-oxopyrazin-1(2H)-yl]acetate

### Example 66a

¹H NMR (400 MHz, CD₃OD): δ 8.74 (t, 1 H), 8.39 (t, 1 H), 8.22 (t, 1 H), 7.39-7.36 (m, 2 H), 7.06-7.02 (m, 2 H), 6.82 (s, 1 H), 4.55 (s, 2 H), 4.43 (s, 2 H), 4.18-4.10 (m, 1 H), 1.37 (s, 9 H), 1.26 (d, 6 H)

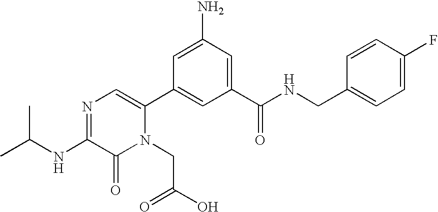

### [6-(3-amino-5-{[(4-fluorobenzyl)amino]carbonyl}phenyl)-3-(isopropylamino)-2-oxopyrazin-1(2H)-yl]acetic Acid

### Example 66b

To a solution of Example 66a (0.82 g, 1.50 mmol) in dichloromethane (20 mL) was added TFA (20 mL) and the reaction mixture stirred overnight at room temperature. The solvent was removed in vacuo to give acid as a brown solid (0.80 g). The crude acid was used without purification in the next step. To the nitro acid in a Parr bottle was added methanol (50 mL) and 10% Pd/c (0.10 g) at room temperature. The reaction was shaken on Parr hydrogenator at 40 psi for 2 hrs and then filtered through Celite. The solvent was removed in vacuo to give Example 66b as an off-white solid (0.65 g, 95%). ¹H NMR (400 MHz, CD₃OD): δ 7.74 (t, 1 H), 7.68 (t, 2 H), 7.38-7.35 (m, 3 H), 7.04 (t, 2 H), 6.70 (s, 1 H), 4.54 (s, 2 H), 4.52 (s, 2 H), 4.06-4.02 (m, 1 H), 1.40 (d, 6 H); MS-ESI (M+H)=454.

### Example 66

¹H NMR (400 MHz, CD₃OD): δ 7.66 (d, 2 H), 7.49-7.21 (m, 5 H), 7.03-6.97 (m, 2 H), 6.84 (t, 1 H), 6.65 (s, 1 H), 5.40 (s, 2 H), 4.63 (s, 2 H), 4.46 (s, 2 H), 4.40 (s, 2 H), 4.05-4.00 (m, 1 H), 1.34 (d, 6 H); ¹⁹F NMR (371 MHz, CD₃OD): δ −77.58 (s, 7.5 F), −118.18 (sep, 1 F); MS-ESI (M+H)=719; Analysis: C₃₉H₃₉FN₈O₅+2.5 TFA+0.95 H₂O calcd: C, 51.76; H, 4.28; N, 10.97; found: C, 51.75; H, 4.28; N, 10.98.

### Example 67

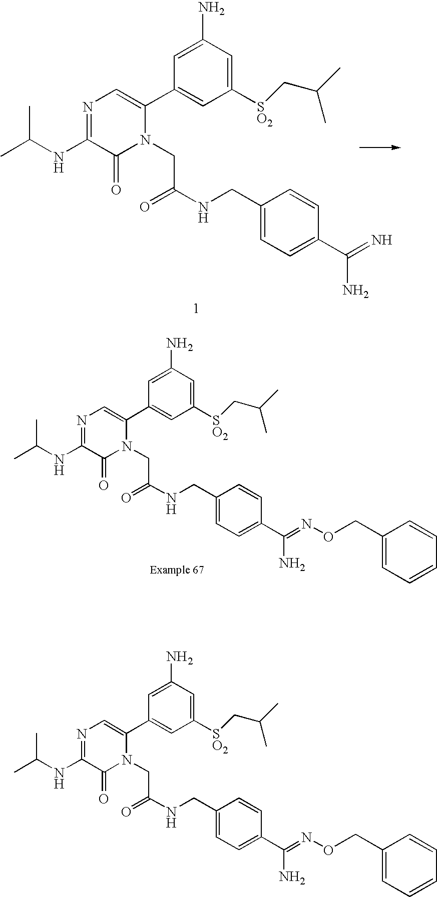

### N-(4-{(Z)-amino[(benzyloxy)imino]methyl}benzyl)-2-[6-[3-amino-5-(isobutylsulfonyl)phenyl]-3-(isopropylamino)-2-oxopyrazin-1(2H)-yl]acetamide

### Example 67

To a 100 mL RBF was added free amidine 1 (0.51 g, 0.66 mmol) in ethanol (30 mL) and DIEA (0.15 mL). To the solution was added benzoxy amine(0.119 g, 0.75 mmol). The reaction stirred at reflux for 7 days. By LCMS the starting material was only 50% consumed. The solution was diluted with 100 mL of 1N HCl and purified by reverse phase chromatography to afford Ex-17 (0.178 g) in 47% yield.

M.S. 659.3(MH+660.7)

Elemental-Isolated as a hydrate with 1.75 TFAs

| | | | |
|---|---|---|---|
| Calculated: | C 61.89 | H 6.26 | N 14.86 |
| Found: | C 52.60 | H 5.08 | N 11.55 |

### Example 68

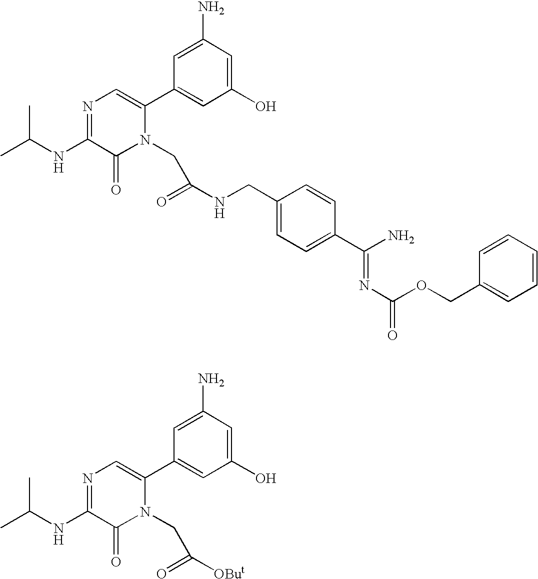

### Tert butyl[6-[3-amino-5-(hydroxy)phenyl]-3-isopropylamino)-2-oxopyrazin-1(2H)-yl]acetate

### Example 68a

1 g (2.02 mmol) of t-butyl[6-[3-nitro-5-(O-benzyl)phenyl]-3-isopropylamino)-2-oxopyrazin-1(2H)-yl]acetate was dissolved in 30 mL MeOH and reduced in the presence of 1 g HCOONH₄ and 0.2 g Pd black with stirring for 12 hours. The catalyst was filtered off and the solvent was evaporated to dryness.

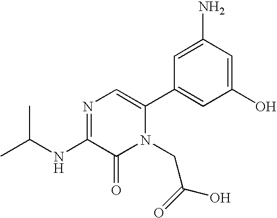

### Example 68b

The residue of EXAMPLE 68a was treated with 35 mL of TFA for 90 minutes stirring and then TFA was evaporated to give a white solid.

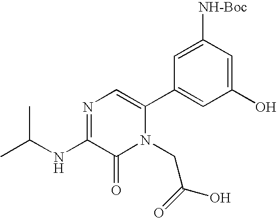

### Example 68c

The white solid of EXAMPLE 68b was dissolved in 20 mL dioxane and 10 mL H₂O and the pH was adjusted to >8 by the addition of 2.5 N NaOH. 0.65 g (3 mmol) (Boc)₂O was added to the mixture and it was stirred for 12 hours. Dioxane was evaporated and the residue was diluted with 50 mL of 10% KHSO₄. It was extracted with 2×100 mL EtOAc. The organic phase was washed with brine, dried over MgSO₄ and the solvent was evaporated. Yield: 0.78 g (1.5 mmol; 75%) solid. MH+=419.1

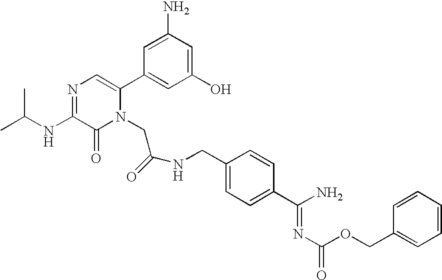

### Example 68d

1.45 g (3 mmol) di-Boc-4-amino-Z-benzamidine was deprotected in 25 mL CH₂Cl₂/TFA (4:1) for 30 minutes stirring and the solvent was thoroughly evaporated to dryness. This solid and 0.78 g (1.5 mmol) of EXAMPLE 68c were dissolved in 25 mL DMF. They were coupled in the presence of 0.7 g (2.2 mmol) TBTU and 0.875 mL (5 mmol) DIPEA for 1 hour. DMF was evaporated and the product was precipitated by addition of 200 mL water and filtered. The crude product was dissolved in the mixture of AcN and H₂O and purified on preparative HPLC using a gradient of acetonitrile (10-50% AcN in 30 minutes), yielding the title product at 48% AcN, 0.614 g (60%) as a white solid.

MH⁺=684.2

### Example 68

0.61 g (0.9 mmol) of EXAMPLE 68d was deprotected in 25 mL CH₂Cl₂/TFA (4:1) for 30 minutes stirring. The solvent was evaporated and the residue was dissolved in AcN/H₂O and lyophilized to yield 0.54 g (0.66 mmol; 74%) white solid.

MH⁺=584.2

¹HNMR: 400 MHz, CD₃OD: 7.78-7.70 (d, 2H), 7.52-7.44 (d, 2H), 7.43-7.32 (m, 5H), 6.64 (s, 1H), 6.40-6.34 (m, 3H), 5.40 (s, 2H), 4.59 (s, 2H), 4.48 (s, 2H), 4.10-4.00 (m, 1H) and 1.36-1.28 (m, 6H).

Elemental analysis: C₃₁H₃₃N₇O₅ + 3xTFA + 1.5xH₂O

| | | | |
|---|---|---|---|
| Found | C: 46.77 | H: 4.17 | N: 10.21 |
| Calc. | C: 46.65 | H: 4.13 | N: 10.29 |

### Example 69

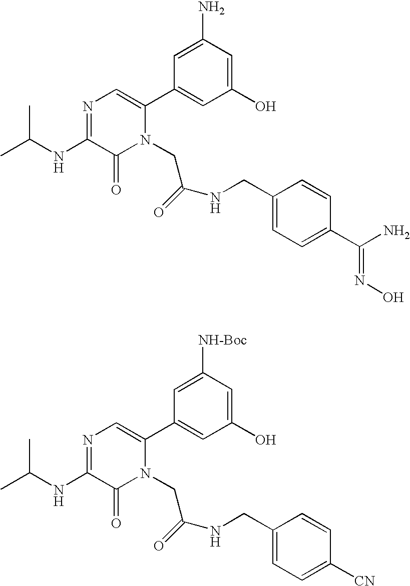

### Example 69a

1 g (3 mmol) N,N-di-Boc-4-aminobenzonitrile was deprotected in 25 mL CH₂Cl₂/TFA (4:1) for 30 minutes and the solvent was evaporated thoroughly to dryness. 0.28 g (0.67 mmol) of the product of EXAMPLE 68c was coupled with the 4-aminobenzo-nitrile in 30 mL DMF in the presence of 0.32 g (1 mmol) TBTU and 1.75 mL (10 mmol) DIPEA with stirring for 12 hours. DMF was evaporated and the product was precipitated by addition of 200 mL water, filtered and dried. Yield: 0.31 g (0.58 mmol; 87%) solid.

MH⁺=533.3

### Example 69b

0.31 g (0.58 mmol) of EXAMPLE 69a was dissolved in 25 mL EtOH and it was refluxed in the presence of 0.347 g (5 mmol) hydroxylamine.HCl and 0.875 mL (5 mmol) DIPEA for 4 hours. The solvent was evaporated and the product was purified on preparative HPLC using a gradient of acetonitrile (10-50% AcN in 30 minutes). Yield: 0.237 g (0.42 mmol; 72%) as a white solid. MH⁺=566.2

### Example 69

0.237 g (0.42 mmol) of EXAMPLE 69b was deprotected in 25 mL CH₂Cl₂/TFA (4:1) with stirring for 30 minutes. The solvent was evaporated and the residue was dissolved in AcN/H₂O and lyophilized to yield 0.277 g (0.4 mmol; 95%) white solid.

MH⁺=466.2

¹HNMR: 400 MHz, CD₃OD: 7.68-7.62 (d, 2H), 7.50-7.44 (d, 2H), 6.61 (s, 1H), 6.54 (s, 1H), 6.48-6.40 (s, 2H), 4.58 (s, 2H), 4.46 (s, 2H), 4.10-3.98 (m, 1H) and 1.40-1.32 (m, 6H).

Elemental analysis: C₂₃H₂₇N₇O₄ + 2.1xTFA + 1.4xH₂O

| | | | |
|---|---|---|---|
| Found | C: 45.04 | H: 4.50 | N: 13.48 |
| Calc. | C: 45.12 | H: 4.46 | N: 13.64 |

### Example 70

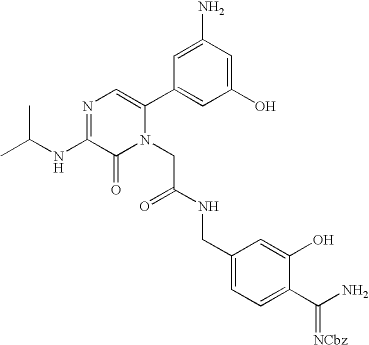

### Benzyl (1E)-amino{4-[({[6-[3-amino-5-(trifluoromethyl)phenyl]-3-(isopropylamino)-2-oxopyrazin-1(2H)-yl]acetyl}amino)methyl]-2-hydroxyphenyl}methylidenecarbamate

### Example 70

To a solution of free amidine (0.098 g, 0.16 mmol) in DMF at room temperature was added benzyl 4-nitrophenyl carbonate (0.06 g, 0.18 mmol) and NMM (0.10 mL, 0.70 mmol). After stirring overnight at room temperature the crude reaction was purified by RP-HPLC to give the desired product (38.9 mg). ¹H NMR (400 MHz, CD₃OD): δ 8.10 (d, 2 H), 7.51-7.37 (m, 5 H), 6.90-6.86 (m, 4 H), 6.65 (s, 1 H), 5.36 (s, 2 H), 4.51 (s, 2 H), 4.35-4.34 (m, 2 H), 4.05-4.01 (m, 1 H), 1.37 (d, 6 H); ¹⁹F NMR (371 MHz, CD₃OD): δ −64.92 (s, 3 F), −77.65 (s, 7.65 F); MS-ESI (M+H)=652; Analysis: C₃₂H₃₂F₃N₇O₅+2.55 TFA+0.05 H₂O calcd: C, 47.23; H, 3.7; N, 10.31; found: C, 47.22; H, 3.67; N, 10.31.

### Example 71

### 3-amino-5-[1-[2-({4-[(Z)-amino({[(2,6-difluorobenzyl)oxy]carbonyl}imino)methyl]benzyl}amino)-2-oxoethyl]-5-(isopropylamino)-6-oxo-1,6-dihydropyrazin-2-yl]benzoic Acid

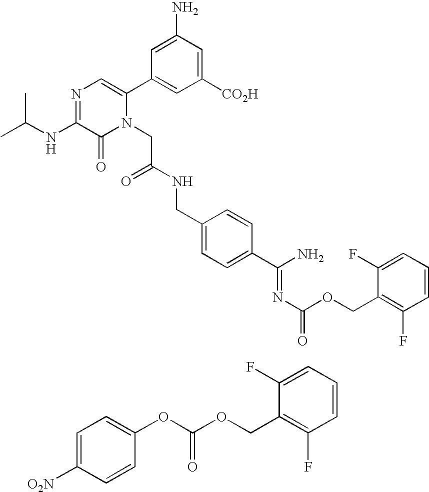

### 2,6-difluorobenzyl 4-nitrophenyl Carbonate

### Example 71a

To a solution of (2,6-difluorophenyl)methanol (2.0 g, 13.87 mmol) in dichloromethane at 0° C. was added NMM (1.73 mL, 16.65 mmol) and 4-nitrophenyl chloridocarbonate (2.8 g, 13.87 mmol). The reaction mixture was stirred at room temperature for 2 hrs and then diluted with ethyl acetate and water. The layers were separated and the organic layer was washed with brine and dried (Na₂SO₄). The solvent was removed in vacuo to give a white solid, which after chromatography (silica 10% ethyl acetate:hexane then 5% ethyl acetate:dichloromethane) to give the desired product as a white solid (3.61 g, 89%). ¹H NMR (400 MHz, CDCl₃): δ 8.26-8.24 (m, 2 H), 7.39-7.36 (m, 3 H), 6.95 (t, 2 H), 5.40 (s, 2 H).

### Example 71

¹H NMR (400 MHz, CD₃OD): δ 7.72 (d, 2 H), 7.53-7.28 (m, 5 H), 7.04 (t, 2 H), 6.89 (t, 1 H), 6.63 (s, 1 H), 5.53 (s, 2 H), 4.58 (s, 2 H), 4.45 (s, 2 H), 4.07-4.00 (m, 1 H), 1.35 (d, 6 H); ¹⁹F NMR (371 MHz, CD₃OD): δ −77.53 (s, 6.6 F), −116.57 (t, 2 F); MS-ESI (M+H)=648; Analysis: C₃₂H₃₁F₂N₇O₆+2.4 TFA+0.65 H₂O calcd: C, 47.37; H, 3.74; N, 10.5; O, 19.63; found: C, 47.37; H, 3.86; N, 10.44; O, 19.64.

### Example 72

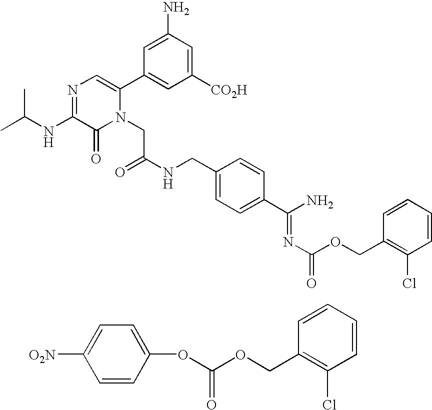

### 2-chlorobenzyl 4-nitrophenyl Carbonate

### Example 72a

To a solution of 2-chlorobenzyl alcohol (3.50 g, 24.6 mmol) and N-methylmorpholine in 200 ml of dichlormethane at 0° C. was added 4-nitrophenyl chloroformate (5.00 g, 24.8 mmol). The solution was allowed to come to room temperature and stirred overnight. The solution was concentrated to a small volume and then purified through a silica gel plug with dichloromethane to give 6.95 g (92% yield).

¹H NMR(400 MHz, CDCl₃): 5.43(2H, s), 7.32-7.52(4H, m), 7.40(2H, d), 8.28(2H, d)

### Example 72

To free amidine (303 mg, 0.59 mmol) and the product from Example 72a (199 mg, 0.65 mmol) in 5 ml of N,N-dimethylformamide was added N-methylmorpholine (0.33 ml, 2.95 mmol). The solution was stirred for 40 hours. The solution was treated with trifluoroacetic acid (0.23 ml, 2.99 mmol) and purified by reverse phase chromatography with 10-50% CH₃CN/H₂O to give a white solid. The solid was purified a second time by reverse phase chromatography with 15-50% CH₃CN/H₂O to give 50 mg (10% yield) of a white solid m/z (M+H)⁺646

Analysis: C₃₂H₃₃ClN₇O₆+2.00 TFA+0.85 H₂O calcd: C, 48.61; H, 4.05; N, 11.02; found: C, 48.64; H, 4.06; N, 10.96.

HRMS calcd: 646.2175; Found: 646.2183

¹H NMR(400 MHz, DMSO): 1.22(6H, d), 4.09(1H, m), 4.36(2H, d), 4.39(2H, s), 5.41(2H, s), 6.70(1H, s), 6.76(1H, t), 7.10(1H, t), 7.26(1H, t), 7.37(2H, d), 7.40-7.47(2H, m), 7.55(1H, m), 7.63(1H, m), 7.76(2H, d), 8.69(1H, t).

### Example 73

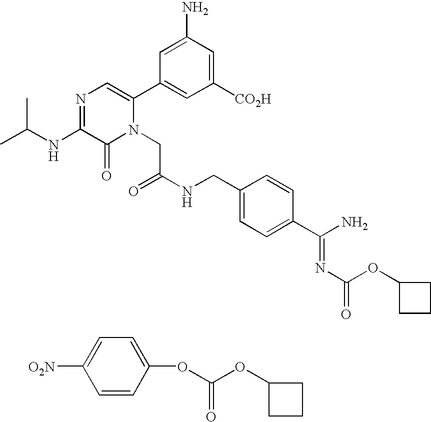

### Cyclobutyl 4-nitrophenyl Carbonate

### Example 73a

To a solution of cyclobutyl alcohol (2.02 g, 28.1 mmol) and N-methylmorpholine in 200 ml of dichlormethane at 0° C. was added 4-nitrophenyl chloroformate (5.71 g, 28.4 mmol). The solution was allowed to come to room temperature and stirred overnight. The mixture was filtered through a silica gel plug with dichloromethane to give 6.7 g (quantitative yield) of a light yellow oil. ¹H NMR(400 MHz, CDCl₃): 1.67(1H, m), 1.87(1H, m), 2.25(2H, m), 2.43(2H, m), 5.05 (1H, m), 7.37(2H, d), 8.26(2H, d).

### Example 73

To free amidine (303 mg, 0.59 mmol) and the product from Example 73a (199 mg, 0.65 mmol) in 5 ml of N,N-dimethylformamide was added N-methylmorpholine (0.33 ml, 2.95 mmol). The solution was stirred for 40 hours. The solution was treated with trifluoroacetic acid (0.23 ml, 2.99 mmol) and purified by reverse phase chromatography with 10-50% CH₃CN/H₂O to give a white solid. The solid was purified a second time by reverse phase chromatography with 15-50% CH₃CN/H₂O to give 50 mg (10% yield) of a white solid m/z (M+H)⁺577

Analysis: C₂₉H₃₃N₇O₆+2.25 TFA+0.10 H₂O calcd: C, 48.25; H, 4.28; N, 11.76; found: C, 48.09; H, 4.49; N, 11.89.

HRMS calcd: 576.2565; Found: 576.2607

### Example 74

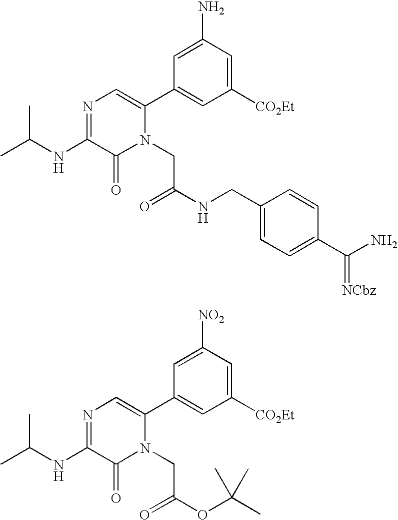

### Example 74a

To the free acid (4.02 g, 9.31 mmol) 4-(dimethylamino)pyridine (0.227 g, 1.86 mmol) and ethanol (2.63 ml, 46.55 mmol) in 50 ml of dichloromethane was added 1-[3-(dimethylamino)propyl]-3-ethylcarbodiimide hydrochloride (2.67 g, 13.97 mmol) and the solution was stirred for 1.5 hours. The solution was concentrated in vacuo and the residue was purified by silica gel chromatography in two batches with 30-50% EA/Hex and 0-10% CH₃OH/CH₂Cl₂. The desired fractions were combined and concentrated. The residue was re-chromatographed with 20, 25% EA/Hex to give 1.95 g (% yield) of an orange solid. m/z (M+H)⁺461

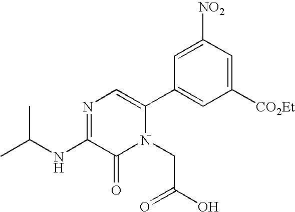

### Example 74b

The product from Example 74a (1.95 g, 4.24 mmol) was dissolved in 4M hydrogen chloride in dioxane (18 ml, 72 mmol) and heated at 60° C. for 6 hours. The mixture was concentrated in vacuo and dried under high vacuum to give 2.05 g (qantitative yield) of a light yellow solid. m/z(M+H)⁺405

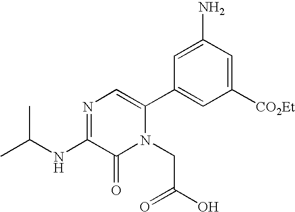

### Example 74c

To the product from Example 74b (2.05 g, mmol) in 50 ml of ethanol was added 0.50 g of 10% palladium on carbon and 0.7 ml of hydrogen chloride (conc.). The mixture was shaken on the Parr apparatus under 45 Psi of hydrogen for 2.5 hours. The mixture was filtered and concentrated in vacuo to give 2.00 g (quantitative yield) of a light yellow solid. m/z(M+H) ⁺375

### Example 74

To the product from Example 74c (1.2 g, 2.74 mmol) and benzyl [4-(aminomethyl)phenyl](imino)methylcarbamate hydrochloride (1.05 g, 3.29 mmol) in 10 ml of N,N-dimethylformamide was added N,N-diisopropylethylamine (2.38 ml, 13.7 mmol) and then benzotriazol-1-yl tetramethyluronium tetrafluoroborate (1.06 g, 3.29 mmol). The solution was stirred for 2 hours. The solution was treated with trifluoroacetic acid (1.27 ml, 16.4 mmol) and purified by reverse phase chromatography with 5-65% CH₃CN/H₂O to give 1.10 g (45% yield) of a white solid. m/z(M+H)⁺640

Analysis: C₃₄H₃₇N₇O₆+2.30 TFA+0.70 H₂O calcd: C, 50.69; H, 4.49; N, 10.72; found: C, 50.70; H, 4.52; N, 10.70.

HRMS calcd: 640.2878; Found: 640.2860

¹H NMR(400 MHz, DMSO): 1.22(6H, d), 1.27(3H, t), 4.09(1H, m), 4.26(2H, q), 4.36-4.39(4H, m), 5.35(2H, s), 6.70(1H, s), 6.78(1H, t), 7.12(1H, t), 7.27(1H, t), 7.37-7.50(7H, m), 7.74(2H, d), 8.70(1H, t).

### Example 75

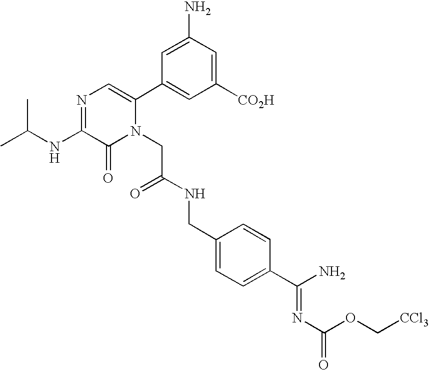

### 3-amino-5-[1-(2-{[4-((Z)-amino{[(2,2,2-trichloroethoxy)carbonyl]imino}methyl)benzyl]amino}-2-oxoethyl)-5-(isopropylamino)-6-oxo-1,6-dihydropyrazin-2-yl]benzoic Acid

### Example 75

To a solution of free amidine (0.30 g, 0.63 mmol) in THF:H₂O (3 mL:1 mL) was added Na₂CO₃ (0.30 g, 2.83 mmol) and succinimidyl 2,2,2-trichloroethyl carbonate (0.20 g, 0.69 mmol) at room temperature. The reaction mixture was stirred for 3 hrs and then decanted and acidified with TFA. The crude reaction mixture was purified by RP-HPLC (CH₃CN: H₂O) to give the desired product (110 mg). ¹H NMR (400 MHz, CD₃OD): δ 7.78 (d, 2 H), 7.45-7.43 (m, 3 H), 7.31-7.29 (m, 1 H), 6.89 (t, 1 H), 6.65 (s, 1 H), 5.08 (s, 2 H), 4.59 (s, 2 H), 4.47 (d, 1 H), 4.06-4.02 (m, 1 H), 1.36 (d, 6 H); MS-ESI (M+H)=654; Analysis: C₂₇H₂₈Cl₃N₇O₆+2.4 TFA+1.35 H₂O calcd: C, 40.16; H, 3.5; N, 10.31; O, 20.44; found: C, 40.21; H, 3.47; H, 3.47; O, 20.48.

### Example 76

### Benzyl (1E)-amino{4-[({[6-{3-amino-5-[(benzylamino)carbonyl]phenyl}-3-(isopropylamino)-2-oxopyrazin-1(2H)-yl]acetyl}amino)methyl]-2-hydroxyphenyl}methylidenecarbamate

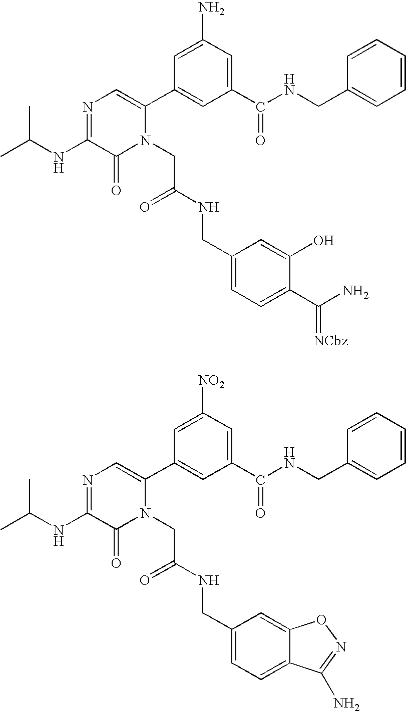

### 3-[1-(2-{[(3-amino-1,2-benzisoxazol-6-yl)methyl]amino}-2-oxoethyl)-5-(isopropylamino)-6-oxo-1,6-dihydropyrazin-2-yl]-N-benzyl-5-nitrobenzamide

### Example 76a

To a solution of free acid (1.55 g, 3.10 mmol) in DMF (20 mL) at 0° C. was added triethylamine (1.9 mL, 13.64 mmol), TBTU (1.49 g, 4.65 mmol), and aminomethyl-1,2-benzisoaxazol-3-amine (1.09 g, 4.65 mmol). The reaction mixture was stirred at room temperature for 2 hrs and then poured into water. The precipitate was filtered and washed with diethyl ether. The solid was dried on high vacuum to give the desired product (1.86 g, 98%). MS-ESI (M+H)=611.

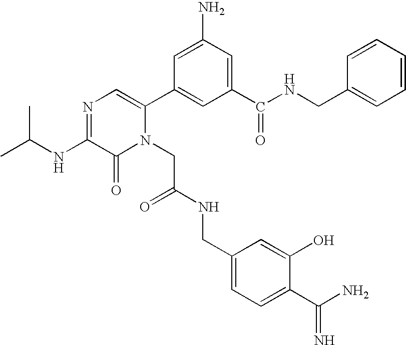

### Example 76b

To a solution of Example 76a (1.86 g, 3.0 mmol) in THF:MeOH (2 mL:20 mL) was added 10% Pd/C (0.4 g) and ammonium formate (0.64 g, 10.1 mmol) at room temperature. The reaction mixture was heated to reflux for 2 hrs and then cooled to room temperature. The reaction was filtered through Celite and the solvent removed to give a crude oil, which was purified by RP-HPLC (CH₃CN: H₂O) to give Example 76b (0.85 g). ¹H NMR (400 MHz, CD₃OD): δ 7.53 (d, 2 H), 7.31-7.21 (m, 5 H), 7.16 (t, 1 H), 6.92 (t, 1 H), 6.87 (s, 1 H), 6.76-6.73 (m, 1 H), 6.64 (s, 1 H), 4.59 (s, 2 H), 4.53 (s, 2 H), 4.29 (s, 2 H), 4.05-4.02 (m, 1 H), 1.37 (d, 6 H); MS-ESI (M+H)=583.

### Example 76c

To a solution of Example 76b (0.30 g, 0.50 mmol) in DMF (3 mL) was added NMM (0.25 mL, 2.2 mmol) and benzyl 4-nitrophenyl carbonate (0.15 g, 0.55 mmol). The reaction mixture was stirred overnight and then acidified with TFA. The crude mixture was purified by RP-HPLC (CH₃CN:H₂O) to give after lypholization the desired product (92 mg). ¹H NMR (400 MHz, CD₃OD): δ 7.47-7.21 (m, 11 H), 7.08 (t, 1 H), 6.86-6.84 (m, 2 H), 6.74-6.72 (m, 2 H), 6.65 (s, 1 H), 5.36 (s, 2 H), 4.59 (s, 2 H), 4.51 (s, 2 H), 4.29 (d, 2 H), 4.05-4.02 (m, 1 H), 1.35 (d, 6 H); MS-ESI (M+H)=717; Analysis: C₃₉H₄₀N₈O₆+2.15 TFA+1.4 H₂O calcd: C, 52.68; H, 4.58; N, 11.35; 0, 18.96; found: C, 52.68; H, 4.58; N, 11.44; O, 19.01.

### Example 77

### Benzyl (1E)-amino{4-[({[6-(3-amino-5-{[(4-fluorobenzyl)amino]carbonyl}phenyl)-3-(isopropylamino)-2-oxopyrazin-1(2H)-yl]acetyl}amino)methyl]-2-hydroxyphenyl}methylidenecarbamate

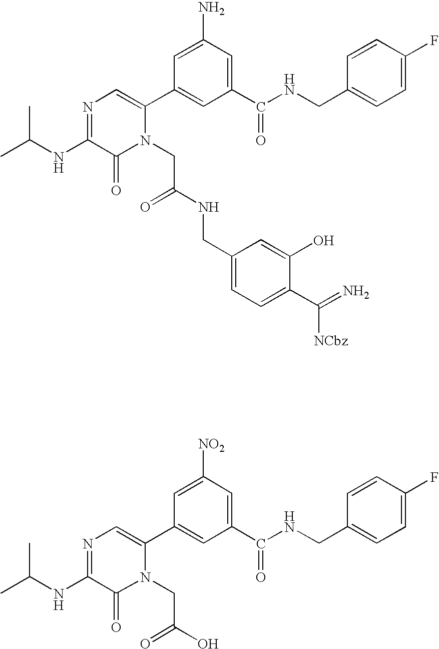

### [6-(3-{[(4-fluorobenzyl)amino]carbonyl}-5-nitrophenyl)-3-(isopropylamino)-2-oxopyrazin-1(2H)-yl]acetic Acid

### Example 77a

To a solution of t-butyl ester (1.7 g, 3.0 mmol) in dichloromethane (20 mL) was added TFA (20 mL) and the reaction mixture stirred overnight at room temperature. The solvent was removed in vacuo to give the acid as a brown solid (1.63 g). MS-ESI (M+H)=484.

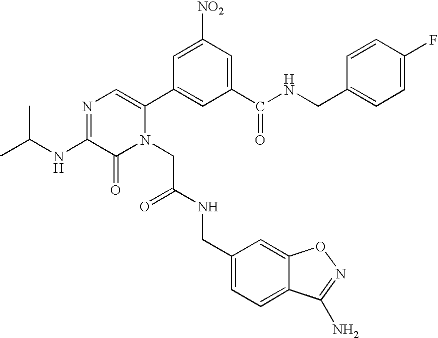

### 3-[1-(2-{[(3-amino-1,2-benzisoxazol-6-yl)methyl]amino}-2-oxoethyl)-5-(isopropylamino)-6-oxo-1,6-dihydropyrazin-2-yl]-N-(4-fluorobenzyl)-5-nitrobenzamide

### Example 77b

To a solution of Example 77a (1.63 g, 3.10 mmol) in DMF (20 mL) at 0° C. was added TBTU (1.49 g, 4.65 mmol), TEA (1.9 mL, 13.64 mmol) and aminomethyl-1,2-benzisoaxazol-3-amine (1.09 g, 4.65 mmol). The reaction was stirred at room temperature for 2 hrs and then poured into water. The precipitate was filtered and washed with diethyl ether to give the desired product as a yellow solid (1.94 g, 100%). MS-ESI (M+H)=629.

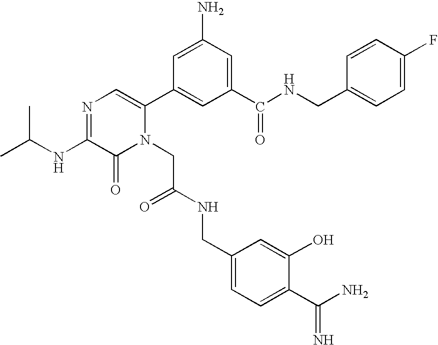

### 3-amino-5-[1-[2-({4-[amino(imino)methyl]-3-hydroxybenzyl}amino)-2-oxoethyl]-5-(isopropylamino)-6-oxo-1,6-dihydropyrazin-2-yl]-N-(4-fluorobenzyl)benzamide

### Example 77c

To a solution of Example 77b (1.94 g, 3.10 mmol) in THF:EtOH (10 mL:40 mL) was added 10% Pd/C and concentrated HCl (3 drops) at room temperature. The reaction mixture was shaken on a Parr hydrogenator at 40 psi for 24 hrs. The reaction was filtered through Celite and the filtrate concentrated to give a brown oil, which after purification by RP-HPLC (CH₃CN:H₂O) and lypholization gave the desired product (787 mg). ¹H NMR (400 MHz, CD₃OD): δ 7.53 (d, 1 H), 7.34-7.30 (m, 2 H), 7.26 (t, 1 H), 7.11 (t, 1 H), 7.0 (t, 2 H), 6.88-6.86 (m, 2 H), 6.76-6.73 (m, 1 H), 6.64 (s, 1 H), 4.59 (s, 2 H), 4.49 (s, 2 H), 4.30 (s, 2 H), 4.06-4.02 (m, 1 H), 1.36 (d, 6 H); ¹⁹F NMR (371 MHz, CD₃OD) δ −77.05 (s, 8.7), −118.16 (sep, 1 F); MS-ESI (M+H)=601; Analysis: C₃₁H₃₃FN₈O₃+2.4 TFA+1.15 H₂O calcd: C, 48.91; H, 4.32; N, 12.74; O, 16.29; found: C, 48.99; H, 4.29; N, 12.68; O, 16.3.

### Example 77d

To a solution of Example 77c (0.30 g, 0.50 mmol) in DMF (3 mL) at room temperature was added NMM (0.25 mL, 2.2 mmol) and benzyl 4-nitrophenyl carbonate (0.15 g, 0.55 mmol). The reaction mixture was stirred overnight and then acidified with TFA. The crude mixture was purified by RP-HPLC (CH₃CN:H₂O) to give after lypholization the desired product (142 mg). ¹H NMR (400 MHz, CD₃OD): δ 8.10 (d, 1 H), 7.46-7.24 (m, 8 H), 7.09 (t, 1 H), 6.98 (t, 2 H), 6.88-6.86 (m, 2 H), 6.75-6.73 (m, 1 H), 6.64 (s, 1 H), 5.36 (s, 2 H), 4.59 (s, 2 H), 4.47 (s, 2 H), 4.30 (s, 2 H), 4.06-4.02 (m, 1 H), 1.35 (d, 6 H); ¹⁹F NMR (371 MHz, CD₃OD): 6-77.47 (s, 6.6 F), −118.10 (sep, 1 F); MS-ESI (M+H)=735; Analysis: C₃₉H₃₉FN₈O₆+2.1 TFA+0.95 H₂O calcd: C, 52.34; H, 4.37; N, 11.3; found: C, 52.31; H, 4.34; N, 11.34.

### Example 78

### 3-amino-5-[1-[2-({4-[(Z)-amino({[(4-fluorobenzyl)oxy]carbonyl}imino)methyl]benzyl}amino)-2-oxoethyl]-5-(isopropylamino)-6-oxo-1,6-dihydropyrazin-2-yl]benzoic Acid

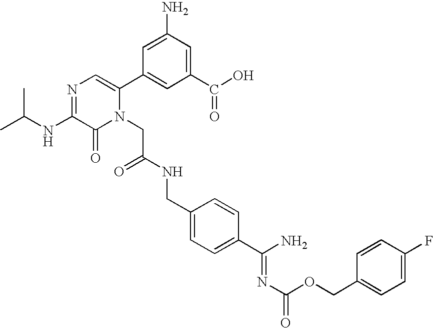

### Example 78

To a solution of free amidine (0.30 g, 0.63 mmol) in DMF (3 mL) at room temperature was added NMM (0.31 mL, 2.8 mmol) and 4-fluorobenzyl 4-nitrophenyl carbonate (0.20 g, 0.69 mmol). The reaction mixture was stirred overnight and then acidified with TFA. The crude reaction mixture was purified by RP-HPLC (CH₃CN: H₂O) to give after lypholization the desired product (151 mg). ¹H NMR (300 MHz, CD₃OD): δ 7.74 (d, 2 H), 7.55-7.42 (m, 6 H), 7.31 (s, 1 H), 7.14 (t, 2 H), 6.91 (s, 1 H), 6.64 (s, 1 H), 5.38 (s, 2 H), 4.59 (s, 2 H), 4.46 (d, 1 H), 4.06-4.02 (m, 1 H), 1.38 (d, 6 H); ¹⁹F NMR (371 MHz, CD₃OD): δ −79.09 (s, 6.8 F), −116.66 (sep, 1 F); MS-ESI (M+H)=630; Analysis: C₃₂H₃₂FN₇O₆+2.4 TFA+1.2 H₂O calcd: C, 47.78; H, 4.01; N, 10.6; found: 47.78; H, 3.96; N, 10.67.

### Example 79

### 4-methoxyphenyl {4-[({[6-(3-amino-5-hydroxyphenyl)-3-(isopropylamino)-2-oxopyrazin-1(2H)-yl]acetyl}amino)methyl]phenyl}(imino)methylcarbamate

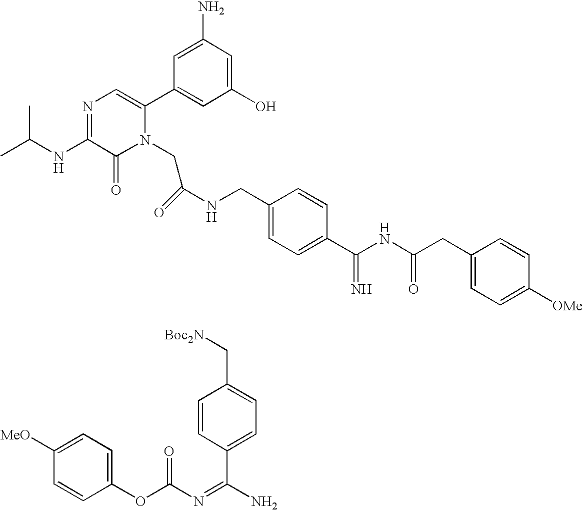

### Di(tert-butyl)4-((E)-amino{[(4-methoxyphenoxy)carbonyl]imino}methyl)benzylimidodicarbonate

### Example 79a

To a 250 mL RBF was added the di-Boc-amidine (5 g, 9.5 mmol) in dioxane(25 mL) and 1 N sodium hydroxide(30 mL). The reaction stirred for 15 minutes. To the reaction was added the 4-methyoxyphenyl chloroformate (4.2 mL, 28.5 mmol) as two separate portions 1 hour apart. After the second addition a precipitate formed and the reaction stirred for an additional hour. The reaction was dumped into 200 mL of water. The aqueous layer was extracted with methylene chloride (2×50 mL). The organics were combined, dried over MgSO4 then concentrated to afford Ex-3a (4.5 g) in 99% yield.

MS 499.56 (MH+ 500.5)

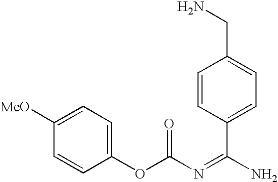

### 4-methoxyphenyl (1E)-amino[4-(aminomethyl)phenyl]methylidenecarbamate

### Example 79b

To a 250 mL RBF was added Example 79a (3.5 g, 7 mmol) in 4 N HCl/dioxane. The reaction stirred for 1 hour and L.C. indicated that the reaction was finished. The solvent and excess HCl was removed in vacuo to afford a white solid that will be used as is.

MS 299.32(MH+300.1)

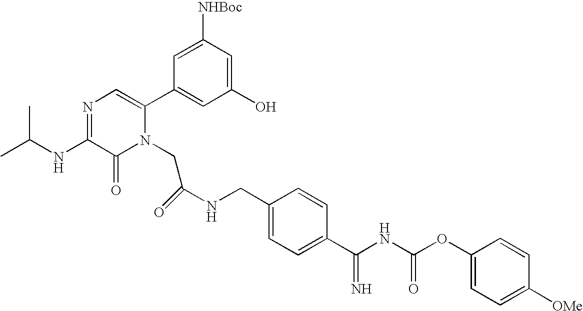

### 4-methoxyphenyl{4-[({[6-{3-[(tert-butoxycarbonyl)amino]-5-hydroxyphenyl}-3-(isopropylamino)-2-oxopyrazin-1(2H)-yl]acetyl}amino)methyl]phenyl}(imino)methylcarbamate

### Example 79c

To a 250 mL RBF was added Example 79b (2.2 g,7.2 mmol) in DMF (25 mL. To the solution was added DIEA (5 mL), the acid (1.5 g, 3.6 mmol) and TBTU (2.3 g, 7.2 mmol). The reaction stirred overnight at room temperature then was dumped into water (250 mL). The organicas were extracted with ethyl acetate which was dried over MgSO4 then concentrated in vacuo to afford Example 79c (1.75 g) in 70% yield. By LC and Mass Spec. the product is pure enough to carry on to the next step.

MS 699.25(MH+700.2)

### Example 79d

To a 100 mL RBF was added Example 79c (1.75 g, 2,5 mmol) in methylene chloride (50 mL) and TFA (10 mL).The reaction stirred for 20 minutes then was checked by L.C. and M.S. and the starting material was observed to be consumed. The reaction was concentrated in vacuo and the resulting oil was triturated with diethyl ether. The resulting tan solid was dried on a high vacuum overnight to afford Example 79d (1.4 g) in 92% yield.

MS 599.64(MH+600.5)

NMR (400 MHz, CDCl₃): ¹H 1.48 ppm (6H, d), 3.80 ppm (3H, s) 4.05 ppm(1H, q), 4.51 ppm (2H, s), 4.61 ppm (2H,s), 6.42 ppm (1H, s), 6.48 ppm (1H, s), 6.53 ppm (1H, t), 6.62 ppm (1H, s), 6.98 ppm (2H, d), 7.20 ppm (2H, d), 7.56 ppm (2H, d), 7.84 ppm (2H, d).

### Example 80

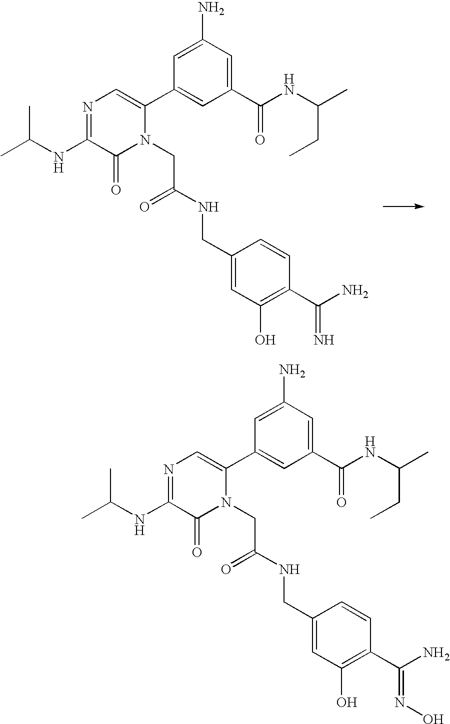

### 3-amino-5-[1-[2-({4-[(Z)-amino(hydroxyimino)methyl]-3-hydroxybenzyl}amino)-2-oxoethyl]-5-(isopropylamino)-6-oxo-1,6-dihydropyrazin-2-yl]-N-(sec-butyl)benzamide

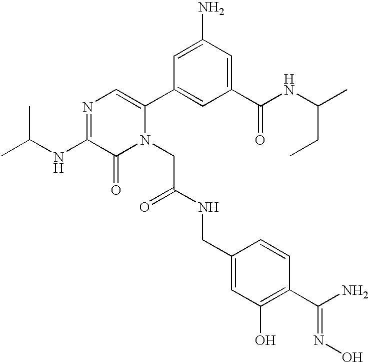

### Example 80

To a 100 mL RBF was added free amidine (0.92 g, 1.68 mmol) in ethanol (20 mL). To the reaction was added hydroxyl amine hydrochloride (0.2 g, 29 mmol) and triethyl amine (0.99 mL).The reaction was heated to 50 C. and checked after 2 hours by L.C. No reaction was observed. The reaction was heated to reflux for 16 hours. By L.C. and M.S. the starting material was consumed and desired product was observed. To the reaction was added water and acetonitrile to the volume of 150 mL and the crude reaction was purified on reverse phase chromatography to afford Example 80 (0.510 g) in 54% yield.

M.S. 564.64(MH+566.7)

Elemental-Isolated as a salt with 2 TFAs

| | | | |
|---|---|---|---|
| Calculated: | C 59.82 | H 6.45 | N 19.85 |
| Found: | C 45.70 | H 4.68 | N 12.90 |

### Example 81

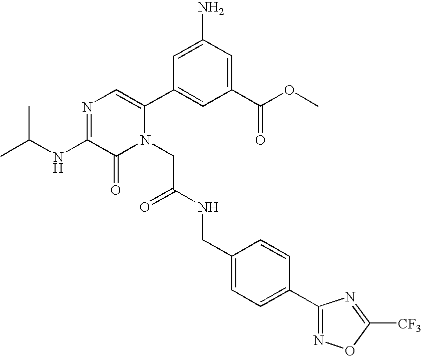

### Example 81

To a stirred solution of free acid (1.0 g, 2.5 mmol), oxadiazol-amine (0.84 g, 3.0 mmol), and N-methylmorpholine (1.37 ml, 8.0 mmol) in N,N-dimethylacetamide (12 ml) cooled in an ice bath was added TBTU (0.88 g, 2.8 mmol). Stirring was continued at ambient temperature for 2 hour. The reaction was diluted with water and the resulting solid was collected by vacuum filtration, washed with water, and air-dried. The solid was crystallized from methanol/water to give 0.88 g (60% yield) of an off-white solid. ¹HNMR (300 MHz, DMSO-d₆) δ 1.23 (d, J=6.3 Hz, 6H), 3.82-3.98 (s, 3H), 4.05-4.20 (m, 1H), 4.39 (d, J=4.8 Hz, 2H), 4.44 (s, 2H), 5.66 (s, 2H), 6.69 (s, 1H), 6.81 (s, 1H), 6.86 (d, J=8.1 Hz, 1H), 7.12 (s, 1H), 7.29 (s, 1H), 7.41 (d, J=7.8 Hz, 2H), 8.01 (d, J=8.1 Hz, 2H), 8.61-8.70 (m, 1H). ¹⁹FNMR (282 MHz, DMSO-d₆) δ −65.08 (s). HRMS (ES) calcd for C₂₇H₂₇N₇O F₃ (M+H): 586.2020. Found: 586.2026. Anal. Calcd for C₂₇H₂₆N₇O₅F₃+0.2 CH₃OH: C, 55.19; H, 4.56; N, 16.56. Found: C, 55.23; H, 4.46; N, 16.48.

### Example 82

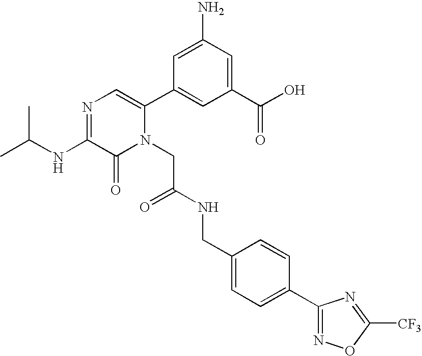

Example 82

To a stirred solution of Example 81 (0.63 g, 1.07 mmol) in methanol (20 ml) and THF (20 ml) cooled in an ice bath was added 2.5 N sodium hydroxide (1.42 ml, 3.54 mmol) and reaction stirred at ambient temperature for 22 h. Additional 2.5 N sodium hydroxide (0.473 ml, 1.18 mmol) added and stirring continued for 4 hours. Reaction was acidified with 6N hydrochloric acid and purification by reverse phase HPLC (5-50% acetonitrile/water) followed by lyophilization yielded 194 mg (25% yield) of an off-white solid. ¹HNMR (300 MHz, DMSO-d6) δ 1.26 (d, J=6.6 Hz, 6H), 4.07-4.18 (m, 1H), 4.36-4.47 (m, 4H), 6.74 (s, 1H), 6.80 (s, 1H), 7.14 (s, 1H), 7.31 (s, 1H), 7.44 (d, J=8.4 Hz, 2H), 8.04 (d, J=8.4 Hz, 2H), 8.63-8.70 (m, 1H), 12.90 (br s, 1H). ¹⁹FNMR (282 MHz, DMSO-d₆) δ −65.05 (s). HRMS (ES) calcd for C₂₆H₂₅N₇O₅F₃ (M+H): 572.1870. Found: 572.1861. Anal. Calcd for C₂₆H₂₄N₇O₅F₃+1.3 TFA+0.7 H₂O: C, 46.9; H, 3.67; N, 13.38. Found: C, 46.9; H, 3.64; N, 13.41.

### Example 83

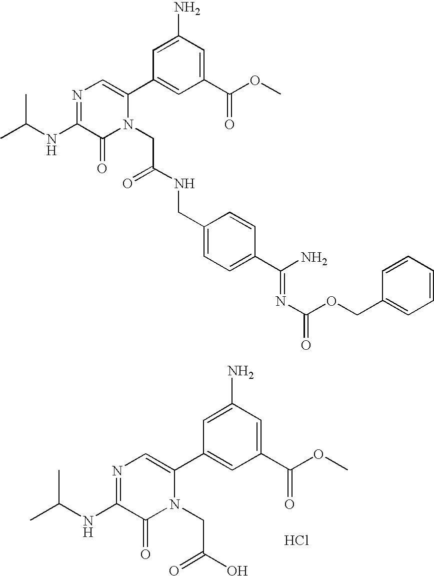

### Example 83a

The nitro compound (2.25 g, 5.3 mmol) was shaken with 10% palladium on carbon (1.25 g) in methanol (50 ml) under 45 psi hydrogen for 1 hour. The reaction was filtered and concentrated in vacuo. The residue was treated as above with the addition of 6N hydrogen chloride (0.88 ml, 5.3 mmol) to give 1.65 g (79% yield) of Example 83a as a light tan solid. LCMS (ES+) m/z M+H 361.

### Example 83b

¹HNMR (300 MHz, DMSO-d₆) δ 1.19 (d, J=6.3 Hz, 6H), 3.78 (s, 3H), 4.00-4.14 (m, 1H), 4.30-4.40 (m, 4H), 5.32 (s, 2H), 6.67 (s, 1H), 6.75 (s, 1H), 7.08 (s, 1H) 7.25 (s, 1H), 7.32-7.50 (mm, 7H), 7.72 (d, J=8.4 Hz, 2H), 8.66 (t, J=5.7 Hz, 1H) 10.41 (br s, 1H). HRMS (ES) calcd for C₃₃H₃₆N₇O₆ (M+H): 626.2722. Found: 626.2723. Anal. Calcd for C₃₃H₃₅N₇O₆+2.05 TFA+0.75 H₂O: C, 51.04; H, 4.45; N, 11.23. Found: C, 51.09; H, 4.49; N, 11.16.

### Example 84

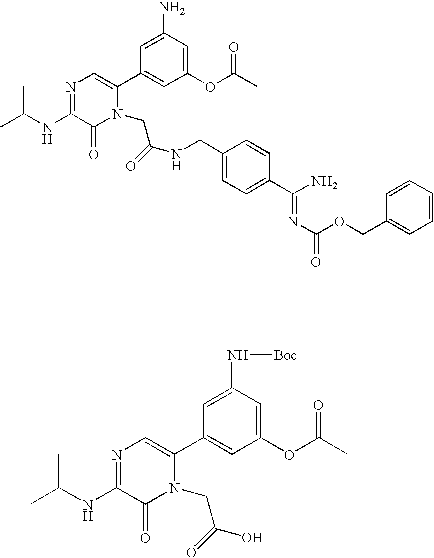

### Example 84a

3.8 g (9.1 mmol) of free alcohol was reacted with 4.08 g (40 mmol; 3.77 mL) acetic anhydride in 50 mL CH₂Cl₂ in the presence of 8.75 mL (50 mmol) DIPEA with stirring for 4 hours. The mixture was washed with 100 mL brine, dried over MgSO₄ and the solvent was evaporated. Yield: 3.3 g (7.2 mmol; 79%) oil. MH⁺=461.3

### Example 84b

1.2 g (2.5 mmol) di-Boc-4-amino-Z-benzamidine was deprotected in 25 mL CH₂Cl₂/TFA (4:1) for 30 minutes stirring and the solvent was thoroughly evaporated to dryness. This solid and 0.8 g (1.74 mmol) of EXAMPLE 84a were dissolved in 15 mL DMF. They were coupled in the presence of 0.64 g (2 mmol) TBTU and 1.75 mL (10 mmol) DIPEA for 1 hour. DMF was evaporated and the product was precipitated by addition of 200 mL water and filtered. The crude product was dissolved in the mixture of AcN and H₂O and purified on preparative HPLC using a gradient of acetonitrile (10-50% AcN in 30 minutes), yielding the title product at 46% AcN, 0.91 g (1.2 mmol; 72%) as a white solid. MH⁺=726.1

### Example 84c

0.61 g (0.9 mmol) of EXAMPLE 84b was deprotected in 25 mL CH₂Cl₂/TFA (4:1) with stirring for 30 minutes. The solvent was evaporated and the residue was dissolved in AcN/H₂O and lyophilized to yield 0.81 g (0.95 mmol; 79%) white solid.

MH⁺=62.2

¹HNMR: 400 MHz, CD₃OD: 7.78-7.70 (d, 2H), 7.52-7.44 (m, 4H), 7.43-7.32 (m, 3H), 6.66-6.60 (m, 3H), 6.50 (s, 1H), 5.40 (s, 2H), 4.62 (s, 2H), 4.46 (s, 2H), 4.10-3.96 (m, 1H), 2.24 (s, 3H) and 1.40-1.30 (m, 6H).

Elemental analysis: C₃₃H₃₅N₇O₆ + 2.5xTFA + 2.1xH₂O

| | | | |
|---|---|---|---|
| Found | C: 48.11 | H: 4.42 | N: 10.09 |
| Calc. | C: 48.12 | H: 4.43 | N: 10.34 |

### Example 85

### 3-amino-5-[1-{2-[(4-{(Z)-amino[(phenoxycarbonyl)imino]methyl}benzyl)amino]-2-oxoethyl}-5-(isopropylamino)-6-oxo-1,6-dihydropyrazin-2-yl]benzoic Acid

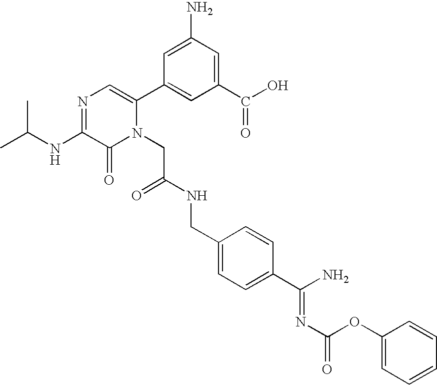

### Example 85

To a solution of free amidine (0.30 g, 0.62 mmol) in DMF (3 mL) was added NMM (0.29 mL, 2.76 mmol) and diphenyl carbonate (0.14 g, 0.69 mmol). After stirring overnight the reaction mixture was acidified with TFA and purified by RP-HPLC (CH₃CN:H₂O) to give desired product after lypholization (85 mg). ¹H NMR (400 MHz, CD₃OD): δ 7.82 (d, 2 H), 7.48-7.32 (m, 8 H), 6.92 (t, 1 H) 6.64 (s, 1 H), 4.60 (s, 2 H), 4.48 (s, 2 H), 4.06-4.02 (m, 1 H), 1.37 (d, 6 H); MS-ESI (M+H)=598; Analysis: C₃₁H₃₁N₇O₆+3.0 TFA+1.15 H₂O calcd: C, 45.27; H, 3.8; N, 10.2; O, 21.9; found: C, 45.35; H, 4.34; N, 11.99; O, 21.49.

### Example 86

### Pyridin-3-ylmethyl {4-[({[6-(3-amino-5-hydroxyphenyl)-3-(isopropylamino)-2-oxopyrazin-1(2H)-yl]acetyl}amino)methyl]phenyl}(imino)methylcarbamate

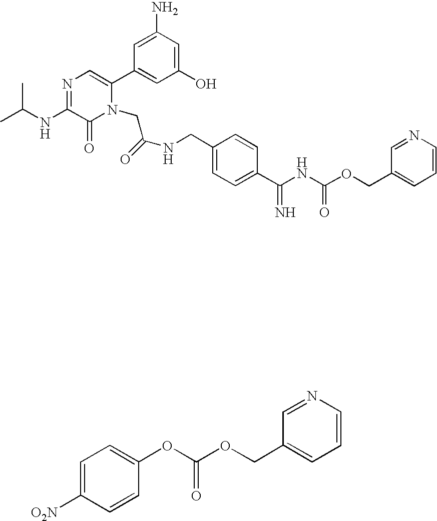

### 4-nitrophenyl Pyridin-3-ylmethyl Carbonate

### Example 86a

To a 250 mL RBF was added the 1 pyridin-3-ylmethanol (10 g, 91.3 mmol) in pyridine (75 mL). The solution was cooled to 0° C. and the p-nitrophenyl chloroformate was added in amall portions over ½ hour. The reaction stirred at 0° C. for 1 hour then allowed to warm to room temperature overnight. To the reaction was added methylene chloride (100 mL) and he mixture was washed with water (3×100 mL). The organics were then washed with brine (2×100 mL) and dried over MgSO4. The organics were concentrated in vacuo to afford a glassy brown solid. The solid was dissolved in 400 mL of hot ethanol and in the freezer overnight. The resulting solid was filtered off and dried in a dessicator to afford (9.5 g) EX-1a in 38% yield.

MS 274.23 (MH+275)

NMR (400 MHz, CDCl₃): ¹H 5.3 ppm (2H, s), 7.39 ppm (3H, m) 7.8 ppm(1H, d), 8.28 ppm (2H, d), 8.62 ppm (1H,s), 8.72 ppm (1H,s). ¹³C 6.280, 121.699, 123.615, 125.323, 129.865, 136.387, 149.998, 150.61, 152.358, 155.306 ppm.

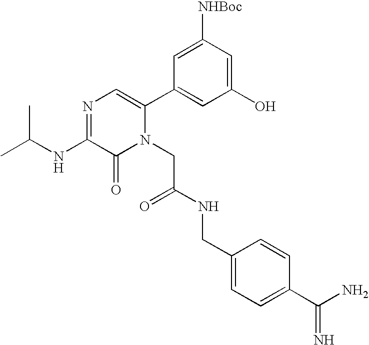

### Tert-butyl 3-[1-[2-({4-[amino(imino)methyl]benzyl}amino)-2-oxoethyl]-5-(isopropylamino)-6-oxo-1,6-dihydropyrazin-2-yl]-5-hydroxyphenylcarbamate

### Example 86b

To a 250 mL RBF was added 4-amino-benzamidine (1.3 g, 4.56 mmol) in DMF (20 mL), free acid and DIEA (2 mL). To the reaction was added TBTU (1.6 g, 5.0 mmol) and the reaction stirred overnight. The reaction was dumped into water and extracted with ethyl acetate (2×50 mL). The organics were then washed with brine, dried over MgSO4 then concentrated in vacuo. The resulting solid was triturated with diethyl ether to afford a white solid (2.55 g) in 82% yield. The solid was then subjected to catalytic hydrogenation in methanol and acetic acid overnight. The palladium was filtered off and the reaction was concentrated in vacuo to afford the deprotected amidine EXAMPLE 86b. MS 549(MH+550)

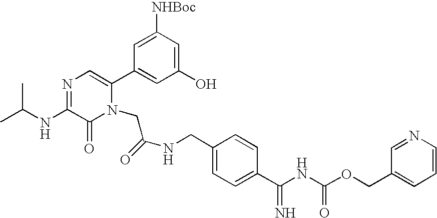

### Pyridin-3-ylmethyl {4-[({[6-{3-[(tert-butoxycarbonyl)amino]-5-hydroxyphenyl}-3-(isopropylamino)-2-oxopyrazin-1(2H)-yl]acetyl}amino)methyl]phenyl}(imino)methylcarbamate

### Example 86c

To a 100 mL RBF was added the EXAMPLE 86b (0.5 g, 0.77 mmol) and EXAMPLE 86a (0.264 g, 0.96 mmol) in DMF (25 mL) and methylene chloride (10 mL). To the reaction was added 1 N sodium hydroxide (5 mL) then the reaction stirred overnight. The reaction was poured into water (50 mL) and methylene chloride (50 mL). The organics were collected and washed with brine (2×50 mL). The organics were dried over MgSO4 and concentrated in vacuo. The resulting solid was dissolved in water and acetonitrile (150 mL) and purified with reverse phase chromatography to afford EXAMPLE 86c (0.225 g) in 43% yield.

MS 684.74 (MH+685.8)

### Example 86d

To a 50 mL RBF was added Ex-1c (0.220 g, 0.32 mmol) in a 20% solution of TFA and methylene chloride. The reaction stirred for 1 hour then was concentrated in vacuo. The resulting solid was dissolved in water (100 mL) and purified by reverse phase chromatography to afford (0.200 g) in 99% yield as the TFA salt.

NMR (400 MHz, CDCl₃): ¹H 1.36 ppm (6H, d), 3.30 ppm (2H, m) 4.03 ppm(1H, q), 4.9 ppm (2H, s), 4.59 ppm (2H,s), 5.53 ppm (2H,s), 6.54 ppm (2H, bs), 6.62 ppm (2H, b), 7.51 ppm (2H, d), 7.77 ppm (3H, d), 8.32 ppm (1H, bs), 8.7 ppm (1H, s), 8.84 ppm (2H, s). ¹³C 6.280, 121.699, 123.615, 125.323, 129.865, 136.387, 149.998, 150.61, 152.358, 155.306 ppm.

### Example 87

### 3-amino-5-[1-{2-[(4-{(Z)-amino[(phenoxycarbonyl)imino]methyl}benzyl)amino]-2-oxoethyl}-5-(isopropylamino)-6-oxo-1,6-dihydropyrazin-2-yl]benzoic Acid

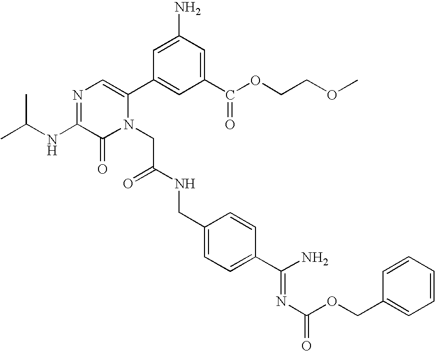

### Example 87

To a round bottom flask containing protected Bromo compound (1.72 g, 3.10 mmol) was added 3-amino-s-[(2-methoxyethoxy)carbonyl]phenylboronic acid (1.72 g, 6.2 mmol), Pd(PPh₃)₄ (0.71 g, 0.62 mmol) and Cs₂CO₃ (2.22 g, 6.82 mmol). The flask was flushed with nitrogen and the solids dissolved in CH₃CN:H₂O (15 mL:1.5 mL). The reaction was heated to 80° C. for 9 hrs and then cooled to room temperature. The mixture was poured into water and ethyl acetate. The layers were separated and organic layer washed with brine and dried (Na₂SO₄). The solvent was removed to give a yellow oil, which after chromatography (silica, 60-100% ethyl acetate:hexane) and RP-HPLC (CH₃CN:H₂O) gave the desired product (0.33 g, 16%). ¹H NMR (400 MHz, CD₃OD): δ 7.73 (d, 2 H), 7.49-7.32 (m, 9 H), 6.92 (t, 1 H), 6.64 (s, 1 H), 5.39 (s, 2 H), 4.58 (s, 2 H), 4.47 (d, 2 H), 4.38 (t, 2 H), 4.06-4.02 (m, 1 H), 3.66 (t, 2 H), 3.29 (s, 3 H), 1.36 (d, 2 H); MS-ESI (M+H)=670; Analysis: C₃₅H₃₉N₇O₇+2.3 TFA+1.4 H₂O calcd: C, 49.68; H, 4.64; N, 10.24; O, 21.72; found: C, 49.68; H, 4.49; N, 10.31; O, 21.77.

### Example 88

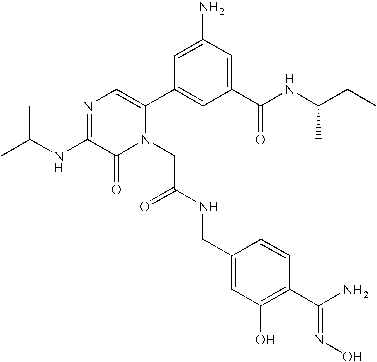

### Example 88

A stirred suspension of free amidine (0.25 g, 0.46 mmol), hydroxylamine hydrochloride (95 mg, 1.37 mmol), and triethylamine (0.19 ml, 1.37 mmol) in absolute ethanol (4 ml) were heated at 90° C. for 4.5 hours (note extended heating resulted in poorer yield). Purification by reverse phase HPLC (5-45% acetonitrile/water) followed by lyophilization yielded 130 mg (38% yield) of Example 88 as an off-white solid. ¹HNMR (300 MHz, DMSO-d₆) δ 0.87 (t, J=7.5 Hz, 3H), 1.12 (d, J=6.6 Hz, 3H), 1.24 (d, J=6.6 Hz, 6H), 1.43-1.59 (m, 2H), 3.82-3.96 (m, 1H), 4.04-4.19 (m, 1H), 4.24 (d, J=5.4 Hz, 2H), 4.40 (s, 2H), 6.70 (s, 1H), 6.72 (s, 1H), 6.76 (d, J=9.0 Hz, 1H), 6.86 (s, 1H), 7.02 (s, 1H), 7.13 (s, 1H), 7.36 (d, J=7.8 Hz, 2H), 8.04 (d, J=8.7 Hz, 2H), 8.63 (t, J=6.0 Hz, 1H) 10.90 (br s, 1H) HRMS (ES) calcd for C₂₈H₃₇N₈O₅ (M+H): 565.2881. Found: 565.2890. Anal. Calcd for C₂₈H₃₆N₈O₅+2.3 TFA+1.1 H₂O: C, 46.24; H, 4.82; N, 13.23. Found: C, 46.25; H, 4.83; N, 13.23.

### Example 89

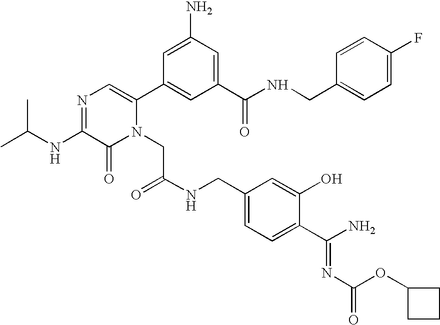

### Example 89

To the free amidine (400 mg, 0.67 mmol) and cyclobutyl-chloroformate (174 mg, 0.73 mmol) in 3 ml of N,N-dimethylformamide was added N-methylmorpholine (0.29 ml, 2.67 mmol). The solution was stirred for 40 hours and then heated at 60° C. for 2 hours. The solution was treated with trifluoroacetic acid (0.26 ml, 3.34 mmol) and purified by reverse phase chromatography with 25-60% CH₃CN/H₂O to give 130 mg (22% yield) of a light yellow solid m/z(M+H)⁺699 Analysis: C₃₆H₃₉FN₈O₆+1.65 TFA+1.40 H₂O calcd: C, 51.75; H, 4.80; N, 12.28; found: C, 51.71; H, 4.70; N, 12.44. HRMS calcd: 699.3049; Found: 699.3057

¹H NMR(400 MHz, DMSO): 1.22(6H, d), 1.63(1H, m), 1.78(1H, m), 2.10(2H, m), 2.33(2H, m), 4.08(1H, m), 4.21(2H, d), 4.40(1H, s), 4.41 (2H, s), 4.98(1H, quintet), 6.71(3H, m), 6.79(1H, s), 7.03(1H, s), 7.11-7.15(3H, m), 7.33(2H, m), 7.61(1H, d), 8.61(1H, t), 8.90(1H, t), 10.05(1H, br s).

### Example 90

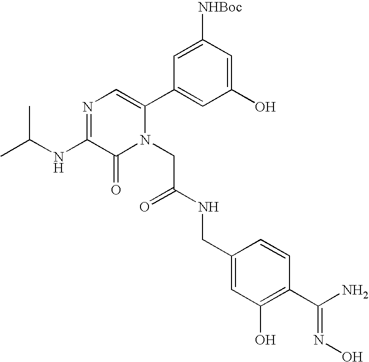

### Example 90

To a 100 mL RBF was added Ex-4c (1.16 g, 2.1 mmol), hydroxylamine hydrochloride (1.06 g, 15.2 mmol) in ethanol (25 mL). To the reaction was added triethylamine (2 mL). The reaction stirred at 80° C. for 4 hours. My M.S. and L.C. the reaction was complete so it was poured onto water (200 mL) and the precipitate was filtered off. The solid was dissolved in ethyl acetate then dried over MgSO4 and concentrated to afford Example 90 (0.4 g) in 32% yield.

M.S. 581.62 (MH + 582.3)

| | | | |
|---|---|---|---|
| Calculated: | C 57.82 | H 6.07 | N 16.86 |
| Found: | C 55.76 | H 6.44 | N 16.45 |

### Example 91

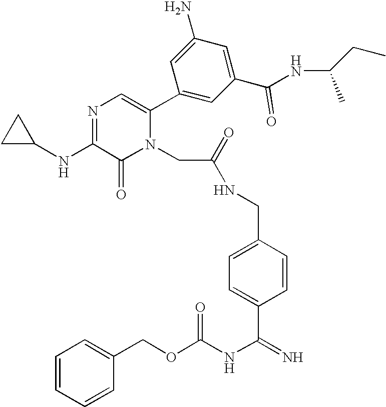

HPLC/LRMS: >97%, 665(M+H)+; HRMS(ES+) calcd. for C₃₆H₄₁N₈O₅ 665.3194, found 665.3220.

### Example 92

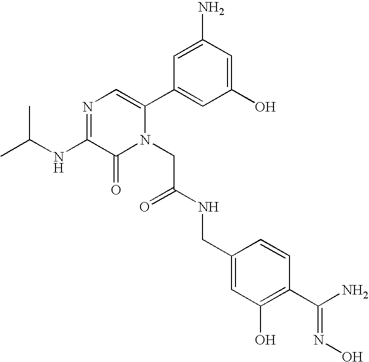

### Example 92

To a 100 mL RBF was added example 90 (0.0200 g, 0.386 mmol) in 20% TFA in methylene chloride (20 mL). The reaction stirred for 1 hour then was concentrated in vacuo. The resulting glassy solid was triturated with diethyl ether then filtered. Upon filtration the solid observed to be deliquescent. The resulting oil was dissolved in water and lyophized to afford Example 92 (0.14 g) in 87% yield.

M.S. 481.21(MH+482.3)

Elemental-Isolated as a salt with 2 TFAs

| | | | |
|---|---|---|---|
| Calculated: | C 51.37 | H 5.65 | N 20. |
| Found: | C 45.90 | H 4.40 | N 13.70 |

### Example 93

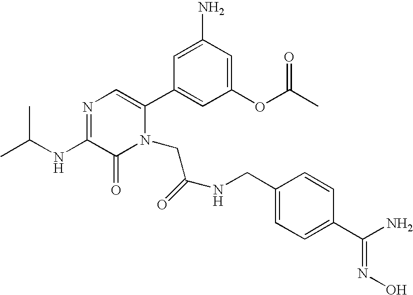

### Example 93a

2.19 g (6 mmol) di-Boc-4-amino-N-hydroxy-benzamidine was deprotected in 25 mL CH₂Cl₂/TFA (4:1) for 30 minutes with stirring and the solvent was thoroughly evaporated to dryness. This solid and 1.5 g (3.2 mmol) of free acid were dissolved in 30 mL DMF. They were coupled in the presence of 1.6 g (5 mmol) TBTU and 3.5 mL (20 mmol) DIPEA for 12 hours. DMF was evaporated and the product was precipitated by addition of 200 mL water and filtered. The crude product was dissolved in the mixture of AcN and H₂O and purified on preparative HPLC using a gradient of acetonitrile (10-50% AcN in 30 minutes), yielding the product EXAMPLE 93a at 42% AcN, 0.82 g (1.35 mmol; 42%) as a white solid. MH⁺=608.3

### Example 93

0.82 g (1.35 mmol) of the boc-protected EXAMPLE 93a was deprotected in 25 mL CH₂Cl₂/TFA (4:1) with stirring for 30 minutes. The solvent was evaporated and the residue was dissolved in AcN/H₂O and purified on preparative HPLC using a gradient of acetonitrile (10-40% AcN in 30 minutes), yielding the title product at 23% AcN, 0.45 g (0.6 mmol; 44%) as a white solid. MH⁺=508.2

¹HNMR: 400 MHz, CD₃OD: 7.68-7.61 (d, 2H), 7.50-7.40 (d, 2H), 6.64 (s, 1H), 6.60-6.56 (d, 2H), 6.45 (s, 1H), 4.60 (s, 2H), 4.46 (s, 2H), 4.10-3.98 (m, 1H), 2,14 (s, 3H) and 1.40-1.32 (m, 6H).

Elemental analysis: C₂₅H₂₉N₇O₅ + 1.8xTFA + 1.8xH₂O

| | | | |
|---|---|---|---|
| Found | C: 46.02 | H: 4.65 | N: 12.59 |
| Calc. | C: 46.10 | H: 5.01 | N: 13.16 |

### Example 94

### Ethyl 3-amino-5-[1-[2-({4-[(Z)-amino(hydroxyimino)methyl]benzyl}amino)-2-oxoethyl]-3-chloro-5-(isopropylamino)-6-oxo-1,6-dihydropyrazin-2-yl]benzoate

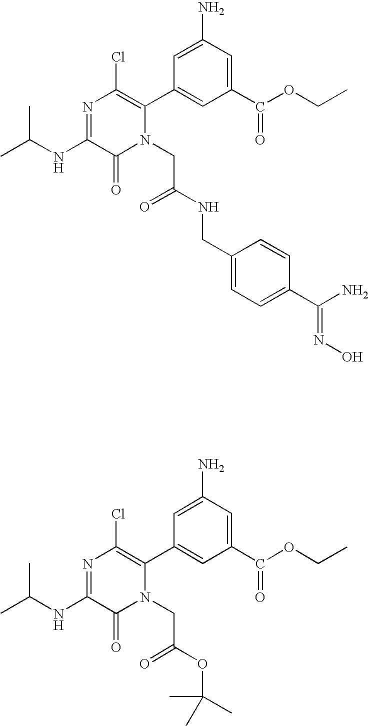

### Ethyl 3-amino-5-[1-(2-tert-butoxy-2-oxoethyl)-3-chloro-5-(isopropylamino)-6-oxo-1,6-dihydropyrazin-2-yl]benzoate

### Example 94a

To a round bottom flask under nitrogen was added THF (300 mL) and H₂O (37 mL). The solvent mixture was degassed for 20 min after which pyrazinone-bromide (10.0 g, 2.62 mmol), 3-(ethoxycarbonyl)-5-nitrophenylboronic acid (Combi-blocks, 7.53 g, 3.15 mmol), Na₂CO₃ (11.0 g, 10.4 mmol) and Pd(PPh₃)₄ (3.00 g, 0.26 mmol) were added. The reaction mixture was heated to reflux for 18 hrs after which 1.5 g of Pd(PPh₃)₄ was added. After heating for 5 hr an additional 1.0 g of Pd(PPh₃)₄ was added. The reaction was heated for 14 hrs and then allowed to cool to room temperature. The reaction mixture was poured into water and ethyl acetate. The layers were separated and the organic layer was washed with brine and dried (Na₂SO₄). The solvent was removed to give a brown foam, which after chromatography (silica, 10-20% ethyl acetate:hexane gave the desired product as a yellow solid (4.80 g, 37%). ¹H NMR (400 MHz, CDCl₃): δ 8.93-8.92 (m, 1 H), 8.38 (t, 1 H), 8.32 (t, 1 H), 6.27 (d, 1 H), 4.42 (q, 2 H), 4.33-4.18 (m, 3 H), 1.41 (t, 3 H), 1.35 (s, 9 H), 1.28 (d, 6 H); MS-ESI (M+H)=495.

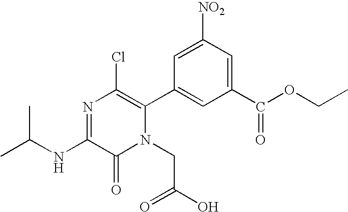

### [5-chloro-6-[3-(ethoxycarbonyl)-5-nitrophenyl]-3-(isopropylamino)-2-oxopyrazin-1(2H)-yl]acetic Acid

### Example 94b

To a solution of Example 94a (4.8 g, 9.7 mmol) in dichloromethane (100 mL) at 0° C. was added TFA (100 mL). After stirring for 4 hrs at room temperature the solvent was removed in vacuo to give a brown solid (5.35 g), which was used without further purification in the next step. MS-ESI (M+H)=439.

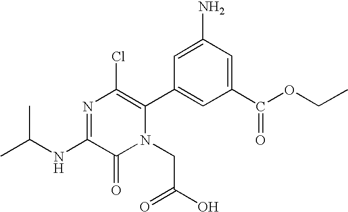

### [5-chloro-6-[3-(ethoxycarbonyl)-5-nitrophenyl]-3-(isopropylamino)-2-oxopyrazin-1(2H)-yl]acetic Acid

### Example 94c

To a solution of Example 94b (5.4 g, 12.0 mmol) in methanol (50 mL) at room temperature was added 10% Pd/C and NH₄CO₂H (2.2 g, 36.0 mmol). The reaction was heated to reflux for 2 hrs and then cooled to room temperature. The mixture was filtered through Celite and the solvent removed to give a yellow solid (6.3 g, 100%). ¹H NMR (400 MHz, CD₃OD): δ 8.48 (s, 1 H), 7.37 (t, 1 H), 7.22 (t, 1 H), 6.85 (t, 1 H), 4.31-4.26 (m, 3 H), 4.16-4.07 (m, 2 H), 3.29 (q, 2 H), 1.34 (t, 3 H), 1.24 (d, 6 H); MS-ESI (M+H)=409.

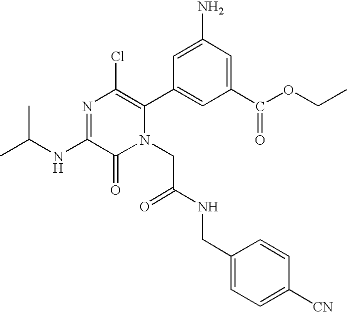

### Ethyl 3-amino-5-[3-chloro-1-{2-[(4-cyanobenzyl)amino]-2-oxoethyl}-5-(isopropylamino)-6-oxo-1,6-dihydropyrazin-2-yl]benzoate

### Example 94d

To a solution of Example 94c (2.45 g, 6.00 mmol) in DMF (30 mL) at 0° C. was added DIEA (4.6 mL, 26.4 mmol), TBTU (2.9 g, 9.0 mmol), and 4-aminobenzonitrile (1.5 g, 9.0 mmol). After stirring overnight at room temperature the reaction mixture was poured into water and the precipitate filtered to give the desired product (3.10, 99%). MS-ESI (M+H)=523.

### Example 94e

To a solution of Example 94d (3.10 g, 6.0 mmol) in ethanol (40 mL) at room temperature was added K₂CO₃ (3.65 g, 26.4 mmol), DIEA (4.6 mL, 26.4 mmol) and H₂NOH*HCl (1.4 g, 19.8 mmol). The reaction was heated to reflux for 3 hrs and then allowed to cool to room temperature. The reaction was filtered and ethanol removed in vacuo to give an oil, which was purified by RP-HPLC (CH₃CN: H₂O) to give the desired product (350 mg). ¹H NMR (400 MHz, CD₃OD): δ 7.62 (d, 2 H), 7.47 (t, 1 H), 7.41 (d, 2 H), 7.27 (t, 1 H), 6.90 (t, 1 H), 4.48-4.37 (m, 4 H), 4.33-4.27 (m, 2H), 4.18-4.15 (m, 1 H), 1.34 (t, 3 H), 1.27-1.25 (m, 6 H); Analysis: C₂₆H₃₀ClN₇O₅+1.9 TFA+0.7 H₂O calcd: C, 45.57; H, 4.27; N, 12.48; O, 19.35; found: C, 45.52; H, 4.32; N, 12.56; O, 19.35.

### Example 95

### Ethyl 3-amino-5-[1-(2-{[4-((E)-amino{[(benzyloxy)carbonyl]imino}methyl)benzyl]amino}-2-oxoethyl)-3-chloro-5-(isopropylamino)-6-oxo-1,6-dihydropyrazin-2-yl]benzoate

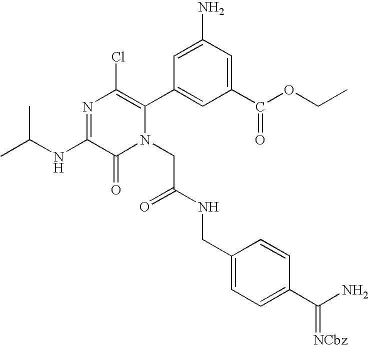

### Example 95

To a solution of Example 94b (2.45 g, 6.0 mmol) in DMF (30 mL) at 0° C. was added DIEA (4.6 mL, 26.4 mmol), TBTU (2.9 g, 9.0 mmol), and 4-amino-Z-benzamidine (2.54 g, 9.0 mmol). After stirring overnight at room temperature the reaction mixture was poured into water and ethyl acetate. The layers were separated and the organic layer washed with sodium bicarbonate, brine and dried (Na₂SO₄). The solvent was removed to give an oil, which was purified by RP-HPLC (CH₃CN:H₂O) to give the desired product (256 mg). ¹H NMR (300 MHz, CD₃OD): δ 7.74 (d, 2 H), 7.51-7.38 (m, 7 H), 7.26 (t, 1 H), 6.88 (t, 1 H), 5.41 (s, 2 H), 4.44 (s, 2 H), 4.42 (s, 2 H), 4.31 (q, 2 H), 4.20-4.15 (m, 1 H), 1.34 (t, 3 H), 1.27 (d, 6 H); MS-ESI (M+H)=674;

Analysis: C₃₄H₃₆ClN₇O₆+1.4 TFA+0.6 H₂O calcd: C, 52.33; H, 4.6; N, 11.6; O, 17.8; found: C, 52.31; H, 4.63; N, 11.71; O, 17.79.

### Example 96

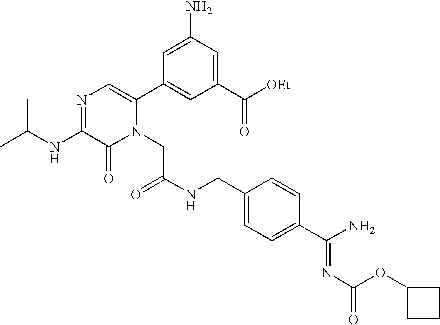

### Example 96

To the free amidine (299 mg, 0.39 mmol) and cyclobutyl-chloroformate (102 mg, 0.43 mmol) in 3 ml of N,N-dimethylformamide was added N-methylmorpholine (0.17 ml, 1.56 mmol). The solution was heated at 60° C. for 20 hours. The solution was then treated with more of the cyclobutyl-chloroformate and heated at 80° C. for 2 hours. The solution was treated with trifluoroacetic acid (0.15 ml, 1.95 mmol) and purified by reverse phase chromatography with 20-50% CH₃CN/H₂O to give 128 mg (41% yield) of a light yellow solid m/z (M+H)⁺604

Analysis: C₃₁H₃₇N₇O₆+1.75 TFA+1.00 H₂O calcd: C, 50.46; H, 5.00; N, 11.94; found: C, 50.47; H, 4.97; N, 11.98 .

HRMS calcd: 604.2878; Found: 604.2911

¹H NMR(400 MHz, DMSO): 1.22(6H, d), 1.29(3H, t), 1.67(1H, m), 1.82 (1H, m), 2.18(2H, m), 2.39(2H, m), 4.01(1H, m), 4.25(2H, q), 4.38(2H, d), 4.40(1H, s), 5.08 (1H, quintet), 6.70(1H, s), 6.79(1H, s), 7.13(1H, s), 7.28(1H, s), 7.38(2H, d), 7.75(1H, d), 8.68(1H, t).

### Example 97

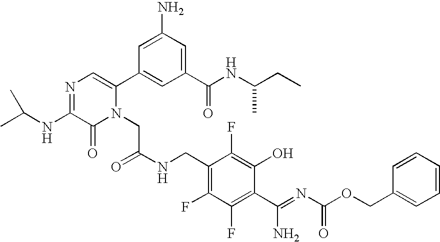

### Example 97

To the free amidine (100 mg, 148 mmol) in THF (5 mL) was added 10% sodium hydroxide (5 mL) at 0° C. Benzyl chloroacetate (0.025 g, 210 μl, 148 mmol) was then added and the mixture was allowed to stir overnight and warm to water bath temperature. LCMS at this time showed a large amount of starting material remaining. The reaction was again cooled to 0° C. and another equivalent (210 μl ) of benzyl chloroacetate was added. After 1 h the reaction was complete. The mixture was transferred to a separatory funnel and the layers were separated. The aqueous layer was extracted with ethyl acetate (3×50 mL) and the combined organic layers were dried (MgSO₄), filtered and concentrated. Purification by flash chromatography (Merck 230-400 mesh SiCO₂, 2% MeOH in Chloroform) afforded Example 97 as a yellow solid. LCMS (RP, 15-90% acetonitrile in 0.1% ammonium acetate over 8 min): retention time =5.82 min; (M+H)⁺=737.

### Example 98

### Benzyl (1E)-amino{4-[({[6-(3-amino-5-{[(2-methoxyethyl)amino]carbonyl}phenyl)-3-(isopropylamino)-2-oxopyrazin-1(2H)-yl]acetyl}amino)methyl]phenyl}methylidenecarbamate

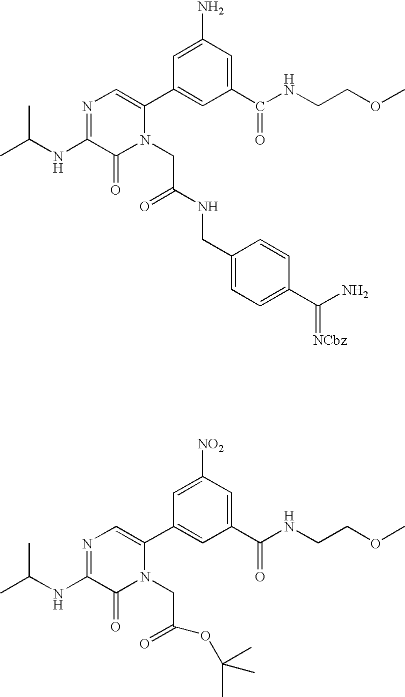

### Tert-butyl [3-(isopropylamino)-6-(3-{[(2-methoxyethyl) amino]carbonyl}-5-nitrophenyl)-2-oxopyrazin-1(2H)-yl]acetate

### Example 98a

To a solution of free acid (0.6 g, 1.0 mmol) in DMF (15 mL) was added DIEA (0.90 mL, 4.4 mmol), TBTU (0.9 g, 1.2 mmol), and 2-methoxyethylamine (0.12 mL, 1.2 mmol). After 2 hrs the reaction mixture was poured into water and ethyl acetate. The layers were separated and the organic layer washed with sodium bicarbonate and dried (Na₂SO₄). The solvent was removed in vacuo to give an oil, which after chromatography (silica, 50-75% ethyl acetate:hexane) gave the desired product (280 mg, 57%). MS-ESI (M+H)=490.

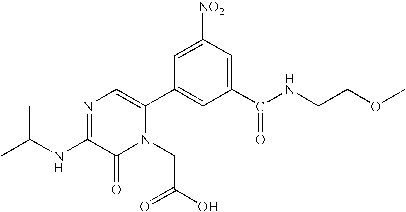

### [3-(isopropylamino)-6-(3-{[(2-methoxyethyl)amino]carbonyl}-5-nitrophenyl)-2-oxopyrazin-1(2H)-yl]acetic Acid

### Example 98b

To a solution of Example 98a (0.28 9, 0.57 mmol) in dichloromethane (10 mL) at room temperature was added TFA (10 mL). The reaction mixture was stirred for 2 hrs and then the solvent was removed in vacuo to give an oil (0.28 g, 99%). MS-ESI (M+H)=434.

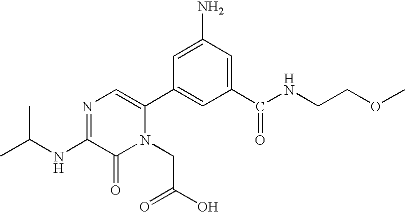

### [3-(isopropylamino)-6-(3-{[(2-methoxyethyl)amino]carbonyl}-5-nitrophenyl)-2-oxopyrazin-1(2H)-yl]acetic Acid

### Example 98c

To a solution of Example 98b (0.24 g, 0.57 mmol) in methanol (10 mL) at room temperature was added 10% Pd/C (0.15 g) and NH₄CO₂H (0.3 g, 4.7 mmol). The reaction was heated to reflux for 2 hrs and then cooled to room temperature. The mixture was filtered through Celite and solvent removed in vacuo to give an oil (0.32 g, 100%). MS-ESI (M+H)=404.

### Example 98d

To a solution of Example 98c (0.4 g, 0.99 mmol) in DMF (5 mL) at 0° C. was added DIEA (1.0 ml, 4.3 mmol), TBTU (0.31 g, 0.99 mmol), and 4-amino-Z-benzamidine (0.28 g, 0.99 mmol). After 1 hr the reaction mixture was purified by RP-HPLC (CH₃CN:H₂O) to give the desired product (94 mg). ¹H NMR (400 MHz, CD₃OD): δ 7.72 (d, 2 H), 7.49-7.37 (m, 7 H), 7.19 (t, 1 H), 7.02 (t, 1 H), 6.83 (t, 1 H), 6.65 (s, 1 H), 5.40 (s, 2H), 4.63 (s, 2 H), 4.45 (d, 2 H), 4.06-4.02 (m, 1 H), 3.48 (s, 3 H), 3.30-3.28 (m, 4 H), 1.35 (d, 6 H); MS-ESI (M+H)=669; Analysis: C₃₅H₄₀N₈O₆+2.0 TFA+1.5 H₂O calcd: C, 50.77; H, 4.66; N, 11.99; found: C, 50.84; H, 4.73; N, 11.88.

### Example 99

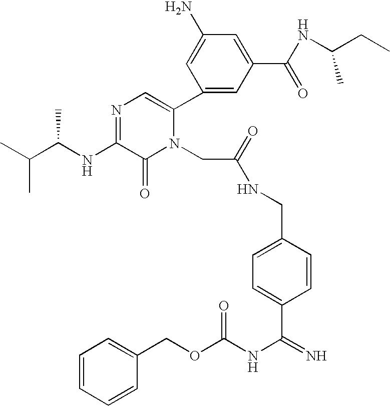

HPLC/LRMS: >97%, 695(M+H)+; HRMS(ES+) calcd. for C₃₈H₄₇N₈O₅ 695.3664, found 695.3688.

### Example 100

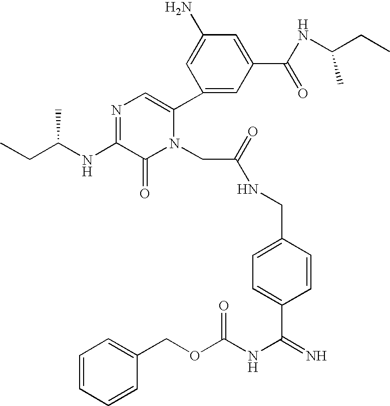

HPLC/LRMS: >95%, 681(M+H)+; HRMS(ES+) calcd. for C₃₇H₄₅N₈O₅ 681.3507, found 681.3505.

### Example 101

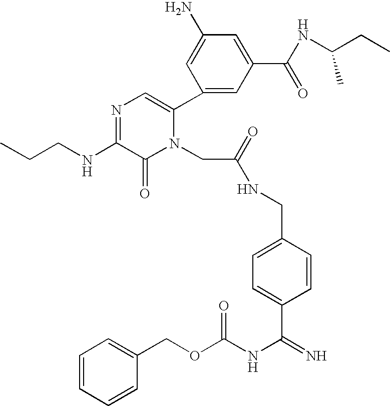

HPLC/LRMS: >96%, 667(M+H)+; HRMS(ES+) calcd. for C₃₆H₄₃N₈O₅ 667.3351, found 667.3331.

### Example 102

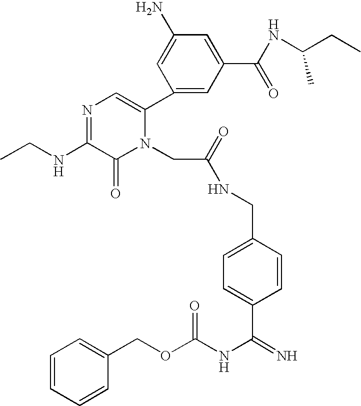

HPLC/LRMS: >98%, 653(M+H)+; HRMS(ES+) calcd. for C₃₅H₄₁N₈O₅ 653.3194, found 653.3216.

### Example 104

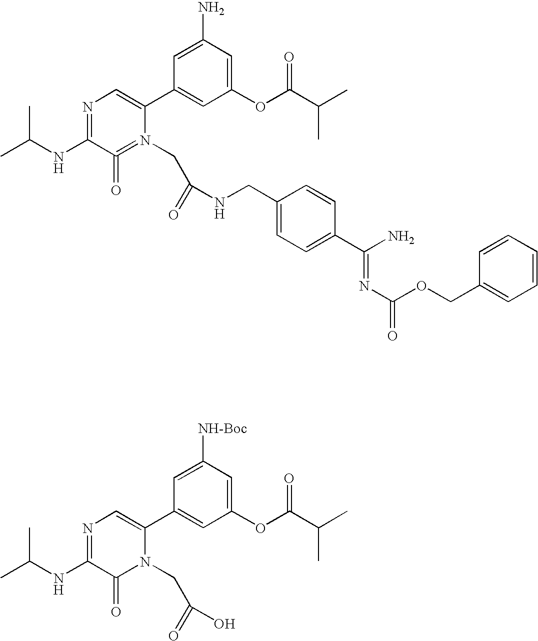

### Example 104a

2.1 g (5 mmol) of Boc-protected phenol was reacted with 1.06 g (10 mmol; 1.05 mL) isobutyryl chloride in 50 mL CH₂Cl₂ in the presence of 2.625 mL (15 mmol) DIPEA with stirring for 30 minutes. The solvent was evaporated and the residue was dissolved in AcN, diluted with H₂O and purified on prep HPLC using a gradient of acetonitrile (20-55% AcN in 30 minutes), yielding the title product at 55% AcN, 1.17 g (2.4 mmol; 48%) as a white solid.

MH⁺=489.2

### Example 104b

1.44 g (3 mmol) di-Boc-4-amino-Z-benzamidine was deprotected in 25 mL CH₂Cl₂/TFA (4:1) for 30 minutes stirring and the solvent was thoroughly evaporated to dryness. This solid and 1.17 g (2.4 mmol) of EXAMPLE 104a were dissolved in 25 mL DMF. They were coupled in the presence of 0.8 g (2.5 mmol) TBTU and 1.75 mL (10 mmol) DIPEA for 12 hour. DMF was evaporated and the product was precipitated by addition of 200 mL water and filtered. The crude product was dissolved in the mixture of AcN and H₂O and purified on preparative HPLC using a gradient of acetonitrile (10-60% AcN in 30 minutes), yielding the product at 55% AcN, 0.38 g (0.5 mmol; 21%) as a white solid.

MH⁺=754.3

### Example 104c

0.38 g (0.5 mmol) of EXAMPLE 104b was deprotected in 25 mL CH₂Cl₂/TFA (4:1) with stirring for 30 minutes. The solvent was evaporated and the residue was dissolved in AcN/H₂O and lyophilized to yield 0.31 g (0.35 mmol; 70%) white solid.

MH⁺=654.2

¹HNMR: 400 MHz, CD₃OD: 7.78-7.70 (d, 2H), 7.52-7.45 (m, 4H), 7.44-7.36 (m, 3H), 6.64 (s, 1H), 6.60-6.52 (d, 2H), 6.40 (s, 1H), 5.40 (s, 2H), 4.60 (s, 2H), 4.50-4.44 (m, 2H), 4.10-3.98 (m, 1H), 2.82-2.72 (m, 1H), 1.40-1.32 (m, 6H) and 1.30-1.22 (m, 6H).

Elemental analysis: C₃₅H₃₉N₇O₆ + 2.5xTFA + 2.1xH₂O

| | | | |
|---|---|---|---|
| Found | C: 53.43 | H: 5.48 | N: 11.48 |
| Calc. | C: 53.32 | H: 5.39 | N: 11.58 |

### Example 105

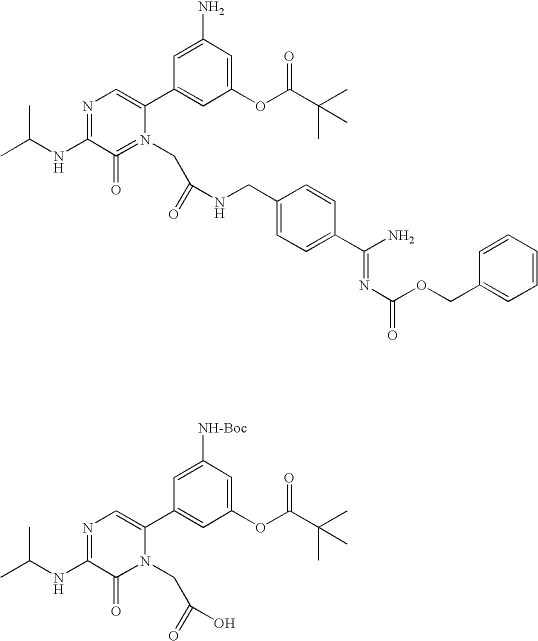

### Example 105a

2.1 g (5 mmol) of Boc-protected phenol was reacted with 1.2 g (10 mmol; 1.23 mL) pivaloyl chloride in 40 mL CH₂Cl₂ in the presence of 3.5 mL (20 mmol) DIPEA with stirring for 30 minutes. The solvent was evaporated and the residue was dissolved in AcN, diluted with H₂O and purified on prep HPLC using a gradient of acetonitrile (20-55% AcN in 30 minutes), yielding the title product at 55% AcN, 1.17 g (2.4 mmol; 48%) as an oil. MH⁺=503.2

### Example 105b

1.44 g (3 mmol) di-Boc-4-amino-Z-benzamidine was deprotected in 25 mL CH₂Cl₂/TFA (4:1) for 30 minutes stirring and the solvent was thoroughly evaporated to dryness. This solid and 1 g (2 mmol) of EXAMPLE 105a were dissolved in 25 mL DMF. They were coupled in the presence of 0.8 g (2.5 mmol) TBTU and 1.75 mL (10 mmol) DIPEA for 12 hour. DMF was evaporated and the product was precipitated by addition of 200 mL water and filtered. The crude product was dissolved in the mixture of AcN and H₂O and purified on preparative HPLC using a gradient of acetonitrile (10-60% AcN in 30 minutes), yielding the product at 58% AcN, 0.628 g (0.82 mmol; 41%) as a white solid. MH⁺=768.2

### Example 105c

0.628 g (0.82 mmol) of EXAMPLE 105b was deprotected in 25 mL CH₂Cl₂/TFA (4:1) with stirring for 30 minutes. The solvent was evaporated and the residue was dissolved in AcN/H₂O and lyophilized to yield 0.67 g (0.75 mmol; 91%) white solid.

MH⁺=668.2

¹HNMR: 400 MHz, CD₃OD: 7.80-7.76 (d, 2H), 7.52-7.42 (m, 4H), 7.42-7.36 (m, 3H), 6.64 (s, 1H), 6.60-6.52 (d, 2H), 6.38 (s, 1H), 5.40 (s, 2H), 4.62 (s, 2H), 4.50-4.44 (m, 2H), 4.10-3.98 (m, 1H) and 1.40-1.26 (m, 15H).

Elemental analysis: C₃₆H₄₁N₇O₆ + 1.3xTFA + 2.1xH₂O

| | | | |
|---|---|---|---|
| Found | C: 54.32 | H: 5.42 | N: 11.37 |
| Calc. | C: 54.30 | H: 5.49 | N: 11.48 |

### Example 106

### 2-(methylsulfonyl)ethyl {4-[({[6-(3-amino-5-hydroxyphenyl)-3-(isopropylamino)-2-oxopyrazin-1(2H)-yl]acetyl}amino)methyl]phenyl}(imino)methylcarbamate

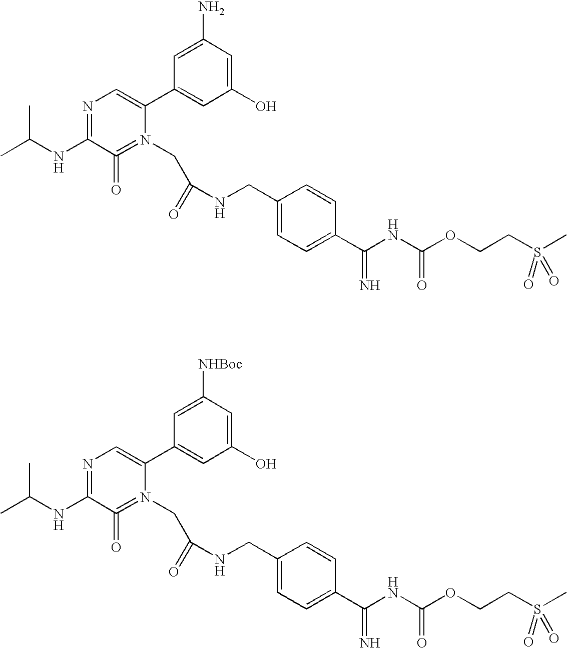

### 2-(methylsulfonyl)ethyl {4-[({[6-(3-[(tert-butoxycarbonyl) amino]-5-hydroxyphenyl}-3-(isopropylamino)-2-oxopyrazin-1(2H)-yl]acetyl}amino)methyl]phenyl}(imino)methylcarbamate

### Example 106a

To a 50 mL RBF was added the free amidine (0.514 g, 0.935 mmol) and 2-(methylsulfonyl)ethyl 4-nitrophenyl carbonate (0.289 g, 1.0 mmol) in NMM (2 mL) and methylene chloride (15 mL). The reaction stirred overnight. The reaction was poured into water (50 mL) and methylene chloride (50 mL). The organics were collected and washed with brine (2×50 mL). The organics were dried over MgSO4 and concentrated in vacuo to afford Example 106a (0.295 g) in 45% yield.

MS 699.78 (MH+700.3)

NMR (400 MHz, CDCl₃): ¹H 1.26 ppm (6H, d), 1.49 ppm (9H, s), 3.10 ppm (3H, s), 3.51 ppm(2H, t), 4.1 ppm (1H, q), 4.43 ppm (2H,s), 4.58 ppm (3H,t), 4.6 ppm (2H, bs), 6.42 ppm (1H, s), 6.72 ppm (1H, s), 6.85 ppm (1H, s), 7.09 ppm (1H, bs), 7.31 ppm (2H, d), 7.80 ppm (2H, d).

### Example 106b

To a 50 mL RBF was added Example 106a (0.295 g, 0.422 mmol) in a 20% solution of TFA and methylene chloride. The reaction stirred for 1 hour then was concentrated in vacuo. The resulting solid was dissolved in water (100 mL) and purified by reverse phase chromatography to afford (0.227 g) in 89% yield as the TFA salt.

MS 599.22(MH+600.3)

NMR (400 MHz, CDCl₃): ¹H 1.26 ppm (6H, d), 3.10 ppm (3H, s), 3.51 ppm(2H, t), 4.1 ppm (1H, q), 4.43 ppm (2H,s), 4.58 ppm (3H,t), 4.6 ppm (2H, bs), 6.42 ppm (1H, s), 6.72 ppm (1H, s), 6.85 ppm (1H, s), 7.09 ppm (1H, bs), 7.31 ppm (2H, d), 7.80 ppm (2H, d).

Isolated as 2.6TFA and1 H2O

| | | | |
|---|---|---|---|
| Found | C: 54.08 | H: 5.55 | N: 16.35 |
| Calc. | C: 42.30 | H: 4.16 | N: 10.18 |

### Example 107

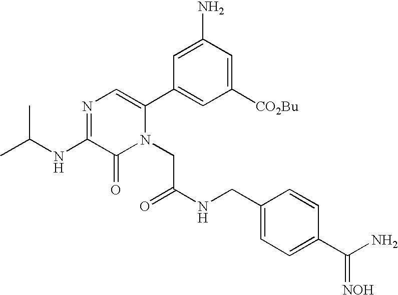

### Example 107

To free amidine (680 mg, 1.28 mmol) and hydroxylamine hydrochloride (266 mg, 3.83 mmol) in 12 ml of ethanol was added N,N-diisopropylethylamine (1.11 ml, 6.38 mmol). The solution was heated at 70° C. for 3 hours. The solution was concentrated, dissolved in CH₃CN/H₂O, and acidified with trifluoroacetic acid. The solution was purified by reverse phase chromatography with 5-45% CH₃CN/H₂O to give 410 mg (40% yield) of a white solid

m/z (M+H) ⁺550

Analysis: C₂₈H₃₅N₇O₅+2.15 TFA+0.55 H₂O calcd: C, 48.21; H, 4.79; N, 12.18; found: C, 48.22; H, 4.77; N, 12.20.

HRMS calcd: 550.2772; Found: 550.2761

¹H NMR(400 MHz, DMSO): 0.91(3H, t), 1.22(6H, d), 1.40(2H, sextet), 1.64(2H, quintet), 4.09(1H, m), 4.21(2H, t), 4.35(2H, d), 4.38(2H, s), 6.70(1H, s), 6.77(1H, t), 7.12(1H, t), 7.27(1H, t), 7.38(2H, d), 7.63(2H, d), 8.67(1H, t).

### Example 109

### Ethyl 3-amino-5-[1-(2-{[4-((E)-amino{[(benzyloxy)carbonyl]imino}methyl)benzyl]amino}-2-oxoethyl)-5-(cyclopropylamino)-6-oxo-1,6-dihydropyrazin-2-yl]benzoate

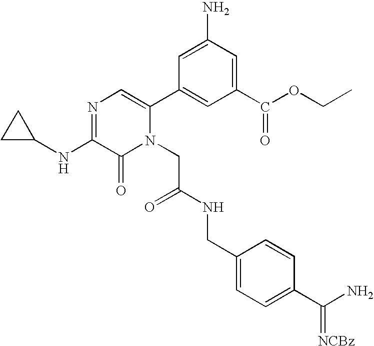

### Example 109

### To a solution of free acid (0.50 g, 0.13 mmol) in DMF (15 mL) at 0° C. was added DIEA (1.0 mL, 0.57 mmol) and TBTU (0.62 g, 0.19 mmol). After 15 min, 4-aminomethyl-Z-benzamidine (0.62 g, 0.19 mmol) was added and the reaction stirred for 1 h. The mixture was poured into water and ethyl acetate and the layers separated. The organic layer was washed with brine and dried (Na₂SO₄). The solvent was removed in vacuo to give a solid, which was purified by RP-HPLC (CH₃CN:H₂O) to give after lyophilization the desired product (390 mg). ¹H NMR (400 MHz, CD₃OD): d 7.73 (d, 2 H), 7.49-7.37 (m, 7 H), 7.29 (t, 1 H), 6.89 (t, 1 H), 6.68 (s, 1 H), 5.39 (s, 2 H), 4.58 (s, 2 H), 4.46 (d, 2 H), 4.30 (q, 2 H), 2.80-2.76 (m, 1 H), 1.32 (t, 3 H), 1.07-1.04 (m, 2 H), 0.87-0.84 (m, 2 H); MS-ESI (M+H)=638;

Analysis: C₃₄H₃₅N₇O₆+2.6 TFA+1.5 H₂O calcd: C, 48.98; H, 4.25; N, 10.2; found: C, 48.99; H, 4.24; N, 10.18.

### Example 110

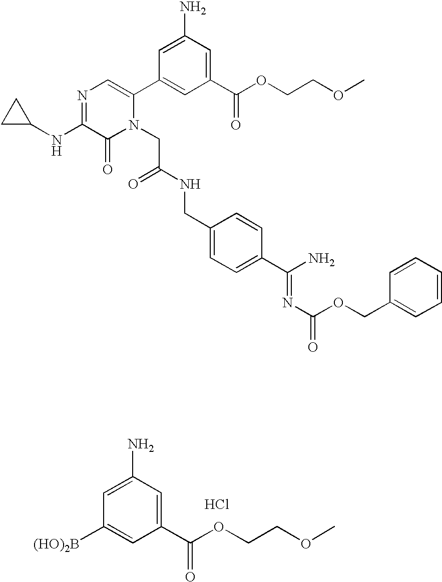

### Example 110a

Into a solution of (3-amino-5-carboxylphenyl) boronic acid (1.0 g, 5.5 mmol) in 2-methoxyethanol (15 ml) was bubbled hydrogen chloride gas for 5 minutes. The reaction was sealed and heated at 85° C. for two hours. The reaction was concentrated in vacuo and the residue crystallized from methanol/diethyl ether to give 1.22 g of Example 110a as an off-white solid. ¹HNMR (300 MHz, DMSO-d₆) δ 3.30 (s, 3H), 3.62-3.68 (m, 2H), 4.36-4.42 (m, 2H), 7.56 (s, 1H), 7.69 (s, 1H), 7.88 (s, 1H). LCMS (ES+) m/z 240.

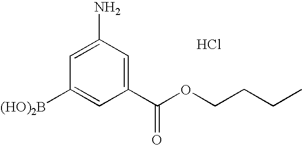

### Example 110b

Into a solution of (3-amino-5-carboxylphenyl) boronic acid (1.0 g, 5.5 mmol) in n-butanol (15 ml) was bubbled hydrogen chloride gas for 5 minutes. The reaction was sealed and heated at 85° C. for two hours. The reaction was diluted with diethyl ether and the resulting crystals collected by vacuum filtration to give 1.2 g of a colorless solid (ca. 40 mole % n-butanol). LCMS (ES+) m/z 238.

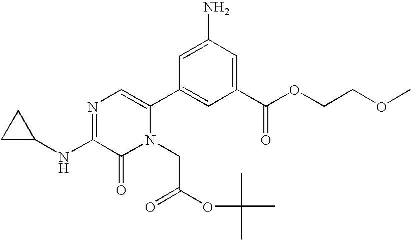

### Example 110c

Into a stirred, nitrogen purged vessel were placed pyrazinone-bromide (0.58 g, 1.7 mmol), Example 110a (0.7 g, 2.5 mmol), cesium carbonate (1.65 g, 5.1 mmol), tetrakis triphenylphosphine palladium (0) (0.39 g, 0.34 mmol), acetonitrile (20 ml), and water (2 ml). The mixture was heated at 75° C. for 18 hours and 90° C. for 2 hours. The organic portion of the reaction was filtered and the filtrate concentrated in vacuo. The residue was mixed with ethyl acetate, washed with water, brine, dried over magnesium sulfate, filtered, and concentrated in vacuo. Purification by silica gel chromatography (33-50% ethyl acetate/hexane) gave 0.51 g (66% yield) of Example 110c as a tan solid. LCMS (ES+) m/z 459.

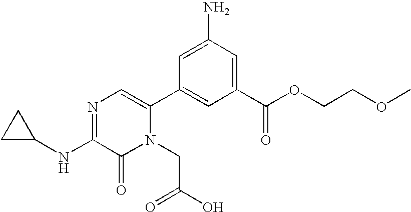

### Example 110d

LCMS (ES+) m/z 403.

### Example 110e

To a stirred solution of Example 110d (200 mg, 0.46 mmol), 4-aminometyl-Z-benzamidine (204 mg, 0.64 mmol), and N-methylmorpholine (0.3 ml, 2.73 mmol) in N,N-dimethylacetamide (4 ml) cooled in an ice bath was added TBTU (161 mg, 0.5 mmol). Stirring was continued at ambient temperature for 1.5 hours. Purification by reverse phase HPLC (10-55% acetonitrile/water) followed by lyophilization yielded 169 mg (38% yield) of Example 110e as an off-white solid. ¹HNMR (300 MHz, DMSO-d₆) δ 0.62-0.79 (m, 4H), 2.70-2.80 (m, 1H), 3.58-3.63 (m, 2H), 4.40-4.50 (m, 6H), 5.33 (s, 2H), 6.74 (s, 1H), 6.77 (s, 1H), 7.10 (s, 1H), 7.27 (s, 1H), 7.39 (d, J=8.4 Hz, 2H), 7.39-7.50 (m, 5H), 7.73 (d, J=8.4 Hz, 2H), 8.67 (t, J=6.0 Hz, 1H), 10.41 (br s, 1H).

HRMS (ES) calcd for C₃₅H₃₈N₇O₇ (M+H): 668.2827. Found: 668.2805. Anal. Calcd for C₃₅H₃₇N₇O₇+2.45TFA+0.75 H₂O: C, 49.88; H, 4.29; N, 10.20. Found: C, 49.90; H, 4.32; N, 10.18.

### Example 111

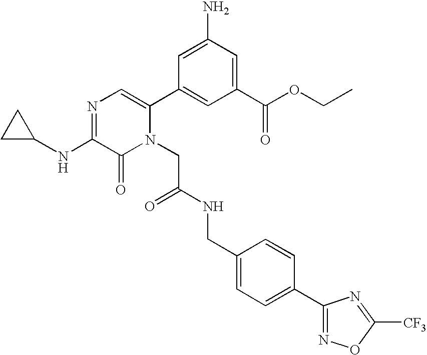

### Example 111

To a stirred solution of free acid (167 mg, 0.41 mmol), amono-oxadiazol(160 mg, 0.57 mmol), and N-methylmorpholine (0.19 ml, 2.04 mmol) in N,N-dimethylacetamide (4 ml) cooled in an ice bath was added TBTU (144 mg, 0.45 mmol). Stirring was continued at ambient temperature for 1.5 hours. Purification by reverse phase HPLC (15-70% acetonitrile/water) followed by lyophilization yielded 134 mg (44% yield) of Example 111 as a pale yellow solid. ¹HNMR (300 MHz, DMSO-d₆) δ 0.68-0.82 (m, 4H), 1.26 (t, J=7.1 Hz, 3H), 2.70-2.82 (m, 1H), 4.25 (q, J=7.2 Hz, 2H), 4.36 (d, J=5.7 Hz, 2H), 4.40 (s, 2H), 6.75 (s, 1H), 6.79 (s, 1H), 7.12 (s, 1H), 7.30 (s, 1H), 7.37 (d, J=8.1 Hz, 2H), 7.98 (d, J=8.4 Hz, 2H), 8.63 (t, J=5.7 Hz, 1H). ¹⁹FNMR (282 MHz, DMSO-d₆) δ −65.11 (s). HRMS (ES) calcd for C₂₈H₂₇N₇O₅F₃ (M+H): 598.2020. Found: 598.1978. Anal. Calcd for C₂₈H₂₆N₇O₅F₃+1.2 TFA+0.1 H₂O: C, 49.59; H, 3,75; N, 13.31. Found: C, 49.65; H, 3.86; N, 13.18.

### Example 112

### Benzyl 3-amino-5-[1-[2-({4-[(E)-amino(hydroxyimino)methyl]benzyl}amino)-2-oxoethyl]-5-(cyclopropylamino)-6-oxo-1,6-dihydropyrazin-2-yl]benzoate

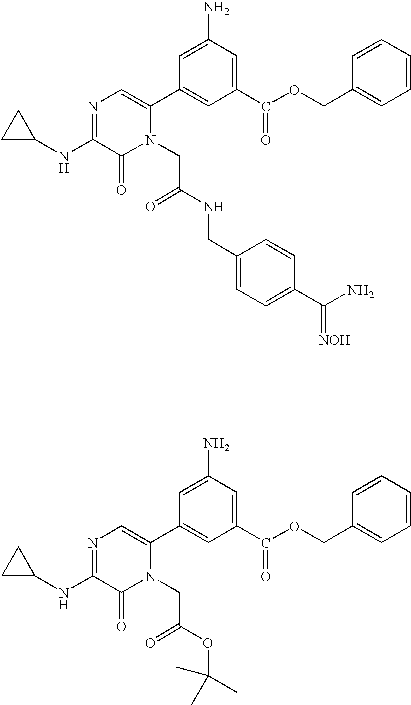

### Benzyl 3-amino-5-[1-(2-tert-butoxy-2-oxoethyl)-5-(cyclopropylamino)-6-oxo-1,6-dihydropyrazin-2-yl]benzoate

### Example 112a

To a solution of pyrazinone-bromide (0.58 g, 0.17 mmol) and 3-amino-5-[(benzyloxy)carbonyl]phenylboronic acid (0.68 g, 0.25 mmol) in CH₃CN: H₂O (30 mL:3 mL) was added Cs₂CO₃ (2.19 g, 0.67 mmol) and Pd(PPh₃)₄ (0.58 g, 0.05 mmol). The reaction mixture was heated to 80° C. for 5 hrs and then allowed to cool to room temperature. The mixture was poured into water and ethyl acetate. The layers were separated and the organic layer washed with sodium bicarbonate and brine. The organic extract was dried (Na₂SO₄) and the solvent removed to give a brown solid, which after chromatography (silica, 60-100% ethyl acetate:hexane) gave the product as a brown solid (0.64 g, 78%). ¹H NMR (300 MHz, CDCl₃): d 7.42-7.37 (m, 5 H), 6.88 (s, 1 H), 6.81 (bs, 1 H), 6.26 (bs, 1 H), 5.34 (s, 2 H), 4.38 (s, 2 H), 3.89 (s, 2 H), 2.82-2.79 (m, 1 H), 1.40 (s, 9 H), 0.89-0.85 (m, 2 H), 0.63-0.61 (m, 2 H); MS-ESI (M+H)=491.

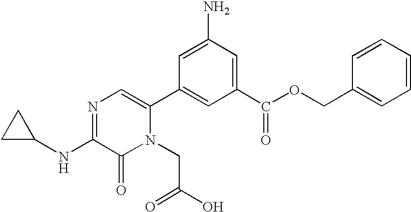

### [6-{3-amino-5-[(benzyloxy)carbonyl]phenyl}-3-(cyclopropylamino)-2-oxopyrazin-1(2H)-yl]acetic Acid

### Example 112b

To a round bottom flask containing Example 112a (0.64 g, 0.10 mmol) was added 4N HCl in dioxane (15 mL) at room temperature. The reaction mixture was heated to 50° C. for 2 hrs and then allowed to cool to room temperature. The precipitate was filtered and the solid collected (0.43 g, 100%). MS-ESI (M+H)=435.

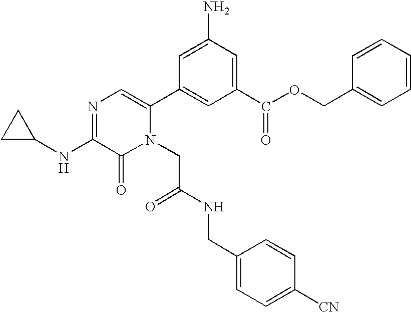

### Benzyl 3-amino-5-[1-{2-[(4-cyanobenzyl)amino]-2-oxoethyl}-5-(cyclopropylamino)-6-oxo-1,6-dihydropyrazin-2-yl]benzoate

### Example 112c

To a solution of Example 112b (0.43 g, 0.10 mmol) in DMF (10 mL) at 0° C. was added DIEA (0.77 mL, 0.44 mmol) and TBTU (0.48 g, 0.15 mmol). After 15 min, 4-aminomethyl-benzonitrile (0.25 g, 0.15 mmol) was added and the reaction stirred for 1 hr. The mixture was poured into water and ethyl acetate. The layers were separated and the organic layer washed with brine and dried (Na₂SO₄). The solvent was removed to give a yellow oil (0.55 g, 100%). MS-ESI (M+H)=549.

### Example 112d

To a solution of Example 112c (0.55 g, 0.10 mmol) in ethanol (20 mL) at room temperature was added K₂CO₃ (0.57 g, 0.44 mmol), DIEA (0.82 mL, 0.44 mmol) and H₂NOH*HCl (0.15 g, 0.22 mmol). The reaction was heated to reflux for 4 hrs and then cooled to room temperature. The mixture was filtered and the solid collected. The solid was purified by RP-HPLC (CH₃CN: H₂O) to give the desired product (260 mg). ¹H NMR (400 MHz, CD₃OD): 7.59 (d, 2 H), 7.49-7.32 (m, 7 H), 6.91 (t, 1 H), 6.68 (s, 1 H), 5.30 (s, 2 H), 4.55 (s, 2 H), 4.36 (d, 2 H), 2.80-2.76 (m, 1 H), 1.08 (m, 2 H), 0.87-0.83 (m, 2 H); MS-ESI (M+H)=582; Analysis: C₃₁H₃₁N₇O₅+2.8 TFA+0.8 H₂O calcd: C, 48.02; H, 3.89; N, 10.71; found: C, 47.99; H, 3.85; N, 10.78.

### Example 113

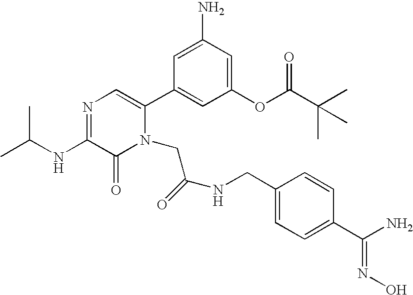

### Example 113a

1.1 g (3 mmol) di-Boc-4-amino-N-hydroxy-benzamidine was deprotected in 25 mL CH₂Cl₂/TFA (4:1) for 30 minutes with stirring and the solvent was thoroughly evaporated to dryness. This solid and 1 g (2 mmol) of the free acid were dissolved in 25 mL DMF. They were coupled in the presence of 0.96 g (3 mmol) TBTU and 1.75 mL (10 mmol) DIPEA for 12 hours. DMF was evaporated and the product was precipitated by addition of 200 mL water and filtered. The crude product was dissolved in the mixture of AcN and H₂O and purified on preparative HPLC using a gradient of acetonitrile (10-50% AcN in 30 minutes), yielding the product at 48% AcN, 0.41 g (0.63 mmol; 63%) as a white solid. MH⁺=650.4

### Example 113b

0.41 g (0.63 mmol) of the boc-protected Example 113a was deprotected in 5 mL CH₂Cl₂/TFA (4:1) with stirring for 30 minutes. The solvent was evaporated and the residue was dissolved in AcN/H₂O and purified on preparative HPLC using a gradient of acetonitrile (10-40% AcN in 30 minutes), yielding the title product at 28% AcN, 0.28 g (0.51 mmol; 81%) as a white solid. MH+=550.4

¹HNMR: 400 MHz, CD₃OD: 7.68-7.60 (d, 2H), 7.50-7.42 (d, 2H), 6.65 (s, 1H), 6.60-6.48 (d, 2H), 6.38 (s, 1H), 4.60 (s, 2H), 4.50-4.42 (m, 2H), 4.10-3.98 (m, 1H) and 1.40-1.22 (m, 15H).

Elemental analysis: C₂₈H₃₅N₇O₅ + 1.8xTFA + 1.8xH₂O

| | | | |
|---|---|---|---|
| Found | C: 47.61 | H: 4.89 | N: 12.18 |
| Calc. | C: 47.50 | H: 4.94 | N: 12.04 |

### Example 114

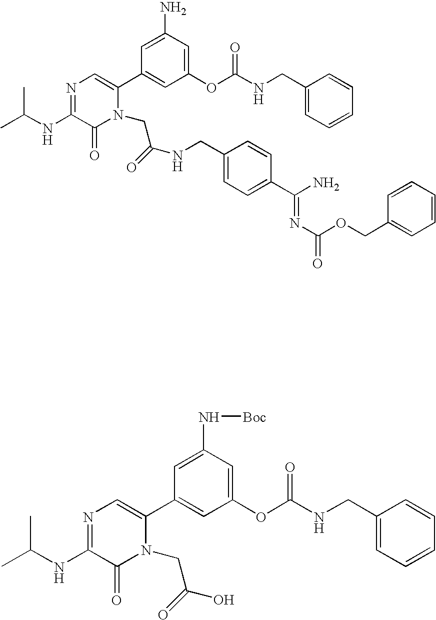

### Example 114a

0.83 g (2 mmol) of Boc-protected phenol was reacted with 0.66 g (5 mmol; 0.9 mL) benzylisocyanate in 25 mL CH₂Cl₂ in the presence of 1.75 mL (10 mmol) DIPEA with stirring for 1 hour. The solvent was evaporated and the residue was dissolved in AcN, diluted with H₂O and purified on prep HPLC using a gradient of acetonitrile (20-55% AcN in 30 minutes), yielding the title product at 48% AcN, 0.609 (1.1 mmol; 55%) as a solid. MH⁺=552.3

### Example 114b

1.06 g (2.2 mmol) di-Boc-4-amino-Z-benzamidine was deprotected in 25 mL CH₂Cl₂/TFA (4:1) for 30 minutes with stirring and the solvent was thoroughly evaporated to dryness. This solid and 0.6 g (1.1 mmol) of Example 114a were dissolved in 25 mL DMF. They were coupled in the presence of 0.64 g (2 mmol) TBTU and 1.75 mL (10 mmol) DIPEA for 12 hour. DMF was evaporated and the product was precipitated by addition of 200 mL water, filtered and purified on prep HPLC using a gradient of acetonitrile (20-60% AcN in 30 minutes), to yield 0.42 g (0.51 mmol; 47%) as a white solid. MH⁺=817.2

### Example 114c

0.42 g (0.51 mmol) of Example 114b was deprotected in 25 mL CH₂Cl₂/TFA (4:1) with stirring for 30 minutes. The solvent was evaporated and the residue was dissolved in AcN/H₂O and purified on prep HPLC using a gradient of acetonitrile (10-50% AcN in 30 minutes), yielding the title product at 35% AcN, 0.255 g (0.35 mmol; 68%) as a white solid. MH⁺=717.2

¹HNMR: 400 MHz, CD₃OD: 7.78-7.68 (d, 2H), 7.50-7.16 (m, 12H), 6.65 (s, 1H), 6.60-6.50 (d, 2H), 6.46 (s, 1H), 5.38 (s, 2H), 4.64 (s, 2H), 4.42 (s, 2H), 4.34 (s, 2H), 4.10-3.96 (m, 1H) and 1.40-1.30 (m, 6H).

Elemental analysis: C₃₉H₄₀N₈O₆ + 2.1xTFA + 0.5xH₂O

| | | | |
|---|---|---|---|
| Found | C: 53.62 | H: 4.59 | N: 11.85 |
| Calc. | C: 53.76 | H: 4.50 | N: 11.61 |

### Example 115

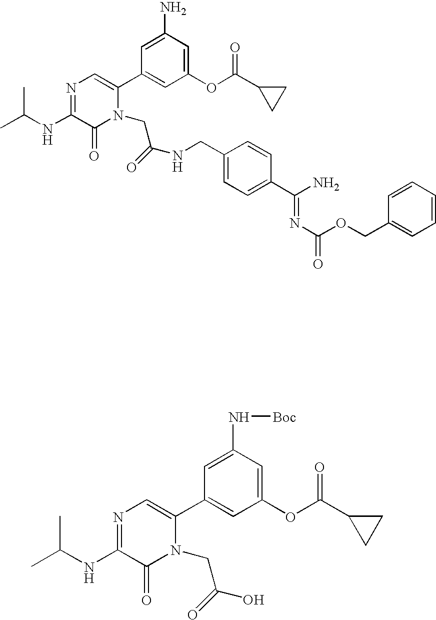

### Example 115a

0.42 g (1 mmol) of Boc-protected phenol was reacted with 0.36 g (3 mmol; 0.32 mL) cyclopropanecarbonyl chloride in 40 mL CH₂Cl₂ in the presence of 0.875 mL (5 mmol) DIPEA with stirring for 30 minutes. The solvent was evaporated and the residue was dissolved in AcN, diluted with H₂O and purified on prep HPLC using a gradient of acetonitrile (20-55% AcN in 30 minutes), yielding the product at 50% AcN, 0.34 g (0.7 mmol; 47%) as a solid. MH⁺=487.2

### Example 115b

1.06 g (2.2 mmol) di-Boc-4-amino-Z-benzamidine was deprotected in 25 mL CH₂Cl₂/TFA (4:1) for 30 minutes stirring and the solvent was thoroughly evaporated to dryness. This solid and 0.34 g (0.7 mmol) of Example 115a were dissolved in 25 mL DMF. They were coupled in the presence of 0.8 g (2.5 mmol) TBTU and 1.75 mL (10 mmol) DIPEA for 12 hour. DMF was evaporated and the product was precipitated by addition of 200 mL water, filtered and dried to yield 0.4 g (0.53 mmol; 76%) as a white solid. MH⁺=752.2

### Example 115c

0.4 g (0.53 mmol) of Example 115b was deprotected in 25 mL CH₂Cl₂/TFA (4:1) with stirring for 30 minutes. The solvent was evaporated and the residue was dissolved in AcN/H₂O and purified on prep HPLC using a gradient of acetonitrile (10-50% AcN in 30 minutes), yielding the title product at 38% AcN, 0.395 g (0.45 mmol; 64%) as a white solid. MH⁺=652.2

¹HNMR: 400 MHz, CD₃OD: 7.78-7.72 (d, 2H), 7.52-7.46 (d, 2H), 7.45-7.25 (m, 5H), 6.64 (s, 1H), 6.60-6.50 (d, 2H), 6.42 (s, 1H), 5.40 (s, 2H), 4.60 (s, 2H), 4.50-4.46 (m, 2H), 4.10-3.97 (m, 1H), 1.90-1.78 (m, 1H), 1.40-1.30 (m, 6H) and 1.10-1.01 (m, 4H).

Elemental analysis: C₃₅H₃₇N₇O₆ + 1.6xTFA + 1.5xH₂O

| | | | |
|---|---|---|---|
| Found | C: 52.84 | H: 5.01 | N: 11.20 |
| Calc. | C: 52.62 | H: 4.95 | N: 11.24 |

### Example 116

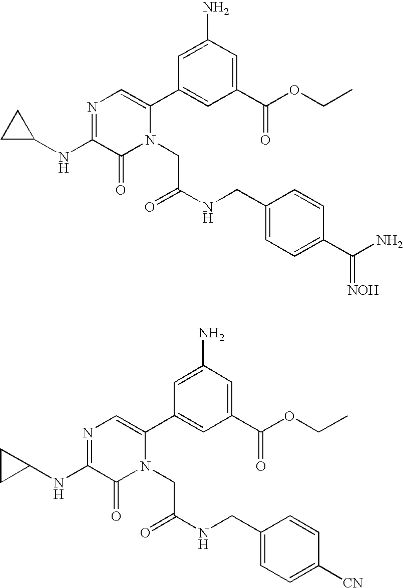

### Example 116a

To the free acid (0.51 g, 1.25 mmol) and 4-aminobenzonitrile (252 mg, 1.5 mmol) in 5 ml of N,N-dimethylformamide was added N,N-diisopropylethylamine (0.87 ml, 5.0 mmol) and then benzotriazol-1-yl tetramethyluronium tetrafluoroborate (482 mg, 1.5 mmol). The solution was stirred for 30 minutes. The solution was added to a stirring solution of 100 ml of water. The resulting precipitate was collected by vacuum filtration and dried over phosphorous pentoxide under high vacuum to give 476 mg (78% yield) of a yellow solid. m/z(M+H)⁺487

### Example 116b

To the product of Example 116a (452 mg, 0.93 mmol), hydroxyl amine hydrochloride (266 mg, 2.33 mmol) and potassium carbonate (0.64 g, 4.65 mmol) in 10 ml of ethanol was added N,N-diisopropylethylamine (0.81 ml, 4.65 mmol). The solution was heated at 70° C. for 2 hours. Another portion of hydroxyl amine hydrochloride (266 mg, 2.33 mmol) and N,N-diisopropylethylamine (0.81 ml, 4.65 mmol) was added and the solution was heated at 70° C. for 6 hours. Acetonitrile was added and the mixture was decanted. The organic layer was concentrated and the residue was treated with acetonitrile. The mixture was acidified with trifluoroacetic acid and purified by reverse phase chromatography with 5-40% CH₃CN/H₂O to give 390 mg (50% yield) of a white solid m/z(M+H)³⁰ 520

Analysis: C₂₆H₂₉N₇O₅+2.60 TFA+1.10 H₂O calcd: C, 44.83; H, 4.08; N, 11.73; found: C, 44.79; H, 4.03; N, 11.81.

HRMS calcd: 520.2303; Found: 520.2303

¹H NMR(400 MHz, DMSO): 0.70 (2H, br s), 0.79(2H, m), 1.28(3H, t), 2.77(1H, m), 4.26(2H, q), 4.35(2H, d), 4.38(2H, s), 6.76(1H, s), 6.78(1H, s), 7.12(1H, s), 7.29(1H, s), 7.36(2H, d), 7.62 (2H, d), 8.68 (1H, t), 9.05 (1H, br s)

### Example 117

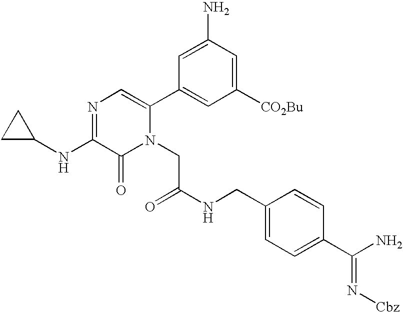

### Example 117

To the free acid (0.25 g, 0.573 mmol) and benzyl [4-(aminomethyl)phenyl](imino)methylcarbamate hydrochloride (219 mg, 688 mmol) in 2.5 ml of N,N-dimethylformamide was added N,N-diisopropylethylamine (0.40 ml, 2.29 mmol) and then benzotriazol-1-yl tetramethyluronium tetrafluoroborate (0.22 g, 0.688 mmol). The solution was stirred for 1.5 hours and then treated with trifluoroacetic acid (0.22 ml, 2.87 mmol). The solution was then purified by reverse phase chromatography with 10-60% CH₃CN/H₂O to give 290 mg (58% yield) of an off-white solid m/z(M+H)⁺666

Analysis: C₃₆H₃₉N₇O₆+1.85 TFA+0.45 H₂O calcd: C, 53.89; H, 4.76; N, 11.08; found: C, 53.91; H, 4.80; N, 11.02.

HRMS calcd: 666.3035; Found: 666.3073

¹H NMR(400 MHz, DMSO): 0.67(2H, m), 0.75(2H, m), 0.90(3H, t), 1.39 (2H, sextet), 1.62(2H, quintet), 2.76(1H, m), 4.21(2H, t), 4.36(2H, d), 4.38(2H, s), 5.32(2H, s), 6.75(1H, s), 6.78(1H, t), 7.12(1H, s), 7.27(1H, s), 7.35-7.49(7H, m), 7.75 (2H, d), 8.68(1H, t)

### Example 118

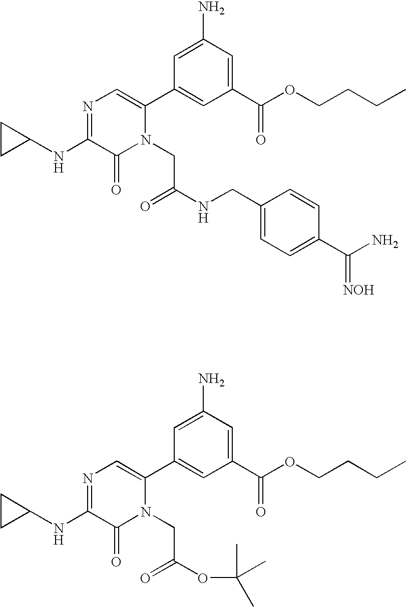

### Example 118a

To bromo-pyrazinone (1.16 g, 3.38 mmol), phenyl-boronic acid(1.18 g, 4.32 mmol), and cesium carbonate (4.38 g, 13.5 mmol) in 55 ml of acetonitrile and 5.5 ml of water was added tetrakis(triphenylphosphine) palladium(0) under nitrogen. The mixture was heated at 75° C. for 9 hours. The aqueous layer was pipetted of and 50 ml of water was added. The mixture was concentrated to a small volume and then extracted with ethyl acetate. The organic layer was dried over magnesium sulfate, filtered and concentrated in vacuo to give ˜4 g of an orange oil. The oil was dissolved in dichloromethane and purified by silica gel chromatography with 10-60% EA/Hex to give 1.6 g of an orange oil. This oil was dissolved in 200 ml ethyl acetate and 5 ml of dichloromethane and stirred overnight. The mixture was filtered and then concentrated. The residue was purified by silica gel chromatography with 45-65% EA/Hex to give 740 mg (48% yield) of a yellow-orange oil. m/z(M+H)⁺457

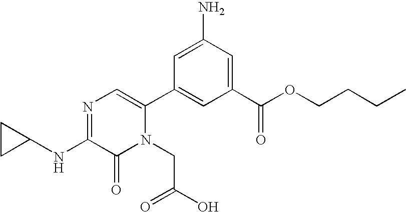

### Example 118b

To the product from Example 118a (0.733 g, 1.61 mmol) was added 4M hydrogen chloride in dioxane (10 ml, 40 mmol). The solution was heated at 60° C. for 45 min. The mixture was diluted with 100 ml of ethyl ether and the resulting precipitate was collected by vacuum filtration and dried over phosphorous pentoxide under high vacuum to give 0.66 g (94% yield) of a yellow solid. m/z(M+H)⁺401

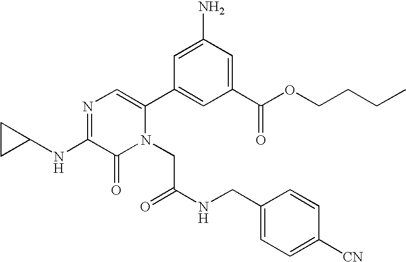

### Example 118c

To the product from Example 118b (0.40 g, 0.917 mmol) and 4-aminobenzonitrile (185 mg, 1.1 mmol) in 4 ml of N,N-dimethylformamide was added N,N-diisopropylethylamine (0.64 ml, 3.67 mmol) and then benzotriazol-1-yl tetramethyluronium tetrafluoroborate (0.353 g, 1.1 mmol). The solution was stirred for 1.5 hours. The solution was added to a stirring solution of 20 ml of water. The resulting precipitate was collected by vacuum filtration and dried over phosphorous pentoxide under high vacuum to give 460 mg (92% yield) of a tan solid m/z (M+H)⁺515

### Example 118d

To the product from Example 118c (460 mg, 0.84 mmol), hydroxyl amine hydrochloride (292 mg, 4.20 mmol) and potassium carbonate (0.58 g, 4.2 mmol) in 10 ml of ethanol was added N,N-diisopropylethylamine (1.02 ml, 5.89 mmol). The mixture was heated at 70° C. for 6 hours. The solution was treated with 10 ml of water and the precipitate was collected. The filtrate was treated with 10 ml of brine and the extracted with ethyl acetate. The organic was dried over sodium sulfate, filtered and econcentrated in vacuo to give a residue. The residue and the precipitate were dissolved in acetonitrile and acidified with trifluoroacetic acid. The solution was purified by reverse phase chromatography with 5-45% CH₃CN/H₂O to give 330 mg (50% yield) of an off-white solid m/z(M+H)⁺548

Analysis: C₂₈H₃₃N₇O₅+2.15 TFA+1.10 H₂O calcd: C, 47.74; H, 4.63; N, 12.06; found: C, 47.72; H, 4.61; N, 12.05 .

HRMS calcd: 548.2616; Found: 548.2608

¹H NMR(400 MHZ, DMSO): 0.71(2H, m), 0.79(2H, m), 0.91(3H, t), 1.40(2H, sextet), 1.62(2H, quintet), 2.76(1H, m), 4.22(2H, t), 4.35(2H, d), 4.38(2H, s), 6.77(1H, s), 6.79(1H, s), 7.12(1H, s), 7.29(1H, s), 7.37(2H, d), 7.62(2H, d), 8.68(1H, t), 9.02(1H, br s).

### Example 119

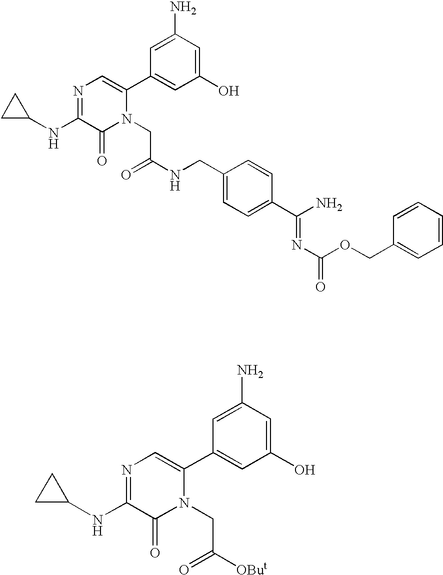

### Tert butyl[6-[3-amino-5-(hydroxy)phenyl]-3-cyclopropylamino)-2-oxopyrazin-1(2H)-yl]acetate

### Example 119a

4.5 g (9.1 mmol) of t-butyl[6-[3-nitro-5-(O-benzyl)phenyl]-3-cyclopropylamino)-2-oxopyrazin-1(2H)-yl]acetate was dissolved in 50 mL MeOH and reduced in the presence of 2.5 g HCOONH₄ and 0.2 g Pd black with stirring for 12 hours. The catalyst was filtered off and the solvent was evaporated to dryness. The crude product was dissolved in the mixture of AcN and H₂O and purified on preparative HPLC using a gradient of acetonitrile (10-50% AcN in 30 minutes), yielding the product at 30% AcN, 3.5 g (7.2 mmol; 79%) as a white solid. MH⁺=373.2

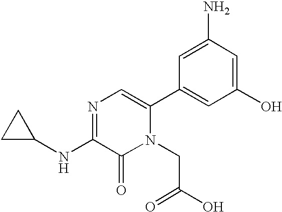

### Example 119b

The residue of Example 119a was treated with 3 mL of TFA for 90 minutes with stirring and then TFA was evaporated to give an oil. MH⁺=317.2

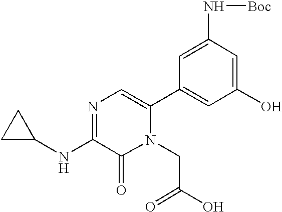

### Example 119c

The of Example 119b was dissolved in 50 mL dioxane and 25 mL H₂O and the pH was adjusted to >8 by the addition of 2.5 N NaOH. 0.65 g (3 mmol) (Boc)₂O was added to the mixture and it was stirred for 12 hours. Dioxane was evaporated and the residue was diluted with 50 mL of 10% KHSO₄. It was extracted with 2×100 mL EtOAc. The organic phase was washed with brine, dried over MgSO₄ and the solvent was evaporated. Yield: 1.3 g (3.1 mmol; 43%) semi solid. MH+=417.3

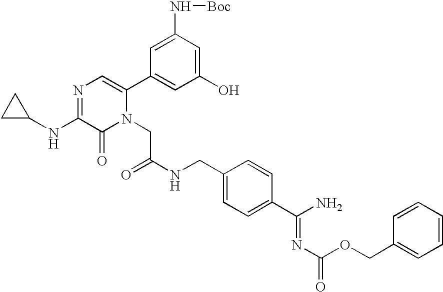

### Example 119d

0.96 g (2 mmol) di-Boc-4-amino-Z-benzamidine was deprotected in 25 mL CH₂Cl₂/TFA (4:1) for 30 minutes with stirring and the solvent was thoroughly evaporated to dryness. This solid and 0.7 g (1.7 mmol) of Example 119c were dissolved in 25 mL DMF. They were coupled in the presence of 0.64 g (2 mmol) TBTU and 1.75 mL (10 mmol) DIPEA for 1 hour. DMF was evaporated and the product was precipitated by addition of 200 mL water and filtered. The crude product was dissolved in the mixture of AcN and H₂O and purified on preparative HPLC using a gradient of acetonitrile (10-50% AcN in 30 minutes), yielding the product at 47% AcN, 1.1 g (1.6 mmol; 65%) as a white solid.

MH⁺=682.4

### Example 119e

0.61 g (0.9 mmol) of Example 119d was deprotected in 25 mL CH₂Cl₂/TFA (4:1) for 30 minutes with stirring. The solvent was evaporated and the residue was dissolved in AcN/H₂O and lyophilized to yield 0.72 g (0.89 mmol; 55%) white solid.

MH⁺=582.4

¹HNMR: 400 MHz, CD₃OD: 7.78-7.72 (d, 2H), 7.54-7.30 (m, 7H), 6.66 (s, 1H), 6.46-6.26 (m, 3H), 5.40 (s, 2H), 4.60 (s, 2H), 4.52-4.44 (m, 2H), 2.80-2.70 (m, 1H), 1.08-1.00 (m, 2H) and 0.90-0.80 (m, 2H).

Elemental analysis: C₃₁H₃₁N₇O₅ + 2.8xTFA + 1.2xH₂O

| | | | |
|---|---|---|---|
| Found | C: 47.52 | H: 4.03 | N: 10.80 |
| Calc. | C: 47.65 | H: 3.96 | N: 10.63 |

### Example 121

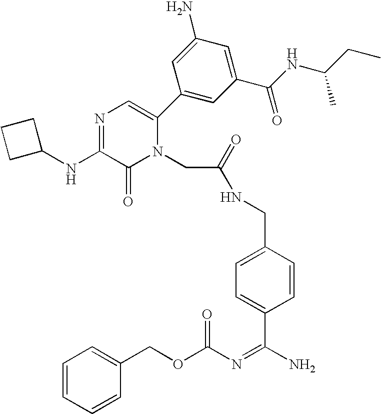

HPLC/LRMS: >98%, 679(M+H)+; HRMS(ES+) calcd. for C₃₇H₄₃N₈O₅ 679.3351, found 679.3380.

### Example 122

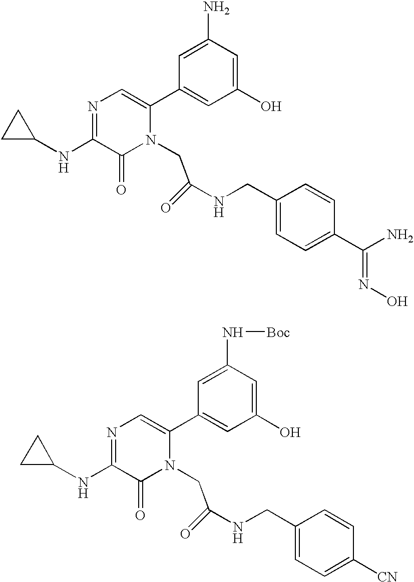

### Example 122a

1 g (3 mmol) N,N-di-Boc-4-aminobenzonitrile was deprotected in 25 mL CH₂Cl₂/TFA (4:1) for 30 minutes and the solvent was evaporated thoroughly to dryness. 0.5 g (1.2 mmol) of the free acid was coupled with the 4-aminobenzo-nitrile in 30 mL DMF in the presence of 0.48 g (1.5 mmol) TBTU and 1.75 mL (10 mmol) DIPEA with stirring for 1 hours. DMF was evaporated and the product was precipitated by addition of 200 mL water, filtered and dried. Yield: 0.38 g (0.72 mmol; 60%) solid.

MH⁺=531.2

### Example 122b

0.38 g (0.72 mmol) of Example 122a was dissolved in 25 mL EtOH and it was refluxed in the presence of 0.7 g (10 mmol) hydroxylamine.HCl and 1.75 mL (10 mmol) DIPEA for 4 hours. The solvent was evaporated and the product was purified on preparative HPLC using a gradient of acetonitrile (10-50% AcN in 30 minutes). Yield: 0.26 g (0.46 mmol; 64%) as a white solid. MH⁺=564.2

### Example 122c

0.26 g (0.46 mmol) of Example 122b was deprotected in 25 mL CH₂Cl₂/TFA (4:1) with stirring for 30 minutes. The solvent was evaporated and the residue was dissolved in AcN/H₂O and lyophilized to yield 0.22 g (0.32 mmol; 69%) white solid.

MH⁺=464.2

¹HNMR: 400 MHz, CD₃OD: 7.70-7.62 (d, 2H), 7.50-7.40 (d, 2H), 6.67 (s, 1H), 6.60 (s, 1H), 6.56 (s, 1H), 6.50 (s, 1H), 4.59 (s, 2H), 4.46 (s, 2H), 2.84-2.74 (m, 1H), 1.10-1.02 (m, 2H) and 0.90-0.80 (m, 2H).

Elemental analysis: C₂₃H₂₅N₇O₄ + 2.4xTFA + 1.5xH₂O

| | | | |
|---|---|---|---|
| Found | C: 43.79 | H: 4.09 | N: 12.73 |
| Calc. | C: 43.69 | H: 4.01 | N: 12.83 |

### Example 123

### Ethyl 3-amino-5-[1-{2-[(4-{(Z)-amino[(phenoxycarbonyl)imino]methyl}benzyl)amino[-2-oxoethyl}-5-(cyclopropylamino)-6-oxo-1,6-dihydropyrazin-2-yl]benzoate

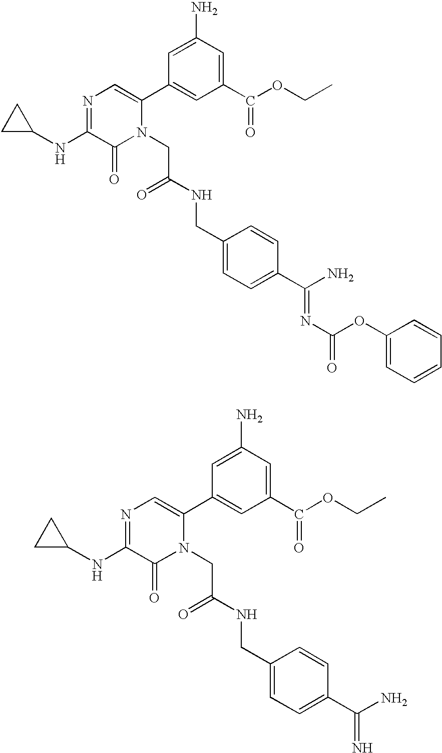

### Ethyl 3-amino-5-[1-[2-({4-[amino(imino)methyl]benzyl}amino)-2-oxoethyl]-5-(cyclopropylamino)-6-oxo-1,6-dihydropyrazin-2-yl]benzoate

### Example 123a

To a Parr bottle containing Z-protected amidine (1.7 g, 2.7 mmol) was added ethanol (40 mL) and concentrated HCl (4 drops). The reaction was shaken on Parr hydrogenator for 2 hrs and 40 psi. The mixture was filtered through Celite and the solvent removed in vacuo to give a yellow solid (0.97 g, 73%). MS-ESI (M+H)=505.

### Example 123b

To a solution of Example 123a (0.48 g, 0.95 mmol) was added NMM (0.46 mL, 4.2 mmol) and diphenyl carbonate (0.22 g, 1.0 mmol). The reaction was stirred overnight at room temperature and then purified by RP-HPLC (CH₃CN:H₂O) to give the desired product (120 mg). ¹H NMR (400 MHz, CD₃OD): δ 7.82 (d, 2 H), 7.49-7.29 (m, 9 H), 6.90 (t, 1 H), 6.69 (s, 1 H), 4.59 (s, 2 H), 4.49 (d, 2 H), 4.32 (q, 2 H), 2.81-2.75 (m, 1 H), 1.35 (t, 3 H), 1.06-1.02 (m, 2 H), 0.86-0.84 (m, 2 H); MS-ESI (M+H)=624; Analysis: C₃₃H₃₃N₇O₆+2.15 TFA+2.05 H₂O calcd: C, 49.45; H, 4.36; N, 10.82; found: C, 49.45; H, 4.32; N, 10.89.

### Example 124

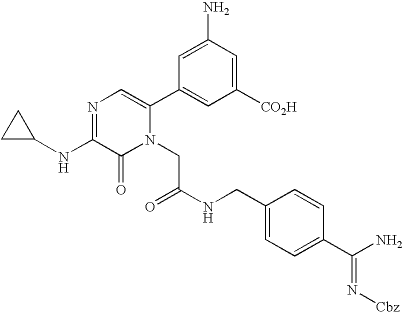

### Example 124

To free amidine (296 mg, 0.62 mmol) and Z-chloride (187 mg, 0.69 mmol) in 4 ml of N,N-dimethylformamide was added N-methylmorpholine (0.274 ml, 2.5 mmol). The solution was stirred for 40 hours. The solution was treated with trifluoroacetic acid (0.24 ml, 3.12 mmol) and purified by reverse phase chromatography with 5-40% CH₃CN/H₂O to give 56 mg (11% yield) of a light yellow solid. m/z(M+H)⁺610

Analysis: C₃₂H₃₁N₇O₆+1.70 TFA+1.95 H₂O calcd: C, 50.70; H, 4.40; N, 11.69; found: C, 50.65; H, 4.33; N, 11.82.

HRMS calcd: 610.2409; Found: 610.2413

¹H NMR(400 MHz, DMSO): 0.66(2H, br s), 0.75(2H, br d), 2.76(1H, m), 4.36(2H, d), 4.38(2H, s), 5.33(2H, s), 6.74(1H, s), 6.75(1H, s), 7.09(1H, s), 7.27(1H, s), 7.36-7.49(8H, m) 7.75 (2H, d), 8.68(1H, br s).

### Example 125

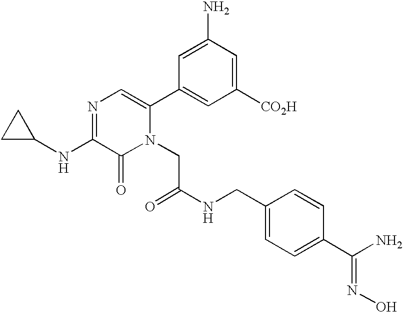

### Example 125

To free amidine (284 mg, 0.60 mmol), hydroxyl amine hydrochloride (124 mg, 1.79 mmol) and potassium carbonate (0.41 g, 2.99 mmol) in 5 ml of ethanol was added N,N-diisopropylethylamine (0.52 ml, 2.99 mmol). The mixture was heated at 70° C. for 3 hours. The solution was acidified with trifluoroacetic acid (1.0 ml, 13 mmol) and purified by reverse phase chromatography with 5-20% CH₃CN/H₂O to give 110 mg (24% yield) of a white solid m/z (M+H)⁺492

Analysis: C₂₄H₂₅N₇O₅+2.55 TFA+1.75 H₂O calcd: C, 42.95; H, 3.85; N, 12.05; found: C, 42.95; H, 3.87; N, 12.05.

HRMS calcd: 492.1990; Found: 492.1987

¹H NMR(400 MHz, DMSO): 0.69(2H, m), 0.78(2H, m), 2.76(1H, m), 4.35(2H, d), 4.38(2H, s), 6.74(1H, s), 6.76(2H, s), 7.10(1H, s), 7.27(1H, s), 7.37 (2H, d), 7.63 (2H, d), 8.67(1H, t).

### Example 126

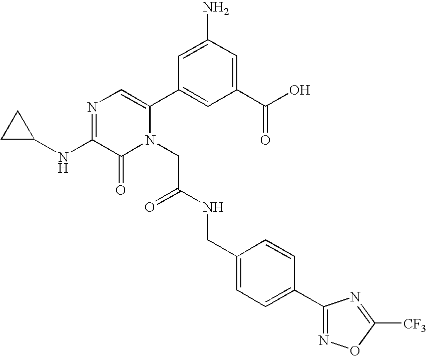

### Example 126

To a stirred solution of ethyl ester (90 mg, 0.12 mmol) in THF (3 ml) and ethanol (2 ml) cooled in ice bath was added 2N lithium hydroxide (0.152 mL, 0.3 mmol). The reaction was stirred at ambient temperature for 48 hours. Purification by reverse phase HPLC (10-80% acetonitrile/water) followed by lyophilization yielded 5.5 mg of an off-white solid. ¹HNMR (300 MHz, DMSO-d₆) δ 0.63-0.78 (m, 4H), 2.70-2.80 (m, 1H), 4.36 (d, J=6.0 Hz, 2H), 4.39 (s, 2H), 6.73 (s, 1H), 6.76 (s, 1H), 7.10 (s, 1H), 7.27 (s, 1H), 7.41 (d, J=8.4 Hz, 2H), 8.00 (d, J=8.1 Hz, 2H), 8.62 (t, J=5.7 Hz, 1H). ¹⁹FNMR (282 MHz, DMSO-d₆) δ −65.08 (s). HRMS calcd for C₂₆H₂₃N₇O₅F₃ (M+H): 570.1707. Found: 570.1698.

### Example 127

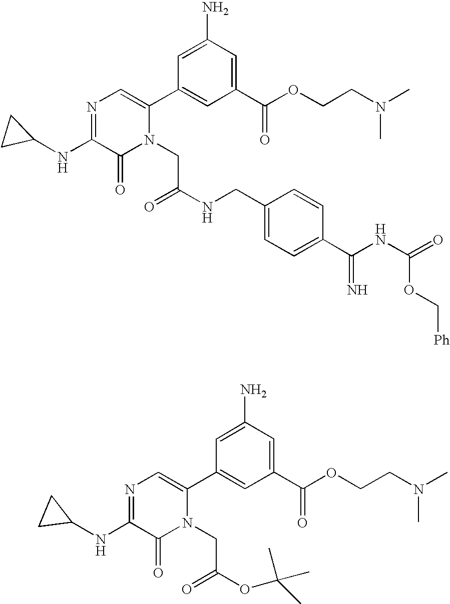

### Example 127a

A solution of tert-butyl [6-bromo-3-(cyclopropylamino)-2-oxopyrazin-1(2H)-yl]acetate(500 mg, 1.46 mmol), 3-amino-5-{[2-(dimethylamino)ethoxy]-carbonyl}phenylboronic acid(0.6 g, 2.2 mmol), Cs2CO3(1.9 g, 5.84 mmol) and [Pd(PPh3)4]( 500 mg, 0.44 mmol) in a mixture of MeCN (10 ml) and water(2 ml) was heated at 75° C. overnight. The solid was filtered. Water(200 ml) was added and MeCN was then removed by rotavapor. Aqueous solution was then extracted with EtOAc(2×300 ml The combined EtOAc was then dried over MgSO4 and concentrated without purification to yield 560 mg solid (81%).

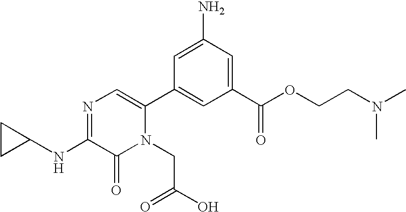

### Example 127b

0.56 g, 1.27 mmol of Example 127a in HCl/Dioxane (4N, 10 ml) was heated at 65° C. for 1 hr and concentrated to yield 0.5 g brown solid (95%).

### Example 127c

0.5 g, 1.2 mmol of Example 127b, benzyl [4-(aminomethyl)phenyl](imino)methylcarbamate (460 mg, 1.45 mmol), TBTU (385 mg, 1.2 mmol) and DIEA (775 mg, 6 mmol) in 20 ml of DMF was kept stirring at RT for 2 hr. The mixture was then purified on RP-HPLC to yield 0.688 g solid (53%).

HRMS calcd for C₃₆H₄₀N₈O₆ (M+H): 681.3144. Found: 681.3142.

Anal. Calcd for C₃₆H₄₀N₈O₆ + 3.45TFA + 2H2O:

| | | | |
|---|---|---|---|
| | C: 46.41; | H: 4.30; | N: 10.09. |
| Found: | C: 46.37; | H: 4.19; | N: 10.28. |

¹H NMR (DMSO-d₆, 300 MHz) δ 0.64 (m, 2H), 0.73 (m, 2H), 2.74 (m, 1H), 2.86 (bs, 7H), 3.47 (bs, 2H), 4.37 (m, 5H), 4.47 (d, 1H), 4.53 (m, 3H), 5.21 (s, 1H), 5.32 (s, 2H), 6.73(s, 1H), 6.81 (m, 1H), 7.19(t, 1H), 7.29 (t, 1H), 7.38-7.46 (m, 6H), 7.76(m, 2H), 7.90 (m, 1H), 8.72(t, 1H).

### Example 128

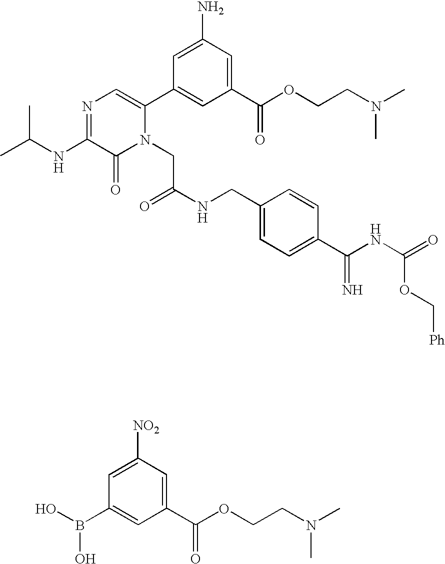

### Example 128a

3-(dihydroxyboryl)-5-nitrobenzoic acid(1.4 g, 5 mmol) in 32 ml of THF was added SOCl2(4 ml) and 2 drops of DMF. The mixture was heated to reflux for 2 hr and concentrated. THF (4 ml) was added and the mixture was cooled to 0° C. Then 2-(dimethylamino)ethanol (0.54 g, 6 mmol) and Et3N(0.3 g, 3 mmol) was added. The mixture was kept stirring at RT for 5 hr, then concentrated and purified with RP-HPLC to yield 1 g white solid (71%).

C₁₁H₁₅B₁N₂O₆ M.W. 282.06.

¹H NMR (DMSO-d₆, 300 MHz) δ 2.90 (s, 6H), 3.58(m, 2H), 4.66(m, 2H), 8.78-8.88 (m, 3H).

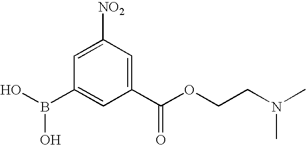

### Example 128b

A solution of Example 128a (0.18 g, 0.64 mmol) in EtOH (20 ml) was added Pd/C(10%). The mixture was set on hydrogenation shake at 40 psi for 3 hr, then filtered and concentrated to yield 0.16 g solid (99%).

C₁₁H₁₇B₁N₂O₄ M.W. 252.07.

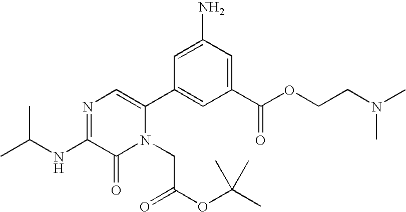

### Example 128c

A solution of tert-butyl [6-bromo-3-(isopropylamino)-2-oxopyrazin-1(2H)-yl]acetate(100 mg, 0.29 mmol), 3-amino-5-{[2-(dimethylamino)ethoxy]-carbonyl}phenylboronic acid(0.14 g, 0.52 mmol), Cs2CO3(0.4 g,1.2 mmol) and [Pd(PPh3)4] (100 mg, 0.087 mmol) in a mixture of MeCN (2 ml) and water(0.5 ml) was heated at 75° C. overnight. The solid was filtered. Water(200 ml) was added and MeCN was then removed by rotavapor. Aqueous solution was then extracted with EtOAc(2×300 ml) and combined EtOAc was then dried over MgSO4 and concentrated without purification to yield 94 mg solid (69%).

C₂₄H₃₅N₅O₅ M.W. 473.57.

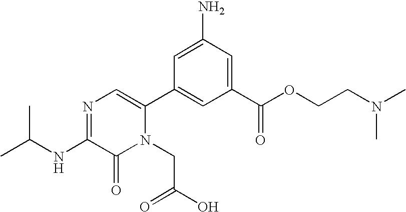

### Example 128d

0.1 g, 0.21 mmol of Example 128c in HCl/Dioxane (4N, 2 ml) was heated at 65° C. for 1 hr and concentrated to yield 80 mg brown solid (91%).

C₂₀H₂₇N₅O₅ M.W. 417.46.

### Example 128e

0.1 g, 0.24 mmol of Example 128d, benzyl [4-(aminomethyl)phenyl](imino)methylcarbamate (92 mg, 0.29 mmol), TBTU (77 mg, 0.24 mmol) and DIEA (155 mg, 1.2 mmol) in 5 ml of DMF was kept stirring at RT for 2 hr. The mixture was then purified on RP-HPLC to yield 0.12 g solid (46%).

HRMS calcd for C₃₆H₄₂N₈O₆ (M+H): 683.3300. Found: 683.3282.

Anal. Calcd for C₃₆H₄₂N₈O₆ + 3.55TFA + 1.4H2O:

| | | | |
|---|---|---|---|
| | C: 46.52; | H: 4.37; | N: 10.06. |
| Found: | C: 46.47; | H: 4.32; | N: 10.18. |

¹H NMR (DMSO-d₆, 300 MHz) 1.20 (d, 6H), 2.85 (bs, 7H), 3.46 (bs, 2H), 4.09 (m, 3H), 4.38 (m, 5H), 4.53 (m, 3H), 5.21 (s, 1H), 5.31 (s, 1H), 6.68(s, 1H), 6.81 (d, 1H), 7.19(s, 1H), 7.29 (s, 1H), 7.38-7.46 (m, 6H), 7.77(m, 2H), 7.90 (m, 1H), 8.71(t, 1H).

### Example 129

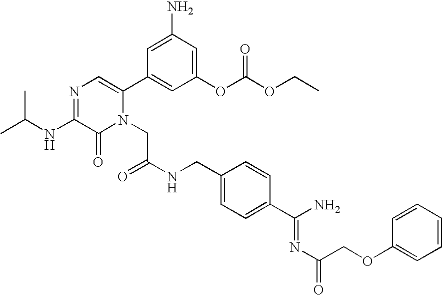

### Example 129

0.4 g (0.58 mmol) of Boc-protected aminophenol was dissolved in 10 mL CH₂Cl₂ and it was reacted with 0.11 g (1 mmol; 0.096 mL) ethylchloroformate in the presence of 0.175 mL (1 mmol) DIPEA for 1 hour with stirring. The solvent was evaporated and the remaining oil was dissolved in 5 mL AcN. The product was precipitated by addition of 200 mL H₂O, filtered and dried. MH⁺=756.4 The white solid was dissolved in 25 mL CH₂Cl₂/TFA (4:1) and stirred for 30 minutes. The solvent was evaporated and the residue was purified on prep HPLC using a gradient of acetonitrile (10-50% AcN in 30 minutes), to yield (product peak at 47% AcN) 0.225 g (0.34 mmol; 59%) white solid

MH⁺=656.4

¹HNMR: 400 MHz, CD₃OD: 7.80-7.72 (d, 2H), 7.52-7.32 (m, 7H), 6.65 (s, 1H), 6.60-6.54 (m, 2H), 6.46 (s, 1H), 5.40 (s, 2H), 4.60 (s, 2H), 4.46 (s, 2H), 4.30-4.18 (m, 2H), 4.10-4.00 (m, 1H) and 1.36-1.26 (m, 9H).

Elemental analysis: C₃₄H₃₇N₇O₇ + 1.3xTFA + 3xH₂O

| | | | |
|---|---|---|---|
| Found | C: 51.45 | H: 5.09 | N: 11.26 |
| Calc. | C: 51.24 | H: 5.20 | N: 11.40 |

### Example 130

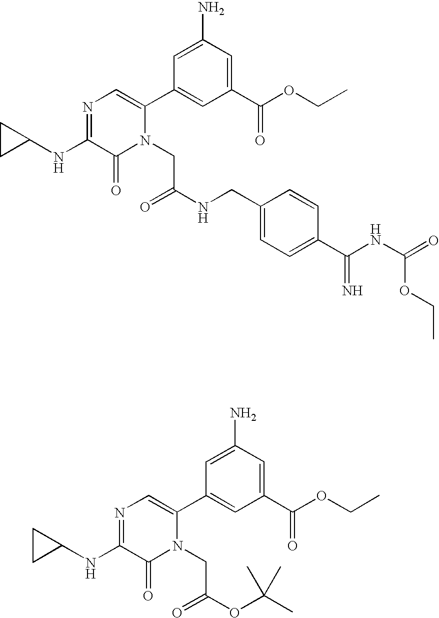

### Example 130a

A solution of tert-butyl [6-bromo-3-(cyclopropylamino)-2-oxopyrazin-1(2H)-yl]acetate(3.6 g, 10.5 mmol), 3-amino-5-(ethoxycarbonyl)phenylboronic acid (4 g, 2.19.1 mmol), Cs₂CO₃(13.6 g, 42 mmol) and [Pd(PPh3)4]( 3.64 g, 3.15 mmol) in a mixture of MeCN (200 ml) and water(20 ml) was heated at 75° C. overnight. The solid was filtered. Water(200 ml) was added and MeCN was then removed by rotavapor. Aqueous solution was then extracted with EtOAc(2×300 ml) and combined EtOAc was then dried over MgSO4 and concentrated without purification to yield 2.9 g solid (65%).

C₂₂H₂₈N₄O₅ M.W. 428.48.

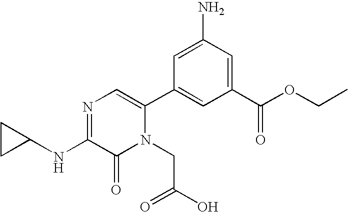

### Example 130b

2.9 g, 6.78 mmol of Example 130a in HCl/Dioxane (4N, 35 ml) was heated at 65° C. for 1 hr and concentrated to yield 2.5 g brown solid (99%).

C₁₈H₂₀N₄O₅ M.W. 372.38.

¹H NMR (DMSO-d₆, 300 MHz) δ 0.84 (m, 2H), 0.90 (m, 2H), 1.30 (t, 3H), 2.80 (m, 1H), 4.28 (q, 2H), 4.36 (s, 2H), 6.79(s, 1H), 6.86(s, 1H), 7.17(s, 1H), 7.40 (s, 1H), 7.59(m, 1H), 9.74 (bs, 1H).

### Example 130c

0.19 g, 0.51 mmol of Example 130b, the product of example 24b (221 mg, 1 mmol), TBTU (193 mg, 0.54 mmol) and DIEA (332 mg, 2.5 mmol) in 3 ml of DMF was kept stirring at RT for 2 hr. The mixture was then purified on RP-HPLC to yield 0.10 g solid (23%).

HRMS calcd for C₂₉H₃₃N₇O₆ (M+H): 576.2565. Found: 576.2541.

Anal. Calcd for C₂₉H₃₃N₇O₆ + 2.4TFA + 1.5H2O:

| | | | |
|---|---|---|---|
| | C: 46.32; | H: 4.41; | N: 11.18. |
| Found: | C: 46.32; | H: 4.44; | N: 11.18. |

¹ HNMR (DMSO-d₆, 300 MHz) δ 0.69 (m, 2H), 0.75 (m, 2H), 1.30 (m, 6H), 2.76 (m, 1H), 4.26-4.39 (m, 10H), 6.75(s, 1H), 6.78 (s, 1H), 7.11(s, 1H), 7.28 (s, 1H), 7.38 (d, 2H), 7.72(d, 2H), 8.68 (t, 1H), 10.46 (bs, 1H).

### Example 131

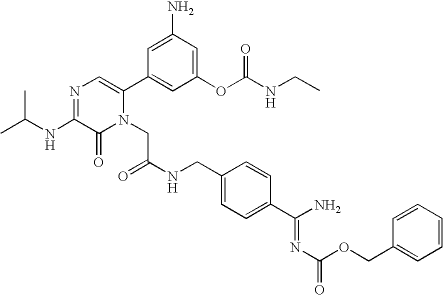

### Example 131

0.2 g (0.3 mmol) of Boc-protected phenol was dissolved in 10 mL CH₂Cl₂ and it was reacted with 0.028 g (0.4 mmol; 0.032 mL) ethylisocyanate in the presence of 0.088 mL (0.5 mmol) DIPEA for 1 hour with stirring. The solvent was evaporated and the remaining oil was dissolved in 5 mL AcN. The product was precipitated by addition of 200 mL H₂O, filtered and dried. MH⁺=755.4

The white solid was dissolved in 5 mL CH₂Cl₂/TFA (4:1) and stirred for 30 minutes. The solvent was evaporated and the residue was purified on prep HPLC using a gradient of acetonitrile (10-50% AcN in 30 minutes), to yield (product peak at 40% AcN) 0.115 g (0.17 mmol; 58%) white solid.

MH⁺=655.4

¹HNMR: 400 MHz, CD₃OD: 7.78-7.72 (d, 2H), 7.50-7.42 (d, 2H), 7.41-7.30 (m, 5H), 6.65 (s, 1H), 6.58-6.50 (m, 2H), 6.42 (s, 1H), 5.40 (s, 2H), 4.62 (s, 2H), 4.44 (s, 2H), 4.10-3.98 (m, 1H), 3.24-3.10 (m, 2H) and 1.40-1.10 (m, 9H).

Elemental analysis: C₃₄H₃₈N₈O₆ + 1.9xTFA + 2.1xH₂O

| | | | |
|---|---|---|---|
| Found | C: 50.09 | H: 4.95 | N: 12.25 |
| Calc. | C: 49.94 | H: 4.89 | N: 12.32 |

### Example 132

### 2-[6-[3-amino-5-(trifluoromethyl)phenyl]-3-(isopropylamino)-2-oxopyrazin-1(2H)-yl]-N-{4-[5-(pentafluoroethyl)-1,2,4-oxadiazol-3-yl]benzyl}acetamide

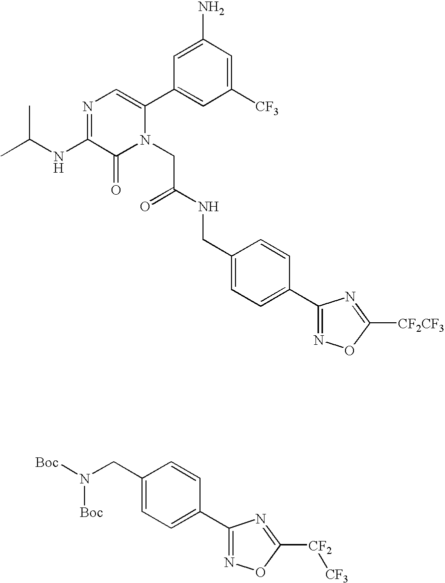

### Di(tert-butyl) 4-[5-(pentafluoroethyl)-1,2,4-oxadiazol-3-yl]benzylimidodicarbonate

### Example 132a

To a solution of di-bocaminobenzyl-4-hydroxamidine (0.51 g, 1.39 mmol) in dichloromethane (20 mL) was added pyridine (0.25 mL, 3.06 mmol) and pentafluoro-propionic anhydride (0.29 mL, 1.46 mmol) at 0° C. The reaction mixture was allowed to warm to room temperature and stirred for 2 hrs. The reaction mixture was diluted with water. The layers were separated and the aqueous layer extracted with dichloromethane (2×). The organic extracts were washed with brine (1×). The organic fractions were dried (Na₂SO₄) and the solvent removed in vacuo to give a white solid, which after chromatography (silica, 10% ethyl acetate/hexanes to 30% ethyl acetate/hexanes) gave Example 132a as a white solid (0.60 g). ¹H NMR (400 MHz, CDCl₃): δ 8.06 (d, 2 H), 7.42 (d, 2 H), 4.83 (s, 2 H), 1.45 (s, 18 H); ¹⁹F NMR (371 MHz, CDCl₃) δ −83.73 (s, 3 F), −116.24 (s, 2 F); MS-ESI (M+H)=494.

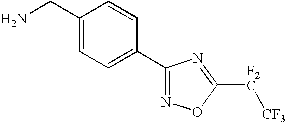

### 4-[5-(pentafluoroethyl)-1,2,4-oxadiazol-3-yl]benzylamine

### Example 132b

To a round bottom containing the product of Example 132a (0.60 g, 1.20 mmol) was added 4.0N HCl in dioxane (20 mL) at room temperature. After stirring for 3 hrs at room temperature the precipitate was filtered and dried on high vacuum to give a white powder Example 132b (0.349 g). ¹H NMR (400 MHz, CD₃OD): δ 8.20 (d, 2 H), 7.66 (d, 2 H), 4.21 (s, 2 H); ¹⁹F NMR (371 MHz, CD₃OD): δ −85.60 (s, 3 F), −118.03 (s, 2 F); MS-ESI (M+H)=294.

### Example 132c

To a solution of free acid (0.18 g, 0.45 mmol) in DMF (10 mL) was added TBTU (0.17 g, 0.53 mmol) at 0° C. After 5 min, Example 132b (0.19 g, 0.64 mmol) and DIEA (0.45 mL, 2.56 mmol) were added. The reaction was stirred for 1 hr and then diluted with water and ethyl acetate. The layers were separated and the organic layer washed with saturated sodium bicarbonate and dried (Na₂SO₄). The solvent was removed to give a semi-solid, which after chromatography (silica, 50:50 ethyl acetate:hexane) gave Example 132c (0.21 g, 72%) as a white solid. ¹H NMR (400 MHz, CD₃OD): δ 8.06 (d, 2 H), 7.43 (d, 2 H), 6.97 (s, 1 H), 6.85 (d, 2 H), 6.71 (s, 1 H), 4.52 (s, 2 H), 4.45 (s, 2 H), 4.12-4.08 (m, 1 H), 1.26 (d, 6 H); ¹⁹F NMR (371 MHz, CD₃OD): δ −64.84 (s, 3 F), −85.61 (s, 3 F), −118.03 (s, 2 H); MS-ESI (M+H)=646.

### Example 133

### 2-[6-[3-amino-5-(trifluoromethyl)phenyl]-3-(isopropylamino)-2-oxopyrazin-1(2H)-yl]-N-{4-[5-(heptafluoropropyl)-1,2,4-oxadiazol-3-yl]benzyl}acetamide

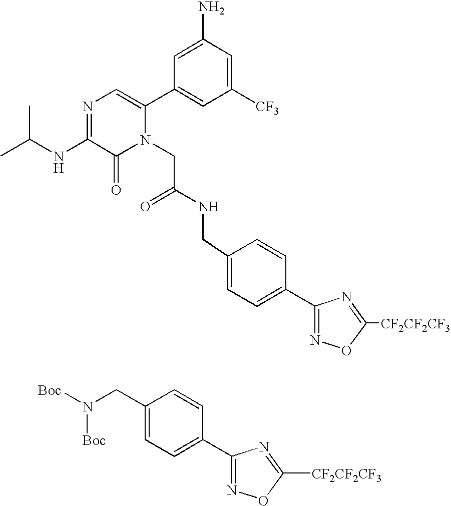

### Di(tert-butyl) 4-[5-(heptafluoropropyl)-1,2,4-oxadiazol-3-yl]benzylimidodicarbonate

### Example 133a

To a solution of di-bocaminobenzyl-4-hydroxamidine (0.75 g, 2.05 mmol) in dichloromethane (20 mL) was added pyridine (0.38 mL, 4.51 mmol) and heptafluoropropionic anhydride (0.53 mL, 2.15 mmol) at 0° C. The reaction mixture was allowed to warm to room temperature and stirred for 2 hrs. The reaction mixture was diluted with water. The layers were separated and the aqeous layer extracted with dichlormethane (2×). The organic extracts were washed with brine (1×). The organic fractions were dried (Na₂SO₄) and the solvent removed in vacuo to give a white solid, which after chromatography (silica, 10% ethyl acetate/hexanes) gave Example 133a as a white solid (0.92 g). ¹H NMR (400 MHz, CDCl₃): δ 8.07 (d, 2 H), 7.42 (d, 2 H) 4.84 (s, 2 H), 1.45 (s, 18 H); ¹⁹F (371 MHz, CD₃OD): δ −80.62 (t, 3 F), −114.16 (q, 2 F), −126.85(bs, 2 F); MS-ESI (M+H)=544.

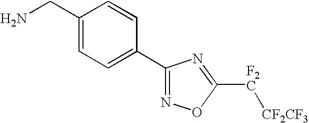

### 4-[5-(heptafluoropropyl)-1,2,4-oxadiazol-3-yl]benzylamine

### Example 133b

To a solution of the product of Example 133a (0.92 g, 1.70 mmol) was added 4.0N HCl in dioxane (20 mL) at room temperature. After stirring for 3 hrs at room temperature the precipitate was filtered and dried on high vacuum to give a white powder Example 133b (0.55 g). ¹H NMR (400 MHz, CD₃OD): δ 8.20 (d, 2 H), 7.67 (d, 2 H), 4.22 (s, 2 H); ¹⁹F NMR (371 MHz, CD₃OD): δ −82.33 (t, 3 F), −115.93 (q, 2 F), −128.41 (bs, 2 F); MS-ESI (M+H)=344.

### Example 133c

To a solution of free acid (0.32 g, 0.78 mmol) in DMF (15 mL) was added TBTU (0.25 g, 0.78 mmol) at 0° C. After 5 min, the product of Example 133b (0.30 g, 0.79 mmol) and DIEA (0.55 mL, 3.12 mmol) were added. The reaction was stirred for 1 hr and then diluted with water and ethyl acetate. The layers were separated and the organic layer washed with saturated sodium bicarbonate and dried (Na₂SO₄). The solvent was removed to give a solid, which after chromatography (silica, 50:50 ethyl acetate:hexanes) gave Example 133c (0.35 g, 65%) as a solid. ¹H NMR (400 MHz, CD₃OD): δ 8.06 (d, 2 H), 7.43 (d, 2 H), 6.97 (s, 1 H), 6.86-6.84 (m, 2 H), 6.71 (s, 1 H), 4.52 (s, 2 H), 4.45 (s, 2 H), 4.12-4.08 (m, 1 H), 1.26 (d, 2 H); ¹⁹F NMR (371 MHz, CD₃OD): δ −64.83 (s, 3 F), −82.36 (t, 3 F), −115.93 (q, 2 F), −128.45 (bs, 2 F); MS-ESI (M+H)=696.

### Example 134

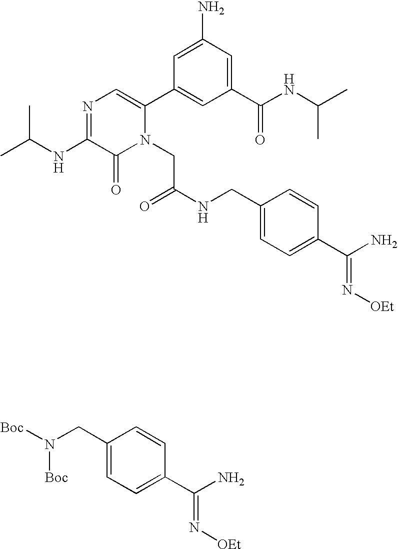

### Example 134a

A solution of di(tert-butyl)-4-[amino(imino)methyl]benzylimidodicarbonate (7.0 g, 17.75 mmol) and O-ethylhydroxylamine hydrochloride (5 g, 51.26 mmol) in 140 ml of ethanol was treated with triethylamine (17.3 ml, 124 mmol) and then heated at reflux for 72 hours. The reaction mixture was allowed to cool and concentrated in vacuo. The residue was treated with 300 ml of ethyl acetate. The organic layer was washed three times with 100 ml of water, dried over sodium sulfate, filtered and concentrated in vacuo to give 6.65 g of a yellow oil. The oil was purified by silica gel chromatography with 20-50% EA/Hex to give 3.85 g (54% yield) of a light yellow solid. m/z(M+H)⁺394

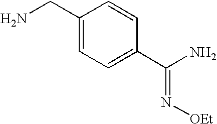

### Example 134b

The product from Example 134a (3.75 g, 9.54 mmol) was dissolved in 4M hydrogen chloride in dioxane (20 ml, 80 mmol) and stirred for 3 hours. The mixture was diluted with 300 ml of ethyl ether and the resulting precipitate was collected by vacuum filtration. The solid was dissolved in methanol, concentrated in vacuo, and dried over phosphorous pentoxide under high vacuum to give 2.54 g (quantitative yield) of a white solid. m/z(M+H)⁺194

### Example 134c

To free acid (0.25 g, 0.646 mmol) and the product from Example 134b (178 mg, .775 mmol) in 3 ml of N,N-dimethylformamide was added N,N-diisopropylethylamine (0.34 ml, 1.94 mmol) and then benzotriazol-1-yl tetramethyluronium tetrafluoroborate (0.25 g, 0.775 mmol). The solution was stirred for 20 minutes and then acidified with trifluoroacetic acid. The solution was then purified by reverse phase chromatography with 5-40% CH₃CN/H₂O to give 290 mg (56% yield) of a white solid. m/z(M+H)⁺563

Analysis: C₂₉H₃₈N₈O₄+2.00 TFA+1.00 H₂O calcd: C, 49.01; H, 5.23; N, 13.86; found: C, 49.39; H, 5.38; N, 14.02.

HRMS calcd: 563.3089; Found: 563.3110

¹H NMR(400 MHz, 9:1-CDCl₃/CD₃O D): 1.14(6H, d), 1.25(6H, d), 1.29(3H, t), 4.09(4H, m), 4.26(2H, s), 4.59(2H, s), 6.72(1H, s), 6.75(1H, t), 7.00(1H, s), 7.06(2H, d), 7.11 (1H, t), 7.48 (2H, d).

### Example 135

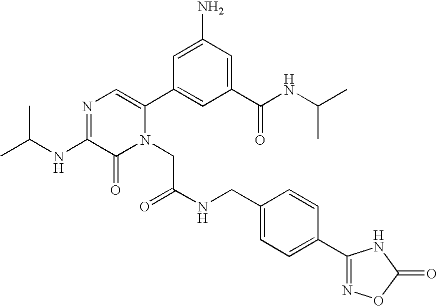

### Example 135

To the free acid (0.25 g, 0.646 mmol) and 4-aminomethyl-benzoxadiazolinone(176 mg, 0.775 mmol) in 3 ml of N,N-dimethylformamide was added N,N-diisopropylethylamine (0.34 ml, 1.94 mmol) and then benzotriazol-1-yl tetramethyluronium tetrafluoroborate (0.25 g, 0.775 mmol). The solution was stirred for 1 hour and then acidified with trifluoroacetic acid (0.28 ml, 3.64). The solution was then purified by reverse phase chromatography with 5-50% CH₃CN/H₂O to give 225 mg (49% yield) of a white solid m/z(M+H)⁺561

Analysis: C₂₈H₃₂N₈O₅+1.20 TFA+1.00 H₂O calcd: C, 51.04; H, 4.96; N, 15.66; found: C, 51.30; H, 5.05; N, 15.75.

HRMS calcd: 561.2568; Found: 561.2593

¹H NMR(400 MHz, DMSO): 1.08(6H, d), 1.19(6H, d), 4.03(2H, m), 4.30(2H, d), 4.37(2H, s), 6.68(2H, s), 6.99(1H, s), 7.12(1H, s), 7.32(2H, d), 7.71 (2H, d), 8.02(1H, d), 8.60(1H, t).

### Example 136

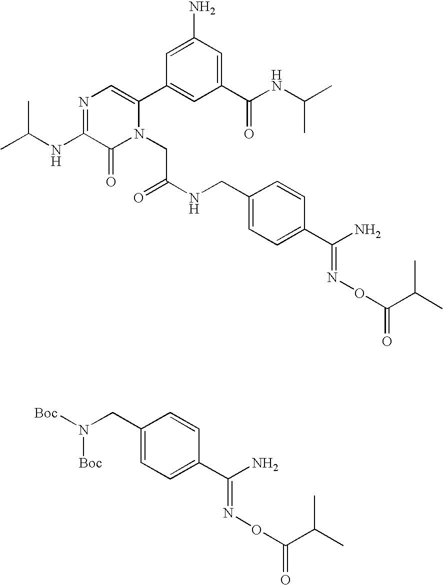

### Example 136a

A mixture of di-Boc-aminobenzyl-hydrox-amidine(1.5 g, 17.75 mmol) and N,N-diisopropylethylamine (0.34 ml, 1.94 mmol) in 10 ml of dichloromethane was treated with isopropyl chloroformate (17.3 ml, 124 mmol) and then stirred overnight. The reaction mixture was treated with ethyl acetate and washed with brine. The organic layer was dried over sodium sulfate, filtered and concentrated in vacuo to give 1.72 g (22% yield) of an off-white solid.

¹H NMR(400 MHz, CDCl₃): 1.26(6H, d, 7.0 Hz), 1.43(9H, s), 2.74(1H, m), 4.78(2H, s), 5.02(2H, br s), 7.30(2H, d, 8.2 Hz), 7.64(2H, d, 8.1 Hz).

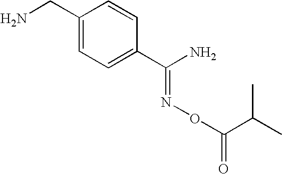

### Example 136b

The product from Example 136a (1.72 g, 3.95 mmol) was dissolved in 4M hydrogen chloride in dioxane (20 ml, 80 mmol) and stirred for 2 hours. The mixture was diluted with 300 ml of ethyl ether and the resulting precipitate was collected by vacuum filtration. The solid was dried over phosphorous pentoxide under high vaccuum to give 1.18 g (quantitative yield) of a light pink solid.

m/z(M+H)⁺236

### Example 136c

To the free acid(0.25 g, 0.646 mmol) and the product from Example 136a (210 mg, 0.775 mmol) in 3 ml of N,N-dimethylformamide was added N,N-diisopropylethylamine (0.34 ml, 1.94 mmol) and then benzotriazol-1-yl tetramethyluronium tetrafluoroborate (0.25 g, 0.775 mmol). The solution was stirred for 1 hour and then acidified with trifluoroacetic acid (0.28 ml, 3.64). The solution was then purified by reverse phase chromatography with 5-60% CH₃CN/H₂O to give 240 mg (56% yield) of a white solid.

m/z (M+H)⁺605

Analysis: C₃₁H₄₀N₈O₅+1.10 TFA+1.00 H₂O calcd: C, 53.30; H, 5.81; N, 14.98; found: C, 53.30; H, 5.76; N, 14.87.

HRMS calcd: 605.3194; Found: 605.3221

¹H NMR(400 MHz, DMSO): 1.09(6H, d), 1.12(6H, d), 1.19(6H, d), 2.70(1H, m), 4.03(2H, m), 4.26(2H, d), 4.36(2H, s), 6.67(3H, s), 6.99(1H, S), 7.11(1H, s), 7.21(2H, d), 7.60 (2H, d), 8.02 (1H, d), 8.56 (1H, t)

### Example 137

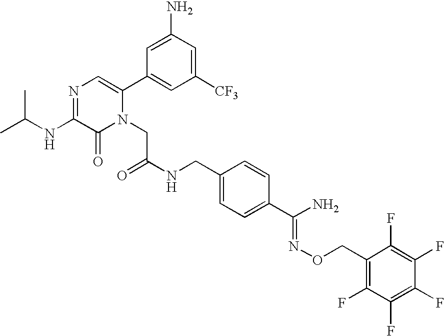

### Example 137

A solution of free amidine (200 mg, 0.33 mmol) and O-(2,3,4,5,6-pentafluorobenzyl)hydroxylamine hydrochloride (328 mg, 1.31 mmol) in 2 ml of ethanol was treated with triethylamine (0.41 ml, 2.95 mmol) and then heated at 85° C. for 72 hours in a sealed vial. The reaction mixture was allowed to cool and concentrated in vacuo. The residue was treated with 2 ml of methyl sulfoxide and methanol was added until everything dissolved. The resulting solution was acidified with 0.28 ml of trifluoroacetic acid and then purified by reverse phase chromatography with 5-90% CH₃CN/H₂O to give 112 mg (36% yield) of a light yellow solid. m/z(M+H)⁺698

Analysis: C₃₁H₂₇N₇O₃+2.10 TFA calcd: C, 45.12; H, 3.13; N, 10.46; found: C, 45.02; H, 3.17; N, 10.44.

HRMS calcd: 698.2120; Found: 698.2111

¹H NMR(400 MHz, DMSO): 1.23(6H, d), 4.07(1H, m), 4.26(2H, d), 4.33 (2H, s), 5.07 (2H, s), 6.75 (1H, s), 6.77 (1H, s), 6.77(1H, s), 6.92(1H, s), 7.17(2H, d), 7.53 (2H, d), 8.59(1H, t).

### Example 138

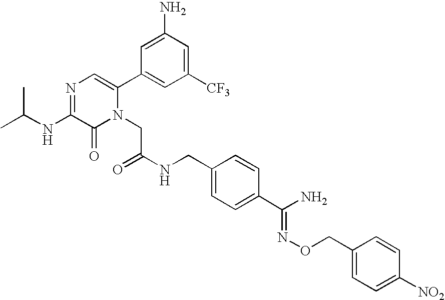

### Example 138

A solution of free amidine (150 mg, 0.246 mmol) and O-(4-nitrobenzyl)hydroxylamine hydrochloride (201 mg, 0.984 mmol) in 2 ml of ethanol was treated with N,N-diisopropylethylamine (0.43 ml, 2.46 mmol) and then heated at 85° C. for 66 hours in a sealed vial. The reaction mixture was allowed to cool and concentrated in vacuo. The residue was dissolved with methyl sulfoxide and acidified with 0.21 ml of trifluoroacetic acid. The solution was then purified by reverse phase chromatography with 10-80% CH₃CN/H₂O to give 106 mg (56% yield) of a yellow solid. m/z(M+H)⁺653

Analysis: C₃₁H₃₁N₈O₅+2.00 TFA+calcd: C, 47.73; H, 3.78; N, 12.72; found: C, 47.91; H, 3.82; N, 12.90.

HRMS calcd: 653.2442; Found: 653.2445

¹H NMR(400 MHz, DMSO): 1.22(6H, d), 4.07(1H, m), 4.26(2H, d), 4.33(2H, s), 5.14(2H, s), 6.74(1H, s), 6.76(1H, s), 6.77(1H, s), 6.92(1H, s), 7.18(2H, d), 7.55 (2H, d), 7.67 (2H, d), 8.21 (2H, d), 8.60 (1H, t)

### Example 139

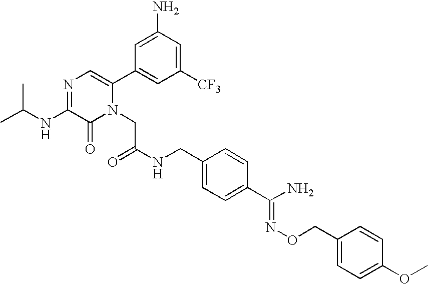

### Example 139

A solution of free amidine (200 mg, 0.328 mmol) and 1-[(ammoniooxy) methyl]-4-methoxybenzene chloride (248 mg, 1.31 mmol) in 2 ml of ethanol was treated with N,N-diisopropylethyl amine(0.57 ml, 3.28 mmol) and then heated at 85° C. for 20 hours in a sealed vial. The reaction mixture was allowed to cool and concentrated in vacuo. The residue was dissolved with methyl sulfoxide and acidified with 0.28 ml of trifluoroacetic acid. The solution was then purified by reverse phase chromatography with 10-80% CH₃CN/H₂O to give 128 mg (44% yield) of a white solid. m/z(M+H)⁺638

Analysis: C₃₂H₃₄N₇O₄+2.00 TFA+0.65 H₂O calcd: C, 49.28; H, 4.28; N, 11.17; found: C, 49.29; H, 4.32; N, 11.18.

HRMS calcd: 638.2697; Found: 638.2688

¹H NMR(400 MHz, DMSO): 1.22(6H, d), 3.74(3H, s), 4.08(1H, m), 4.29(2H, d), 4.34(2H, s), 4.94(2H, s), 6.74(1H, s), 6.77 (1H, s), 6.79 (1H, s), 6.92 (2H, d), 6.93 (1H, s), 7.25 (2H, d), 7.38 (2H, d), 7.57 (2H, d), 8.64 (1H, t).

### Example 140

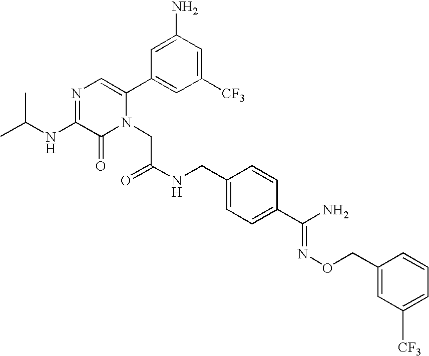

### Example 140

A solution of free amidine (200 mg, 0.328 mmol) and 1-[(ammoniooxy)methyl]-3-(trifluoromethyl)benzene chloride (299 mg, 1.31 mmol) in 2 ml of ethanol was treated with N,N-diisopropylethyl amine (0.57 ml, 3.28 mmol) and then heated at 85° C. for 44 hours in a sealed vial. The reaction mixture was allowed to cool and concentrated in vacuo. The residue was dissolved with methyl sulfoxide and acidified with 0.28 ml of trifluoroacetic acid. The solution was then purified by reverse phase chromatography with 30-90% CH₃CN/H₂O to give 141 mg (49% yield) of a white solid. m/z(M+H)⁺676

Analysis: C₃₂H₃₁N₇O₃+1.80 TFA calcd: C, 48.54; H, 3.75; N, 11.13; found: C, 48.54; H, 3.83; N, 11.07.

HRMS calcd: 676.2465; Found: 676.2468

¹H NMR(400 MHz, DMSO): 1.22(6H, d), 4.08(1H, m), 4.27(2H, d), 4.33(2H, s), 5.10(2H, s), 6.73(1H, s), 6.77(1H, s), 6.78(1H, s), 6.92(1H, s), 7.20(2H, d), 7.20-7.77(4H, m), 8.60(1H, t).

### Example 141

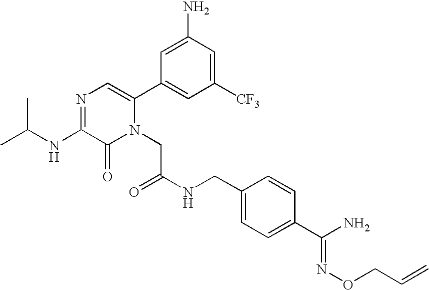

### Example 141

A solution of free amidine (200 mg, 0.328 mmol) and O-allylhydroxylamine hydrochloride (144 mg, 1.31 mmol) in 2 ml of ethanol was treated with N,N-diisopropylethyl amine(0.57 ml, 3.28 mmol) and then heated at 85° C. for 37 hours in a sealed vial. The reaction mixture was allowed to cool and was treated with 0.28 ml of trifluoroacetic acid. The solution was then purified by reverse phase chromatography with 20-50% CH₃CN/H₂O to give 104 mg (41% yield) of a white solid. m/z(M+H)⁺558

Analysis: C₂₇H₃₀N₇O₃+1.80 TFA+0.50 H₂O calcd: C, 47.62; H, 4.28; N, 12.70; found: C, 47.62; H, 4.29; N, 12.75.

HRMS calcd: 558.2435; Found: 558.2423

¹H NMR(400 MHz, DMSO): 1.22(6H, d), 4.07(1H, m), 4.29(2H, d), 4.34(2H, s), 4.48(2H, d), 5.23(1H, d), 5.36(1H, d), 6.02(1H, m), 6.73(1H, s), 6.77(1H, s), 6.79(1H, s), 6.92(1H, s), 7.26(2H, d), 7.59(2H, d), 8.63(1H, t)

### Example 142

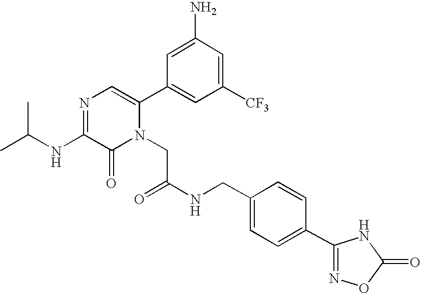

### Example 142

To free acid (150 mg, 0.369 mmol) and 4-aminomethyl-benzoxadiazolinone (101 mg, 0.442 mmol) in 2 ml of N,N-dimethylformamide was added N,N-diisopropylethylamine (0.32 ml, 1.84 mmol) and then benzotriazol-1-yl tetramethyluronium tetrafluoroborate(141 mg, 0.442 mmol). The solution was stirred for 1 hour, acidified with trifluoroacetic acid and purified by reverse phase chromatography with 5-70% CH₃CN/H₂O to give 35 mg (14% yield) of a light yellow solid. m/z(M+H)⁺544

Analysis: C₂₅H₂₄N₇O₄+1.00 TFA calcd: C, 49.32; H, 3.83; N, 14.91; found: C, 49.46; H, 4.06; N, 14.67.

HRMS calcd: 544.1915; Found: 544.1914

¹H NMR(400 MHz, DMSO): 1.19(6H, d), 4.05(1H, m), 4.31(2H, d), 4.34(2H, s), 6.69(1H, s), 6.74(1H, s), 6.76(1H, s), 6.72(1H, s), 7.33(2H, d), 7.71(2H, d), 8.65(1H, t).

### Example 143

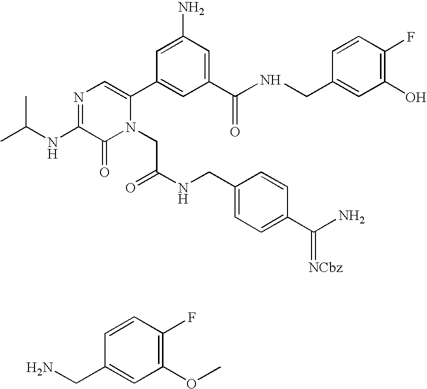

### 4-fluoro-3-methoxybenzylamine

### Example 143a

To 4-fluoro-3-methoxybenzonitrile (2.55 g, 16.9 mmol) in 75 ml of ethanol was added 0.85 g of 10% palladium on carbon and 7.5 ml of hydrogen chloride (conc.). The mixture was shaken on the Parr apparatus under 20 Psi of hydrogen for 5.5 hours. The mixture was filtered and concentrated in vacuo to give 3.19 g (99% yield) of a light pink solid. m/z(M+H)⁺156

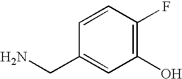

### 5-(aminomethyl)-2-fluorophenol

### Example 143b

The product from Example 143a (3.07 g, 16.1 mmol) in 9 ml of hydrogen chloride (conc.) was heated at 125° C. for 8 hours in a sealed tube. The solution was treated with 75 ml of ethanol and concentrated in vacuo to give 2.88 g (quantitative yield) of a tan solid. m/z(M+H) ⁺142

### Example 143c

To free acid(2.5 g, 3.90 mmol) in 15 ml of N,N-dimethylformamide was added N,N-diisopropylethylamine (5.4 ml, 31.2 mmol), 1-[3-(dimethylamino)propyl]-3-ethylcarbodiimide hydrochloride (0.82 g, 4.29 mmol), the product from Example 143b (0.76 g, 4.29 mmol) and the solution was stirred over the weekend. The solution was added to a solution of 40 ml of 1M HCl and 210 ml of water. The solution was neutralized with N,N-diisopropylethylamine and the precipitate was collected by vacuum filtration. The solid was purified by silica gel chromatography with 2-5% CH₃OH/CH₂Cl₂ to give 0.87 g (% yield) of an orange solid. m/z(M+H)⁺765

### Example 146

### N-{4-[(Z)-amino(hydroxyimino)methyl]-3-fluorobenzyl}-2-[6-[3-amino-5-(trifluoromethyl)phenyl]-3-(isopropylamino)-2-oxopyrazin-1(2H)-yl]acetamide

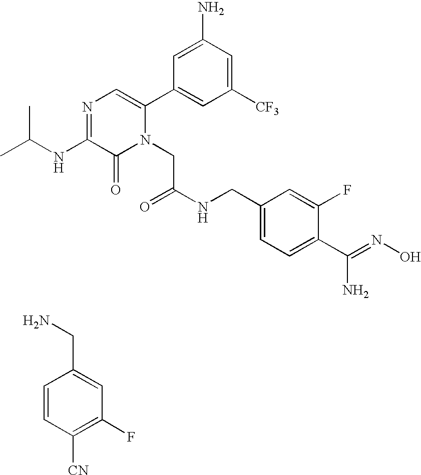

### Example 146a

To a 250 mL RBF was added the di-Boc-4-amino-2-fluoro-benzonitrile (5.4 g, 21.4 mmol) in 4 N HCl in dioxane (15 mL). The reaction stirred at room temperature for 1 hour. Checked by L.C. and M.S., the starting material was consumed and a new product that had a mass that corresponded to the product was observed. The excess HCl and dioxane was removed in vacuo to afford the HCl salt of the desired product. The resulting white solid was used with no further purification.

M.S. 151.01(MH+152.2)

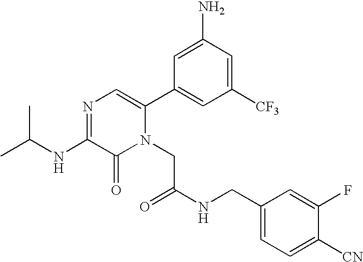

### 2-[6-[3-amino-5-(trifluoromethyl)phenyl]-3-(isopropylamino)-2-oxopyrazin-1(2H)-yl]-N-(4-cyano-3-fluorobenzyl)acetamide

### Example 146b

To a 250 mL RBF was added the amine(4.0 g, 21.4 mmol) and the free acid(4.0 g, 8.26 mmol) in DMF(50 mL).

To the reaction was added DIEA (8 mL). The reaction stirred for 15 minutes then TBTU (2.89 g, 9.0 mmol) was added. The reaction was stirred at room temperature overnight. The reaction was then poured into water(500 mL) and extracted with ethyl acetate(200 mL). The ethylacetate was washed again with 10% KHSO4 then dried over MgSO4. The organics were then concentrated in vacuo to afford Example 146b in 73% yield.

### Example 146c

To a 25 mL RBF was added Example 146b (1.3 g, 2.59 mmol) triethylamine (1 mL) and hydroxylamine hydrochloride (0.191 g, 2.75 mmol). The reaction was refluxed in ethanol (10mL) overnight. The resulting solution was diluted to 50 mL with water and methanol. The reaction was purified using reverse phase chromatography to afford the title compound (1.21 g) as a TFA salt in 87% yield.

NMR (400 MHZ, CDCL3): 1H 1.4 ppm (3H, d), 3.34 ppm (1H, m), 4.11 ppm(1H, q), 4.5 ppm (2H, d), 4.51 ppm (1H, s), 6.72 ppm (1H, s), 6.91 ppm (2H, m), 7.08 ppm (1H, s), 7.29 ppm (1H, d), 7.31 ppm (1H, s), 7.6 ppm (2H, m).

| | | | |
|---|---|---|---|
| Found | C: 44.30 | H: 3.71 | N: 13.09 |
| Calc. | C: 53.83 | H: 4.71 | N: 18.31 |

### Example 147

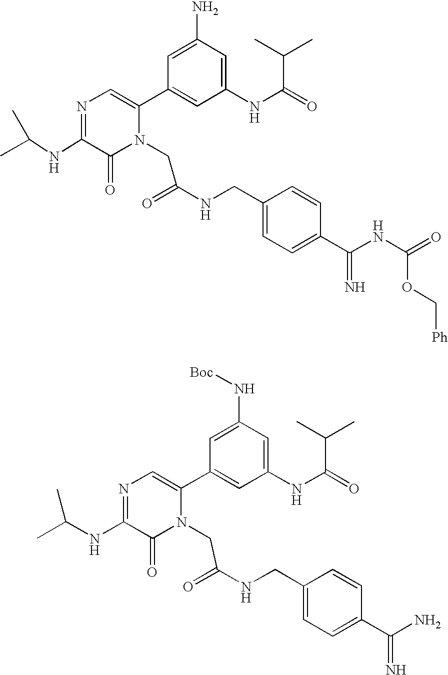

### Example 147a

The product of Example 19d (300 mg, 0.56 mmol) in AcOH (5 ml) was added Pd/C (100 mg). The mixture was set on hydrogenation shake at 40 psi for 10 hr, then filtered and concentrated to yield 300 mg of solid (99%).

### Example 147b

0.3 g, 0.58 mmol of Example 147a and benzyl chloridocarbonate(0.13 g, 0.75 mmol) in THF (2 ml) was added NMM(75 mg, 0.75 mmol). The mixture was kept stirring for 3 hr. The mixture was concentrated and then TFA/CH2Cl2 was added. The mixture was kept stirring at RT for 1 hr, then concentrated and purified on RP-HPLC to yield 85mg of solid (13%).

HRMS calcd for C₃₅H₄₀N₈O₅ (M+H): 653.3194. Found: 653.3221.

Anal. Calcd for C₃₅H₄₀N₈O₅ + 1.95TFA + 1.2H2O:

| | | | |
|---|---|---|---|
| | C: 52.10; | H: 4.98; | N: 12.49. |
| Found: | C: 52.11; | H: 4.98; | N: 12.45. |

¹H NMR (DMSO-d₆, 300 MHz) δ 1.10 (d, 6H), 1.24 (d, 6H), 4.11 (m, 2H), 4.39 (d, 2H), 4.46 (s, 2H), 5.36 (s, 2H), 6.32 (s, 1H), 6.68 (s, 1H), 6.82 (s, 1H), 7.12 (s, 1H), 7.41-7.51 (m, 6H), 7.79 (d, 2H), 7.93 (t, 1H), 8.70 (t, 1H), 9.74 (s, 1H).

### Example 148

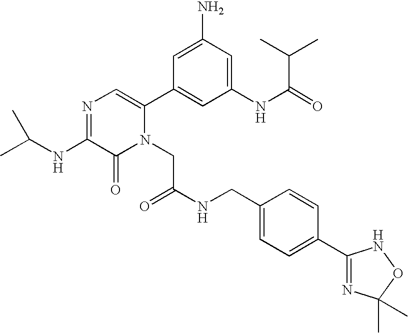

### Example 148

The product of Example 19d (300 mg, 0.47 mmol) in AcOH (2 ml) and 2,2-dimethoxypropane(2 ml) was heated to 70° C. for 3 hr. The mixture was concentrated and then TFA/CH2Cl2 (2 ml/1 ml) was added. The mixture was kept stirring at RT for 1 hr, then concentrated and purified on RP-HPLC to yield 100 mg of solid (27%).

HRMS calcd for C₃₀H₃₈N₈O₄ (M+H): 575.3089. Found: 575.3061.

Anal. Calcd for C₃₀H₃₈N₈O₄ + 1.6TFA + 1.05H2O:

| | | | |
|---|---|---|---|
| | C: 51.32; | H: 5.38; | N: 14.51. |
| Found: | C: 51.38; | H: 5.41; | N: 14.43. |

¹H NMR (DMSO-d₆, 300 MHz) δ 1.10 (d, 6H), 1.27 (d, 6H), 1.47 (s, 6H), 2.62 (m, 1H), 4.12 (m, 3H), 4.35(d, 2H), 4.44(s, 2H), 6.40 (s, 1H), 6.70 (s, 1H), 6.93 (s, 1H), 7.22(s, 1H), 7.30 (d, 2H), 7.40 (bs, 1H), 7.62(d, 2H), 8.61(t, 1H), 9.82 (s, 1H).

### Example 149

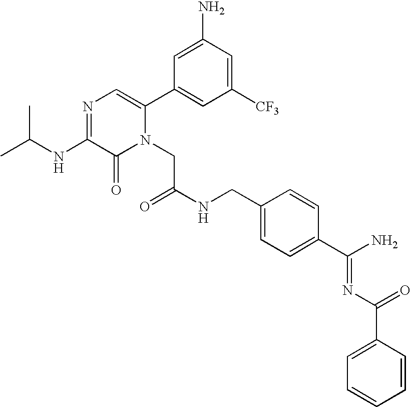

### Example 149

0.5 g (1.1 mmol) N,N-di-Boc-4-amino-(benzoyl)benzamidine was deprotected in 25 mL CH₂Cl₂/TFA (4:1) for 30 minutes and the solvent was evaporated thoroughly to dryness. 0.48 g (1 mmol) of the free amidine was coupled with the 4-amino-(benzoyl)benzamidine in 25 mL DMF in the presence of 0.35 g (1.1 mmol) TBTU and 0.525 mL (3 mmol) DIPEA with stirring for 2 hours. DMF was evaporated and the product was precipitated by addition of 200 mL water and filtered. The crude product was dissolved in the mixture of AcN and H₂O and purified on preparative HPLC using a gradient of acetonitrile (10-50% AcN in 30 minutes), yielding the product at 45% AcN, 0.365 g (51%) as a white solid.

MH⁺=606.2

¹HNMR: 400 MHz, CD₃OD: 8.08-8.02 (d, 1H), 7.96-7.84 (m, 3H), 7.80-7.72 (m, 1H), 7.66-7.50 (m, 4H), 7.40-7.34 (d, 1H), 7.05-6.80 (m, 3H), 4.58-4.49 (m, 4H), 4.14-4.00 (m, 1H) and 1.40-1.30 (m, 6H).

⁹FNMR: 400 MHz, CD₃OD: −64.9 (s, 3F)

Elemental analysis: C₃₁H₃₀N₇O₃F₃ + 1.5xTFA + 2xH₂O

| | | | |
|---|---|---|---|
| Found | C: 50.20 | H: 4.41 | N: 11.59 |
| Calc. | C: 50.25 | H: 4.40 | N: 12.06 |

### Example 150

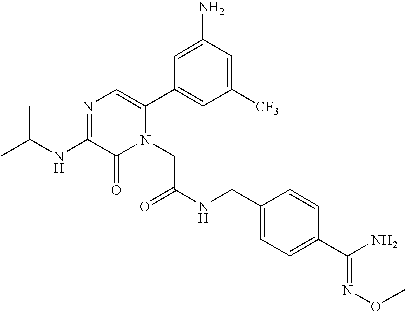

### Example 150a

3.65 g (10 mmol) N,N-di-Boc-4-amino-benzamidoxime was dissolved in 11 mL 1N NaOH and cooled in ice bath. 1.32 g (10.5 mmol) dimethyl sulphate was added slowly and the mixture was stirred for 4 hours in ice bath. The solvent was evaporated and the product was isolated on preparative HPLC using a gradient of acetonitrile (10-50% AcN in 30 minutes), yielding 1.3 g (34%) product at 50% AcN, as an oil. MH⁺=380.6

### Example 150b:

0.42 g (1.1 mmol) N,N-di-Boc-4-amino-O-methyl-benzamidoxime was deprotected in 25 mL CH₂Cl₂/TFA (4:1) for 30 minutes and the solvent was evaporated thoroughly to dryness. 0.48 g (1 mmol) of the free acid was coupled with the 4-amino-O-methyl-benzamidoxime in 25 mL DMF in the presence of 0.35 g (1.1 mmol) TBTU and 0.7 mL (4 mmol) DIPEA with stirring for 16 hours. DMF was evaporated and the product was precipitated by addition of 200 mL water and filtered. The crude product was dissolved in the mixture of AcN and H2O and purified on preparative HPLC using a gradient of acetonitrile (10-50% AcN in 30 minutes), yielding the title product at 40% AcN, 0.13 g (17%) as a white solid.

MH⁺=532.3

¹HNMR: 400 MHz, CD₃OD: 7.70-7.64 (d, 2H), 7.46-7.38 (d, 2H), 7.02 (s, 1H), 6.90-6.84 (d, 2H), 6.68 (s, 1H), 4.54 (s, 2H), 4.42 (s, 2H), 4.14-4.00 (m, 1H), 3.32-3.28 (m, 3H) and 1.40-1.32 (m, 6H).

¹⁹FNMR: 400 MHz, CD₃OD: −65.0 (s, 3F)

Elemental analysis: C₂₅H₂₈N₇O₃F₃ + 1.5xTFA + 2.1xH₂O

| | | | |
|---|---|---|---|
| Found | C: 45.41 | H: 4.24 | N: 12.92 |
| Calc. | C: 45.42 | H: 4.59 | N: 13.24 |

### Example 151

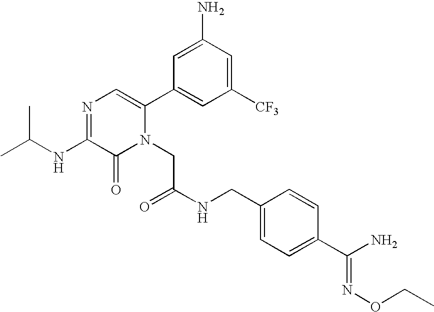

### Example 151

0.51 g (1.3 mmol) N,N-di-Boc-4-amino-O-ethyl-benzamidoxime was deprotected in 25 mL CH2Cl2/TFA (4:1) for 30 minutes and the solvent was evaporated thoroughly to dryness. 0.58 g (1.3 mmol) of the free acid was coupled with the 4-amino-O-ethyl-benzamidoxime in 25 mL DMF in the presence of 0.48 g (1.5 mmol) TBTU and 0.7 mL (4 mmol) DIPEA with stirring for 16 hours. DMF was evaporated and the crude product was dissolved in the mixture of AcN and H2O and purified on preparative HPLC using a gradient of acetonitrile (10-50% AcN in 30 minutes), yielding the title product at 42% AcN, 0.39 g (50%) as a white hygroscopic solid.

MH⁺=546.4

¹HNMR: 400 MHz, CD₃OD: 7.70-7.64 (d, 2H), 7.48-7.42 (d, 2H), 7.02 (s, 1H), 6.90-6.84 (d, 2H), 6.68 (s, 1H), 4.56 (s, 2H), 4.49 (s, 2H), 4.20-4.10 (m, 2H), 4.09-4.00 (m, 1H) and 1.43-1.34 (m, 9H).

¹⁹FNMR: 400 MHz, CD₃OD: -64.8 (s, 3F)

Elemental analysis: C₂₆H₃₀N₇O₃F₃ + 2xTFA + 1.8xH₂O

| | | | |
|---|---|---|---|
| Found | C: 44.73 | H: 4.23 | N: 12.06 |
| Calc. | C: 44.70 | H: 4.45 | N: 12.16 |

### Example 152

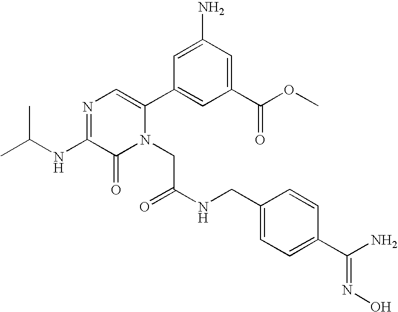

### Example 152

0.31 g (0.65 mmol) nitrile compound was dissolved in 25 mL EtOH and it was refluxed in the presence of 0.14 g (2 mmol) hydroxylamine.HCl and 0.7 mL (4 mmol) DIPEA for 3 hours. The solvent was evaporated and the product was purified on preparative HPLC using a gradient of acetonitrile (10-50% AcN in 30 minutes). Yield: 0.22 g (46%) as a white solid.

MH⁺=508.4

¹HNMR: 400 MHz, CD₃OD: 7.66-7.60 (d, 2H), 7.48-7.37 (m, 3H), 7.30 (s, 1H), 6.90-6.86 (t, 1H), 6.64 (s, 1H), 4.58 (s, 2H), 4.42 (s, 2H), 4.10-4.00 (m, 1H), 3.86 (s, 3H) and 1.40-1.34 (m, 6H).

Elemental analysis: C₂₅H₂₉N₇O₅ + 1.5xTFA + 1.2H₂O

| | | | |
|---|---|---|---|
| Found | C: 48.19 | H: 4.70 | N: 13.95 |
| Calc. | C: 48.03 | H: 4.74 | N: 14.00 |

### Example 153

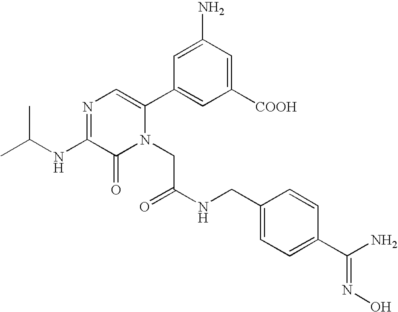

### Example 153

0.33 g (0.45 mmol) of methyl ester of Example 152 was dissolved in 15 mL MeOH and hydrolyzed by adding 1.5 mL 1N LiOH. After 10 minutes stirring 3 mL AcOH was added and the mixture was purified on preparative HPLC using a gradient of acetonitrile (0-40% AcN in 30 minutes). Product peak was collected at 19% AcN, yielding 0.22 g (69%) as a white solid.

MH⁺=494.3 and MNa⁺=516.2

¹HNMR: 400 MHz, CD₃OD: 7.52 (s, 1H), 7.51-7.44 (d, 2H), 7.36 (s, 1H), 7.16-7.10 (d, 2H), 7.08 (s, 1H), 6.60 (s, 1H), 4.58 (s, 2H), 4.22 (s, 2H), 3.95-3.84 (m, 1H) and 1.30-1.21 (m, 6H).

Elemental analysis: C₂₄H₂₇N₇O₅ + 2.8xTFA +1.5xH₂O

| | | | |
|---|---|---|---|
| Found | C: 42.30 | H: 4.00 | N: 11.80 |
| Calc. | C: 42.33 | H: 3.94 | N: 11.67 |

### Example 155a

To a 250 mL RBF was added NaH(0.54 g,14 mmol, 60% in mineral oil) in THF(25 mL). The reaction was cooled to 0° C. To the slurry was added the oxime (5 g, 13.7 mmol). The reaction stirred for 1 hour at 0° C. To the reaction was added the (−)-menthyl chloroformate(2.99 g, 13.7 mmol). The reaction stirred 4 hours as it warmed to room temperature. To the reaction was quenched with water and the organics were extracted with ethyl acetate(100 mL). The combined organics were then dried over MgSO4 and concentrated in vacuo. The resulting oil was purified on silica to afford Example 155a (3.8 g) in 50% yield.

M.S.547.68(MH+548.7)

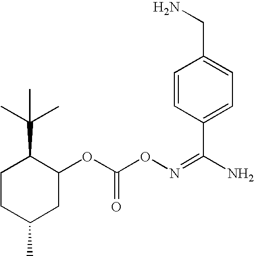

### 4-(aminomethyl)-N′-[({[(2S,5R)-2-tert-butyl-5-methylcyclohexyl]oxy}carbonyl)oxy]benzenecarboximidamide

### Example 155b

To a 250 mL RBF was added Ex-10a(1.8, 3.29 mmol) in 4 N HCl/dioxane(20 mL). The reaction stirred for 2 hours and was complete by L.C. and M.S.. The excess HCl and dioxane was remove in vacuo to afford Example 155b as an HCl salt and a white powder. The product was used with no further purification.

M.S. 285.43(MH+286.5)

### Example 155c

### To a 250 mL RBF was added Example 155b (1.38 g, 3.29 mmol) was added the acid(1,3 g, 2.7 mmol),and DIEA (8 mL) in DMF(30 mL). To the solution was added TBTU (1.3 g, 4.11 mmol) The reaction stirred over night. To the reaction was added 10% KHSO4 (100 mL) and ethyl acetate (200 mL). The organics were collected and washed with brine then dried over MgSO4. The organics were then concentrated in vacuo and the resulting oil was purified on silica to afford Ex-10 (1.3 g) in 69% yield. NMR (400 MHZ, CDCL3): 1H 0.85 ppm (3H, d), 0.98 ppm (6H, t), 1.12 ppm(2H, m), 1.2 ppm (6H, d), 1.38 ppm (1H,m), 1.45 ppm (1H, m), 1.63 ppm (2H, d), 2.0 ppm (1H, s), 3.30 ppm (1H, s), 4.0 ppm (1H, m), 4.15 ppm (1H, m), 4.26 ppm (2H, d), 4.31 ppm (2H, s), 4.55 ppm (1H, dt) ), 5.8 ppm (2H, bs), 6.69 ppm (1H, s), 6.80 ppm (4H, d), 6.85 ppm (2H, m), 7.21 ppm (2H, d), 7.62 ppm (2H, d), 8.6 ppm (1H, t).

### Example 156

### N-{4-[(Z)-amino({[(benzyloxy)carbonyl]oxy}imino) methyl]benzyl}-2-[6-[3-amino-5-(trifluoromethyl)phenyl]-3-(isopropylamino)-2-oxopyrazin-1(2H)-yl]acetamide

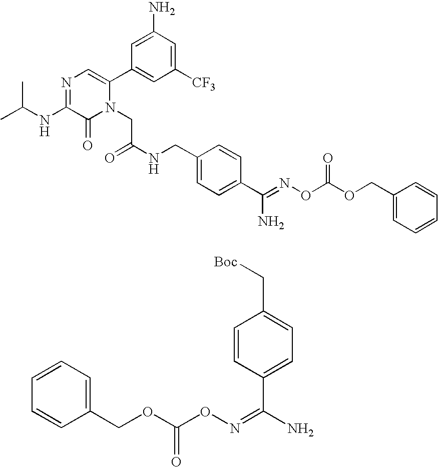

### Di(tert-butyl) 4-[(E)-amino({[(benzyloxy)carbonyl]oxy}imino)methyl]benzylimidodicarbonate

### Example 156a

To a 250 mL RBF was added NaH(0.54 g,14 mmol, 60% in mineral oil) in THF(25 mL). The reaction was cooled to 0° C. To the slurry was added the oxime (5 g, 13.7 mmol). The reaction stirred for 1 hour at 0° C. To the reaction was added the Cbz-OSu(3.4 g, 13.7 mmol). The reaction stirred 4 hours as it warmed to room temperature. To the reaction was quenched with water and the organics were extracted with ethyl acetate(100 mL). The combined organics were then dried over MgSO4 and concentrated in vacuo. The resulting oil was purified on silica to afford Ex-15a(4.81 g) in 70% yield.

M.S.499.56(MH+500.8)

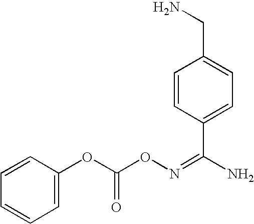

### 4-(aminomethyl)-N′-{[(benzyloxy)carbonyl]oxy}benzenecarboximidamide

### Example 156b

To a 250 mL RBF was added Example 156a (1.17 g, 2.34 mmol) in 4 N HCl/dioxane(10 mL). The reaction stirred for 2 hours and was complete by L.C. and M.S. The excess HCl and dioxane was removed in vacuo to afford Example 156b as an HCl salt and a white powder. The product was used with no further purification.

M.S. 299.45(MH+300.2)

### Example 156c

To the 250 mL flask from Example 156b was added the acid(1.07 g, 2.2 mmol),and DIEA (8 mL) in DMF(50 mL). To the solution was added TBTU(1.3 g, 4.11 mmol) The reaction stirred over night. To the reaction was added 10% KHSO4 (100 mL) and ethylacetate(200 mL). The organics were collected and washed with brine then dried over MgSO4. The organics were then concentrated in vacuo and the resulting oil was purified on silica to afford Ex-15 (0.75 g) in 52% yield.

M.S. 651.65(MH+652.6)

NMR (400 MHZ, CDCL3): 1H 1.19 (6H, d), 4.08 ppm (1H, q), 4.28 ppm (2H, d), 4.35 ppm (2H, s), 6.68 ppm (1H, s), 6.79 ppm (4H, m), 6.9 ppm (1H, s), 7.22 ppm (2H, d), 7.4 ppm (6H, m), 7.60 ppm (2H, d), 8.61 ppm (1H, t).

### Example 157

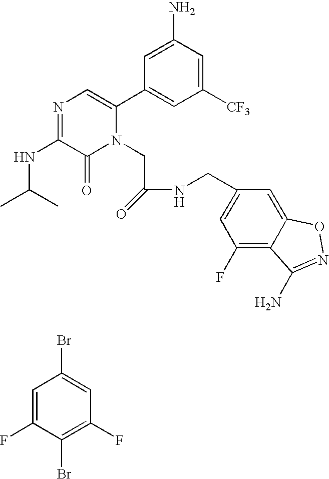

### 2,5-dibromo-1,3-difluorobenzene

### Example 157a

To a solution of 4-Bromo-2,6-difluoraniline (100.0 g,0.48 mol) in CH₃CN (600 ml) was added CuBr₂ (214.0 g,0.95 mol). The resulting mixture was cooled to 5° C. and t-butylnitrite (99.0 g, 0.99 mol) was added dropwise over 20 min. The reaction was stirred for an additional 2 hours at room temperature then the entire mixture was partitioned between a mixture of CHCl₃ (1.5 L) and 2N HCl (1.0 L). The layers were separated and the aqueous was extracted again with CHCl₃. The chloroform layers were combined, dried over Na₂SO₄, filtered and concentrated to give 96.0 g of Example 157a (73%) as a white solid.

MS(EI,70 ev): m/z 272(M⁺,100), 191(40), 112(80); Calcd for C6H₂Br₂F₂=271.88

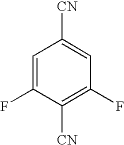

### 2,6-difluoroterephthalonitrile

### Example 157b

To a solution of Example 157a, 2,5-dibromo-1,3-difluorobenzene (49.0 g,180 mmol) in DMF (500 ml), was added Cu(I)CN (48.0 g, 0.54 mol). The reaction was stirred at 185° C. for 2 hours. The reaction mixture was cooled to room temperature and filtered. The filtrate was concentrated and the crude residue was purified by flash chromatography (Merck 230-400 mesh SiO₂, chloroform:hexane; 8:2) to afford 19.0 g of Example 157b (64%) as light yellow solid.

MS(EI,70 ev): m/z 164(M⁺,100), 137(12), 113(01); Calcd for C₈H₂F₂N₂=164.0

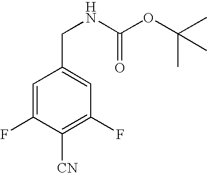

### Tert-butyl 4-cyano-3,5-difluorobenzylcarbamate

### Example 157c

To a solution of Example 157b, 2,6-difluoro-terephthalonitrile, (18.87 g,115 mmol) in 95% Ethanol (200 ml), was added PtO₂ (2.0 g, 10% by w/w) followed by di-tert-butyl-pyrocarbonate ( 27.59 g, 126 mmol). The mixture was stirred under 60 psi of hydrogen at room temperature for 16 hours. The catalyst was removed by filtration through Celite. The filtrate was concentrated and the crude residue was purified by flash chromatography (Merck 230-400 mesh SiO₂, Hexane:Ethyl acetate; 8:2) to give 15.0 g of Example 157c as white solid.

HRMS 269.1069 found for C₁₃H₁₄F₂N₂O₂; 269.1096 calcld.

¹H NMR, 300 MHz, DMSO-d₆ δ 7.55 (bt, 1H), 7.28 (s, 1H), 7.22 (s, 1H), 4.24 (d, J=6.0 Hz, 2H), 1.39 (s, 9H).

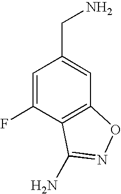

### 6-(aminomethyl)-4-fluoro-1,2-benzisoxazol-3-amine

### Example 157d

A solution of potassium tert-butoxide (1.08 g,9.7 mmol) and acetoxime (707 mg,9.7 mmol) in dry tetrahydrofuran (40 ml) was stirred at room temperature for 30 min. To this solution was added a solution of Example 157c, tert-butyl 4-cyano-3,5-difluorobenzylcarbamate (2.36 g,8.8 mmol) in dry tetrahydrofuran (20 ml). The resulting reaction mixture was stirred at room temperature for 3 hours. The reaction was concentrated and partitioned between ethyl acetate (250 ml) and saturated aqueous ammonium chloride (150 ml). The ethyl acetate layer was separated and dried over magnesium sulfate. The solids were removed by filtration and the filtrate was concentrated. The residue was treated with 95% ethanol (40 ml), water (35 ml) and conc. HCl (20 ml) and this mixture was heated at 80° C. for 3 hours. The reaction mixture was concentrated and the pH was adjusted to ˜9 with 4 N sodium hydroxide. The solids obtained were extracted with ethyl acetate. The ethyl acetate layer was dried with magnesium sulfate, filtered and the filtrate concentrated to give 840 mg of Example 157d (53 %) as a tan solid.

MS(EI,70 ev): m/z 181(M⁺100), 161(85), 153(37), 83 (37) Calcd for C₈H₈FN₃O=181.17

¹H NMR, 400 MHz, DMSO-d₆ δ 7.28 (s, 2H), 7.05 (s, 1H), 7.03 (s, 1H), 6.22 (s, 2H), 3.82 (s, 2H).

### Example 157e

The carboxylic acid, [6-[3-amino-5-(trifluoromethyl)phenyl]-3-(isopropylamino)-2-oxopyrazin-1(2H)-yl]acetic acid, (890 mg,2 mmol), the benzyl amine, 6-(aminomethyl-4-fluoro-1,2-benzisoxazol-3-amine, Example 157d (400 mg, 2.2 mmol) and HOBT-H₂O (2.7 g, 20 mmol) were placed in a flask. DMF (10 ml) and CH₂Cl₂ (40 ml) were added. To this stirred solution was added polymeric DCC resin (8.28 g, loading 1.38 mmol/g, 14 mmol) and triethylamine (1.39 ml) and the resulting mixture stirred over night. The resin was removed by filtration. The filtrate was concentrated and the residue was purified by prep HPLC (RP, 10-90% gradient, acetonitrile in 0.1% TFA) to give 380 mg of Example 157e as light yellow solid.

LCMS (5-95% acetonitrile in 0.1% TFA over 14 min): Retention time=4.15 min; (M+H)⁺=534

¹H-NMR, 400 MHz, DMSO-d₆ δ 8.71 (t, J=5.6 Hz, 1H), 7.14 (s, 1H), 6.90 (s, 1H), 6.78 (s 1H), 6.70 (s, 1H), 6.27 (s, 1H), 5.79 (s, 1H), 4.38 (d, J6.0 Hz, 2H), 4.36 (s, 1H), 4.07 (m, 1H) 1.20 (d, J=7.3 Hz, 6H)

### Example 158

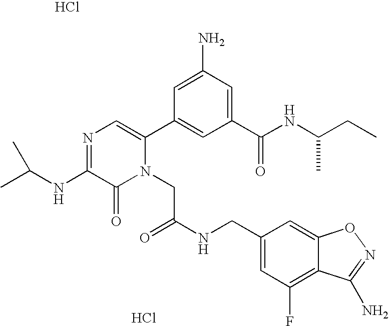

### Example 158

The carboxylic acid, [3-(isopropylamino)-6-[3-({[(1S)-1-methylpropyl]amino}carbonyl)-5-aminophenyl]-2-oxopyrazin-1(2H)-yl]acetic acid (tan solid, M+H=475) (400 mg, 0.84 mmol), the benzyl amine, 6-(aminomethyl)-4-fluoro-1,2-benzisoxazol-3-amine, Example 157d (236 mg, 0.93 mmol) and HOBt-H₂O (153 mg, 1.00 mmol) were dissolved in DMF (16 mL) and CH₂Cl₂ (100 mL). To this gently stirred solution was added polymeric DCC resin (4.00 g, loading 1.38 mmol/g, 5.52 mmol) and triethylamine (0.5 mL) and the resulting mixture stirred over night. The resin was removed by filtration. The filtrate was concentrated and the residue was purified by prep HPLC (RP, acetonitrile gradient in 0.1% TFA). Co-evaporation with 1N HCl afforded 120 mg of Example 158 (22%) as a white solid.

LCMS (RP, 15-50% gradient acetonitrile in 0.1% ammonium acetate over 14 min): retention time=5.72; (M+H)⁺=565.

### Example 159

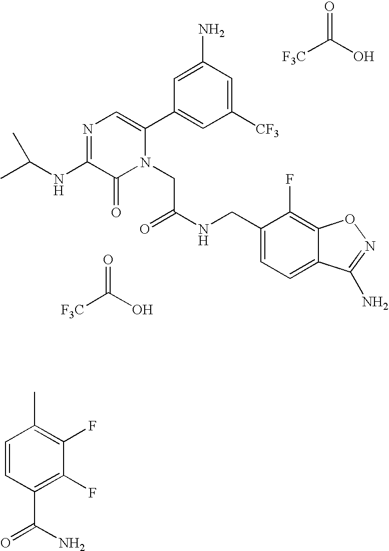

### 2,3-difluoro-4-methylbenzamide

### Example 159a

To a solution of NH₄OH (350 mL) and toluene (450 mL) at 0° C. was added 2,3-difluoro-4-methylbenzoyl chloride (35 g, 184 mmol). The reaction mixture stirred for 16 hours at ambient temperature. The precipitate was filtered and dried to give Example 159a as a white solid.

LCMS (RP, 15-90% gradient acetonitrile in 0.1% ammonium acetate): retention time=3.43; (M+H)⁺=172

¹H NMR, 300 MHz, DMSO-d₆ δ 7.62-7.78 (broad m, 2 H), 7.36-7.30 (m, 1H), 7.17-7.11 (m, 1H), 2.29 (d, J=2.10-long range F-coupling, 3H).

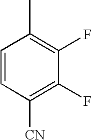

### 2,3-difluoro-4-methylbenzonitrile

### Example 159b

To 2,3-difluoro-4-methylbenzamide, Example 159a (28.9 g, 169 mmol) in CH₂Cl₂ (1 L) and TEA (47.1 mL, 338 mmol) at 0° C. was added triflic anhydride (45.4 mL, 338 mmol) dropwise. The reaction was allowed to reach ambient temperature overnight. The mixture was quenched with brine and H₂O (1 L each), the organic layer was separated followed by extraction of the aqueous layer with CH₂Cl₂ (3×1 L). The organic layers were combined, dried over MgSO₄, filtered and concentrated. The mixture was filtered through silica gel with 10:1 hexane/ethyl acetate. The product which eluted first, was collected and concentrated to give Ex-23b. LCMS (RP, 15-90% acetonitrile gradient in 0.1% ammonium acetate over 14 min): retention time=6.43 min; (M+MeOH+H)⁺=186.

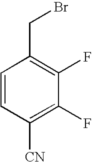

### 4-(bromomethyl)-2,3-difluorobenzonitrile

### Example 159c

To 2,3-difluoro-4-methylbenzonitrile, Example 159b (3.23 g, 21.1 mmol) in CCl₄ (75 mL) was added benzoyl peroxide (1.05 g, 4.22 mmol) and N-bromosuccinimide (4.55 g, 25.3 mmol) and the resulting mixture was heated to reflux for 8 hours. The reaction was allowed to cool, diluted with CH₂Cl₂ (75 mL), washed with H₂O and brine (75 mL each). The organic layer was separated and dried over MgSO₄, filtered and concentrated. The crude material was purified by flash chromatography (Merck 230-400 mesh SiO₂, 80:1 hexane/ethyl acetate) to give Example 159c as a white solid.

LCMS (RP, 15-90% acetonitrile gradient in 0.1% ammonium acetate over 14 min): retention time=6.83 min; (M+H)⁺=232

¹H-NMR, 300 MHz, CDCl₃ δ 7.42-7.27 (m, 2H), 4.49 (d, J=1.2 Hz—long range F-coupling, 2H).

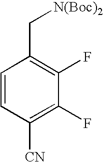

### Di(tert-butyl) 4-cyano-2,3-difluorobenzylimidodicarbonate

### Example 159d

To a solution of NaH (1.09 g, 27.2 mmol) and di-tert-butyliminodicarboxylate in THF (50 mL) was added 4-(bromomethyl)-2,3-difluorobenzonitrile, Example 159c (5.73 g, 24.7 mmol) in THF (50 mL). The reaction was stirred at ambient temperature for 16 hours and concentrated. The residue was taken up in ether (100 mL) and washed with H₂O and brine (100 mL each). The organic layer was dried over MgSO₄, filtered and concentrated. The material was purified by flash chromatography (Merck 230-400 mesh SiO₂100:1 CHCl₃/MeOH) to give Example 159d as a white solid. LCMS (RP, 15-90% acetonitrile gradient in 0.1% ammonium acetate over 14 min): retention time=9.19 min; negative ion mode (M−H)⁻=367

¹H-NMR, 300 MHz, CDCl₃ δ 7.37-7.32 (m, 1H), 7.13-7.08 (m, 1H) 4.89 (s, 2H), 1.46 (s, 18H).

¹⁹F-NMR, 282 MHz, CDCl₃ 67 −131.40 (m, 1F), −140.09 (m, 1F).

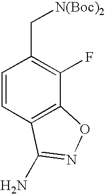

### Di (tert-butyl) (3-amino-7-fluoro-1,2-benzisoxazol -6-yl)methylimidodicarbonate

### Example 159e

A solution of acetohydroxamic acid (2.46 g, 32.8 mmol) and Potassium tert-butoxide (3.87 g, 32.8 mmol) in DMF (150 mL) was stirred at ambient temperature for 0.5. To this mixture was added di(tert-butyl) 4-cyano-2,3-difluorobenzylcarbamate, Example 159d, in DMF (20 mL) and the reaction was stirred for 16 hours at room temperature. The mixture was diluted with brine (20 mL) and ethyl acetate (20 mL). The organic layer was separated and the aqueous layer extracted with ethyl acetate (3×20 mL). The organic layers were combined dried over magnesium sulfate, filtered and concentrated to give Example 159e.

LCMS (RP, 15-90% acetonitrile gradient in 0.1% ammonium acetate over 14 min): retention time=8.24; (M+H)⁺=382

HRMS (M+Na)⁺404.1594 found for C₁₈H₂₄FN₃O₅Na; 404.1592 calc'd.

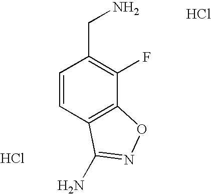

### 6-(aminomethyl)-7-fluoro-1,2-benzisoxazol-3-amine dihydrochloride

### Example 159f

A solution of di(tert-butyl) 4-cyano-2,3 difluorobenzylimidodicarbonate, Example 159e, and 4 N HCl in dioxane was stirred at room temperature for 16 hours. The reaction was concentrated to give Ex-23f as a white solid.

LCMS (RP, 5-90% acetonitrile gradient in 0.1% TFA over 14 min): retention time=1.14 min; (M+H)⁺=182. HRMS

(M+H)⁺=82.0734 found for C₈H₉FN₃O; 182.0724 calc'd.

### Example 159 g

The carboxylic acid, [3-amino-5-(trifluoromethyl)phenyl]-3-(isopropylamino)-2-oxopyrazin-1(2H)-yl]acetic acid, (461 mg, 1.00 mmol) and HOBt-H₂O (153 mg, 1.00 mmol) were placed in a flask. DMF (20 mL) and CH₂Cl₂ (100 mL) were added. To this gently stirred solution was added polymeric DCC resin (4 g, loading 1.4 mmol/g, 5.6 mmol). The mixture was allowed to stir for 1 hour at ambient temperature followed by addition of the benzyl amine, 6-(aminomethyl)-7-fluoro-1,2-benzisoxazol-3-amine dihydrochloride, Example 159f (350 mg, 1.40 mmol) in DMF (75 mL), CH₂Cl₂ (25 mL) and excess TEA (3 mL). The resulting mixture was stirred over night followed by gentle heating to 40° C. for 1 hour. The resin was filtered and the residue was purified by prep HPLC (RP, acetonitrile gradient in 0.1% TFA) to afford 261 mg (34%) of Example 159 g as an amorphous solid.

LCMS (RP, 15-90% acetonitrile in 0.1% ammonium acetate over 14 min): retention time: 6.72 min; (M+H)⁺=534, Negative Ion mode (M−H)⁻=532.

### Example 160

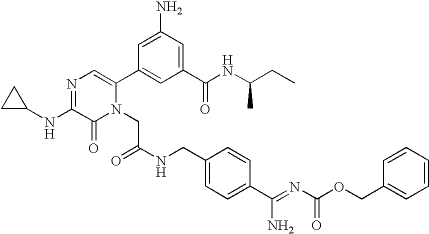

### Example 160

¹H NMR (400 MHz, DMF-d₇) δ 9.51 (br s, 2H), 8.56 (t, J=5.9 Hz, 1H), 8.06-8.00 (m, 3H), 7.48-7.31 (m, 7H), 7.14-7.11 (m, 2H), 6.87 (s, 1H), 6.78 (s, 1H), 5.55 (br s, 2H), 5.21 (s, 2H), 4.59 (s, 2H), 4.46 (d, J=5.6 Hz, 2H), 4.05-3.98 (m, 1H), 2.90-2.85 (m, 1H), 1.63-1.49 (m, 2H), 1.17 (d, J=6.6 Hz, 3H), 0.89 (t, J=7.4 Hz, 3H), 0.78-0.65 (m, 4H); ¹³C NMR (100 MHz, DMF-d₇) δ 167.95, 167.50, 166.9, 165.0, 152.15, 151.95, 149.9, 144.3, 138.2, 137.4, 134.1, 133.8, 130.4, 129.0, 128.59, 128.49, 128.38, 127.7, 122.0, 117.8, 116.7, 114.2, 66.9, 48.6, 47.4, 42.9, 29.8, 24.4, 20.5, 11.0, 6.7; HRMS (ES) calcd for C₃₆H₄₁N₈O₅ 665.3194, found 665.3230.

### Example 161

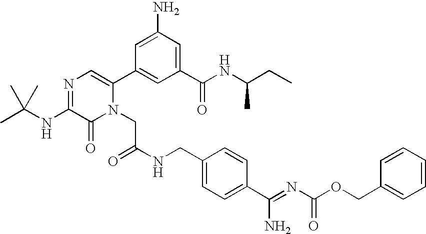

### Example 161

¹H NMR (400 MHz, DMF-d₇) δ 9.50 (br s, 1H), 9.12 (br s, 1H), 8.56 (t, J=5.8 Hz, 1H), 8.07-8.01 (m, 4H), 7.48-7.31 (m, 7H), 7.13 (s, 1H), 6.85 (s, 1H), 6.75 (s, 1H), 6.25 (s, 1H), 5.53 (s, 2H), 5.21 (s, 2H), 4.60 (s, 2H), 4.46 (d, J=5.8 Hz, 2H), 4.06-3.96 (m, 1H), 1.64-1.44 (m, 11H), 1.17 (d, J=6.7 Hz, 3H), 0.89 (t, J=7.5 Hz, 3H); 13C NMR (100 MHz, DMF-d₇) δ 168.0, 167.45, 166.95, 165.2, 152.5, 150.0, 149.9, 144.3, 138.3, 137.4, 134.1, 133.9, 129.81, 129.01, 128.58, 128.51, 128.37, 127.7, 121.6, 117.9, 116.6, 114.2, 66.9, 51.5, 48.8, 47.4, 43.0, 29.8, 28.5, 20.5, 11.0; HRMS (ES) calcd for C₃₇H₄₅N₈O₅ 681.3507, found 681.3498.

### Example 162

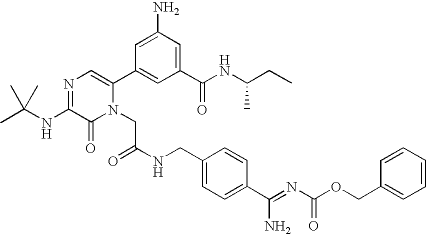

### Example 162

¹H NMR (400 MHz, DMF-d₇) δ 9.50 (br s, 1H), 9.11 (br s, 1H), 8.56 (t, J=5.8 Hz, 1H), 8.07-8.00 (m, 4H), 7.48-7.30 (m, 11H), 7.13 (s, 1H), 6.85 (s, 1H), 6.25 (s, 1H), 5.52 (s, 2H), 5.21 (s, 2H), 4.60 (s, 2H), 4.46 (d, J=5.8 Hz, 2H), 4.07-3.96 (m, 1H), 1.64-1.44 (m, 11H), 1.17 (d, J=6.7 Hz, 3H), 0.89 (t, J=7.4 Hz, 3H); ¹³C NMR (100 MHz, DMF-d₇) δ 168.0, 167.45, 166.96, 165.2, 152.5, 150.02, 149.93, 144.3, 138.3, 137.4, 134.1, 133.9, 129.82, 129.02, 128.58, 128.52, 128.38, 127.8, 121.6, 117.9, 116.6, 114.2, 66.9, 51.5, 48.8, 47.4, 43.0, 29.8, 28.5, 20.5, 11.0; HRMS (ES) calcd for C₃₇H₄₅N₈O₅ 681.3507, found 681.3494.

### Example 163

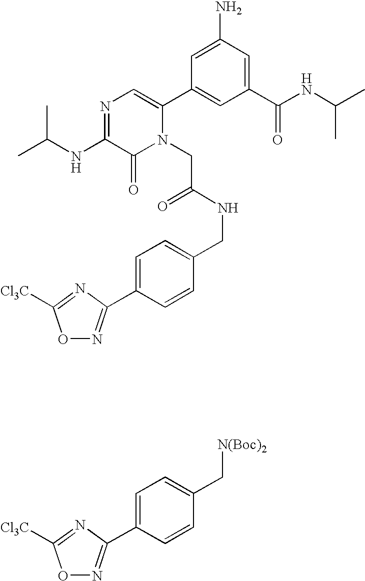

### Example 163a

To a stirred mixture of di-Boc-4-amino-hydroxybezamidine (3.0 g, 8.2 mmol) and pyridine (3.3 ml) in dichloromethane (9 ml) cooled in an ice bath was added trichloroacetic acid anhydride (3.3 ml, 18.1 mmol) slowly in three portion over 5 minutes. The reaction was stirred at ambient temperature for 20 hours. The reaction was purified by silica gel chromatography 80-100% dichloromethane/hexane) to give 3.17 g colorless solid. ¹HNMR (300 MHz, CDCl₃) δ 1.51 (s, 18H), 4.89 (s, 2H), 7.47 (d, J=8.4 Hz, 2H), 8.12 (d, J=8.1 Hz, 2H).

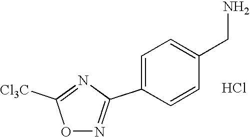

### Example 163b

To a solution of Example 163a (3.07 g, 6.2 mmol) in dioxane (60 ml) was added 4 N hydrogen chloride/dioxane (40 ml) with stirring at ambient temperature for three hours. The reaction was concentrated in vacuo to give 2.05 g of an off-white solid. ¹HNMR (300 MHz, DMSO-d₆) δ 4.2 (s, 2H), 7.77 (d, J=8.4 Hz, 2H), 8.13 (d, J=8.1 Hz, 2H), 8.64 (br s, 3H).

### Example 163c

To a stirred solution of free acid (0.3 g, 0.77 mmol), Example 163b (0.317 g, 0.86 mmol), and N-methylmorpholine (0.3 ml, 2.73 mmol) in N, N-dimethylformamide (3 ml) was added TBTU (0.31 g, 0.96 mmol). Stirring was continued at ambient temperature for 18 hours. The reaction was diluted with water and the resulting solid was collected by vacuum filtration, washed with water, and air-dried. The solid was crystallized from ethyl acetate/chloroform/hexane to give 0.15 g (30% yield) of an off-white solid. ¹HNMR (300 MHz, DMSO-d6) δ 1.15 (d, J=6.6 Hz, 6H), 1.24 (d, J=6.3 Hz, 6H), 4.04-4.19 (m, 2H), 4.40 (d, J=5.4 Hz, 2H), 4.45 (s, 2H), 6.69 (s, 1H), 6.72 (s, 1H), 7.01 (s, 1H), 7.12 (s, 1H), 7.45 (d, J=8.4 Hz, 2H), 8.00-8.08 (m, 3H), 8.66 (t, J=5.7 Hz, 1H). HRMS (ES) calcd for C₂₉H₃₂N₈O₄Cl₃ (M+H): 661.1607. Found: 661.1633. Anal. Calcd for C₂₉H₃₁N₈O₄Cl₃: C, 52.62; H, 4.72; N, 16.93. Found: C, 52.86; H, 4.74; N, 16.17.

### Example 164

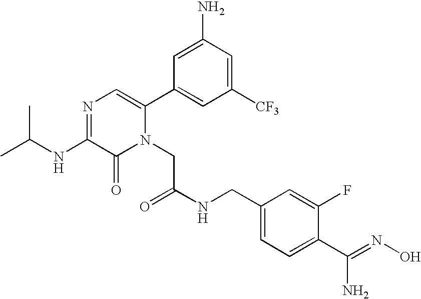

### Example 164

To a 25 mL RBF was added free amidine (1.3 g, 2.59 mmol) triethylamine (1 mL) and hydroxylamine hydrochloride (0.191 g, 2.75 mmol). The reaction was refluxed in ethanol (10 mL) overnight. The resulting solution was diluted to 50 mL with water and methanol. The reaction was purified using reverse phase chromatography to afford the title compound(1.21 g) as a TFA salt in 87% yield.

NMR (400 MHZ, CDCL3): ¹H 1.4 ppm (3H, d), 3.34 ppm (1H, m), 4.11 ppm(1H, q), 4.5 ppm (2H, d), 4.51 ppm (1H, s), 6.72 ppm (1H, s), 6.91 ppm (2H, m), 7.08 ppm (1H, s), 7.29 ppm (1H, d), 7.31 ppm (1H, s), 7.6 ppm (2H, m).

| | | | |
|---|---|---|---|
| Found | C: 44.30 | H: 3.71 | N: 13.09 |
| Calc. | C: 53.83 | H: 4.71 | N: 18.31 |

### What is claimed is:

1. A compound having the structure 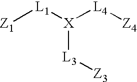
wherein:
X comprises a 5- or 6-membered heterocyclic or aromatic ring, the ring atoms being X₁, X₂, X₃, X₄, and X₅ for 5-membered heterocyclic rings and X₁, X₂, X₃, X₄, X₅ and X₆ for 6-membered heterocyclic or aromatic rings, wherein X₂ is alpha to each of X₁ and X₃, X₃ is alpha to each of X₂ and X₄, X₄ is alpha to each of X₃ and X₅, X₅ is alpha to X₄ and alpha to X₁ if X is a 5-membered ring or to X₆ if X is a 6-membered ring, and X₆, when present, is alpha to each of X₁ and X₅, wherein X₁, X₂, X₃, X₄, X₅ and X₆ are carbon, nitrogen, oxygen or sulfur;
L₁, L₃ and L₄ are linkages through which Z₁, Z₃, and Z₄, respectively, are covalently bonded to different ring atoms of the 5- or 6-membered heterocyclic or aromatic ring of X, wherein Z₁ is covalently bonded to X₁, Z₃ is covalently bonded to X₃, and Z₄ is covalently bonded to X₄, each of L₁, L₃ and L₄ independently being a covalent bond or comprising one or more atoms through which Z₁, Z₃, and Z₄ are covalently bonded to X₁, X₃ and X₄, respectively;
Z₁ is hydrocarbyl or substituted hydrocarbyl;
Z₃ comprises a 5- or 6-membered heterocyclic or aromatic ring substituted with a derivatized amidine which, upon hydrolysis, oxidation, reduction or elimination yields an amidine group, and optionally further substituted with a halogen or hydroxy, the ring atoms of the 5- or 6-membered heterocyclic or aromatic ring of Z₃ being carbon, sulfur, nitrogen, or oxygen;
Z₄ comprises a 5- or 6-membered heterocyclic or carbocyclic ring having two substituents, R₄₂ and R₄₄, and two ring atoms each of which is in the beta position relative to the ring atom of Z₄ through which Z₄ is covalently bonded to X, wherein one of R₄₂ and R₄₄ is covalently bonded to one of said beta positions and the other of R₄₂ and R₄₄ is covalently bonded to the other of said beta positions, the ring atoms of the 5- or 6-membered heterocyclic or carbocyclic ring of Z₄ being carbon, nitrogen, oxygen, or sulfur;
R₄₂ is amino; and
R₄₄ is selected from the group consisting of hydrogen, hydrocarbyl, substituted hydrocarbyl, heterocyclo, halogen, or a substituted or unsubstituted heteroatom selected from nitrogen, oxygen, sulfur and phosphorus; provided, however, the derivatized amidine is other than amidine derivatized with t-butoxycarbonyl.

2. The compound of claim 1 wherein:
each of X₁, X₂, X₃, X₄, X₅ and X₆ is carbon or nitrogen;
X₂ is a hydrogen bond acceptor;
L₁ is −X₉NH− wherein X₉ is covalently bonded directly to Z₁ and X₉ is a direct bond or —(CH₂)ₘ— wherein m is 1 to 5;
L₃ is a glycine derivative;
L₄ is a direct bond;
Z₁ is selected from the group consisting of C₁-C₈ alkyl, C₂-C₈ alkenyl, and C₂-C₈ alkynyl, the alkyl, alkenyl, or alkynyl being optionally substituted at any substitutable position with a halogen;
Z₃ comprises a phenyl, furanyl or thienyl ring, the phenyl, furanyl or thienyl ring being substituted with a derivatized amidine which, upon hydrolysis, oxidation, reduction or elimination yields an amidine group, and optionally further substituted with fluorine or hydroxy;
Z₄ comprises a phenyl or thienyl ring having two substituents, R₄₂ and R₄₄, and two ring atoms each of which is in the beta position relative to the ring atom of Z₄ through which Z₄ is covalently bonded to X, wherein one of R₄₂ and R₄₄ is covalently bonded to one of said beta positions and the other of R₄₂ and R₄₄ is covalently bonded to the other of said beta positions;
R₄₂ is amino; and
R₄₄ is selected from the group consisting of hydrocarbyl, substituted hydrocarbyl, heterocyclo, halogen, and an optionally substituted heteroatom selected from nitrogen, oxygen, sulfur and phosphorus.

3. The compound of claim 1 wherein L₁ is —X₉NH— wherein X₉ is covalently bonded directly to Z₁ and X₉ is a direct bond or —(CH₂)ₘ— wherein m is 1 to 5.

4. The compound of claim 1 wherein L₃ is selected from the group consisting of a glycine derivative, an alanine derivative, an amino derivative, and a sulfonyl derivative.

5. The compound of claim 1 wherein L₃ is a glycine derivative.

6. The compound of claim 5 wherein L₃ is —CH₂CONHCH₂—and Z₃ is covalently bonded to the methylene bonded to the amine nitrogen of L₃.

7. The compound of claim 1 wherein L₄ is selected from the group consisting of a direct bond, methylene, ethylene and an optionally substituted heteroatom selected from the group consisting of nitrogen, oxygen, sulfur and phosphorus.

8. The compound of claim 7 wherein L₄ is a direct bond.

9. The compound of claim 1 having the structure: 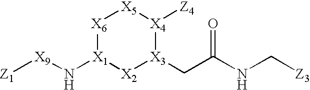
wherein
each of X₁, X₂, X₃, X₄, X₅ and X₆ is carbon or nitrogen;
X₂ is a hydrogen bond acceptor;
X₉ is a direct bond or —(CH₂)ₘ— where m is 1 to 5;
R₄₂ and R₄₄ are as defined in claim 1; and
Z₁, Z₃, and Z₄ are as defined in claim 1.

10. The compound of claim 9 wherein:
X₉ is selected from the group consisting of a direct bond, methylene, and ethylene;
Z₁ is selected from the group consisting of C₁-C₈ alkyl, C₂-C₈ alkenyl, and C₂-C₈ alkynyl, the alkyl, alkenyl, or alkynyl being optionally substituted at any substitutable position with a halogen;
Z₃ comprises a phenyl, furanyl or thienyl ring, the phenyl, furanyl or thienyl ring being substituted with a derivatized amidine which, upon hydrolysis, oxidation, reduction or elimination yields an amidine group, and optionally further substituted with fluorine or hydroxy;
Z₄ comprises a phenyl or thienyl ring having two substituents, R₄₂ and R₄₄, and two ring atoms each of which is in the beta position relative to the ring atom of Z₄ through which Z₄ is covalently bonded to X, wherein one of R₄₂ and R₄₄ is covalently bonded to one of said beta positions and the other of R₄₂ and R₄₄ is covalently bonded to the other of said beta positions;
R₄₂ is amino; and
R₄₄ is selected from the group consisting of hydrocarbyl, substituted hydrocarbyl, heterocyclo, halogen, and an optionally substituted heteroatom selected from nitrogen, oxygen, sulfur and phosphorus.

11. The compound of claim 2 or 10 wherein:
Z₁ is selected from the group consisting of cyclopropyl, isopropyl, methyl, ethyl, cyclobutyl, isobutyl, tert-butyl and sec-butyl optionally substituted at any substitutable position with fluorine, hydroxy, carboxy, or alkoxycarbonyl;
Z₃ is —R₃₀₀C(═NR₃₀)NR₃₀₂R₃₀₃, wherein R₃₀₀ is a phenyl ring, R₃₀₁, R₃₀₂, R₃₀₃ are independently selected from the group consisting of hydrogen, halogen, optionally substituted hydrocarbyl, and an optionally substituted heteroatom selected from the group consisting of oxygen, nitrogen, phosphorus and sulfur, provided at least one of R₃₀₁, R₃₀₂, R₃₀₃ is other than hydrogen;
Z₄ is a substituted phenyl ring; and
R₄₄ is selected from the group consisting of hydroxy, isobutylsulfonyl, trifluoromethyl, carboxamidobenzyl, carboxamidobutyl-2-yl, isobutyramido, isobutoxy, carboethoxy, carboxyl, amino, 3-aminomethylthiophene, benzylamine, phenethylamine, isobutylamine, methoxyethylamide, 1-carboxylbenzylamide, p-fluorobenzylamide, cyclobutylamide, m-fluorobenzylamide, 1-methylbenzylamide, sec-butylamide, benzylacylamine, isobutylamide, sec-pentylamine, cyclopentylacylamine, 1-carboxyl-2-methylbutylamide, isobutylacylamine, isobutylsulfoxyl, 2-cyclohexylamide, methoxy, sulfonamide, isobutylsulfonamide, aminoacyltrifluoromethyl, and carbmethoxy.

12. The compound of each of claims 1, 2, 9 or 10 wherein each of X₁, X₂, X₃, X₄, X₅ and X₆ is carbon or nitrogen, each of X₁, X₂, X₄, X₅ and X₆ is sp² or sp³ hybridized, and X₃ is sp³ hybridized.

13. The compound of each of claims 1, 2, 9 or 10 wherein:
X₁, X₄ and X₅ are carbon;
X₂ is carbonyl; and
X₃ and X₆ are nitrogen.

14. The compound of each of claims 1, 2, 9 or 10 wherein:
X₁, X₄ and X₆ are carbon;
X₂ is carbonyl; and
X₃ and X₅ are nitrogen.

15. The compound of each of claims 1, 2, 9 or 10 wherein:
X₁, X₄, X₅ and X₆ are carbon;
X₂ is carbonyl; and
X₃ is nitrogen.

16. The compound of each of claims 1, 2, 9 or 10 wherein X₁, X₂, X₃, ₄, X₅, and X₆ are selected to provide a heterocyclic or carbocyclic ring selected from the group consisting of a pyrazinone, pyrimidinone, 2-pyridone, 4-pyrone, 4-pyridone, pyridine, 1,4-quinone, benzene, and uracil.

17. The compound of claim 16 wherein the heterocyclic ring is selected from the group consisting of pyrazinone, pyrimidinone, and 2-pyridone.

18. The compound of claim 17 wherein the heterocyclic ring is a pyrazinone.

19. The of each of claims 1, 2, 9 or 10 wherein X₂ is a hydrogen bond acceptor.

20. The compound of claim 19 wherein X₂ is selected from the group consisting of (i) carbon substituted with hydrogen, fluorine, oxygen, or sulfur, (ii) nitrogen, optionally substituted with hydrogen or oxygen, (iii) oxygen and (iv) sulfur.

21. The compound of claim 20 wherein X₂ is a carbonyl.

22. The compound of each of claims 1, 2, 9 or 10 wherein X₅ is a hydrogen bond acceptor.

23. The compound of claim 22 wherein X₅ is selected from the group consisting of oxygen, sulfur, nitrogen, carbonyl, and carbon, the carbon being optionally substituted with a halogen.

24. The compound of claim 2 or 9 wherein X₉ is a direct bond.

25. The compound of claim 2 or 9 wherein X₉ is methylene or ethylene.

26. The compound of claim 1 or 9 wherein Z₁ is selected from the group consisting of C₁-C₈ alkyl, C₂-C₈ alkenyl, and C₂-C₈ alkynyl, the alkyl, alkenyl, or alkynyl being optionally substituted at any substitutable position with a halogen.

27. The compound of each of claims 1, 2, 9 or 10 wherein Z₁ is selected from the group consisting of cyclopropyl, isopropyl, methyl, ethyl, cyclobutyl, isobutyl, tert-butyl and sec-butyl optionally substituted at any substitutable position with fluorine, hydroxy, carboxy, or alkoxycarbonyl.

28. The compound of claim 27 wherein Z₁ is selected from the group consisting of cyclopropyl, isopropyl, cyclobutyl, isobutyl, and sec-butyl optionally substituted at any substitutable position with fluorine, hydroxy, carboxy, or alkoxycarbonyl.

29. The compound of claim 28 wherein Z₁ is isopropyl or cyclobutyl optionally substituted at any substitutable position with fluorine, hydroxy, carboxy, or alkoxycarbonyl.

30. The compound of claim 1 or 9 wherein Z₃ is —R₃₀₀C(═NR₃₀₁)NR₃₀₂R₃₀₃, wherein R₃₀₀ is a 6-membered carbocyclic aromatic ring, R₃₀₁, R₃₀₂, R₃₀₃ are independently selected from the group consisting of hydrogen, halogen, optionally substituted hydrocarbyl, and an optionally substituted heteroatom selected from the group consisting of oxygen, nitrogen, phosphorus and sulfur, provided at least one of R₃₀₁, R₃₀₂, R₃₀₃ is other than hydrogen.

31. The compound of claim 30 wherein Z₃ is —R₃₀₀C(═NR₃₀₁)NR₃₀₂R₃₀₃, wherein R₃₀₀ is a 6-membered carbocyclic aromatic ring, and at least two of R₃₀₁, R₃₀₂, R₃₀₃ are ring atoms of a heterocyclic ring.

32. The compound of claim 30 wherein Z₃ is —R₃₀₀C(═NR₃₀₁)NR₃₀₂R₃₀₃, R₃₀₀ is a 6-membered carbocyclic aromatic ring, and at least one of R₃₀₁, R₃₀₂, R₃₀₃ are ring atoms of a heterocyclic ring fused to R₃₀₀.

33. The compound of claim 1 or 9 wherein Z₃ is a benzamidine derivative which hydrolyzes under physiological conditions to form benzamidine, the benzamidine being derivatized with one or more groups selected from carbonyl, thiocarbonyl, imino, enamino, phosphorus, and sulfur.

34. The compound of claim 1 or 9 wherein Z₃ is a benzamidine derivative which oxidizes under physiological conditions to form benzamidine, the benzamidine being derivatized with one or more groups selected from the group consisting of (i) optionally substituted hydrocarbyl provided that the carbon atom directly bonded to the amidine is sp³ hybridized, and (ii) aryl.

35. The compound of claim 1 or 9 wherein Z₃ is a benzamidine derivative which is reduced under physiological conditions to form benzamidine, the benzamidine being derivatized with one or more heteroatoms selected from the group consisting of oxygen, nitrogen in its most reduced state, and sulfur in its most reduced state.

36. The compound of claim 1 or 9 wherein Z₃ is a benzamidine derivative which is eliminated under physiological conditions to form benzamidine, the benzamidine being derivatized with one or more substituents selected from the group consisting of a hydrocarbyl substituted at the beta carbon with carbonyl, sulfonyl, sulfinyl, cyano, nitro and an alkyl, aryl, or heterocyclic group substituted with oxygen, nitrogen, or sulfur at the carbon directly bonded to the amidine group.

37. The compound of claim 1 or 9 wherein Z₃ is a benzamidine derivative which hydrolyzes under physiological conditions to form benzamidine, the benzamidine derivative having the formula 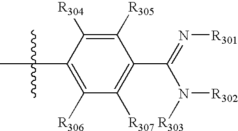
wherein:
R₃₀₁, R₃₀₂, and R₃₀₃ are independently selected from the group consisting of hydrogen, —C(═O)Rₐ, —C(═O)ORₐ, —S(═O)ORₐ, —S(═O)SRₐ, —S(═O)₂ORₐ, —S(═O)₂SRₐ and alkenyl, wherein Rₐ is selected from the group consisting of hydrocarbyl, substituted hydrocarbyl, and heterocyclo, provided, however, at least one of R₃₀₁, R₃₀₂, and R₃₀₃ is other than hydrogen and that the carbon atom of R₃₀₁, R₃₀₂, and R₃₀₃ directly bonded to the amidine is sp² hybridized when R₃₀₁, R₃₀₂, and R₃₀₃ is alkenyl;
R₃₀₄ is selected from the group consisting of halogen, hydrogen, hydroxyl, alkyl, sulfhydryl, alkoxy, and alkylthio;
R₃₀₅ is selected from the group consisting of oxygen, sulfur, halogen, hydrogen, hydroxyl, alkyl, sulfhydryl, alkoxy, and alkylthio;
R₃₀₆ is selected from the group consisting of halogen, hydrogen, hydroxyl, alkyl, sulfhydryl, alkoxy, and alkylthio; and
R₃₀₇ is selected from the group consisting of oxygen, sulfur, halogen, hydrogen, hydroxyl, alkyl, sulfhydryl, alkoxy, and alkylthio.

38. The compound of claim 1 or 9 wherein Z₃ is a benzamidine derivative which oxidizes under physiological conditions to form benzamidine, the benzamidine derivative having the formula 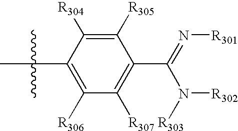
wherein:
R₃₀₁, R₃₀₂, and R₃₀₃ are independently selected from the group consisting of hydrogen, optionally substituted hydrocarbyl and aryl, provided, however, at least one of R₃₀₁, R₃₀₂, and R₃₀₃ is other than hydrogen and the carbon atom of R₃₀₁, R₃₀₂, and R₃₀₃ directly bonded to the amidine is sp³ hybridized when R₃₀₁, R₃₀₂, and R₃₀₃ is optionally substituted hydrocarbyl;
R₃₀₄ is selected from the group consisting of halogen, hydrogen, hydroxyl, alkyl, sulfhydryl, alkoxy, and alkylthio;
R₃₀₅ is selected from the group consisting of oxygen, sulfur, halogen, hydrogen, hydroxyl, alkyl, sulfhydryl, alkoxy, and alkylthio;
R₃₀₆ is selected from the group consisting of halogen, hydrogen, hydroxyl, alkyl, sulfhydryl, alkoxy, and alkylthio; and
R₃₀₇ is selected from the group consisting of oxygen, sulfur, halogen, hydrogen, hydroxyl, alkyl, sulfhydryl, alkoxy, and alkylthio.

39. The compound of claim 1 or 9 wherein Z₃ is a benzamidine derivative which is reduced under physiological conditions to form benzamidine, the benzamidine derivative having the formula 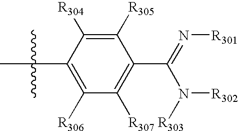
wherein:
R₃₀₁, R₃₀₂, and R₃₀₃ are independently selected from the group consisting of hydrogen, —OR_{b}, —SR_{b}, —NR_{b}, or —N(R_{b})₁, wherein each R_{b} is independently optionally substituted hydrocarbyl, and heterocyclo, provided, however, at least one of R₃₀₁, R₃₀₂, and R₃₀₃ is other than hydrogen;
R₃₀₄ is selected from the group consisting of halogen, hydrogen, hydroxyl, alkyl, sulfhydryl, alkoxy, and alkylthio;
R₃₀₅ is selected from the group consisting of oxygen, sulfur, halogen, hydrogen, hydroxyl, alkyl, sulfhydryl, alkoxy, and alkylthio;
R₃₀₆ is selected from the group consisting of halogen, hydrogen, hydroxyl, alkyl, sulfhydryl, alkoxy, and alkylthio; and
R₃₀₇ is selected from the group consisting of oxygen, sulfur, halogen, hydrogen, hydroxyl, alkyl, sulfhydryl, alkoxy, and alkylthio.

40. The compound of claim 1 or 9 wherein Z₃ is a benzamidine derivative which undergoes an elimination reaction under physiological conditions to form benzamidine, the benzamidine derivative having the formula 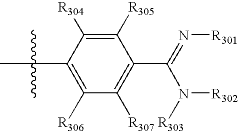
wherein:
R₃₀₁, R₃₀₂, and R₃₀₃ are independently selected from the group consisting of (i) hydrogen, (ii) substituted hydrocarbyl wherein the carbon bonded to the amidine group is substituted with —OR_{c}, —SR_{c}, —NR_{c}, or —N(R_{c})₂, wherein each R_{c} is independently —C(O)R_{d}, —C(O)OR_{d}, —C(O)NR_{d}, —C(O)N(R_{d})₂ and each R_{d} is independently hydrocarbyl, substituted hydrocarbyl or heterocyclo, and (iii) substituted alkyl with the carbon atom beta to the point of attachment to the amidine group being an unsaturated electron withdrawing group, provided, at least one of R₃₀₁, R₃₀₂, and R₃₀₃ is other than hydrogen;
R₃₀₄ is selected from the group consisting of halogen, hydrogen, hydroxyl, alkyl, sulfhydryl, alkoxy, and alkylthio;
R₃₀₅ is selected from the group consisting of oxygen, sulfur, halogen, hydrogen, hydroxyl, alkyl, sulfhydryl, alkoxy, and alkylthio;
R₃₀₆ is selected from the group consisting of halogen, hydrogen, hydroxyl, alkyl, sulfhydryl, alkoxy, and alkylthio; and
R₃₀₇ is selected from the group consisting of oxygen, sulfur, halogen, hydrogen, hydroxyl, alkyl, sulfhydryl, alkoxy, and alkylthio.

41. The compound of claim 37 wherein R₃₀₁ and R₃₀₅ together with the benzene ring of which R₃₀₅ is a substituent form a fused ring.

42. The compound of claim 38 wherein R₃₀₁ and R₃₀₅ together with the benzene ring of which R₃₀₅ is a substituent form a fused ring.

43. The compound of claim 39 wherein R₃₀₁ and R₃₀₅ together with the benzene ring of which R₃₀₅ is a substituent form a fused ring.

44. The compound of claim 40 wherein R₃₀₁ and R₃₀₅ together with the benzene ring of which R₃₀₅ is a substituent form a fused ring.

45. The compound of claim 37 wherein R₃₀₁ and one of R₃₀₂ and R₃₀₃ together with the nitrogen atoms to which they are bonded form a 5- or 6-membered heterocyclic ring.

46. The compound of claim 38 wherein R₃₀₁ and one of R₃₀₂ and R₃₀₃ together with the nitrogen atoms to which they are bonded form a 5- or 6-membered heterocyclic ring.

47. The compound of claim 39 wherein R₃₀₁ and one of R₃₀₂ and R₃₀₃ together with the nitrogen atoms to which they are bonded form a 5- or 6-membered heterocyclic ring.

48. The compound of claim 40 wherein R₃₀₁ and one of R₃₀₂ and R₃₀₃ together with the nitrogen atoms to which they are bonded form a 5- or 6-membered heterocyclic ring.

49. The compound of each of claims 45 to 48 wherein the ring atoms of the 5- or 6-membered heterocyclic ring are selected from the group consisting of carbon, nitrogen and oxygen.

50. The compound of claim 1 or 9 wherein Z₃ is selected from the group consisting of:

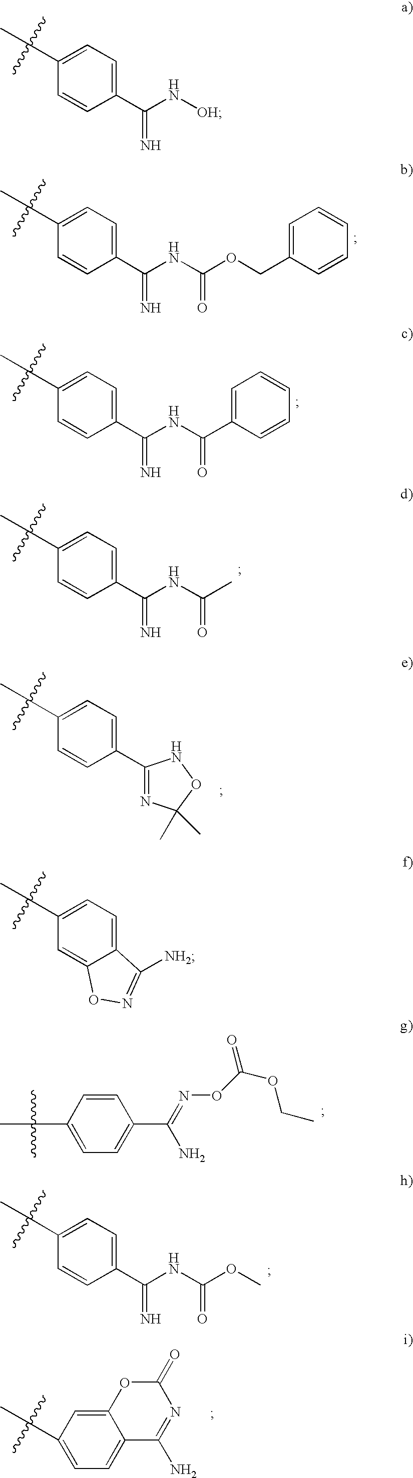

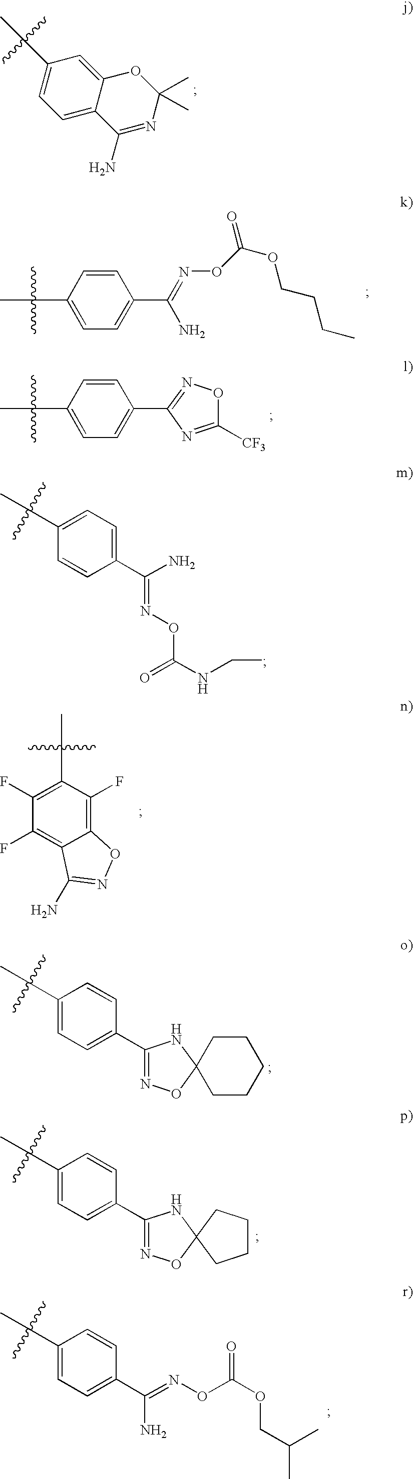

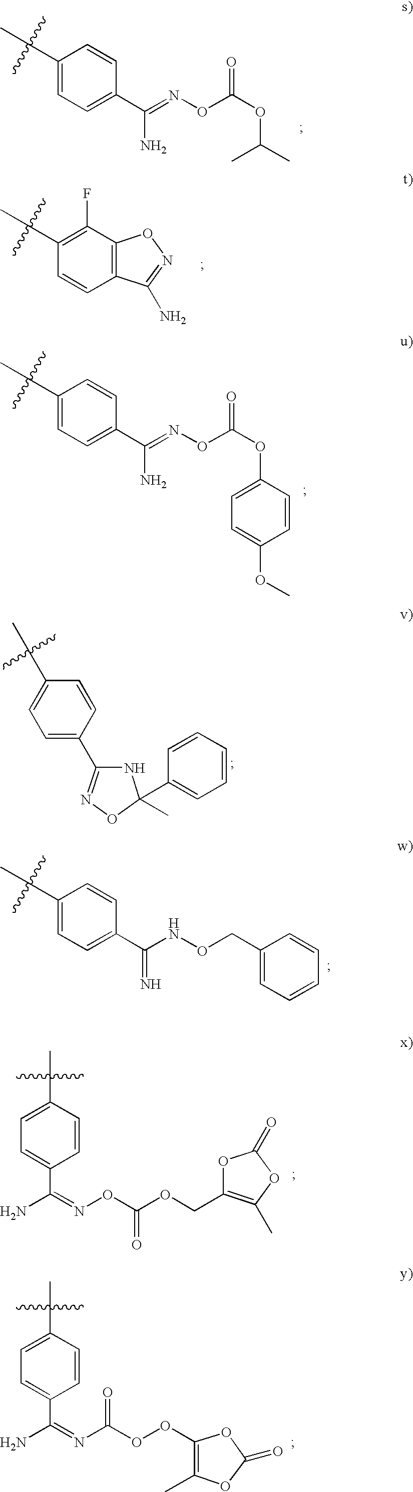

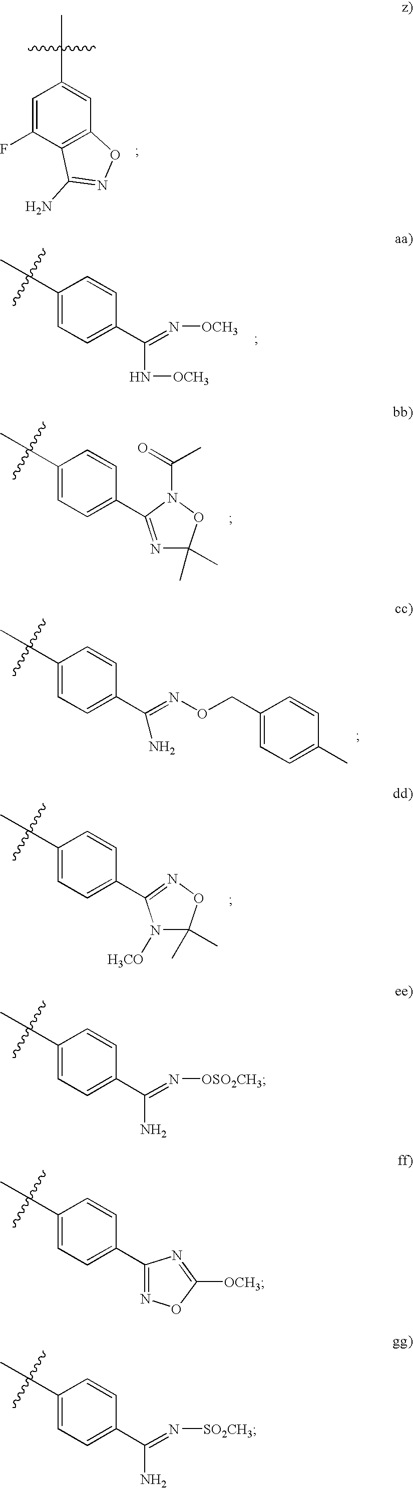

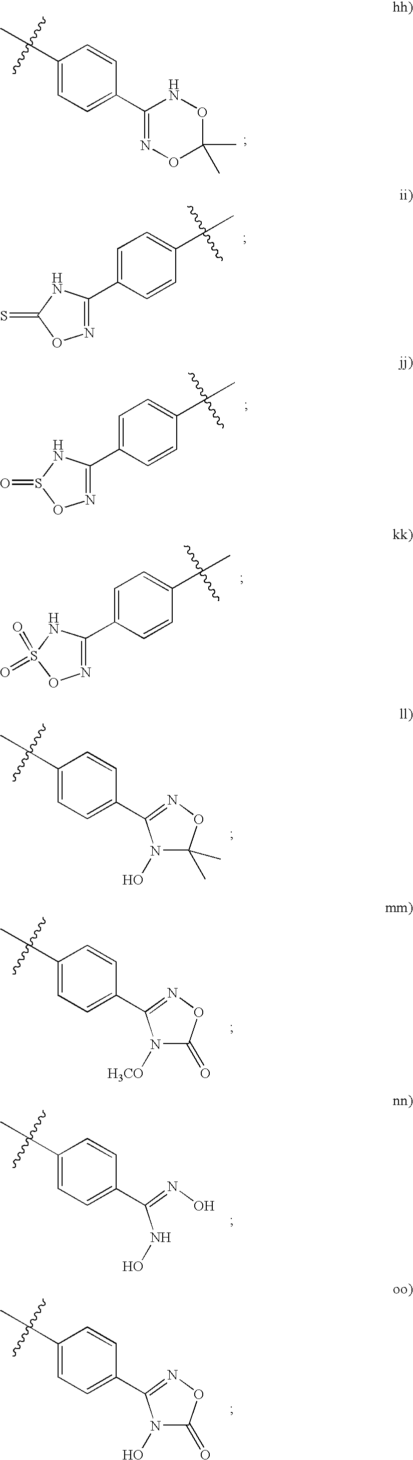

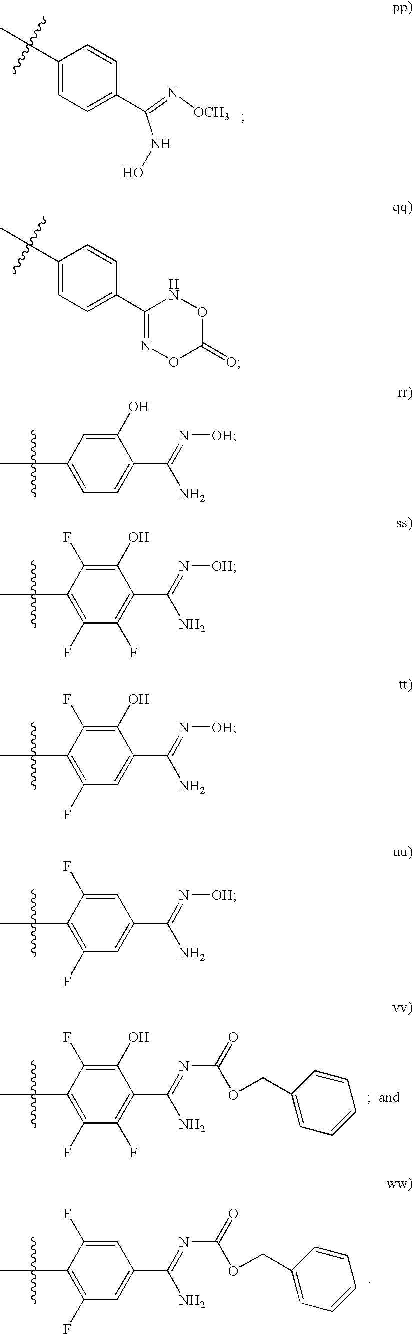

51. The compound of claim 1 or 9 wherein Z₄ is a substituted, 6-membered, carbocyclic aromatic ring.

52. The compound of claim 1 or 9 wherein Z₄ has the structure: 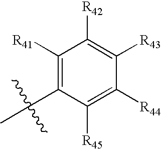
wherein
R₄₂ is amino;
R₄₄ is selected from the group consisting of hydrocarbyl, substituted hydrocarbyl, halogen and an optionally substituted heteroatom selected from the group consisting of oxygen, nitrogen, and sulfur; and
R₄₁, R₄₃ and R₄₅ are independently selected from the group consisting of hydrogen, halogen, hydrocarbyl, substituted hydrocarbyl, and an optionally substituted heteroatom selected from the group consisting of oxygen, nitrogen, and sulfur.

53. The compound of claim 52 wherein R₄₄ is selected from the group consisting of hydrocarbyl, substituted hydrocarbyl, acetamido, alkoxy, hydroxy, amino, alkylsulfonyl, haloalkyl, haloalkoxy, haloalkylthio, carboalkoxy, carboxy, carboxamidoalkyl, and carboxamidoalkylaryl.

54. The compound of claim 52 wherein R₄₄ is selected from the group consisting of hydrogen, hydrocarbyl, substituted hydrocarbyl, heteroaryl, heterocyclo, halogen, acetamido, guanidino, hydroxy, nitro, amino, amidosulfonyl, acylamido, hydrocarbyloxy, substituted hydrocarbyloxy, hydrocarbylthio, substituted hydrocarbylthio, hydrocarbylsulfonyl, and substituted hydrocarbylsulfonyl.

55. The compound of claim 52 wherein R₄₄ is selected from the group consisting of hydroxy, isobutylsulfonyl, trifluoromethyl, carboxamidobenzyl, carboxamidobutyl-2-yl, isobutyramido, isobutoxy, carboethoxy, carboxyl, amino, 3-aminomethylthiophene, benzylamine, phenethylamine, isobutylamine, methoxyethylamide, 1-carboxylbenzylamide, p-fluorobenzylamide, cyclobutylamide, m-fluorobenzylamide, 1-methylbenzylamide, sec-butylamide, benzylacylamine, isobutylamide, sec-pentylamine, cyclopentylacylamine, 1-carboxyl-2-methylbutylamide, isobutylacylamine, isobutylsulfoxyl, 2-cyclohexylamide, methoxy, sulfonamide, isobutylsulfonamide, aminoacyltrifluoromethyl, and carbmethoxy.

56. The compound of claim 52 wherein each of R₄₁, R₄₃ and R₄₅ is hydrogen.

57. The compound of claim 1 or 9 wherein Z₄ has the structure 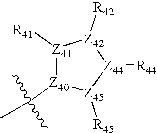
wherein:
Z₄₀, Z₄₁, Z₄₂, Z₄₄, and Z₄₅ are independently selected from the group consisting of carbon, nitrogen, oxygen and sulfur;
R₄₂ is amino;
R₄₄ is selected from the group consisting of is selected from the group consisting of hydrocarbyl, substituted hydrocarbyl, halogen and an optionally substituted heteroatom selected from the group consisting of oxygen, nitrogen, and sulfur; and
R₄₁ and R₄₅ are independently selected from the group consisting of hydrogen, halogen, hydrocarbyl, substituted hydrocarbyl, and an optionally substituted heteroatom selected from the group consisting of oxygen, nitrogen, and sulfur.

58. The compound of claim 57 wherein R₄₄ is selected from the group consisting of hydrocarbyl, substituted hydrocarbyl, acetamido, alkoxy, hydroxy, amino, alkylsulfonyl, haloalkyl, haloalkoxy, haloalkylthio, carboalkoxy, carboxy, carboxamidoalkyl, and carboxamidoalkylaryl.

59. The compound of claim 57 wherein R₄₄ is selected from the group consisting of hydrogen, hydrocarbyl, substituted hydrocarbyl, heteroaryl, heterocyclo, halogen, acetamido, guanidino, hydroxy, nitro, amino, amidosulfonyl, acylamido, hydrocarbyloxy, substituted hydrocarbyloxy, hydrocarbylthio, substituted hydrocarbylthio, hydrocarbylsulfonyl, and substituted hydrocarbylsulfonyl.

60. The compound of claim 57 wherein R₄₄ is selected from the group consisting of hydroxy, isobutylsulfonyl, trifluoromethyl, carboxamidobenzyl, carboxamidobutyl-2-yl, isobutyramido, isobutoxy, carboethoxy, carboxyl, amino, 3-aminomethylthiophene, benzylamine, phenethylamine, isobutylamine, methoxyethylamide, 1-carboxylbenzylamide, p-fluorobenzylamide, cyclobutylamide, m-fluorobenzylamide, 1-methylbenzylamide, sec-butylamide, benzylacylamine, isobutylamide, sec-pentylamine, cyclopentylacylamine, 1-carboxyl-2-methylbutylamide, isobutylacylamine, isobutylsulfoxyl, 2-cyclohexylamide, methoxy, sulfonamide, isobutylsulfonamide, aminoacyltrifluoromethyl, and carbmethoxy.

61. The compound of claim 57 wherein each of R₄₁, R₄₂ and R₄₅ is hydrogen.

62. The compound of claim 9 having the structure 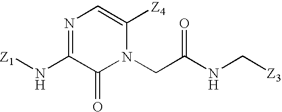
wherein:
each of Z₁, Z₃, Z₄, R₄₂ and R₄₄ are as defined in claim 9.

63. The compound of claim 9 having the structure 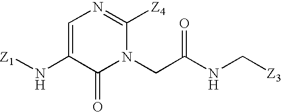
wherein:
each of Z₁, Z₃, Z₄, R₄₂ and R₄₄ are as defined in claim 9.

64. The compound of claim 9 having the structure 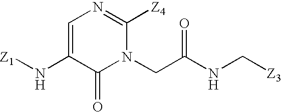
wherein:
each of Z₁, Z₃, Z₄, R₄₂ and R₄₄ are as defined in claim 9.

65. The compound of each of claims 62 to 64 wherein:
Z₁ is selected from the group consisting of cyclopropyl, isopropyl, methyl, ethyl, cyclobutyl, isobutyl, tert-butyl and sec-butyl optionally substituted at any substitutable position with fluorine, hydroxy, carboxy, or alkoxycarbonyl;
Z₃ is —R₃₀₀C(═NR₃₀₁)NR₃₀₂R₃₀₃, wherein R₃₀₀ is a phenyl ring, R₃₀₁, R₃₀₂, R₃₀₃ are independently selected from the group consisting of hydrogen, halogen, optionally substituted hydrocarbyl, and an optionally substituted heteroatom selected from the group consisting of oxygen, nitrogen, phosphorus and sulfur, provided at least one of R₃₀₁, R₃₀₂, R₃₀₃ is other than hydrogen; and
Z₄ is a phenyl ring having the structure 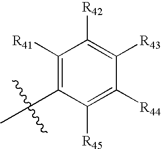
wherein:
R₄₂ is amino;
R₄₄ is selected from the group consisting of hydroxy, isobutylsulfonyl, trifluoromethyl, carboxamidobenzyl, carboxamidobutyl-2-yl, isobutyramido, isobutoxy, carboethoxy, carboxyl, amino, 3-aminomethylthiophene, benzylamine, phenethylamine, isobutylamine, methoxyethylamide, 1-carboxylbenzylamide, p-fluorobenzylamide, cyclobutylamide, m-fluorobenzylamide, 1-methylbenzylamide, sec-butylamide, benzylacylamine, isobutylamide, sec-pentylamine, cyclopentylacylamine, 1-carboxyl-2-methylbutylamide, isobutylacylamine, isobutylsulfoxyl, 2-cyclohexylamide, methoxy, sulfonamide, isobutylsulfonamide, aminoacyltrifluoromethyl, and carbmethoxy; and
R₄₁, R₄₃ and R₄₅ are independently selected from the group consisting of hydrogen, halogen, hydrocarbyl, substituted hydrocarbyl, and an optionally substituted heteroatom selected from the group consisting of oxygen, nitrogen, and sulfur.

66. The compound of claim 65 wherein:
Z₁ is selected from the group consisting of cyclopropyl, isopropyl, methyl, ethyl, cyclobutyl, isobutyl, and sec-butyl optionally substituted at any substitutable position with fluorine, hydroxy, carboxy, or alkoxycarbonyl; and
Z₃ is as defined in claim 50.

67. The compound of claim 9 having the structure: 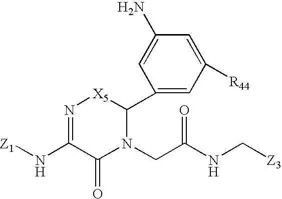
wherein
X₅ is CH, C(Cl) or C(F)
Z₁ is isopropyl, cyclopropyl, cyclobutyl or cycylopentyl optionally substituted by fluorine, hydroxy, carboxy, or alkoxycarbonyl;
Z₃ is —R₃₀₀C (═NR₃₀₁)NR₃₀₂R₃₀₃ wherein R₃₀₀ is a 6-membered carbocyclic aromatic ring, R₃₀₁, R₃₀₂, R₃₀₃ are independently selected from the group consisting of hydrogen, halogen, optionally substituted hydrocarbyl, and an optionally substituted heteroatom selected from the group consisting of oxygen, nitrogen, phosphorus and sulfur, provided at least one of R₃₀₁, R₃₀₂, R₃₀₃ is other than hydrogen; and
R₄₄ is selected from the group consisting of hydroxy, isobutylsulfonyl, trifluoromethyl, carboxamidobenzyl, carboxamidobutyl-2-yl, isobutyramido, isobutoxy, carboethoxy, carboxyl, amino, 3-aminomethylthiophene, benzylamine, phenethylamine, isobutylamine, methoxyethylamide, 1-carboxylbenzylamide, p-fluorobenzylamide, cyclobutylamide, m-fluorobenzylamide, 1-methylbenzylamide, sec-butylamide, benzylacylamine, isobutylamide, sec-pentylamine, cyclopentylacylamine, 1-carboxyl-2-methylbutylamide, isobutylacylamine, isobutylsulfoxyl, 2-cyclohexylamide, methoxy, sulfonamide, isobutylsulfonamide, aminoacyltrifluoromethyl, and carbmethoxy.

68. The compound of claim 67 having the structure:
wherein
X₅ is CH, C(Cl) or C(F); 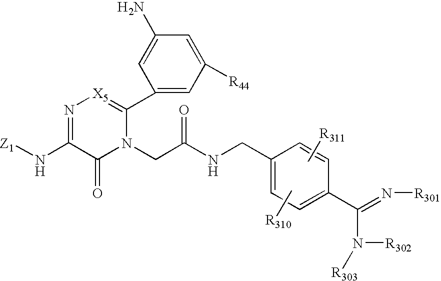
Z₁ is isopropyl, cyclopropyl, cyclobutyl or cycylopentyl optionally substituted by fluorine, hydroxy, carboxy, or alkoxycarbonyl;
R₄₄ is selected from the group consisting of hydroxy, isobutylsulfonyl, trifluoromethyl, carboxamidobenzyl, carboxamidobutyl-2-yl, isobutyramido, isobutoxy, carboethoxy, carboxyl, amino, 3-aminomethylthiophene, benzylamine, phenethylamine, isobutylamine, methoxyethylamide, 1-carboxylbenzylamide, p-fluorobenzylamide, cyclobutylamide, m-fluorobenzylamide, 1-methylbenzylamide, sec-butylamide, benzylacylamine, isobutylamide, sec-pentylamine, cyclopentylacylamine, 1-carboxyl-2-methylbutylamide, isobutylacylamine, isobutylsulfoxyl, 2-cyclohexylamide, methoxy, sulfonamide, isobutylsulfonamide, aminoacyltrifluoromethyl, and carbmethoxy;
R₃₀₁, R₃₀₂, and R₃₀₃ are independently selected from the group consisting of:
(i) hydrogen, —C(═O)Rₐ, —C(═O)ORₐ, —S(═O)ORₐ, —S(═O)SRₐ, —S(═O)₂ORₐ, —S(═O)₂SRₐ and alkenyl, wherein Rₐ is selected from the group consisting of hydrocarbyl, substituted hydrocarbyl, and heterocyclo, provided, however, that the carbon atom of R₃₀₁, R₃₀₂, and R₃₀₃ directly bonded to the amidine is sp² hybridized when R₃₀₁, R₃₀₂, and R₃₀₃ is alkenyl,
(ii) hydrogen, optionally substituted hydrocarbyl and aryl, provided, however, the carbon atom of R₃₀₁, R₃₀₂, and R₃₀₃ directly bonded to the amidine is sp³ hybridized when R₃₀₁, R₃₀₂, and R₃₀₃ is optionally substituted hydrocarbyl,
(iii) hydrogen, —OR_{b}, —SR_{b}, —NR_{b}, or —N(R_{b})₂, wherein each R_{b} is independently optionally substituted hydrocarbyl, and heterocyclo, and
(iv) hydrogen, substituted hydrocarbyl wherein the carbon bonded to the amidine group is substituted with —OR_{c}, —SR_{c}, —NR_{c}, or —N(R_{c})₂, wherein each R_{c} is independently —C(O)R_{d}, —C(O)NR_{d}, —C(O)OR_{d}, —C(O)N(R_{d})₂ and each R_{d} is independently hydrocarbyl, substituted hydrocarbyl or heterocyclo, and substituted alkyl with the carbon atom beta to the point of attachment to the amidine group being an unsaturated electron withdrawing group, provided, however, at least one of R₃₀₁, R₃₀₂, and R₃₀₃ is other than hydrogen; and
R₃₁₀ and R₃₁₁ are independently selected from the group consisting of hydrogen, fluorine, hydroxy, alkoxy, and carboxy, provided at least one of R₃₁₀ and R₃₁₁ is other than fluorine and hydrogen.

69. The compound of claim 68 having the structure:
wherein: 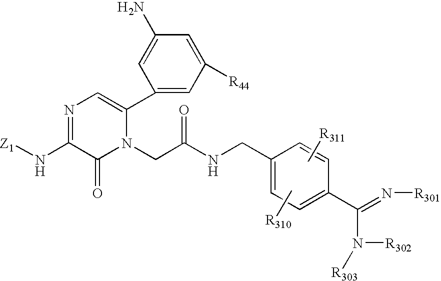
Z₁ is isopropyl or cyclopropyl optionally substituted with fluorine, hydroxy, carboxy, or alkoxycarbonyl;
R₄₄ is selected from the group consisting of hydroxy, isobutylsulfonyl, trifluoromethyl, carboxamidobenzyl, carboxamidobutyl-2-yl, isobutyramido, isobutoxy, carboethoxy, carboxyl, amino, 3-aminomethylthiophene, benzylamine, phenethylamine, isobutylamine, methoxyethylamide, 1-carboxylbenzylamide, p-fluorobenzylamide, cyclobutylamide, m-fluorobenzylamide, 1-methylbenzylamide, sec-butylamide, benzylacylamine, isobutylamide, sec-pentylamine, cyclopentylacylamine, 1-carboxyl-2-methylbutylamide, isobutylacylamine, isobutylsulfoxyl, 2-cyclohexylamide, methoxy, sulfonamide, isobutylsulfonamide, aminoacyltrifluoromethyl, and carbmethoxy;
R₃₀₁, R₃₀₂, and R₃₀₃ are independently selected from the group consisting of:
(i) hydrogen, —C(═O)Rₐ, —C(═O)ORₐ, —S(=O)ORₐ, —S(═O)SRₐ, —S(═O)₂ORₐ, —S(═O)₂SRₐ and alkenyl, wherein Rₐ is selected from the group consisting of hydrocarbyl, substituted hydrocarbyl, and heterocyclo, provided, however, that the carbon atom of R₃₀₁, R₃₀₂, and R₃₀₃ directly bonded to the amidine is sp² hybridized when R₃₀₁, R₃₀₂, and R₃₀₃ is alkenyl,
(ii) hydrogen, optionally substituted hydrocarbyl and aryl, provided, however, the carbon atom of R₃₀₁, R₃₀₂, and R₃₀₃ directly bonded to the amidine is sp³ hybridized when R₃₀₁, R₃₀₂, and R₃₀₃ is optionally substituted hydrocarbyl,
(iii) hydrogen, —OR_{b}, —SR_{b}, —NR_{b}, or —N(R_{b})₂, wherein each R_{b} is independently optionally substituted hydrocarbyl, and heterocyclo, and
(iv) hydrogen, substituted hydrocarbyl wherein the carbon bonded to the amidine group is substituted with —OR_{c}, —SR_{c}, —NR_{c}, or —N(R_{c})₂, wherein each R_{c} is independently —C(O)R_{d}, —C(O)NR_{d}, —C(O)OR_{d}, —C(O)N(Rd)₂ and each R_{d} is independently hydrocarbyl, substituted hydrocarbyl or heterocyclo, and substituted alkyl with the carbon atom beta to the point of attachment to the amidine group being an unsaturated electron withdrawing group, provided, however, at least one of R₃₀₁, R₃₀₂, and R₃₀₃ is other than hydrogen; and
R₃₁₀ and R₃₁₁ are independently selected from the group consisting of hydrogen, fluorine, hydroxy, alkoxy, and carboxy, provided at least one of R₃₁₀ and R₃₁₁ is other than fluorine and hydrogen.

70. The compound of claim 68 having the structure: 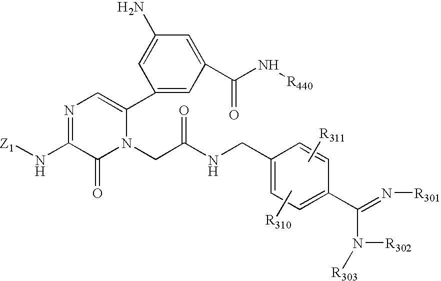
wherein:
Z₁ is isopropyl or cyclopropyl optionally substituted with fluorine, hydroxy, carboxy, or alkoxycarbonyl;
R₃₀₁, R₃₀₂, and R₃₀₃ are independently selected from the group consisting of:
(i) hydrogen, —C(═O)Rₐ, —C(═O)ORₐ, —S(═O)ORₐ, —S(═O)SRₐ, —S(═O)₂ORₐ, —S(═O)₂SRₐ and alkenyl, wherein Rₐ is selected from the group consisting of hydrocarbyl, substituted hydrocarbyl, and heterocyclo, provided, however that the carbon atom of R₃₀₁, R₃₀₂, and R₃₀₃ directly bonded to the amidine is sp² hybridized when R₃₀₁, R₃₀₂, and R₃₀₃ is alkenyl,
(ii) hydrogen, optionally substituted hydrocarbyl and aryl, provided, however, the carbon atom of R₃₀₁, R₃₀₂, and R₃₀₃ directly bonded to the amidine is sp³ hybridized when R₃₀₁, R₃₀₂, and R₃₀₃ is optionally substituted hydrocarbyl,
(iii) hydrogen, —OR_{b}, —SR_{b}, —NR_{b}, or —N(R_{b})₂, wherein each R_{b} is independently optionally substituted hydrocarbyl, and heterocyclo, and
(iv) hydrogen, substituted hydrocarbyl wherein the carbon bonded to the amidine group is substituted with —OR_{c}, —SR_{c}, —NR_{c}, or —N(R_{c})₂, wherein each R_{c} is independently —C(O)R_{d}, —C(O)OR_{d}, —C(O)NR_{d}, —C(O)N(R_{d})₂ and each R_{d} is independently hydrocarbyl, substituted hydrocarbyl or heterocyclo, and substituted alkyl with the carbon atom beta to the point of attachment to the amidine group being an unsaturated electron withdrawing group, provided, however, at least one of R₃₀₁, R₃₀₂, and R₃₀₃ is other than hydrogen;
R₃₁₀ and R₃₁₁ are independently selected from the group consisting of hydrogen, fluorine, hydroxy, alkoxy, and carboxy, provided at least one of R₃₁₀ and R₃₁₁ is other than fluorine and hydrogen; and
R₄₄₀ is C₁-C₆ alkyl, aryl, aralkyl, carboxy, or carboxyalkyl, wherein the alkyl, aryl, aralkyl, carboxy, or carboxyalkyl is optionally further substituted by fluorine.

71. The compound of claim 68 having the structure: 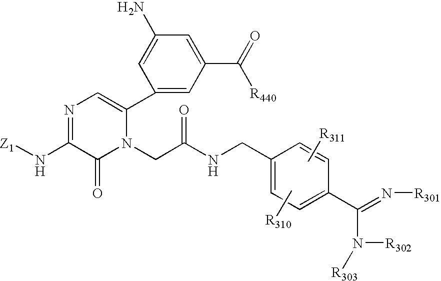
wherein:
Z₁ is isopropyl or cyclopropyl optionally substituted with fluorine, hydroxy, carboxy, or alkoxycarbonyl;
R₃₀₁, R₃₀₂, and R₃₀₃ are independently selected from the group consisting of:
(i) hydrogen, —C(═O)Rₐ, —C(═O)ORₐ, —S(═O)ORₐ, —S(═O)SRₐ, —S(═O)₂ORₐ, —S(═O)₂SRₐ and alkenyl, wherein Rₐ is selected from the group consisting of hydrocarbyl, substituted hydrocarbyl, and heterocyclo, provided, however, that the carbon atom of R₃₀₁, R₃₀₂, and R₃₀₃ directly bonded to the amidine is sp² hybridized when R₃₀₁, R₃₀₂, and R₃₀₃ is alkenyl,,
(ii) hydrogen, optionally substituted hydrocarbyl and aryl, provided, however, the carbon atom of R₃₀₁, R₃₀₂, and R₃₀₃ directly bonded to the amidine is sp³ hybridized when R₃₀₁, R₃₀₂, and R₃₀₃ is optionally substituted hydrocarbyl,
(iii) hydrogen, —OR_{b}, —SR_{b}, —NR_{b}, or —N(R_{b})₂, wherein each R_{b} is independently optionally substituted hydrocarbyl, and heterocyclo, and
(iv) hydrogen, substituted hydrocarbyl wherein the carbon bonded to the amidine group is substituted with —OR_{c}, —SR_{c}, —NR_{c}, or —N(R_{c})₂, wherein each R_{c} is independently —C(O)R_{d}, —C(O)OR_{d}, —C(O)NR_{d}, —C(O)N(R_{d})₂ and each R_{d} is independently hydrocarbyl, substituted hydrocarbyl or heterocyclo, and substituted alkyl with the carbon atom beta to the point of attachment to the amidine group being an unsaturated electron withdrawing group, provided, however, at least one of R₃₀₁, R₃₀₂, and R₃₀₃ is other than hydrogen;
R₃₁₀ and R₃₁₁ are independently selected from the group consisting of hydrogen, fluorine, hydroxy, alkoxy, and carboxy, provided at least one of R₃₁₀ and R₃₁₁ is other than fluorine and hydrogen; and
R₄₄₀ is C₁-C₆ alkyl, aryl, aralkyl, carboxy, hydroxy or carboxyalkyl, wherein the alkyl, aryl, aralkyl, carboxy, hydroxy or carboxyalkyl is optionally further substituted by fluorine.

72. The compound of claim 67 wherein Z₃ is as defined in claim 50.

73. The compound of claim 67 wherein the compound is selected from the group consisting of: 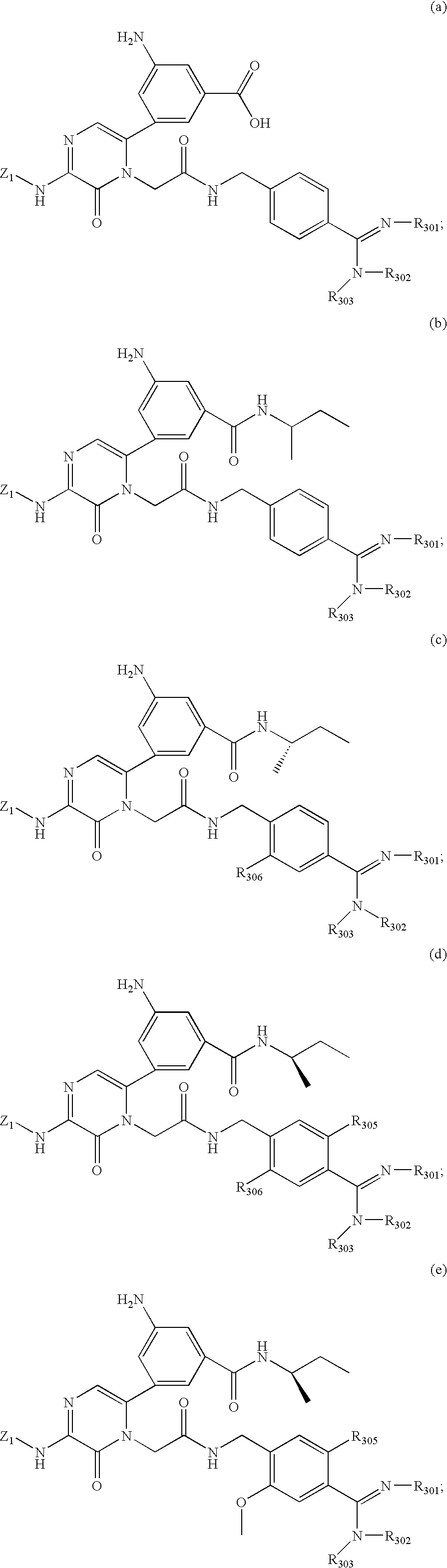 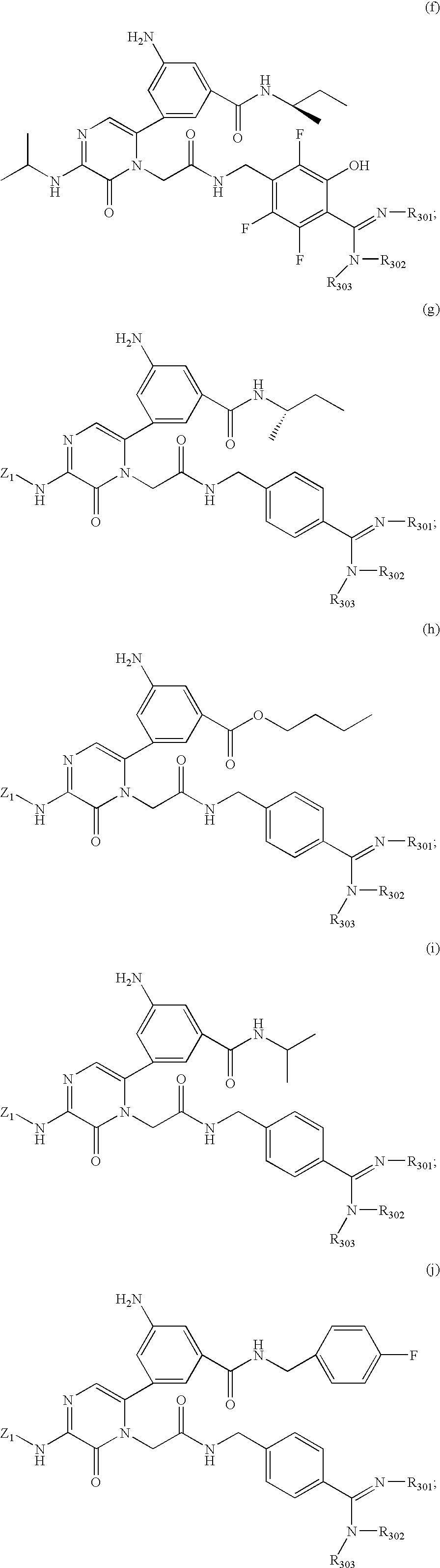 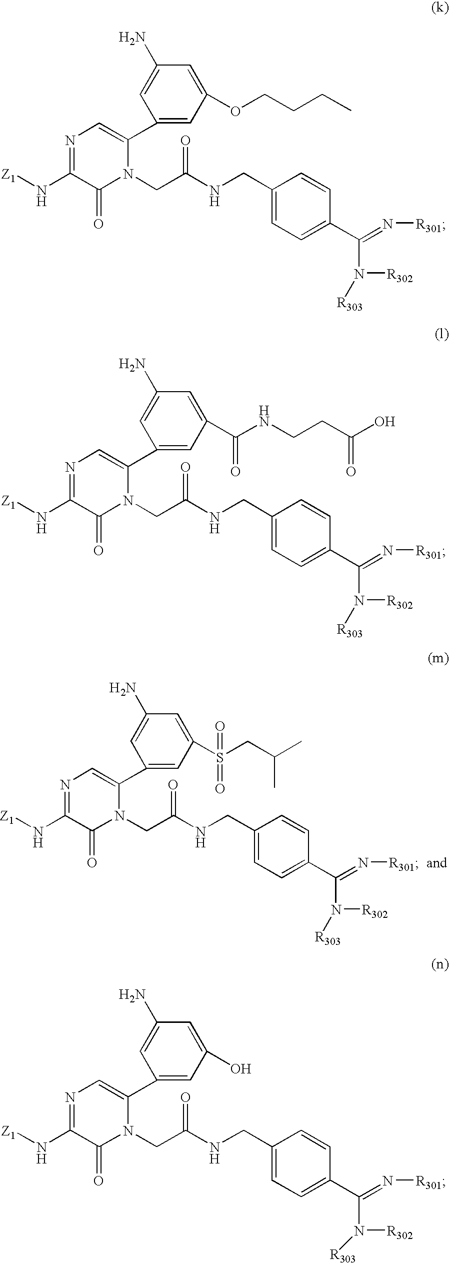
wherein:
Z₁ is isopropyl or cyclopropyl optionally substituted with fluorine, hydroxy, carboxy, or alkoxycarbonyl;
R₃₀₁, R₃₀₂, and R₃₀₃ are independently selected from the group consisting of:
(i) hydrogen, —C(═O)Rₐ, —C(═O)ORₐ, —S(═O)ORₐ, —S(═O)SRₐ, —S(═O)₂ORₐ, —S(═O)₂SRₐ and alkenyl, wherein Rₐ is selected from the group consisting of hydrocarbyl, substituted hydrocarbyl, and heterocyclo, provided, however, that the carbon atom of R₃₀₁, R₃₀₂, and R₃₀₃ directly bonded to the amidine is sp² hybridized when R₃₀₁, R₃₀₂, and R₃₀₃ is alkenyl,
(ii) hydrogen, optionally substituted hydrocarbyl and aryl, provided, however, the carbon atom of R₃₀₁, R₃₀₂, and R₃₀₃ directly bonded to the amidine is sp³ hybridized when R₃₀₁, R₃₀₂, and R₃₀₃ is optionally substituted hydrocarbyl,
(iii) hydrogen, —OR_{b}, —SR_{b}, —NR_{b}, or —N(R_{b})₂, wherein each R_{b} is independently optionally substituted hydrocarbyl, and heterocyclo, and
(iv) hydrogen, substituted hydrocarbyl wherein the carbon bonded to the amidine group is substituted with —OR_{c}, —SR_{c}, —NR_{c}, or —N(R_{c})₂, wherein each R_{c} is independently —C(O)R_{d}, —C(O)NR_{d}, —C(O)OR_{d}, —C(O)N(R_{d})₂ and each R_{d} is independently hydrocarbyl, substituted hydrocarbyl or heterocyclo, and substituted alkyl with the carbon atom beta to the point of attachment to the amidine group being an unsaturated electron withdrawing group, provided, however, at least one of R₃₀₁, R₃₀₂, and R₃₀₃ is other than hydrogen;
R₃₀₅, when present, is hydroxy or hydrogen; and
R₃₀₆, when present, is hydroxy or hydrogen, provided if R₃₀₅ is hydroxy then R₃₀₆ is hydrogen and if R₃₀₅ is hydrogen then R₃₀₆ is hydroxy.

74. The compound of claim 67 wherein the compound is selected from the group consisting of: 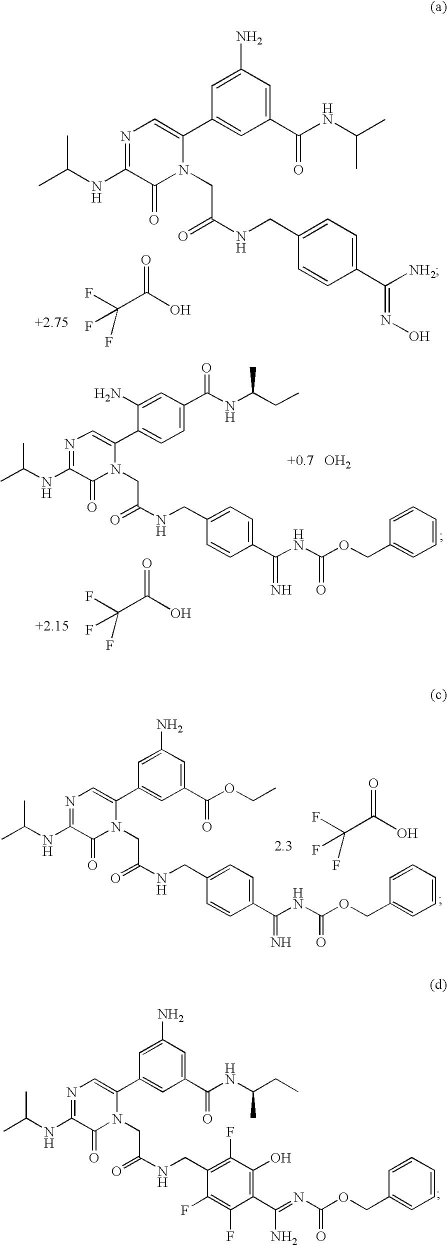 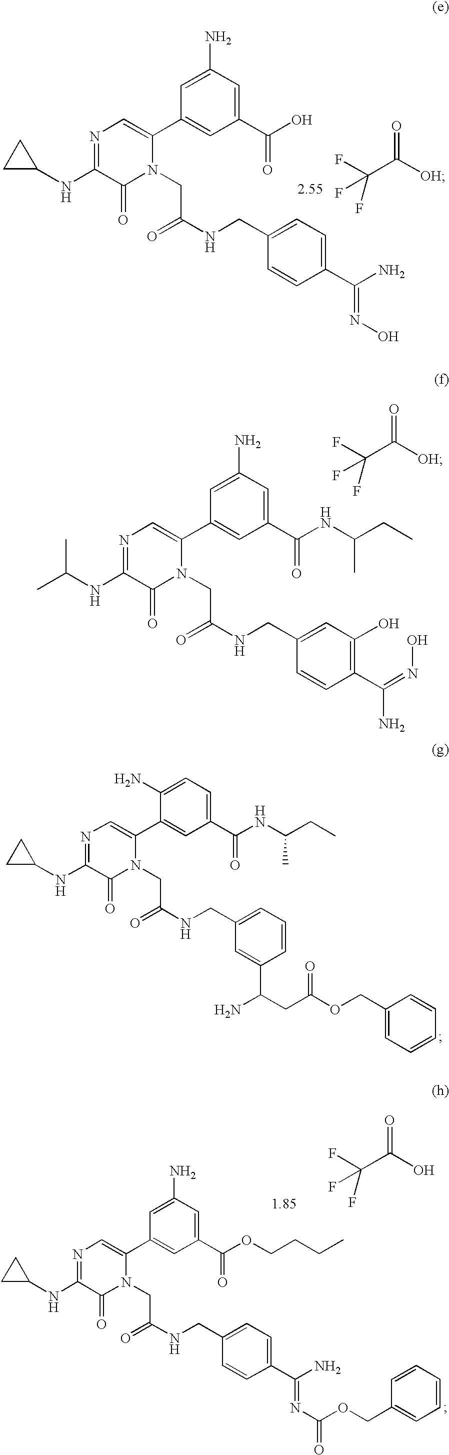 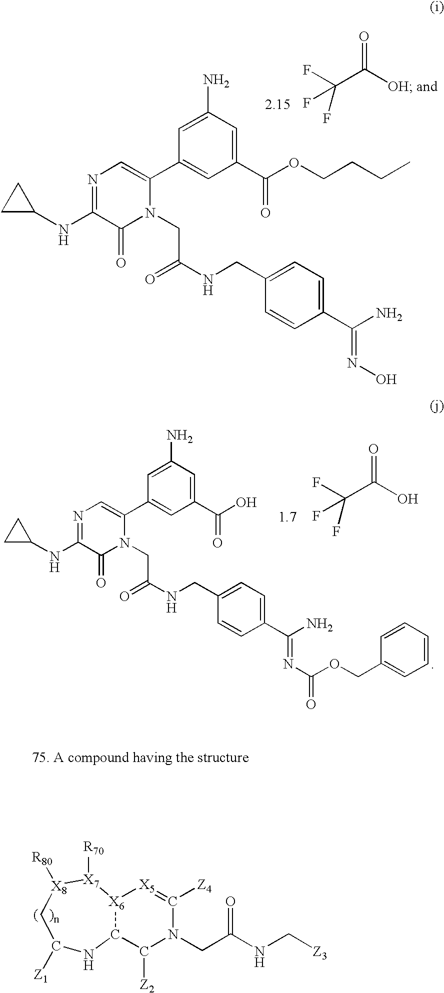
wherein:
X₅ is nitrogen, CH, C(F), C(Cl), or C(Br);
X₆ is carbon or nitrogen, provided the dashed line represents a double bond when X₆ is carbon and the dashed line represents a single bond when X₆ is nitrogen;
X₇ and X₈ are independently carbon, nitrogen, oxygen or sulfur;
Z₁ is selected from the group consisting of C₁-C₈ alkyl, C₂-C₈ alkenyl, and C₂-C₈ alkynyl, the alkyl, alkenyl, or alkynyl being optionally substituted at any substitutable position with a halogen;
Z₂ is a hydrogen bond acceptor covalently or datively bonded to the carbon gamma to X₅.
Z₃ comprises a substituted phenyl, thienyl, or furanyl ring, the phenyl, thienyl or furanyl ring being substituted with a derivatized amidine group and optionally substituted at any substitutable position with fluorine, hydroxy, carboxy, alkoxycarbonyl, or hydrocarbyloxy;
Z₄ comprises a 5- or 6-membered heteroaryl or aryl ring, the ring atoms of Z₄ being Z₄₀, Z₄₁, Z₄₂, Z₄₄ and Z₄₅ when Z₄ is a 5-membered ring and Z₄₀1, Z₄₁, Z₄₂, Z₄₃, Z₄₄ and Z₄₅ when Z₄ is a 6-membered ring, Z₄₀, Z₄₁, Z₄₂, Z₄₃, Z₄₄ and Z₄₅, being carbon, nitrogen, oxygen or sulfur, Z₄₀ being the ring atom through which Z₄ is attached to the heterocyclic core ring, Z₄₁ and Z₄₅ each being in an alpha position relative to Z₄₀, Z₄₂ and Z₄₄ each being in a beta position relative to Z₄₀, Z₄₃ being in the gamma position relative to Z₄₀ when Z₄ is a 6-membered ring, Z₄ having a substituent R₄₂ covalently attached to Z₄₂, and a second substituent bonded to one of Z₄₁, Z₄₃, Z₄₄, or Z₄₅, the substituent being R₄₁ when bonded to Z₄₁, the substituent being R₄₃ when bonded to Z₄₃, the substituent being R₄₄ when bonded to Z₄₄, and the substituent being R₄₅ when bonded to Z₄₅;
R₄₂ is amino;
R₄₁, R₄₃, R₄₄ and R₄₅ are independently hydrogen, hydrocarbyl, substituted hydrocarbyl, heterocyclo, halogen, or a substituted or unsubstituted heteroatom selected from nitrogen, oxygen, sulfur and phosphorus, provided at least one of R₄₁, R₄₃, R₄₄ or R₄₅ is other than hydrogen;
R₇₀ and R₈₀ are independently selected from the group consisting of hydrogen, halogen, amino, hydrocarbyl, substituted hydrocarbyl, aryl, wherein aryl is phenyl optionally substituted by hydroxy, amino, C₁-C₈ alkyl, or halogen provided that R₇₀ is not present when X₇ is a bond and R₈₀ is not present when X₈ is a bond; or R₇₀ and R₈₀, along with the ring atoms to which each is attached, form a 5- or 6-membered saturated ring; and
n is 0 to 2.

76. The compound of claim 75 having the structure 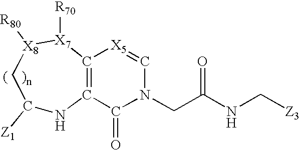
wherein X₅, X₇, X₈, Z₁, Z₃, Z₄, R₇₀, R₈₀ and n are as defined in claim 75.

77. A composition for substantially inhibiting thrombotic conditions in blood comprising a compound of claim 1 and a pharmaceutically acceptable carrier.

78. A method for substantially inhibiting thrombotic conditions in blood comprising adding to blood the composition of claim 77.

79. A method for substantially inhibiting formation of platelet aggregates in blood comprising adding to blood the composition of claim 77.

80. A method for substantially inhibiting thrombus formation in blood comprising adding to blood the composition of claim 77.

81. A method for treating or preventing venous thromboembolism and pulmonary embolism in a mammal comprising administering to the mammal the composition of claim 77.

82. A method for treating or preventing deep vein thrombosis in a mammal comprising administering to the mammal the composition of claim 77.

83. A method for treating or preventing cardiogenic thromboembolism in a mammal comprising administering to the mammal the composition of claim 77.

84. A method for treating or preventing thromboembolic stroke in a mammal comprising administering to the mammal the composition of claim 77.

85. A method for treating or preventing thrombosis associated with cancer and cancer chemotherapy in a mammal comprising administering to the mammal the composition of claim 77.

86. A method for treating or preventing unstable angina in a mammal comprising administering to the mammal the composition of claim 77.

87. A method for inhibiting thrombus formation in blood comprising adding to blood the composition of claim 77 along with a fibrinogen receptor antagonist.
